(19) United States
(12) Patent Application Publication
(10) Pub. No.: US 2008/0108511 A1
(43) Pub. Date: May 8, 2008

(54) GENES AND GENE PRODUCTS DIFFERENTIALLY EXPRESSED DURING HEART FAILURE

(76) Inventor: Judith K. Gwathmey, Cambridge, MA (US)

Correspondence Address: LOWRIE, LANDO & ANASTASI, LLP, ONE MAIN STREET, SUITE 1100, CAMBRIDGE, MA, 02142, US

(21) Appl. No.: 11/840,828

(22) Filed: Aug. 17, 2007

Related U.S. Application Data
(60) Provisional application No. 60/838,522, filed on Aug. 17, 2006.
(60) Provisional application No. 60/948,906, filed on Jul. 10, 2007.

(51) Int. Cl. C40B 30/04; C12N 15/11; C40B 40/08; C12N 9/00; C07K 16/18; C12N 15/00; C12N 5/06; C12Q 1/68

(52) U.S. Cl. 506/9; 536/23.5; 506/17; 435/183; 536/24.33; 435/320.1; 435/325; 435/6; 530/387.9; 536/24.5

(57) ABSTRACT

Certain examples disclosed herein are directed to genes and gene products that are differentially expressed during heart failure. In particular, certain examples are directed to genes which are up-regulated or down-regulated in heart failure. Primers, kits, arrays, antibodies and methods of using the genes are also disclosed.

### PRIORITY APPLICATIONS

This application claims priority to U.S. Provisional Application No. 60/838,522 filed on Aug. 17, 2006 and to U.S. Provisional Application No. 60/948,906 filed on Jul. 10, 2007, the entire disclosure of each of which is hereby incorporated herein by reference for all purposes.

### GOVERNMENT FUNDING

Certain embodiments disclosed herein may have been funded, at least in part, under Grant No. R43 and R44 HL67516 awarded by The Heart, Lung, and Blood Institute. The federal government may have certain rights.

### TECHNOLOGICAL FIELD

Certain examples disclosed herein relate generally to isolated polynucleotides, and uses thereof, that are differentially expressed in a heart disease such as dilated idiopathic cardiomyopathy.

### BACKGROUND

The American Heart Association has estimated the cost of cardiovascular disease in the United States in 2000 to be at $326.6 billion. This figure includes health expenditures (direct costs, which include the cost of physicians and other professionals, hospital and nursing home services, the cost of medications, home health and other medical durables) and lost productivity resulting from morbidity and mortality (indirect costs). One in five females has some form of cardiovascular disease and one in three men can expect to develop some major cardiovascular disease before age 60. Cardiovascular disease claimed 953,110 lives in the United States in 1997. Since 1900, cardiovascular disease has been the No. 1 killer in the United States. More than 2,600 Americans die each day of heart failure—an average of 1 death every 33 seconds.

Heart failure is not only a disease of the elderly or of persons who live unhealthy lifestyles. The highest incidence occurs between 25-45 years of age. Although more patients are surviving their first myocardial infarction, they often go on to develop progressive left ventricular dysfunction and end stage heart failure. As a result, the incidence of congestive heart failure is increasing.

Idiopathic dilated cardiomyopathy (DCM) has emerged as one of the most pressing problems in medical care. Deaths from dilated cardiomyopathy have increased by 127.8 percent over the past three years. Other statistics reveal that DCM is becoming a true epidemic in the United States. About 4,700,000 Americans (2,300,000 males and 2,400,000 females) have DCM. The incidence of DCM approaches 10 per 1,000 after age 65. During the course of the disease, the heart's pumping function steadily decreases, and while patients may sometimes remain stable for years, they eventually die due to a decline in heart muscle function or arrhythmias, unless they undergo heart transplantation.

In addition, little is known about gender related differences in the etiology of heart failure despite it being well accepted that women with heart failure most often have differing clinical presentations than men with a similar cardiac condition. Heart disease is the leading killer of women, responsible for one-third of all deaths of U.S. women (more than all cancers combined) (American Heart Association. Heart Disease and Stroke Statistics—2005 Update Dallas, Tex.: American Heart Association; 2004). Approximately 2.5 million women are living with a diagnosis of congestive heart failure. Following diagnosis of non-ischemic heart failure, women fare somewhat better than men, but less than 15 percent survive beyond 8-12 years after diagnosis (Kirkwood F. Adams, Jr et al). Research has suggested that there may be myocardial properties and/or hormonal environments unique to women that contribute to heart failure (or their clinical outcomes). There remains a need for better methods to diagnose and treat heart disease in both men and women.

### SUMMARY

In accordance with a first aspect, an isolated polynucleotide selected from the group consisting of SEQ. ID NOS.: 1-1143 or SEQ. ID NOS.: 1144-1233 (see attached Appendices A and B) is provided. In some examples, the isolated polynucleotide further comprises a complementary polynucleotide of the isolated polynucleotide such that a double stranded polynucleotide is provided. In yet other examples, the complementary polynucleotide may be separated and isolated by itself.

In accordance with an additional aspect, an array comprising a substrate, e.g., a solid support, and at least one polynucleotide disposed on the substrate that is selected from the group consisting of SEQ. ID NOS.: 1-1143 or SEQ. ID NOS.: 1144-1233 is disclosed. In some examples, the array may take the form of a chip such as a cDNA chip. In certain examples, an array comprising at least one polynucleotide that is complementary to a polynucleotide that is selected from the group consisting of SEQ. ID NOS.: 1-1143 or SEQ. ID NOS.: 1144-1233 is provided.

In accordance with another aspect, a kit comprising at least one polynucleotide selected from the group consisting of SEQ. ID NOS.: 1-1143 or SEQ. ID NOS.: 1144-1233 and at least one enzyme is provided. In some examples, the kit may further include buffers, substrates, additional enzymes and the like. In certain examples, a kit comprising at least one polynucleotide that is complementary to a polynucleotide that is selected from the group consisting of SEQ. ID NOS.: 1-1143 or SEQ. ID NOS.: 1144-1233 and at least one enzyme is disclosed.

In accordance with an additional aspect, a primer comprising an effective amount of contiguous nucleotides from a polynucleotide selected from the group consisting of SEQ. ID NOS.: 1-1143 or SEQ. ID NOS.: 1144-1233 is provided. In other examples, the primer comprises at least 50 contiguous nucleotides of the polynucleotide. In some examples, the primer comprises at least 50 contiguous nucleotides that are complementary to a polynucleotide selected from the group consisting of SEQ. ID NOS.: 1-1143 or SEQ. ID NOS.: 1144-1233.

In accordance with another aspect, a kit configured for determining the presence of heart failure is disclosed. In certain examples, the kit comprises at least one primer comprising an effective amount of contiguous nucleotides from a polynucleotide selected from the group consisting of SEQ. ID NOS.: 1-1143 or SEQ. ID NOS.: 1144-1233. In some examples, the kit may comprise a cDNA chip configured with one or more contiguous nucleotides from a polynucleotide selected from the group consisting of SEQ. ID NOS.: 1-1143 or SEQ. ID NOS.: 1144-1233. In certain examples, the kit may include a cDNA chip configured with one or more contiguous nucleotides that are complementary to a polynucleotide selected from the group consisting of SEQ. ID NOS.: 1-1143 or SEQ. ID NOS.: 1144-1233.

In accordance with another aspect, a kit configured to follow the progression or reversal of heart failure is disclosed. In certain examples, the kit comprises at least one primer comprising an effective amount of contiguous nucleotides from a polynucleotide selected from the group consisting of SEQ. ID NOS.: 1-1143 or SEQ. ID NOS.: 1144-1233. In some examples, the kit may comprise a cDNA chip configured with one or more contiguous nucleotides from a polynucleotide selected from the group consisting of SEQ. ID NOS.: 1-1143 or SEQ. ID NOS.: 1144-1233. In certain examples, the kit may include a cDNA chip configured with one or more contiguous nucleotides that are complementary to a polynucleotide selected from the group consisting of SEQ. ID NOS.: 1-1143 or SEQ. ID NOS.: 1144-1233.

In accordance with an additional aspect, a kit configured to determine responders and non-responders to a heart failure treatment is provided. In certain examples, the kit comprises at least one primer comprising an effective amount of contiguous nucleotides from a polynucleotide selected from the group consisting of SEQ. ID NOS.: 1-1143 or SEQ. ID NOS.: 1144-1233. In some examples, the kit may comprise a cDNA chip configured with one or more contiguous nucleotides from a polynucleotide selected from the group consisting of SEQ. ID NOS.: 1-1143 or SEQ. ID NOS.: 1144-1233. In certain examples, the kit may include a cDNA chip configured with one or more contiguous nucleotides that are complementary to a polynucleotide selected from the group consisting of SEQ. ID NOS.: 1-1143 or SEQ. ID NOS.: 1144-1233.

In accordance with another aspect, a vector comprising at least one polynucleotide selected from the group consisting of SEQ. ID NOS.: 1-1143 or SEQ. ID NOS.: 1144-1233 is disclosed. In certain examples, the vector may take numerous forms of which some illustrative forms are described herein. In certain examples, a vector comprising at least one polynucleotide that is complementary to a polynucleotide selected from the group consisting of SEQ. ID NOS.: 1-1143 or SEQ. ID NOS.: 1144-1233 is provided.

In accordance with an additional aspect, a host cell comprising a vector comprising at least one polynucleotide selected from the group consisting of SEQ. ID NOS.: 1-1143 or SEQ. ID NOS.: 1144-1233 is provided. In certain examples, the host cell may be a mammalian cell or a non-mammalian cell, and illustrative host cells are disclosed herein. In certain examples, a host cell may include a vector comprising at least one polynucleotide that is complementary to a polynucleotide selected from the group consisting of SEQ. ID NOS.: 1-1143 or SEQ. ID NOS.: 1144-1233.

In accordance with another aspect, a method of determining non-responders and responders to a heart failure treatment is disclosed. In certain examples, the method comprises exposing a patient sample to a polynucleotide selected from the group consisting of SEQ. ID NOS.: 1-1143 or SEQ. ID NOS.: 1144-1233, and determining if a gene or gene product in the patient sample binds to the polynucleotide. In some examples, the method further comprises determining if the gene or gene product in the patient sample is up-regulated or down-regulated.

In accordance with an additional aspect, a method of diagnosing heart failure is disclosed. In certain examples, the method comprises exposing a patient sample to a polynucleotide selected from the group consisting of SEQ. ID NOS.: 1-1143 or SEQ. ID NOS.: 1144-1233, and determining if a gene or gene product in the patient sample binds to the polynucleotide. In some examples, the method further comprises determining if the gene or gene product in the patient sample is up-regulated or down-regulated.

In accordance with another aspect, a method of diagnosing idiopathic cardiomyopathy is provided. The method comprises exposing a patient sample to at least one polynucleotide selected from the group consisting of SEQ. ID NOS.: 1-1143 or SEQ. ID NOS.: 1144-1233, and determining if a gene or gene product in the patient sample binds to the polynucleotide. In some examples, the method further comprises determining if the gene or gene product in the patient sample is up-regulated or down-regulated.

In accordance with an additional aspect, a method of treating heart disease is disclosed. In certain examples, the method comprises administering an effective amount of a compound that enhances, reduces or inhibits transcription of a polynucleotide selected from the group consisting of SEQ. ID NOS.: 1-1143 or SEQ. ID NOS.: 1144-1233. In some examples, the compound that is administered may be a polynucleotide selected from the group consisting of SEQ. ID NOS.: 1-1143 or SEQ. ID NOS.: 1144-1233.

In accordance with another aspect, a method of treating heart disease is provided. In certain examples, the method comprises administering an effective amount of a compound that enhances, reduces or inhibits translation of a gene product from a polynucleotide selected from the group consisting of SEQ. ID NOS.: 1-1143 or SEQ. ID NOS.: 1144-1233. In some examples, the compound that is administered may be a polynucleotide selected from the group consisting of SEQ. ID NOS.: 1-1143 or SEQ. ID NOS.: 1144-1233.

In accordance with an additional aspect, a method of diagnosing heart failure in a female human is disclosed. In certain examples, the method comprises determining if at least one female heart failure gene is up-regulated (or down-regulated) using at least one of the polynucleotides disclosed herein. In other examples, the method may comprise determining if at least one female heart failure gene is down-regulated.

In accordance with an additional aspect, a method of diagnosing heart failure in a male human is disclosed. In certain examples, the method comprises determining if at least one male heart failure gene is up-regulated (or down-regulated) using at least one of the polynucleotides disclosed herein. In other examples, the method may comprise determining if at least one male heart failure gene is down-regulated.

In accordance with another aspect, an antibody effective to bind to a polynucleotide selected from the group consisting of SEQ. ID NOS.: 1-1143 or SEQ. ID NOS.: 1144-1233 is disclosed. In some examples, an antibody effective to bind to a polynucleotide selected from the group consisting of SEQ. ID NOS.: 1144-1233 is provided. In certain examples, the antibody may be administered in an effective amount to a mammal in need of treatment for heart failure.

In accordance with an additional aspect, a ribonucleic acid molecule is provided. In certain examples, the ribonucleic acid molecule is effective to bind to and reduce or inhibit translation of a second ribonucleic acid molecule transcribed from a polynucleotide selected from the group consisting of SEQ. ID NOS.: 1-1143 or SEQ. ID NOS.: 1144-1233.

In accordance with an additional aspect, a ribonucleic acid molecule is provided. In certain examples, the ribonucleic acid molecule is effective to bind to and enhance translation of a second ribonucleic acid molecule transcribed from a polynucleotide selected from the group consisting of SEQ. ID NOS.: 1-1143 or SEQ. ID NOS.: 1144-1233.

It will be recognized by the person of ordinary skill in the art, given the benefit of this disclosure, that additional features, aspects and embodiments are possible using the technology disclosed herein. For illustrative purposes only and without limitation, certain examples are described in more detail below to facilitate a better understanding of the technology.

### BRIEF DESCRIPTION OF THE FIGURES

Certain illustrative embodiments are described below with reference to the accompanying drawings in which:

FIG. 1 shows a summary of the similarities of human DCM and avian DCM, in accordance with certain examples;

FIG. 2 shows a typical control heart and a furazolidone induced dilated cardiomyopathy (Fz-DCM heart), in accordance with certain examples;

FIG. 3 shows hybridized blots from a forward subtracted sample (left panel) and a control sample (right panel), in accordance with certain examples;

FIG. 4A is a pie chart showing the functional categories of up-regulated genes in female samples with DCM, and FIG. 4B is a pie chart showing the functional categories of down-regulated genes in female samples with DCM, in accordance with certain examples;

FIG. 5A is a pie chart showing the functional categories of up-regulated genes in male samples with DCM, and FIG. 5B is a pie chart showing the functional categories of down-regulated genes in male samples with DCM, in accordance with certain examples;

FIGS. 6A and 6B are pie charts showing the functional groups for subtracted libraries, in accordance with certain examples;

FIG. 7A and FIG. 7B are bar graphs showing the results of a quantitative RT-PCR example, in accordance with certain examples;

FIG. 8 is a graph showing a comparison of avian QRT-PCR and human male microarray data, in accordance with certain examples;

FIGS. 9A-9H show various Western blots, in accordance with certain examples; and

FIG. 10 shows an overlap diagram of genes found to be differentially expressed >2 fold up or down (compared to normal hearts) in 3 alcohol DCM Hearts, in accordance with certain examples.

### DETAILED DESCRIPTION

It will be recognized by the person of ordinary skill in the art, given the benefit of this disclosure, that the polynucleotides disclosed herein, and their methods of use, represent a significant technological advance in the understanding and treatment of heart disease. Using the illustrations disclosed herein, effective therapies may be designed to alleviate symptoms from heart disease and/or heart failure and to diagnose heart failure at an earlier stage.

While certain examples are described below with respect to heart failure caused by idiopathic dilated cardiomyopathy or alcohol induced heart failure, the devices and methods disclosed herein may be used to generate a fingerprint for any disease state or condition that may cause heart failure, e.g., arrays of nucleic acid sequences representative of another disease state or condition leading to heart failure may be produced and used in the devices and methods disclosed herein.

As used herein, the term “heart failure gene” or “HF gene” refers to a deoxyribonucleic acid sequence that may display a different expression profile in heart failure, or the development of heart failure, when compared to the normal expression profile present in a healthy state. A sub-class of HF genes is a “DCM gene,” which is a gene that is differentially expressed during idiopathic dilated cardiomyopathy, a specific disease that can lead to heart failure. An “up-regulated gene” refers to a gene that is over expressed, e.g., expression products are present at higher levels or more copies of the gene are present, when compared to the expression levels in a healthy state. A “down-regulated gene” refers to a gene that is under expressed, e.g., expression products are present at lower levels or fewer copies of the gene are present, when compared to the expression levels in a healthy state.

As used herein, a “gene product” refers to products transcribed or translated from a gene. Illustrative gene products include, but are not limited to, RNAs, amino acids, proteins and the like. The term “HF protein” refers to a polypeptide that is produced from transcription and translation of a HF gene. It is intended that HF protein include any moieties which may be added to the HF protein from post-translational modification or other post-translational processes, e.g., packaging, secretion, etc.

As used herein, a “female heart failure gene” refers to a gene that is up-regulated or down-regulated differentially in females as compared to males. As used herein, a “male heart failure gene” refers to a gene that is up-regulated or down-regulated differentially in males as compared to females. For example and as discussed in more detail herein, different genes may be differentially expressed in heart failure, e.g., certain genes may be up-regulated while other genes may be down-regulated. In addition, certain genes may be up-regulated or down-regulated to a larger degree in a female than in a male or vice versa. In some examples, genes may be regulated to a similar degree on both males and females. Such male and female heart failure genes are suitable targets for designing therapies and diagnoses specific for treating heart disease and heart failure in females and males.

Heart failure represents any abnormality in the pumping action of the heart, e.g., idiopathic dilated cardiomyopathy, hypertension with concentric hypertrophy of the left ventricular wall, viral, bacterial or drug induced myocarditis, alcohol induced, genetic based, amyloid, or valvular disease. Only a minority of heart failure is caused by primary abnormalities of the heart muscle itself (primary cardiomyopathy). Idiopathic dilated cardiomyopathy (DCM) is the most common type of cardiomyopathy. It is characterized by the unexplained dilatation of one or more chambers of the heart, and by systolic dysfunction with depressed ejection fraction (EF) or fractional shortening. There is a marked increase in cardiac mass without wall thickening, myocyte hypertrophy, and polyploidy. Echocardiographically, there is an increase in end-diastolic and systolic diameter and end-diastolic and systolic left ventricle heart volume. People and animals with heart failure become cyanotic and are hypotensive. During the course of the disease, the heart's pumping function steadily decreases, and while patients may sometimes remain stable for years, they eventually die due to a decline in heart muscle strength or arrhythmias, unless they undergo heart transplantation. About 50% of all heart transplant cases are performed on DCM patients. By the time patients become symptomatic, their heart disease has already progressed to a late stage. As a result of late diagnosis and insufficient understanding of the underlying disease etiology, the prognosis of DCM remains poor.

The incidence rate of DCM is 5-8/100,000 across several populations and in the United States alone, and 10,000-20,000 people die each year as a result of DCM. The incidence rate, following the general trend for heart failure, is increasing. DCM occurs mostly in middle-aged people, but also in children, more often in men than women, and although, by definition, the specific cause underlying DCM remains unknown, several risk factors have been recognized. Among these risk factors are alcohol, viral infections, toxins, certain drugs and genetic predisposition. Currently, there is no cure or prevention for DCM, and treatment is largely directed at controlling the symptoms. Therefore, the need for a thorough understanding of the early changes and underlying causes of DCM is great, as is the need for the development of early diagnostic and prognostic markers. The structural and functional changes that occur in the heart during the early stages of heart disease may lead to changes in gene expression.

Altered gene expression may be the basis of the structural and functional changes that accompany the development of heart disease, and changes in gene expression profiles may be important indicators of specific disease stages of heart failure. Changes in the expression profile of one or more HF genes may be important indicators and diagnostic markers of heart disease and may also serve to identify genes encoding proteins, e.g., HF proteins, that are drug target or molecular therapy candidates which can, for example, interfere with disease development or treat heart disease.

In accordance with certain examples, dilated cardiomyopathy genes that are differentially expressed during DCM may be identified using an animal model. The identified DCM genes may be candidate drug targets and/or diagnostic markers. Although DCM is the most common type of cardiomyopathy, little is known about its underlying etiology, and to date, treatment of DCM is largely directed towards the alleviation of symptoms. An animal model that is highly congruent, e.g. at the functional, anatomical, biochemical, and molecular levels can support molecular and drug targeting strategies. By the time patients present with symptoms, the disease has usually progressed to an advanced stage and only 50% of patients diagnosed with DCM are alive 5 years after diagnosis. Therefore, early detection and elucidation of the causes of DCM are crucial to improve the life quality and expectancy of DCM patients. The DCM model may be used to generate gene expression profiles from different stages of DCM in lieu of performing such studies in HF patients and to identify genes that are de-regulated during the initiation and progression of DCM.

In accordance with certain examples, human heart tissues of normal and patients with idiopathic dilated cardiomyopathy (1-DCM) may be used to determine differential expression of genes. Similarly, samples from patients with other forms of HF, e.g., ischemic heart disease and post-partum cardiomyopathy, may be used to determine differential expression of genes. Such normal and DCM tissue may be obtained directly from patients, may be obtained from frozen samples or may be obtained from other sources. One particular source that is useful is tissue banks. Many hearts or heart tissue samples in tissue banks have been extensively characterized. For example, it is possible to obtain heart tissue from patients who have been diagnosed with DCM. As discussed in more detail herein, determination of differential gene expression may be performed using many different techniques, e.g., subtraction of express profiles of DCM patients and control patients without DCM.

In accordance with certain examples, hearts freshly removed from subjects may be used to identify differentially expressed genes. The hearts may be handled as if being used for cardiac transplantation, e.g., they may be shipped in cardioplegic solution on ice. It will be within the ability of the person of ordinary skill in the art, given the benefit of this disclosure, to use a selected heart tissue in the methods and devices disclosed herein.

Previously, initial knowledge of human heart failure was mostly derived from studies of animal models. However, with the availability of tissue from failing and non-failing human hearts, many of the postulations derived from animal studies have been challenged (Gwathmey and Hajjar, 1993). Nevertheless, studies of human samples also have their limitations. Samples from diseased hearts are usually obtained from end-stage DCM patients at the time of cardiac transplantation. At that point, numerous factors, among them multiple drug therapies, may obscure true pathogenic changes, and samples from earlier disease stages are not available for study. Material from non-failing hearts may be derived from brain-dead organ donors, which may have been exposed to a variety of factors that could influence gene expression, such as increased sympathetic activity and inotropic drugs that maintain heart function and circulation (Lowes et al., 1997, White et al., 1995). Furthermore, human samples from end-stage patients may reflect adaptive changes to the disease as much as disease mechanisms. A well-characterized animal model that correlates well with human disease is, therefore, invaluable in elucidating the underlying problems and disease etiology of human DCM.

Two of the most common animal models used are a surgically induced rat model of myocardial infarction (MI) and aortic banding of transgenics. These models were not selected for use here, as several key markers of human heart failure have not been identified in the models (Kass et al. 1998, James et al., 1998) and the avian model has been demonstrated to be highly congruent with the human condition as well as predictive of clinical observations and outcomes with cardiotonic agents. Furthermore, the physiology of the rat or mouse heart (e.g., transgenics) as well as the developmentally induced isoform switching of key signaling pathways involved in excitation-contraction coupling make these models less than ideal (Gwathmey et al, 1994, Gwathmey and Davidoff 1993, Davidoff and Gwathmey 1994, Gwathmey and Davidoff 1994).

An avian model of DCM may be used to identify DCM genes. In particular, a well-characterized avian animal model of drug-induced DCM results when turkey poults are administered the drug furazolidone (Fz). Additional avian models, such as, for example, spontaneous dilated cardiomyopathy, are described in the various publications by Gwathmey et al. referred to herein and hereby incorporated herein by reference in their entirety for all purposes. Administration of furazolidone leads to the development of DCM (Fz-DCM), which mimics human DCM at the organ, cellular, biochemical and receptor level Fz is a growth promoter and coccidostat used primarily in poultry medicine. However, when given at high concentrations (700 ppm or greater), animals develop dilated cardiomyopathy. Measurements of cardiac morphology obtained from animals treated with Fz for one week show no difference between untreated and treated animals (Glass et al., 1993). After two weeks, Fz-treated animals weigh less than untreated animals with some animals developing mild DCM, and after three weeks of Fz treatment all animals suffer from advanced DCM that is manifested by an increased heart size and weight (Hajjar et a 1993). The heart weight, as well as the heart to body weight ratio has about doubled at that point (and heart volume can increase by as much as nine fold), and the EF and fractional shortening are severely reduced (Gwathmey et al., 1999, Hajjar et al., 1993). To establish a consistent and progressive expression profile that includes early changes in gene expression, time points may be selected, e.g., one week, two weeks, three weeks, and five weeks, and the expression profile at each of the times points may be determined.

There is a substantial correlation between human DCM and Fz-DCM. It has been demonstrated that avian DCM exhibits significant similarities to human heart failure at the organ, cellular, protein, receptor and biochemical level and now at the genomic level. At the organ level, the observed similarities to human DCM include ventricular dilatation, thinning of the left ventricular (LV) wall and impairment of systolic function. At the cellular level, turkey poults, like humans, exhibit cardiac myocyte hypertrophy, enlargement of nuclei and reorientation of subepicardial fibers. The biochemical characterization of the turkey Fz-DCM model and comparison to human DCM was the subject of a ROI granted to Dr. Gwathmey (ROI-1-HL49574 confirm grant number). Subcellular targets for adenoviral gene transfer experiments (e.g. SERCA, parvalbumin, sodium-calcium exchanger, phospholamban) in isolated myocytes were identified in non-failing and failing human hearts. It was found that the avian model has similarities to human DCM including reduced sarcoplasmic reticulum Ca²⁺-ATPase activity (SERCA), troponin T isoform switching, reduced β-receptor-adenylyl cyclase transmembrane signaling, reduced β1-adrenergic receptor expression with no change in β2 receptor number, prolonged calcium transients, no change in peak calcium currents, reduced myofibrillar ATPase activity and myofibril protein content, reduced creatine kinase activity and myocardial creatine content, and reduced ATP and creatine phosphate content. Furthermore, as in humans, citrate synthase and lactate dehydrogenase activity and norepinephrine content were reduced. Studies of Fz-DCM also show similarities in contractile function, force-pCa²⁺ relations, slowed cross bridge cycling rates, reduced peak systolic pressure, and a negative force frequency relationship as reported in failing human myocardium. The observed correlation of turkey Fz-induced DCM with human DCM not only exists at the morphological, biochemical, receptor, protein and cellular levels, but also extends to similar responses to pharmacological interventions (Gwathmey et al., 1999, Kim et al., 1999, Chapados et al., 1992). For example, β-adrenergic blocking agents have been shown to provide long-term benefits in patients with heart failure but not in several animal models, such as the Syrian hamster model (Jasmin and Proschek, 1984). In contrast, treatment of turkey poults with DCM to β-adrenergic blocking agents had beneficial effects similar to reports in humans and furthermore we first reported a cardioprotective effect of β-blockers (Gwathmey et al., 1999, Glass et al., 1993). Based on the above, Fz-induced DCM model in turkey poults was used in certain examples described herein as a model of human DCM for the gene profiling studies discussed herein. It is expected that treatments, gene sequences, proteins, antibodies, gene therapies and the like which are effective in the treatment of turkey poults with heart failure will also be effective in treating humans with heart failure due to the similar physiological and morphological changes that turkey poults and humans share in common with respect to heart failure.

In accordance with certain examples, there are distinct advantages to using an avian model for drug testing: 1) cost compared to dogs or pigs is low, 2) it expresses similar isoforms to adult human hearts in key contractile proteins and calcium regulatory proteins, 3) it does not undergo isoform switching as is seen in small rodent models, 4) non-invasive measurements can be easily obtained in non-sedated, quietly resting animals, and 5) to date the model has been a better predictor of clinical outcomes in humans than several rodent and large animal models including the dog. For example, calcium channel blockers were very beneficial in rodent models, but not in humans or turkeys. Beta-blockers failed in several models such as the Syrian Hamster, rodent and dog models of heart failure, yet in human studies and in turkeys it has significantly reduced mortality.

Several techniques allow the detection of genes that are differentially expressed in cells or tissues under different conditions. One of the most recent technologies is DNA chip technology, which enables the screening of thousands of genes in a single experiment. Currently, however, there are no avian cDNA arrays available or human heart failure cDNA arrays. Other methods such as differential display (Liang and Pardee 1992, Sokolov and Prockop 1994), representational difference analysis (Lisitsyn et al., 1993), enzymatic degradation subtraction or linker capture subtraction (Yang and Sytowsli, 1996, Akopian and Wood, 1995, Deleersnijder et al., 1996) have all been used to isolate differentially expressed sequences. Some of these techniques may have certain drawbacks. For instance, all these techniques strongly favor the isolation of abundant transcripts as the disproportion of rare versus abundant transcripts is maintained throughout the isolation procedure. Furthermore, these techniques are very labor intensive and the subtraction efficiency (the removal of sequences common to both pools) is often low. Another drawback of conventional differential display methods is that they restrict the analysis of differentially expressed genes to differences at the 3′-end of cDNAs.

In accordance with certain examples, a differential screening technique that combines subtractive hybridization (SH) and suppressive PCR, suppression subtractive screening (SSS), with a high throughput differential screen (HTDS) is used in certain embodiments disclosed herein. This screening technique is generally described, for example, in Diatchenko et al. (1996). This experimental strategy allows the efficient and rapid cloning of hundreds of differentially expressed (abundant and rare genes) in one single hybridization experiment and reduces the possibility of isolating false positive clones. In contrast to the usual 10 to 20 fold enrichment of differentially expressed sequences, SSS/HTDS yields 1000-fold enrichment in a single experiment and the efficiency of subtraction can be monitored. This method has been used successfully to isolate 625 differentially expressed cDNAs from the metastatic cell line Bsp73-ASML when subtracted from its non-metastatic counterpart i.e., Bsp73-ASML (von Stein et al., 1997). Sequence analysis of the authors' data revealed that of the 625 clones obtained, 92 scored near perfect or perfect matches with known sequences in the database, 281 clones scored between 60% and 90% homology and 252 clones encoded novel genes. Other successful applications of this method have also been published (Wong et al., 1997, Yokomizo et al, 1997), among them the identification of 332 cDNAs from estrogen receptor (ER) positive versus ER negative cell lines (Kuang et al., 1998), and differentially expressed clones from activated T cells (Wong et al.; 1996).

In accordance with certain examples, a suitable experiment to identify differentially expressed genes may include one or more of the following steps. mRNA(s) from samples under comparison may be prepared and a cDNA(s) may be produced from the mRNA(s) using techniques well known in the art. The cDNA of the sample containing the differentially expressed genes is called tester cDNA, and the cDNA of the sample containing the common genes that will be subtracted is called driver cDNA. Both, the tester and driver cDNAs are then digested into small fragments with a four-nucleotide cutting restriction enzyme that generates blunt ends. Suitable restriction enzymes will be readily selected by the person of ordinary skill in the art, given the benefit of this disclosure, and illustrative enzymes may be found, for example, in Maniatis et al., 1989, Molecular Cloning: A Laboratory Manual, Cold Spring Harbor Laboratory, N.Y. The tester cDNA may be divided into two pools, each of which may be ligated to a different adaptor. The driver cDNA is typically not ligated to adaptors. In two sequential hybridization reactions between the tester and driver cDNA, only the differentially expressed genes of the tester cDNA will generate PCR templates that can be amplified exponentially during suppression PCR. Further enrichment for differentially expressed genes and reduction of background may be achieved in a second PCR reaction that uses nested primers.

For the first hybridization, an excess of driver cDNA may be added to each tester cDNA pool. The samples are denatured and allowed to anneal. Several types of molecules may be generated in each hybridization mix. Type A molecules are differentially expressed sequences that did not hybridize to anything and are thus single stranded. Type B molecules are re-annealed double stranded tester molecules, type C molecules are double stranded hybrids of tester and driver molecules, and type D molecules are single stranded and double stranded driver molecules without adaptors. At the first hybridization step, rare and abundant molecules are equalized due to hybridization kinetics. During a second hybridization, the reaction mixes from the first hybridization samples are combined without denaturing, and fresh denatured driver cDNA is added to enrich further differentially expressed genes. The remaining, differentially expressed molecules will be free to associate and form type E molecules, which are double stranded differentially expressed sequences with a different adaptor at the 3′ and 5′ ends, respectively. The overhanging ends of the adaptors are next filled in to create primer sites and two sequential PCR reactions are performed. Other types of molecules resulting from this hybridization are type A, B, C and D. Only type E molecules can be amplified exponentially. To further reduce the background and enrich for differentially expressed sequences, nested primers are used for a second PCR reaction. For a complete description of this process, see Clontech PCR. Select cDNA Subtraction User Manual published on Dec. 20, 2004.

To establish a stage-specific expression profile, cDNA from animals in different disease stages that have been fed a higher dose of Fz and sacrificed after one week, two weeks, and three weeks may each be subtracted from cDNA of normal lower-dose Fz-treated animals that were sacrificed after one week, two weeks or three weeks respectively. This screening can identify genes that are uniquely turned on and off during the development of heart disease, e.g., DCM, at specific stages. For example, in a first series of experiments, the cDNA from the control animals may be the driver, and the cDNA from the diseased animals may be the tester. The tester contains the differentially expressed sequences, and the driver cDNA will be subtracted. This series of experiments will identify sequences that are expressed uniquely in the diseased tissues. In a second series of experiments, the cDNA derived from normal animals may be the tester, and the cDNA from the diseased animals may be the driver. Now the normal cDNA will contain differentially expressed sequences, and the diseased cDNA will be subtracted. This second series of experiments can identify sequences that are uniquely turned off during DCM development.

In accordance with certain examples, libraries may be constructed based on the differential gene expression in normal versus heart failure (e.g., DCM) subjects. These libraries can reflect differential gene expression in any stage of DCM development, e.g., stage-specific libraries may be constructed. For example, a secondary PCR product from each of the subtracted pools may be cloned into a vector for further amplification and usage. This may be accomplished using a T/A-based cloning system, such as the AdvanTAge PCR cloning kit (Clontech). Since cloning efficiency is extremely important, ultra-competent cells may be used for transformation of the cloning products. Although this subtraction method greatly enriches for differentially expressed genes, the subtracted samples may contain some cDNAs that correspond to mRNAs common to both the tester and the driver samples, in particular, if few mRNAs are differentially expressed. To minimize background even further, a differential screening step may be performed on the subtracted samples.

In accordance with certain examples, in order not to lose low-abundance sequences, the generated subtracted cDNA libraries may be hybridized with probes made from the forward and reverse-cDNA probes. Alternatively, unsubtracted probes from the tester and driver cDNAs could be used, but this approach may be less sensitive and rare transcripts could be undetected. Truly differentially expressed clones from the forward libraries should hybridize only with the specific forward subtracted probe, but not to the reverse subtracted probes. A more complete description of this process may be found in the Clontech PCR Select DNA Differential Screening Kit User Manual. Table 1 below shows expected results from this screening where high Fz equals 700 ppm Fz in the feed and lower Fz equals 500 ppm Fz in the feed.

TABLE 1

| | | | Probes | | Probes that |
|---|---|---|---|---|---|
| | | | made | Probes used | should |
| Subtracted cDNA | Library | Array made from | from | to screen | hybridize to |
| Libraries | Name | Libraries | Libraries | library | Array |
| 1. High Fz (1 | F1 | AF1.1-5 | f1 | f1 and r1 | f1 (not r1) |
| week)-Lower Fz | | | | | |
| (1 week) | | | | | |
| 2. Lower Fz (1 | R1 | AR1.1-5 | r1 | f1 and r1 | r1 (not f1) |
| week)-High Fz (1 | | | | | |
| week) | | | | | |
| 3. High Fz (2 | F2 | AF2.1-5 | f2 | f2 and r2 | f2 (not r2) |
| weeks)-Lower Fz | | | | | |
| (2 weeks) | | | | | |
| 4. Lower Fz (2 | R2 | AR2.1-5 | r2 | f2 and r2 | r2 (not f2) |
| weeks)-High Fz | | | | | |
| (2 weeks) | | | | | |
| 5. High Fz (3 | F3 | AF3.1-5 | f3 | f3 and r3 | f3 (not r3) |
| week)-Lower Fz | | | | | |
| (3 week) | | | | | |
| 6. Lower Fz (3 | R3 | AR3.1-5 | r3 | f3 and r3 | r3 (not f3) |
| week)-High Fz (3 | | | | | |
| week) | | | | | |

In Table 1, F refers to forward, R refers to reverse, AR refers to array made from libraries, r refers to reverse probes used to screen a library and f refers to forward probes that may be used to screen a library. The number appended to the abbreviation refers to a random number for a selected item.

DNA and RNA may be isolated using numerous techniques that will be selected by the person of ordinary skill in the art, given the benefit of this disclosure. For example, total RNA may be isolated using the thiocyanate-phenol-chloroform method (Chomczynski & Sacohi, 1987) following standard protocols. Poly(A) RNA may be isolated using a poly(A) isolation kit (Ambion). After RNA isolation, the integrity of the RNA may be tested by electrophoresis of the RNA on 1% agarose gels stained with ethidium bromide. Total mammalian RNA exhibits 2 bright bands at 4.5 and 1.9 kb of a DNA standard which corresponds to the 28S and 18S RNA respectively. Poly(A) RNA runs as a smear from 0.5 to 12 kb with faint ribosomal bands. Poly(A) RNA may be isolated from age-matched control Fz-treated groups of animals. These groups may include male and female animals to account for gender-specific variations. The RNA of each group may be pooled and used for the suppression subtractive screening (SSS procedure) described herein.

In accordance with certain examples, the SSS procedure may be performed using a Clontech PCR-Select™ DNA subtraction kit, following the manufacturer instructions. First strand and second strand synthesis may be performed on the isolated nRNA pools. A control for the procedure human skeletal muscle tester and driver cDNA is provided by the manufacturer. Using these controls, a complete control subtraction experiment may be performed. Each tester cDNA pool may be ligated to the appropriate adaptor. The ligation products may be used in the differential screening of a subtracted cDNA library. To monitor the success of the procedure, the ligation efficiency may be tested before proceeding. This test may be performed by verifying that at least 25% of the cDNAs have adaptors using PCR. Fragments may be amplified that span the adaptor/cDNA junction of a known gene, e.g., the turkey α-tubulin gene (see below), and compared to fragments amplified with two gene-specific primers. In a typical experiment, if the band intensity for both products differs by four-fold, the ligation is less than 25% complete and should be repeated. Adaptors are not typically ligated to the driver cDNA. For each stage, two subtraction experiments may be performed (forward and reverse subtraction: tester as driver and driver as tester). Following the ligation, two hybridization reactions and two PCR reactions may be performed. The two hybridization reactions generate the PCR templates. Only the differentially expressed sequences of the tester cDNA pool will provide the correct primer sites and be amplified exponentially during the first PCR reaction. The second PCR reaction serves two purposes: first, to further amplify the differentially expressed sequences and second to further eliminate false positives by using nested primers. Analysis of the PCR products may be performed after each PCR reaction with the sample reactions and the control reactions, and subtraction efficiency may be determined.

In accordance with certain examples, to monitor the successful completion of the subtraction and suppressive PCR reaction, the efficiency of the PCR subtraction may be tested. This procedure may be performed by comparing the abundance of known cDNAs before and after subtraction. Ideally, both a non-differentially expressed gene (e.g., a housekeeping gene) and a known differentially expressed gene may be used. The test described by Clontech uses glycerol-3-phosphate dehydrogenase (G3PDH) as a housekeeping control gene. Although G3PDH is subtracted efficiently from most tissues and cells, there are some exceptions, including heart and skeletal muscle. Furthermore, the provided controls for PCR analysis of the subtraction efficiency may only be faithful for human, rat or mouse cDNA. Turkey primers are not yet available. A primer set that has been shown to work in heart and skeletal muscle tissues is an α-tubulin set. The α-tubulin gene of turkey may be cloned by reverse transcription-PCR (RT-PCR), using the primers provided for the human, rat and mouse α-tubulin gene and sequentially lower annealing temperatures (lower stringency). The turkey α-tubulin gene may be cloned into a T/A-based vector (Clontech), and sequenced to confirm its identity. The resulting sequence may be used to design primers for PCR analysis and hybridization analysis of subtraction efficiency. The abundance of house keeping genes should drop after subtraction. Care should be taken to distinguish background bands from true bands by using nested primers for a second PCR amplification.

In accordance with certain examples, a small percentage, e.g., 1-2%, of the clones identified by differential screening with subtracted probes may be false positives. A final confirmation step using Virtual Northern blots may be performed to confirm differential screening results. For example, cDNA is prepared from tester and driver total RNA or mRNA. The cDNA may then be electrophoresed through an agarose gel, transferred to a nylon membrane and hybridized with individual probes to confirm the differential expression. Even though not all mRNAs may appear ultimately as a single band due to incomplete reverse transcription, a differential signal should be detectable.

In accordance with certain examples, the differentially expressed genes may be sequenced using methods known to those skilled in the art. For example, in certain embodiments, the cDNAs may be inserted into a T/A vector. Primers designed to this vector may be used for the initial sequencing reactions. A portion of the identified differentially expressed sequences is expected to consist of genes of known sequence and function. Based on the deduced protein sequence from the 3′ and 5′ DNA sequence, these genes can most likely be identified based on their homology to genes in the human gene database. Genes of unknown sequence may be sequenced fully. Sequencing may be accomplished, for example, with a medium throughput ABI PRISM 310 Genetic Analyzer from PE Biosystems. This DNA sequencer uses automated fluorescent analysis and capillary electrophoresis technology, which provides a much higher degree of automation than analysis using polyacrylamide gels, as the time consuming steps of gel pouring and sample loading may be eliminated.

In accordance with certain examples, data analysis may be performed using commercially available algorithms and the sequences may then be grouped according to their function based on a previously established classification scheme (Adams, Md.). Sequences may be identified using publicly accessible gene data banks (Entrez, PASTA), grouped by functional roles if possible, and stage-specific expression profiles of the cDNAs that are specifically turned on and off during the development and progression of Fz-DCM may be established. Sequences may be identified for turkeys, human or other selected animals or subjects.

In accordance with certain examples, the avian model may be used to identify genes that are differentially expressed in DCM, and such identified avian genes may be used to identify the human homologs. For example, sequence homology comparisons between identified avian genes and unknown human genes may be performed to identify human genes that may be differentially expressed during DCM as well as to narrow the focus of genes that contribute to the occurrence of HF.

In accordance with certain examples, for those protein products where antibodies are available, quantitative Western blots may be used to test whether the human proteins are differentially present in the same manner as the mRNAs. For proteins where antibodies are not already available, the full-length cDNA encoding the protein may be cloned into appropriate expression vectors for protein production. The purified proteins may then be used to produce antibodies for the quantitative Western blots. All of the above techniques use standard molecular biology and protein methodologies that are well known to those of ordinary skill in the art. Those genes that show differential expression in diseased human hearts compared to normal hearts, and that show differential levels of the encoded protein, may then be used to check for functional effects by overexpression (or underexpression as the case may be) in cardiac myocytes from turkey as well as human hearts.

In accordance with certain examples, traditional techniques, such as Northern blot analysis and RT-PCR, allow the examination of single genes. Using these techniques several differentially expressed genes have been identified, among them atrial natriuretic peptide, sarcoplasmic/endoplasmic Ca-ATPase, β1-adrenergic receptors, collagen and fibronectin (Yue et al., 1998, Murakami et al., 1998, Hanatani et al., 1998, Mendez et al., 1987). Subtractive hybridization and differential display have also been used to identify new genes that might be involved in heart failure, and in combination with microarray technology provide a powerful tool to analyze different sets of cDNAs. An example of such an analysis is the application of cDNA microarrays to determine the molecular phenotype in cardiac growth and development and response to injury after subtracting mRNA from sham-operated and six week post-MI samples from rats (Sehl et at, 1999). One thousand and nine hundred sixty three non-mitochondrial cDNAs were identified, and 1000 were used to manufacture a cDNA array of differentially expressed genes (Sehl et at, 1999). This array was then used to further profile cDNA expression in different tissues. If applicable, cDNA microarray techniques may be used to identify differentially expressed genes (Stanton et al., 2000). More than 400 differentially expressed genes were identified from rat myocardium in response to myocardial infarction. Stanton et al. surveyed approximately 7000 genes, which correspond to less than 5% of rat genes. Randomly identified cDNAs from rat cDNA libraries were applied to microarrays and profited for expression in the LV free wall and the interventricular septum (IVS) at 2, 4, 8, 12, and 16 weeks after surgically induced myocardial infarction. Patterns of gene expression were then determined using newly developed clustering algorithms, and their expression pattern was organized within functional groups. Examples of such groups are genes encoding structural, metabolic, and cell signaling proteins. While expression information alone may not be sufficient to establish firm functional associations among proteins, it is very useful in generating testable hypotheses and guiding further research and molecular therapy approaches. For example, signaling molecules may be involved in mediating the remodeling process, and a few transcription factors may orchestrate the changes in expression of many genes. The identification of differentially expressed genes by comparative analysis of tissues under different conditions is a valuable and crucial step in identifying possible drug targets and diagnostic markers.

In accordance with certain examples, the identified genes and gene products may be used to produce an array, which can be used, for example, to screen a patient sample to identify patients having up-regulated or down-regulated HF genes. For example, one or more polynucleotides may be disposed on a suitable substrate, e.g., a solid support, to provide an array or chip that can be exposed to a patient sample, e.g., blood, plasma, urine, saliva, sweat, RNA from biopsies, etc. In certain examples, the substrate may be selected from common substrates used to produce arrays, e.g., plastics such as polydimethylsiloxane, rubbers, elastomers and the like. It will be within the ability of the person of ordinary skill in the art, given the benefit of this disclosure, to select suitable substrates for producing arrays. In certain examples, the patient sample may be a tissue biopsy or other body fluid sample, e.g., which has been homogenized and treated to release the patient's DNA (or RNA) for exposure to the array.

In accordance with certain examples, a selected number of cDNAs, or a single cDNA, may be selected and arrayed on a suitable substrate, e.g., a nylon membrane. For example, about 1000 cDNA clones from a subtracted library may be placed on a nylon membrane and can be used, for example, to identify or screen drug candidates or chemical libraries. The arrays could also be used, for example, for cDNA dot blots. For high-throughput screening, bacteria (TOP1O or DH5α) may be grown in 96-well or larger dishes (e.g., up to 10 per library) and the PCR reactions may be performed in special 96 well or larger PCR dishes and a multiplate thermocycler (MJ Research Multiplate 96). The PCR reactions may be performed with nested primers that are also used in the second PCR reaction described herein. Two identical blots may be prepared for hybridization with the subtracted forward and reverse cDNA probes. The DNA may be cross-linked using a UV linker (e.g., Stratagene: UV Stratalinke). The resulting arrays may then be hybridized to subtracted probes as described herein. An illustrative set of expected results is shown in Table 2 below.

TABLE 2

| | Forward | Reverse | |
|---|---|---|---|
| Sample | Subtracted | Subtracted | |
| Array | (f1) | (R1) | Interpretation |
| AF1 | + | − | Strong candidate for differential |
| | | | expression. |
| | +++ | + | Clones that hybridize to both subtracted |
| | | | probes but with different intensities: |
| | | | If the difference is >5-fold, it is |
| | | | probably a differentially expressed |
| | | | clone. |
| | + | + | Almost never differentially expressed. |
| | − | − | Usually a non-differentially expressed |
| | | | clone. |

In Table 2, the symbols represent the same items as discussed above in reference to Table 1.

In accordance with certain examples, the identified polynucleotides may be used to diagnose heart disease or heart failure. For example, a patient sample may be exposed, to a polynucleotide selected from the group consisting of SEQ. ID NOS.: 1-1143 or SEQ. ID NOS.: 1144-1233. If a gene or gene product in the patient sample is present at a selected level, then the patient may be at risk for heart disease or heart failure. In some examples, the method further comprises determining if the gene or gene product in the patient sample is up-regulated or down-regulated using the methods described herein. Depending on the exact polynucleotides of the array, one or more particular heart diseases may be diagnosed. For example, polynucleotides that can bind to up-regulated or down-regulated genes in idiopathic cardiomyopathy patients may be arrayed to diagnose for idiopathic cardiomyopathy. The person of ordinary skill in the art, given the benefit of this disclosure, will be able to select suitable polynucleotides for diagnosing a selected heart disease.

In accordance with certain examples, the identified polynucleotides may be used to monitor the progression and/or treatment of heart disease or heart failure. For example, a patient may be placed on one or more drug regimens or other selected treatment. The patient may periodically provide a sample that may be exposed, for example, to a polynucleotide selected from the group consisting of SEQ. ID NOS.: 1-1143 or SEQ. ID NOS.: 1144-1233. If a particular drug or treatment regimen is working, then the level of the gene or gene product in the patient sample may go up or down. The increase or decrease in the level of a particular gene or gene product may be monitored to provide feedback regarding the effectiveness of a particular drug or treatment regimen. The person of ordinary skill in the art, given the benefit of this disclosure, will be able to select suitable polynucleotides for monitoring the progression and/or treatment of a selected heart disease.

In accordance with certain examples, in a study by Carroll et al examining left ventricle hypertrophy (LVH) caused by aortic stenosis, women had smaller, thicker-walled ventricles despite similar outflow obstruction, suggesting that female ventricles may respond differently to a pressure-overload state (Carroll et al., Circulation. 1992; 86(4):1099-1107). In additional studies using transgenic murine and rat models of heart failure, it was shown that overall, females have less cardiac remodeling, dysfunction, and pathology and an increased survival advantage over males (Tamura et al. Hypertension. 1999; 33:676-680; Xiao-Jun Du. Cardiovascular Research. 2004; 63:510-519; Kadokami T et al. J. Clin. Invest. 2000; 106:589-597; Haghighi K et al. J Biol. Chem. 2001; 276 (26):24145-24152; Li et al. Endocrinology. 2004; 145(2):951-958; Du X-J et al. Cardiovascular Research. 2003; 57:395-404; Gao X M et al. Endocrinology. 2003; 144(9):4097-4105). Two studies have suggested that female sex hormones may play a protective role in heart failure showing that female-related phenotypes can be mimicked by the use of estradiol in males or in ovariectomized female transgenic heart failure models (Xiao-Jun Du. Cardiovascular Research. 2004; 63:510-519; Van Eickels M et al. Circulation. 2001; 104:1419-23). Conversely, a murine study using testosterone infusion in ovariectomized transgenic females increased cardiac mass and fibrosis (Li, Y et al. Endocrinology. 2004; 145(2):951-958). An additional study using male mice with cardiac overexpression of β₂-adrenergic receptors showed a reduction in heart failure phenotype from orchiectomy (Gao X M et al. Endocrinology. 2003; 144(9):4097-4105). These results suggest an additional contribution by testicular hormones to the progression of the cardiomyopathic phenotype in these transgenic models. Despite animal studies, gender-related differences that would enable better diagnosis and prognosis of human females and human males with heart failure have not yet been clearly established.

The structural and functional changes that occur in the heart during prolonged heart failure are most likely due to changes in gene and protein expression that is ultimately responsible for the restructuring and damage heart muscle leading to heart failure. To address this issue, the gene expression profile of diseased myocardium in both female and male patients with end-stage idiopathic dilated cardiomyopathy (IDCM) by means of subtractive hybridization and gene microarray technology may be performed (see Examples section below). Microarray technology is capable of screening vast numbers of genes, or entire genomes, for differential expression. To increase and focus the number of genes on the array that are potentially involved in DCM, a heart-specific array may be developed and used with subtractive hybridization in order to pre-select differentially expressed clones, which may be used to produce a microarray. By using this approach a focused microarray containing both potentially up and down-regulated genes including rare genes expressed at low levels in the non-failing and failing heart may be produced. This focused microarray may be used to identify gender-specific differences in the gene expression pattern consequent to DCM. These gene expression differences in the cohorts of female and male samples may be indicative of sex-linked disparities in the pathophysiology and potentially even the pathogenesis of heart failure.

In accordance with certain examples, nucleic acid molecules, preferably DNA molecules, that hybridize to, and are therefore complementary to, the DNA sequences SEQ. ID NOS.: 1-1143 or SEQ. ID NOS.: 1144-1233 are provided. Suitable hybridization conditions will be readily selected by the person of ordinary skill in the art, given the benefit of this disclosure. In instances wherein the complimentary nucleic acid molecules are oligonucleotides (“oligos”), highly stringent conditions may refer, for example, to washing in 6×SSC/0.05% sodium pyrophosphate at 37° C. (for less than 14-base oligos), 48° C. (for 14-17-base oligos), 55° C. (for 17-20-base oligos), and 60° C. (for greater than 23-base oligos). These nucleic acid molecules may act as HF gene antisense molecules, useful, for example, in HF gene regulation and/or as antisense primers in amplification reactions of HF nucleic acid sequences. Further, such sequences may be used as part of ribozyme and/or triple helix sequences, which may also be useful for HF gene regulation. Still further, such molecules may be used as components of diagnostic methods and prognostic outcomes in response to a particular therapy whereby the level of a HF transcription product may be deduced. Further, such sequences can be used to screen for and identify HF gene homologs from, for example, other species.

In accordance with certain examples, vectors may be used with the HF genes, e.g. molecular therapies, disclosed herein. For example, DNA vectors that contain any of the HF nucleic acid sequences and/or their complements (i.e., an antisense strand) may be used to produce large quantities of expression products, e.g., mRNAs and polypeptides. In certain examples, DNA expression vectors may include any of the HF coding sequences operatively associated with a regulatory element that directs the expression of the HF coding sequences. In some examples, a genetically engineered host cell may include any of the HF coding sequences operatively associated with a regulatory element that directs the expression of the coding sequences in the host cell. As used herein, regulatory elements include but are not limited to inducible and non-inducible promoters, enhancers, operators and other elements that will be selected by the person of ordinary skill in the art, given the benefit of this disclosure, that drive and regulate expression. For example, such regulatory elements may include CMV immediate early gene regulatory sequences, SV40 early or late promoter sequences on adenovirus, retro-viral rectors, lentivectors, adeno-associated vectors, lac system, trp system, tac system or the trc system sequences. In certain examples, one or more fragment of the HF coding sequences may be included in a vector instead of an entire HF coding sequence. For example, where a single HF coding sequence may encode a polypeptide with several subunits or domains, it may be desirable to include only one of the subunits or domains (or omit one or more subunits or domains) to determine the role of that subunit or domain in protein function.

In addition to the HF gene sequences described above, homologs of the HF gene sequences, as may, for example be present in other species, may be identified and isolated by molecular biological techniques that will be selected by the person of ordinary skill in the art, given the benefit of this disclosure. For example, small probes of a few, e.g., 12 bp, to several, e.g., 30 bp, may be used to identify homologs of the HF gene sequences in genera such as Gallus, Homo or non-human mammals. Further, mutant HF alleles and additional normal alleles of the human HF genes disclosed herein, may be identified using such techniques. Still further, there may exist genes at other genetic loci within the human genome that encode proteins which have extensive homology to one or more domains of the HF gene product. Such genes may also be identified, for example, by such techniques. In other examples, an antisense strand of an HF gene sequence may be identified. In yet other examples, one or more gene products, e.g., RNA, protein, etc. may be identified.

In accordance with certain examples, a targeting agent may be identified using the HF gene sequences disclosed herein. In certain examples, the targeting agent may be a small organic molecule, e.g., a molecule that can bind to a HF gene sequence or some product thereof. Alternatively, the targeting agent may be a test polypeptide (e.g., a polypeptide having a random or predetermined amino acid sequence or a naturally-occurring or synthetic polypeptide) or a nucleic acid, such as a DNA or RNA molecule. The targeting agent may be a naturally-occurring compound or it may be synthetically produced, if desired. Synthetic libraries, chemical libraries, and the like can be screened to identify compounds that bind the HF gene sequences or products thereof. More generally, binding of a target compound to a HF polypeptide, homolog, or ortholog may be detected either in vitro or in vivo. If desired, the above-described methods for identifying targeting agents that modulate the expression of HF polypeptides can be combined with measuring the levels of the polypeptides expressed in the cells, e.g., by performing a Western blot analysis using antibodies that bind to a HF polypeptide.

In accordance with certain examples, a HF gene product, e.g., a HF protein expressed from a HF gene, may be substantially purified from natural sources (e.g., purified from cardiac tissue) using protein separation techniques well known by those of ordinary skill in the art. The term “substantially purified” refers to a polypeptide being purified away from at least about 90% (on a weight basis) of other proteins, glycoproteins, and other macromolecules normally found in such natural sources. Such purification techniques may include, but are not limited to, ammonium sulfate precipitation, molecular sieve chromatography, ion exchange chromatography, high performance liquid chromatography (HPLC), fast protein liquid chromatography (FPLC), size-exclusion chromatography, capillary electrophoresis, polyacrylamide gel electrophoresis, agarose gel electrophoresis, isoelectric focusing, immunoelectrophoresis, dialysis, ultrafiltration, ultracentrifguation, hydrophobic interaction chromatography or the like. Alternatively, or additionally, the HF gene product may be purified by affinity chromatography, e.g., immunoaffinity chromatography using an immunoabsorbent column to which an antibody, or antibodies, is immobilized which is capable of binding the HF gene product. Such an antibody may be monoclonal or polyclonal in origin. If the HF gene product is specifically glycosylated, or modified in some other manner, the glycosylation pattern may be utilized as part of a purification scheme via, for example, lectin chromatography.

In accordance with certain examples, the cellular sources from which the HF gene product may be purified may include, but are not limited to, those cells that are expected, by Northern and/or Western blot analysis, to express the HF genes, e.g., cardiac myocytes, vascular smooth muscle cells, endothelial cells, fibroblasts, connective tissue cells, neuronal cells, glial cells, bone cells, bone marrow cells, chrondocytes, adipocytes, inflammatory cells, pancreatic cells, cancer cells, connective tissue matrix, epithelial cells, skeletal muscle cells and stem cells. Preferably, such cellular sources include, but are not limited to, excised hearts, tissue from heart biopsies, heart cells grown in tissue culture, biological samples and the like.

In accordance with certain examples, one or more forms of a HF gene product may be secreted or transported out of or into the cell or nucleus, e.g., may eventually be extracellular or intracellular or nuclear. Such extracellular or intracellular or nuclear forms of HF gene products may preferably be purified from whole tissue or biological samples as well as cells, utilizing any of the techniques described above. Preferable tissues include, but are not limited to those tissues than contain cell types such as those described above, e.g., heart tissue or brain tissue. Alternatively, HF expressing cells such as those described above may be grown in cell culture, under conditions well known to those of skill in the art. The HF gene product(s) may then be purified from the cell media using any of the techniques discussed above.

In accordance with certain examples, methods for the chemical synthesis of polypeptides (e.g., HF gene products) or fragments thereof, are well-known to those of ordinary skill in the art, e.g., peptides can be synthesized by solid phase techniques, cleaved from the resin and purified by preparative high performance liquid chromatography (see, e.g., Merrifield, B. 1986, Solid phase Synthesis. Science 232: 219-224; Creighton, 1983, Proteins: Structures and Molecular Principles, W. H. Freeman & Co., N.Y., pp. 50-60). The composition of the synthetic peptides may be confirmed by amino acid analysis or sequencing, e.g., using the Edman degradation procedure (see e.g., Creighton, 1983, supra at pp. 34-49), mass spectrometry or the like. Thus, a protein may be chemically synthesized in whole or in part.

In accordance with certain examples, an HF polypeptide may additionally be produced by recombinant DNA technology using one or more HF nucleotide sequences (SEQ. ID NOS: 1-1143 or SEQ. ID NOS.: 1144-1233) as described herein, coupled with techniques well known to those of ordinary skill in the art. Thus, methods for preparing the HF polypeptides and by expressing nucleic acid encoding HF sequences are described herein. Methods which will be selected by those of ordinary skill in the art, given the benefit of this disclosure, can be used to construct expression vectors containing HF protein coding sequences and appropriate transcriptional/translational control signals. These methods include, for example, in vitro recombinant DNA techniques, synthetic techniques and in vivo recombination/genetic recombination. See, for example, the techniques described in Maniatis et al., 1989, Molecular Cloning A Laboratory Manual, Cold Spring Harbor Laboratory, N.Y. and Ausubel et al., 1989, Current Protocols in Molecular Biology, Greene Publishing Associates and Wiley Interscience, N.Y. Alternatively, RNA capable of encoding HF protein sequences may be chemically synthesized using, for example, automated or semi-automated synthesizers. See, for example, the techniques described in “Oligonucleotide Synthesis”, 1984, Gait, M. J. ed., IRL Press, Oxford.

In accordance with certain examples, a variety of host-expression vector systems may be used to express the HF genes. Such host-expression systems represent vehicles by which the coding sequences of interest may be produced and subsequently purified, but also represent cells which may, when transformed or transfected with the appropriate nucleotide coding sequences, exhibit a HF polypeptide in situ. These include but are not limited to microorganisms such as bacteria (e.g., E. coli, B. subtilis) transformed with recombinant bacteriophage DNA, plasmid DNA, phasmid DNA or cosmid DNA expression vectors containing HF genes; yeast (e.g., Saccharomyces, Pichia) transformed with recombinant yeast expression vectors containing the HF gene; insect cell systems infected with recombinant virus expression vectors (e.g., Baculovirus-insect cell expression systems) containing the HF gene; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the HF gene; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter) containing the HF gene. Additional host and vector systems for expression of a HF gene product will be readily selected by the person of ordinary skill in the art, given the benefit of this disclosure.

In accordance with certain examples, in bacterial systems, a number of expression vectors may be advantageously selected depending upon the use intended for the HF polypeptide being expressed. For example, when a large quantity of such a protein is to be produced, e.g., for the generation of antibodies or to screen peptide libraries, vectors which direct the expression of high levels of fusion protein products that are readily purified may be desirable. Such vectors include, but are not limited to, the E. coli expression vector pUR278 (Ruther et al., 1983, EMBO J. 2:1791), in which a HF gene may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned HF polypeptide may be released from the GST moiety.

In accordance with certain examples, in an insect system, Autographa californica nuclear olyhedrosis virus (AcNPV) may be used as a vector to express foreign genes. The virus grows in Spodoptera frugiperda cells. A HF gene may be cloned individually into non-essential regions (for example the polyhedrin gene) of the virus and placed under control of an AcNPV promoter (for example the polyhedrin promoter). Successful insertion of a HF gene will result in inactivation of the polyhedrin gene and production of non-occluded recombinant virus (i.e., virus lacking the proteinaceous coat coded for by the polyhedrin gene). These recombinant viruses may then be used to infect Spodoptera frugiperda cells in which the inserted gene is expressed (e.g., see Smith et al., 1983, J. Viol. 46:584; Smith, U.S. Pat. No. 4,215,051).

In accordance with certain examples, in mammalian host cells, a number of viral-based expression systems may be used. In cases where an adenovirus, adeno-associated virus, lentivirus or retrovirus is used as an expression vector, a HF gene may be ligated to an adeno\adenoassociated\lenti\retro\virus transcription\translation control complex, e.g., the late promoter and tripartite leader sequence. This chimeric gene may then be inserted in the adeno\adenoassociated\lenti\retrovirus genome by in vitro or in vivo recombination. Insertion in a non-essential region of the viral genome (e.g., region E1, E4 or E3) will result in a recombinant virus that is viable and capable of expressing HF polypeptide in infected hosts (e.g., See Logan & Shenk, 1984, Proc. Natl. Acad. Sci. USA 81:3655-3659). Specific initiation signals may also be required for efficient translation of inserted HF genes. These signals may include, for example, the ATG initiation codon and adjacent sequences. In cases where an entire HF gene, including its own initiation codon and adjacent sequences, is inserted into the appropriate expression vector, no additional translational control signals may be needed. However, in cases where only a portion of the HF gene is inserted, exogenous translational control signals, including, perhaps, the ATG initiation codon, may be provided. Furthermore, the initiation codon may be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In accordance with certain examples, a host cell strain may be chosen which modulates the expression of the inserted sequences, or modifies and processes the gene product in the specific fashion desired. Such modifications, e.g., glycosylation or post-translational modification and processing, e.g., cleavage, of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, 3T3, W138, etc.

In accordance with certain examples, for long-term, high-yield production of recombinant proteins, stable expression may be desirable. For example, cell lines which stably express a HF protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in a suitable media, and then may be switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a HF gene product. Such engineered cell lines may be particularly useful in screening and evaluation of compounds that affect the endogenous activity of a HF gene product.

In accordance with certain examples, a number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cells 22:817) genes can be employed in tk⁻, hgprt⁻ or aprt⁻ cells, respectively. Also, anti-metabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 15 78:1527); gpt, which confers resistance to mycophenolic acid Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes. Additional selection systems suitable for use in cells will be readily selected by the person of ordinary skill in the art, given the benefit of this disclosure.

In accordance with certain examples, whether produced by molecular cloning methods or by, chemical synthetic methods, the amino acid sequence of a HF protein which may be used in one or more assays disclosed herein need not be identical to the amino acid sequence encoded by a HF gene reported herein. The HF protein used may comprise altered sequences in which amino acid residues are deleted, added, or substituted, while still resulting in a gene product functionally equivalent to the HF gene product. “Functionally equivalent,” refers to peptides capable of interacting with other cellular, nuclear, or extracellular molecules in a manner substantially similar to the way in which a corresponding portion of an endogenous HF gene product would interact. For example, functionally equivalent amino acid residues may be substituted for residues within the sequence resulting in a change of amino acid sequence. Such substitutes may be selected from other members of the class (i.e., non-polar, positively charged or negatively charged) to which the amino acid belongs; e.g., the nonpolar (hydrophobic) amino acids include alanine, leucine, isoleucine, valine, proline, phenylalanine, tryptophan, and methionine; the polar neutral amino acids include glycine, serine, threonine, cysteine, tyrosine, asparagine, and glutamine; the positively charged (basic) amino acids include arginine, lysine, and histidine; the negatively charged (acidic) amino acids include aspartic and glutamic acid. In certain examples, it may be possible to substitute one or more amino acids with a similarly sized and/or charged amino acid without a substantial alteration in the activity of the protein.

In accordance with certain examples, when used as a component in the assay systems described herein, a HF gene product or peptide (e.g., a gene product fragment) may be labeled, either directly or indirectly, to facilitate detection of a complex formed between a HF gene product and a targeting agent. Any of a variety of suitable labeling systems may be used including, but not limited to, radioisotopes such as ¹²⁵I, enzyme labeling systems that generate a detectable colorimetric signal or light when exposed to substrate, paramagnetic labels, magnetically active labels or luminescent labels, e.g., fluorescent, phosphorescent or chemiluminescent labels. The person of ordinary skill in the art, given the benefit of this disclosure will be able to select suitable additional labels.

In accordance with certain examples, where recombinant DNA technology is used to produce a HF gene product for use in the assays described herein, it may be desirable to engineer fusion proteins that can facilitate labeling, immobilization and/or detection. For example, the coding sequence of the viral or host cell protein can be fused to that of a heterologous protein that has enzyme activity or serves as an enzyme substrate in order to facilitate labeling and detection. The fusion constructs may be designed so that the heterologous component of the fusion product does not interfere with binding of the host cell and viral protein. Indirect labeling involves the use of a third protein, such as a labeled antibody, which specifically binds to one of the binding partners, i.e., either the HF protein or a binding partner. Such antibodies include but are not limited to polyclonal, monoclonal, chimeric, single chain, Fab fragments and fragments produced by a Fab expression library.

In accordance with certain examples, antibodies capable of specifically recognizing one or more HF gene product epitopes may be used in the methods described herein. In particular, antibodies may be used to identify HF gene products as well as treat patients with heart failure. Such antibodies may include, but are not limited to polyclonal antibodies, monoclonal antibodies (mAbs), humanized or chimeric antibodies, single chain antibodies, Fab fragments, F(ab′)₂ fragments, fragments produced by a FAb expression library, anti-idiotypic (anti-Id) antibodies, and epitope-binding fragments of any of the above. Such antibodies may be used, for example, in the detection of a HF gene product in a biological sample, or, alternatively, as a method for the inhibition of abnormal HF gene product activity, e.g., in the case where a HF gene product is up-regulated or down-regulated. Thus, such antibodies may be utilized as part of treatment methods, and/or may be used as part of diagnostic techniques whereby patients may be tested for abnormal levels of a HF gene product, or for the presence of abnormal forms of a HF polypeptide. In certain examples, the antibody may be administered in an effective amount to a patient in need of treatment for heart disease or heart failure.

In accordance with certain examples, for the production of antibodies to a HF gene product, various host animals may be immunized by injection with a HF protein, or a portion thereof. Such host animals may include but are not limited to, rabbits, mice, and rats. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (Bacille Calmette-Guerin) and Corynebacteriumparvum.

In accordance with certain examples, polyclonal antibodies are heterogeneous populations of antibody molecules derived from the sera of animals immunized with an antigen, such as a HF protein, or an antigenic functional derivative thereof. For the production of polyclonal antibodies, host animals such as those described above, may be immunized by injection with a HF protein supplemented with adjuvants as also described above. Monoclonal antibodies which are substantially homogeneous populations of antibodies to a particular antigen, may be obtained by any technique which provides for the production of antibody molecules by continuous cell lines in culture. These include, but are not limited to, the hybridoma technique of Kohler and Milstein (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class, including, for example, IgG, IgM, IgE, IgA, IgD and any subclass thereof. The hybridoma producing the mAb may be cultivated in vitro or in vivo. In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454; U.S. Pat. No. 4,816,567, which is incorporated by reference herein in its entirety) by splicing the genes from a mouse antibody molecule of appropriate antigen specificity together with genes from a human antibody molecule of appropriate biological activity can be used. A chimeric antibody is a molecule in which different portions are derived from different animal species, such as those having a murine variable region and a human immunoglobulin constant region. Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce HF-single chain antibodies. Single chain antibodies are formed by linking the heavy and light chain fragment of the Fv region via an amino acid bridge, resulting in a single chain polypeptide. Further, HF-humanized monoclonal antibodies may be produced using standard techniques (see, for example, U.S. Pat. No. 5,225,539, which is incorporated herein by reference in its entirety).

In accordance with certain examples, antibody fragments which recognize specific epitopes may be generated by known techniques. For example, such fragments include but are not limited to the F(ab′)₂ fragments which can be produced by pepsin digestion of the antibody molecule, and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

In accordance with certain examples, numerous assays may be used along with the polynucleotides disclosed herein to identify agents, e.g., small organic compounds, that bind to a HF gene product, other cellular proteins that interact with a HF gene product, and compounds that interfere with the interaction of a HF gene product with other cellular proteins or cellular structures, e.g., cellular membranes or organelles. Compounds identified via assays such as those described herein may be useful, for example, in elaborating the biological function of a HF gene product, and for ameliorating symptoms caused by up-regulation or down-regulation of a HF gene. For example, in instances whereby a mutation in a HF gene causes a lower level of expression and therefore results in an overall lower level of HF gene product activity in a cell or tissue, compounds that interact with the HF gene product may include ones which accentuate or amplify the activity of the HF gene product. Thus, such compounds would bring about an effective increase in the level of HF gene product activity, thus ameliorating HF symptoms. In instances whereby mutations with the HF gene cause aberrant HF proteins to be made which have a deleterious effect that leads to heart failure or heart disease, compounds that bind an aberrant HF protein may be identified that inhibit the activity of the aberrant HF protein. This decrease in the aberrant HF gene activity can therefore, serve to ameliorate heart failure or heart disease symptoms. In instances whereby a mutation in a HF gene causes a higher level of expression and therefore results in an overall higher level of HF gene product activity in a cell or tissue, compounds that interact with the HF gene product may include ones which reduce the activity of the HF gene product. Thus, such compounds would bring about an effective decrease in the level of HF gene product activity, thus ameliorating HF symptoms. Assays for testing the effectiveness of compounds, identified by, for example, techniques such as those described herein, will be readily selected by the person of ordinary skill in the art, given the benefit of this disclosure.

In accordance with certain examples, in vitro systems may be constructed to identify compounds capable of binding a HF gene. Such compounds may include, but are not limited to, peptides made of D- and/or L-configuration amino acids (in, for example, the form of random peptide libraries; see Lam, K. S. et al., 1991, Nature 354:82-84), phosphopeptides (in, for example, the form of random or partially degenerate, directed phosphopeptide libraries; see, for example, Songyang, Z. et al., 1993, Cell 72:767-778), antibodies, and small or large organic or inorganic molecules. Compounds identified may be useful, for example, in modulating the activity of HF proteins or HF genes may be useful in elaborating the biological function of the HF protein, may be used in screens for identifying compounds that disrupt or enhance normal HF protein or HF gene interactions, or may in themselves disrupt or enhance such interactions.

In accordance with certain examples, an assay useful in identifying compounds that bind to an HF protein involves preparing a reaction mixture of the HF protein and a test agent under conditions and for a time sufficient to allow the two components to interact and bind, thus potentially forming a complex which can be removed and/or detected in the reaction mixture, e.g., using the luminescent or calorimetric labels disclosed herein. These assays can be conducted in a heterogeneous or homogeneous format. Heterogeneous assays involve anchoring a HF protein or the test agent onto a solid phase and detecting HF protein-test agent complexes anchored on the solid phase at the end of the reaction. In homogeneous assays, the entire reaction is carried out in a liquid phase, e.g., in a single reaction vessel. In either approach, the order of addition of reactants can be varied to obtain different information about the agents being tested. In a heterogeneous assay system, the HF protein may be anchored onto a solid surface, and the test agent, which is typically not anchored, is labeled, either directly or indirectly. In practice, microtiter plates may be conveniently used. The anchored component may be immobilized by non-covalent or covalent attachments. Non-covalent attachment may be accomplished simply by coating the solid surface with a solution of the protein and drying. Alternatively, an immobilized antibody, preferably a monoclonal antibody, specific for the protein may be used to anchor the protein to the solid surface. The surfaces may be prepared in advance and stored. The labeled component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the labeled compound is pre-labeled, the detection of label immobilized on the surface indicates that complexes were formed. Where the labeled component is not pre-labeled, an indirect label can be used to detect complexes anchored on the surface; e.g., using a labeled antibody specific for the binding partner (the antibody, in turn, may be directly labeled or indirectly labeled with a secondary antibody, such as, for example, a labeled anti-Ig antibody). Alternatively, a heterogenous reaction can be conducted in a liquid phase, the reaction products separated from unreacted components, and complexes detected, e.g., using an immobilized antibody specific for a HF protein or the test substance to anchor any complexes formed in solution, and a labeled antibody specific for the other binding partner to detect anchored complexes.

In an alternate embodiment, a homogeneous assay can be used. In this approach, a preformed complex of the HF protein and a known binding partner is prepared in which one of the components is labeled, but the signal generated by the label is quenched due to complex formation (see, e.g., U.S. Pat. No. 4,109,496 by Rubenstein which uses this approach for immunoassays). The addition of a test substance that competes with and displaces one of the binding partners from the preformed complex will result in the generation of a signal above a background signal.

In accordance with certain examples, any method suitable for detecting protein-protein interactions may be employed for identifying novel HF-cellular, nuclear, or extracellular protein interactions. For example, some traditional methods which may be employed are co-immunoprecipitation, cross-linking and co-purification through gradients or chromatographic columns may be used. Additionally, methods which result in the simultaneous identification of the genes coding for the protein interacting with a target protein may be employed. These methods include, for example, probing expression libraries with labeled target protein. One such method which detects protein interactions in vivo, the yeast two-hybrid system, is described in detail for illustration only and without limitation. One version of this system has been described (Chien et al., 1991, Proc. Natl. Acad. Sci. USA, 88:9578-9582) and is commercially available from Clontech (Palo Alto, Calif.). Briefly, using such a system, plasmids are constructed that encode two hybrid proteins: one consists of the DNA-binding domain of a transcription activator protein fused to one test protein “X” and the other consists of the activator protein's activation domain fused to another test protein “Y”. Thus, either “X” or “Y” in this system may be wild type or mutant HF protein, while the other may be a test protein or peptide. The plasmids are transformed into a strain of the yeast Saccharomyces cerevisiae that contains a reporter gene (e.g., lacZ) whose regulatory region contains the activator's binding sites. Either hybrid protein alone cannot activate transcription of the reporter gene, the DNA-binding domain hybrid, because it does not provide activation function and the activation domain hybrid because it cannot localize to the activator's binding sites. Interaction of the two proteins reconstitutes the functional activator protein and results in expression of the reporter gene, which is detected by an assay for the reporter gene product. The two-hybrid system or related methodology can be used to screen activation domain libraries for proteins that interact with a HF protein. Total genomic or cDNA sequences may be fused to the DNA encoding an activation domain. This library and a plasmid encoding a hybrid of the HF protein fused to the DNA-binding domain may be co-transformed into a yeast reporter strain, and the resulting transformants may be screened for those that express the reporter gene. These colonies may be purified and the plasmids responsible for reporter gene expression are isolated. DNA sequencing may then be used to identify the proteins encoded by the library plasmids. For example, the HF gene may be cloned into a vector such that it is translationally fused to the DNA encoding the DNA-binding domain of the GAL4 protein. A cDNA library of the cell line from which proteins that interact with HF protein are to be detected can be made using methods routinely practiced by those of ordinary skill in the art. According to this particular system, for example, the cDNA fragments can be inserted into a vector such that they are translationally fused to the activation domain of GAL4. This library can be co-transformed along with the HF-GAL4 DNA binding domain fusion plasmid into a yeast strain which contains a lacZ gene driven by a promoter which contains GAL4 activation sequences. A cDNA encoded protein, fused to GAL4 activation domain, that interacts with a HF protein will reconstitute an active GAL4 protein and thereby drive expression of the lacZ gene. Colonies which express lacZ can be detected by their blue color in the presence of X-gal. The cDNA can then be extracted from strains derived from these and used to produce and isolate the HF protein—interacting protein using techniques routinely practiced in the art.

In accordance with certain examples, the HF gene products may, in vivo or in vitro, interact with one or more cellular, nuclear, or extracellular proteins to cause symptoms present in heart failure or heart disease. Such cellular proteins are referred to herein in some instances as “binding partners.” Compounds that disrupt such interactions may be useful in regulating the activity of the HF protein, especially up-regulated HF proteins. Such compounds may include, but are not limited to molecules such as antibodies, peptides, and the like described herein. In instances whereby heart failure or heart disease symptoms are caused by a mutation within a HF gene which produces HF gene products having aberrant, gain-of-function activity, compounds identified that disrupt such interactions may, therefore inhibit the aberrant HF activity. Preferably, compounds may be identified which disrupt the interaction of mutant HF gene products with cellular, nuclear, or extracellular proteins, but do not substantially effect the interactions of the normal HF protein. Such compounds may be identified by comparing the effectiveness of a compound to disrupt interactions in an assay containing normal HF protein to that of an assay containing mutant HF protein.

In accordance with certain examples, an assay to identify a compound that interferes with the interaction between a HF protein and a cellular, nuclear or extracellular protein binding partner may include preparing a reaction mixture containing a HF protein and the binding partner under conditions and for a time sufficient to allow the HF protein and the binding partner to interact and bind, thus forming a complex. In order to test a compound for inhibitory activity, the reaction may be conducted in the presence and absence of the test compound, i.e., the test compound may be initially included in the reaction mixture, or added at a time subsequent to the addition of HF and its cellular, nuclear, or extracellular binding partner; controls are incubated without the test compound or with a placebo. The formation of any complexes between the HF protein and the cellular, nuclear, or extracellular binding partner is then detected. The formation of a complex in the control reaction, but not in the reaction mixture containing the test compound indicates that the compound interferes with the interaction of the HF protein and the binding partner. As noted above, complex formation within reaction mixtures containing the test compound and normal HF protein may also be compared to complex formation within reaction mixtures containing the test compound and a mutant HF protein. This comparison may be important in those cases wherein it is desirable to identify compounds that disrupt interactions of mutant but not normal HF proteins. The assay for compounds that interfere with the interaction of the binding partners can be conducted in a heterogeneous or homogeneous format. For example, test compounds that interfere with the interaction between the binding partners, e.g., by competition, can be identified by conducting the reaction in the presence of the test substance; i.e., by adding the test substance to the reaction mixture prior to or simultaneously with a HF gene product and interactive cellular, nuclear or extracellular protein. On the other hand, test compounds that disrupt preformed complexes, e.g., compounds with higher binding constants that displace one of the binding partners from the complex, may be tested by adding the test compound to the reaction mixture after complexes have been formed. In a heterogeneous assay system, one binding partner, e.g., either the HF gene product or the interactive cellular or extracellular protein, is anchored onto a solid surface, and its binding partner, which is not anchored, is labeled, either directly or indirectly. In practice, microtiter plates may be used. The anchored species may be immobilized by non-covalent or covalent attachments. Non-covalent attachment may be accomplished simply by coating the solid surface with a solution of the protein and drying. Alternatively, an immobilized antibody specific for the protein may be used to anchor the protein to the solid surface. The surfaces may be prepared in advance and stored.

In accordance with certain examples, in order to conduct the assay, the binding partner of the immobilized species may be added to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the binding partner was pre-labeled, the detection of label immobilized on the surface indicates that complexes were formed. Where the binding partner is not pre-labeled, an indirect label can be used to detect complexes anchored on the surface, e.g., using a labeled antibody specific for the binding partner (the antibody, in turn, may be directly labeled or indirectly labeled with a secondary antibody such as, for example, labeled anti-Ig antibody). Depending upon the order of addition of reaction components, test compounds which inhibit complex formation or which disrupt preformed complexes can be detected.

In accordance with certain examples, the reaction can alternatively be conducted in a liquid phase in the presence or absence of the test compound, the reaction products separated from unreacted components, and complexes detected, e.g., using an immobilized antibody specific for one binding partner to anchor any complexes formed in solution, and a labeled antibody specific for the other binding partner to detect anchored complexes. Again, depending upon the order of addition of reactants to the liquid phase, test compounds which inhibit complex or which disrupt preformed complexes can be identified.

In accordance with certain examples, a homogeneous assay can be used. In this approach, a preformed complex of a HF protein and the interactive cellular, nuclear, or extracellular protein may be prepared in which one of the binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the binding partners from the preformed complex may result in the generation of a signal above background. In this way, test substances which disrupt HF protein-cellular, nuclear, or extracellular protein interaction can be identified. In a specific embodiment, the HF protein can be prepared for immobilization using recombinant DNA techniques described herein. For example, the HF coding region can be fused to the glutathione-S-transferase (GST) gene using the fusion vector pGEX-5X-1, in such a manner that its binding activity is maintained in the resulting fusion protein. The interactive cellular, nuclear, or extracellular protein can be purified and used to raise a monoclonal antibody, using methods routinely practiced in the art and described above. This antibody can be labeled with the radioactive isotope ¹²⁵I, for example, by methods routinely practiced by those of ordinary skill in the art. In a heterogeneous assay, the GST-HF fusion protein can be anchored to glutathione-agarose beads. The interactive cellular, nuclear, or extracellular protein can then be added in the presence or absence of the test compound in a manner that allows interaction and binding to occur. At the end of the reaction period, unbound material may be washed away, and the labeled monoclonal antibody may be added to the system and allowed to bind to the complexed binding partners. The interaction between the HF protein and the interactive cellular, nuclear, or extracellular protein can be detected by measuring the amount of radioactivity that remains associated with the glutathione-agarose beads. A successful inhibition of the interaction by the test compound may result in a decrease in measured radioactivity. Alternatively, the GST-HF fusion protein and the interactive cellular, nuclear, or extracellular protein may be mixed together in liquid in the absence of the solid glutathione-agarose beads. The test compound may be added either during or after the binding partners are allowed to interact. This mixture may then be added to the glutathione-agarose beads and unbound material is washed away. Again the extent of inhibition of the binding partner interaction can be detected by adding the labeled antibody and measuring the radioactivity associated with the beads.

In accordance with certain examples, these same techniques can be employed using peptide fragments that correspond to a binding domain of a HF protein and the interactive cellular, nuclear or extracellular protein, respectively, in place of one or both of the full length proteins. Any number of methods routinely practiced in the art can be used to identify and isolate the protein's binding site. These methods include, but are not limited to, mutagenesis of one of the genes encoding the proteins and screening for disruption of binding in a co-immunoprecipitation assay. Compensating mutations in a HF gene can be selected. Sequence analysis of the genes encoding the respective proteins may reveal the mutations that correspond to the region of the protein involved in interactive binding. Alternatively, one protein can be anchored to a solid surface using methods described herein and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the cellular, nuclear, or extracellular protein is obtained, short gene segments can be engineered to express peptide fragments of the protein, which can then be tested for binding activity and purified or synthesized.

For example, and not by way of limitation, a HF protein can be anchored to a solid material as described above by making a GST-HF fusion protein and allowing it to bind to glutathione agarose beads. The interactive cellular protein can be labeled with a radioactive isotope, such as ³⁵S, and cleaved with a proteolytic enzyme such as trypsin. Cleavage products can then be added to the anchored GST-HF fusion protein and allowed to bind. After washing away unbound peptides, labeled bound material, representing the cellular or extracellular protein binding domain, can be eluted, purified, and analyzed for amino acid sequence by methods well known to those or ordinary skill in the art. Peptides so identified can be produced synthetically or fused to appropriate facilitative proteins using, for example, recombinant DNA technology.

In accordance with certain examples, cells that contain and express mutant HF gene sequences which encode mutant HF protein, and thus exhibit cellular phenotypes associated with heart failure, may be used to identify compounds that may be used to treat heart failure. Such cells may include cell lines consisting of naturally occurring or engineered cells which express mutant or express both normal and mutant HF gene products. Such cells include, but are not limited to cardiac myocytes, vascular smooth muscle cells, endothelial cells, fibroblasts, connective tissue cells, neuronal cells, glial cells, bone cells, bone marrow cells, chrondocytes, adipocytes, inflammatory cells, pancreatic cells, cancer cells, connective tissue matrix, epithelial cells, skeletal muscle cells and stem cells. Cells, such as those described above, which exhibit or fail to exhibit HF-like cellular phenotypes, may be exposed to a compound suspected of inhibiting (or increasing as the case may be) one or more HF gene products at a sufficient concentration and for a time sufficient to elicit such inhibition (or increase) in the exposed cells. Alternatively, cells, such as those described above, which exhibit or fail to exhibit HF-like cellular phenotypes, may be exposed to a compound suspected of stimulating production or inhibition of production of one or more HF gene products at a sufficient concentration and for a time sufficient to elicit such stimulation in the exposed cells. After exposure, the cells may be examined to determine whether one or more of the HF-like cellular phenotypes has been altered to resemble a more wild type, non-HF phenotype.

In accordance with certain examples, one or more markers associated with up-regulation or down-regulation of a HF gene may be used to assess whether or not a compound inhibits or stimulates a cell. For example, certain cellular products may be lost when a HF gene is down-regulated, e.g., ATPases, membrane proteins, receptors, etc., and, if a compound can stimulate a HF gene, the re-appearance of such lost cellular products may be observed. Such markers may be examined using, for example, standard immunohistology techniques using antibodies specific to the marker(s) of interest in conjunction with procedures that are well known to those of ordinary skill in the art. Additionally, assays for the function of a HF gene product can, for example, include a measure of extracellular matrix (ECM) components, such as proteoglycans, laminin, fibronectin and the like in the case where such ECM components are present at higher or lower amounts. Thus, any compound which serves to create an extracellular matrix environment which more fully mimics the normal ECM could be tested for its ability to ameliorate HF symptoms. In certain examples, a particular profile may be altered during and/or after development of a particular heart disease or heart failure. For example, in female human patients who develop heart disease or heart failure, the energetic profile (as discussed herein) may be altered, e.g., up-regulated or down-regulated.

In accordance with certain examples, the ability of a compound, such as those identified in the foregoing binding assays, to prevent or inhibit disease may be assessed in animal models of HF such as, for example, animal models involving idiopathic cardiomyopathy, as discussed herein. Additionally, animal models exhibiting HF-like symptoms may be engineered by utilizing the HF sequences (SEQ. ID. NOS.: 1-1143 or SEQ. ID NOS.: 1144-1233) in conjunction with techniques for producing transgenic animals that are well known to those of skill in the art, e.g., U.S. Pat. No. 4,736,866. In other examples, HF knock-out animals may be engineered. In yet other examples, HF knock-in animals may be engineered. For example, in certain situations overexpression of a HF gene product may occur if one or more of HF genes are not present to down-regulate expression. In other situations, underexpression of a HF gene product may occur if one or more HF genes are not present to up-regulate or control expression. Animals of any species, including, but not limited to, mice, rats, rabbits, guinea pigs, pigs, micro-pigs, goats, chickens, turkeys, other avian species and non-human primates, e.g., baboons, squirrels, monkeys, and chimpanzees may be used to generate such HF animal models.

In accordance with certain examples, in instances wherein the HF mutation leading to HF symptoms causes a drop in the level of a HF protein or causes an ineffective HF protein to be made (i.e., the HF mutation is a dominant loss-of-function mutation) various strategies may be utilized to generate animal models exhibiting HF-like symptoms. For example, HF knockout animals, such as mice, rats, pigs, chickens or turkeys, may be generated and used to screen for compounds which exhibit an ability to ameliorate HF systems. Animals may be generated whose cells contain one inactivated copy of a HF-homolog. In such a strategy, human HF gene sequences may be used to identify a HF homolog within the animal of interest. Once such a HF homolog has been identified, well-known techniques may be used to disrupt and inactivate the endogenous HF homolog, and further, to produce animals which are heterozygous for such an inactivated HF homolog. Such animals may then be observed for the development of HF-like symptoms.

In accordance with certain examples, in instances wherein a HF mutation causes a HF protein having an aberrant HF activity which leads to HF symptoms (i.e., the HF mutation is a dominant gain-of-function mutation) strategies such as those now described may be utilized to generate HF animal models. First, for example, a human HF gene sequence containing such a gain-of-function HF mutation, and encoding such an aberrant HF protein, may be introduced into the genome of the animal of interest by utilizing well known techniques. Such a HF nucleic acid sequence may be controlled by a regulatory nucleic acid sequence which allows the mutant human HF sequence to be expressed in the cells, preferably cardiac myocytes, of the animal of interest. The human HF regulatory promoter/enhancer sequences may be sufficient for such expression. Alternatively, the mutant HF gene sequences may be controlled by regulatory sequences endogenous to the animal of interest, or by any other regulatory sequences which are effective in bringing about the expression of the mutant human HF sequences in the animal cells of interest.

In accordance with certain examples, one or more genes may be introduced into an animal system to counteract the effects of a HF mutation. Such an introduced gene, for example, may replace a non-functioning gene, may down-regulate an aberrant gene or may up-regulate a non-functioning gene. In some examples, the gene may produce a gene product that can bind to an aberrant HF protein to prevent the aberrant HF protein from exerting any unwanted effects. Additional uses of introduced genes will be readily selected by the person of ordinary skill in the art, given the benefit of this disclosure.

In accordance with certain examples, expression of the mutant human HF gene product may be assayed, for example, by standard Northern or Western analysis, and the production of the mutant human HF gene product may be assayed by, for example, detecting its presence by using techniques whereby binding of an antibody directed against the mutant human HF gene product is detected. Those animals found to express the mutant human HF gene product may then be observed for the development of heart failure or heart disease symptoms. Alternatively, animal models of HF may be produced by engineering animals containing mutations within one copy of their endogenous HF-homolog which correspond to gain-of-function mutations within the human HF gene. Utilizing such a strategy, a HF homolog may be identified and cloned from the animal of interest, using well-known techniques, such as those described herein. One or more gain-of-function mutations (or loss-of-function mutations as the case may be) may be engineered into such a HF homolog which corresponds to gain-of-function mutations (or loss-of-function mutations) within the human HF gene. By “corresponding”, it is meant that the mutant gene product produced by such an engineered HF homolog may exhibit an aberrant HF activity which is substantially similar to that exhibited by the mutant human HF protein. The engineered HF homolog may then be introduced into the genome of the animal of interest, using techniques such as those described herein. Because the mutation introduced into the engineered HF homolog is expected to be a dominant gain-of-function mutation integration into the genome need not be via homologous recombination, although such a route is preferred.

In accordance with certain examples, once transgenic animals have been generated, the expression of the mutant HF homolog gene and protein may be assayed utilizing standard techniques, such as Northern and/or Western analyses. Animals expressing mutant HF homolog proteins in cells or tissues, such as, for example, cardiac myocytes, of interest, may be observed for the development of heart failure or heart disease symptoms.

In accordance with certain examples, any of the HF animal models described herein may be used to test compounds for an ability to ameliorate HF symptoms. In addition, as described in detail herein, such animal models may be used to determine the LD₅₀ and the ED₅₀ in animal subjects, and such data may be used to determine the in vitro and/or in vivo efficacy of potential HF treatments.

In accordance with certain examples, any technique used by those of ordinary skill in the art may be used to introduce a HF gene into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82:6148-6152); gene targeting in embryonic stem cells (Thompson et al., 1989, Cell 56:313-321); electroporation of embryos (Lo, 1983, Mol. Cell. Biol. 3:1803-1814); and sperm-mediated gene transfer (Lavitrano et al., 1989, Cell 57:717-723); etc. For a review of such techniques, see Gordon, 1989, Transgenic Animals, Intl. Rev. Cytol. 115:171-229. When it is desired that the HF transgene be integrated into the chromosomal site of the endogenous HF, gene targeting is preferred. Briefly, when such a technique is to be used, vectors containing some nucleotide sequences homologous to the endogenous HF gene of interest are designed for the purpose of integrating, via homologous recombination with chromosomal sequences, into and disrupting the function of, the nucleotide sequence of the endogenous HF gene.

In accordance with certain examples, once the HF founder animals are produced, they may be bred, inbred, outbred, or crossbred to produce colonies of the particular animal. Examples of such breeding strategies include but are not limited to, outbreeding of founder animals with more than one integration site in order to establish separate lines, inbreeding of separate lines in order to produce compound HF transgenics that express the HF transgene at higher levels because of the effects of additive expression of each HF transgene, crossing of heterozygous transgenic animals to produce animals homozygous for a given integration site in order to both augment expression and eliminate the possible need for screening of animals by DNA analysis, crossing of separate homozygous lines to produce compound heterozygous or homozygous lines, and breeding animals to different inbred genetic backgrounds so as to examine effects of modifying alleles on expression of the HF transgene and the development of HF symptoms. One such approach is to cross the HF founder animals with a wild type strain to produce a first generation that exhibits HF symptoms, such as the development of enlarged hearts. The first generation may then be inbred in order to develop a homozygous line, if it is found that homozygous HF transgenic animals are viable. In certain examples, one or more HF founders may be produced that include one or more genes that counter the effects of an HF gene, and such HF founders may be bred using any selected breeding method known to those of ordinary skill in the art to provide a desired HF animal line.

In accordance with certain examples, transgenic animals that carry the transgene in all their cells, as well as animals which carry the transgene in some, but not all their cells, i.e., mosaic animals, may be used. The transgene may be integrated as a single transgene or in concatamers, e.g., head-to-head tandems or head-to-tail tandems.

In accordance with certain examples, the HF transgenic animals that are produced in accordance with the procedures detailed, may be screened and evaluated to select those animals which may be used as suitable animal models for HF. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (RT-PCR). Samples of HF-expressing tissue, cardiac tissue, for example, may be evaluated immunocytochemically using antibodies specific for the HF transgene gene product. The HF transgenic animals that express a HF gene product, which may be detected, for example, by immunocytochemical techniques using antibodies directed against HF tag epitopes, at easily detectable levels may then be further evaluated histopathologically to identify those animals which display characteristic heart failure symptoms. Such transgenic animals serve as suitable model and testing systems for heart failure.

In accordance with certain examples, the HF animal models disclosed herein may be used as model systems for HF, e.g., for dilated idiopathic cardiomyopathy, and/or to generate cell lines that can be used as cell culture models for HF. The HF transgenic animal model systems for HF may be used to identify drugs, pharmaceuticals, therapies and interventions which may be effective in treating heart failure. Potential therapeutic agents may be tested by systemic or local administration. Suitable routes may include oral, rectal, or intestinal administration, parenteral delivery, including intramuscular, subcutaneous, intramedullary injections, as well as intrathecal, direct intraventricular, intravenous, intraperitoneal, intranasal, intraocular injections, or other known methods of administering drugs in solid, liquid or other form. The response of the animals to the treatment may be monitored by assessing the reversal of disorders associated with heart failure. With regard to intervention, any treatments which reverse any aspect of HF-like symptoms may be considered as candidates for human HF therapeutic intervention. However, treatments or regimens which reverse the constellation of pathologies associated with any of these disorders may be preferred. Dosages of test agents may be determined by deriving dose-response curves using methods well known by those of ordinary skill in the art.

In accordance with certain examples, HF transgenic animals may be used to derive a cell line which may be used as a test substrate in culture, to identify agents that ameliorate HF-like symptoms. While primary cultures derived from the HF transgenic animals may be utilized, the generation of continuous cell lines is preferred. For examples of techniques which may be used to derive a continuous cell line from the transgenic animals, see Small et al., 1985, Mol. Cell. Biol. 5:642-648. In certain examples, such cell lines may be used, for example, to establish the in vitro and/or in vivo efficacy of a particular agent.

In accordance with certain examples, dominant mutations in a HF gene that cause HF symptoms may act as gain-of-function (or loss-of-function as the case may be) mutations which produce a form of the HF protein which exhibits an aberrant activity that leads to the formation of HF symptoms (or prevents HF symptoms). A variety of techniques may be used to inhibit (or enhance) the expression, synthesis, or activity of such mutant HF genes and gene products (i.e., proteins). For example, compounds such as those identified through assays described herein, which exhibit inhibitory activity may be used to ameliorate HF symptoms. In other examples, compounds may be used to provide synergistic effects to enhance activity of a particular gene to ameliorate HF symptoms. Such compounds and molecules may include, but are not limited to, small and large organic molecules, peptides, oligonucleotides (e.g., post-transcriptional gene silencers such as RNAi's) and antibodies. Illustrative inhibitory antibody techniques are described herein. Among the compounds which may exhibit anti-HF activity are antisense, ribozyme, RNAi's, and triple helix molecules. Such molecules may be designed to enhance, reduce or inhibit HF protein activity. Techniques for the production and use of such molecules are well known to those of ordinary skill in the art.

In accordance with certain examples, antisense RNA and DNA molecules may act to block directly the translation of mRNA by binding to targeted mRNA and preventing protein translation. With respect to antisense DNA, oligodeoxyribonucleotides derived from the translation initiation site, e.g., between the −10 and +10 regions of the HF nucleotide sequence of interest, are preferred. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules may include one or more sequences complementary to the target HF mRNA, preferably the mutant HF mRNA, and may include the well known catalytic sequence responsible for mRNA cleavage. For this sequence, see, for example, U.S. Pat. No. 5,093,246, which is incorporated by reference herein in its entirety. As such, within the scope of this disclosure are engineered hammerhead motif ribozyme molecules that specifically and efficiently catalyze endonucleolytic cleavage of RNA sequences encoding HF proteins, preferably mutant HF proteins. Specific ribozyme cleavage sites within any potential RNA target are initially identified by scanning the target molecule for ribozyme cleavage sites which include the following sequence: GUA, GUU and GUC. Once identified, short RNA sequences of between 15 and ribonucleotides corresponding to the region of the target gene containing the cleavage site may be evaluated for predicted structural features, such as secondary structure, that may render the oligonucleotide sequence unsuitable. The suitability of candidate targets may also be evaluated by testing their accessibility to hybridization with complementary oligonucleotides, using ribonuclease protection assays.

In accordance with certain examples, nucleic acid molecules to be used in triplex helix formation may be single stranded and composed of deoxyribonucleotides. The base composition of these oligonucleotides may be designed to promote triple helix formation via Hoogsteen base pairing rules, which generally require sizeable stretches of either purines or pyrimidines to be present on one strand of a duplex. Nucleotide sequences may be pyrimidine-based, which can result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of guanidine residues. These molecules may form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation may be increased by creating a “switchback” nucleic acid molecule. Switchback molecules are synthesized in an alternating 5′-3′,3′-5′ manner, such that they base pair with one strand of a duplex first and then the other, eliminating the necessity for a sizeable stretch of either purines or pyrimidines to be present on one strand of a duplex.

In accordance with certain examples, it is possible that the antisense, ribozyme, RNAi and/or triple helix molecules described herein may enhance, reduce or inhibit the translation of mRNA produced by both normal and mutant HF alleles. In order to ensure that substantial normal levels of HF activity are maintained in the cell, nucleic acid molecules that encode and express HF proteins exhibiting normal HF activity may be introduced into cells which do not contain sequences susceptible to such antisense, ribozyme, or triple helix treatments. Such sequences may be introduced via gene therapy methods such as those described herein. Alternatively, it may be preferable to co-administer normal HF protein into the cell or tissue in order to maintain the requisite level of cellular or tissue HF activity.

In accordance with certain examples, antisense RNA and DNA molecules, ribozyme molecules, RNAi's and triple helix molecules may be prepared by methods well known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

In accordance with certain examples, various well-known modifications to the DNA molecules may be introduced as a means of increasing intracellular stability and half-life. Possible modifications include, but are not limited to, the addition of flanking sequences of ribo- or deoxyribonucleotides to the 5′ and/or 3′ ends of the molecule or the use of phosphorothioate or 2′-O-methyl rather than phosphodiesterase linkages within the oligodeoxyribonucleotide backbone.

In accordance with certain examples, antibodies that are both specific for mutant HF gene product and interfere with its activity may be used. Such antibodies may be generated using standard techniques such as the illustrative techniques described herein, against the proteins themselves or against peptides corresponding to the binding domains of the proteins. Such antibodies include but are not limited to polyclonal, monoclonal, Fab fragments, F(ab′)₂ fragments, single chain antibodies, chimeric antibodies, humanized antibodies, etc. In instances where a HF protein appears to be an extracellular protein, any of the illustrative administration techniques described herein which are appropriate for peptide administration may be utilized to effectively administer inhibitory HF antibodies to their site of action.

In accordance with certain examples, dominant mutations in a HF gene may lower the level of expression of the HF gene or alternatively, may cause inactive or substantially inactive HF gene products to be formed. In either instance, the result is an overall lower level of normal activity in the tissues or cells in which HF gene products are normally expressed. This lower level of HF gene product activity may contribute, at least in part, to HF symptoms. Thus, such HF mutations represent dominant loss-of-function mutations. The level of normal HF gene product activity may be increased to levels wherein HF symptoms are ameliorated. For example, normal HF protein, at a level sufficient to ameliorate HF symptoms may be administered to a patient exhibiting such symptoms. Any of the techniques discussed herein may be used for such administration. The person of ordinary skill in the art, given the benefit of this disclosure, will be able to determine the concentration of effective, non-toxic doses of the normal HF protein, using well known techniques. Additionally, DNA sequences encoding normal HF protein may be directly administered to a patient exhibiting HF symptoms, at a concentration sufficient to produce a level of HF protein such that HF symptoms are ameliorated. Any of the techniques discussed herein that achieve intracellular administration of compounds, such as, for example, liposome administration, may be utilized for the administration of such DNA molecules. The DNA molecules may be produced, for example, by recombinant techniques such as those described herein or using other techniques well known by those of ordinary skill in the art.

In accordance with certain examples, dominant mutations in a HF gene may increase the level of expression of the HF gene or alternatively, may cause overactive or substantially overactive HF gene products to be formed. In either instance, the result is an overall higher level of normal activity in the tissues or cells in which HF gene products are normally expressed. This higher level of HF gene product activity may contribute, at least in part, to HF symptoms. Thus, such HF mutations represent dominant gain-of-function mutations. The level of HF gene product activity may be decreased to levels wherein HF symptoms are ameliorated. For example, an antibody may be administered to bring the levels of HF protein to a level sufficient to ameliorate HF symptoms by administering such antibody to a patient exhibiting such symptoms. Any of the techniques discussed herein may be used for such administration. Any of the techniques discussed herein that achieve intracellular administration of compounds, such as, for example, liposome administration, may be utilized for the administration of such antibodies. The antibodies may be produced, for example, by techniques such as those described herein or using other techniques well known by those of ordinary skill in the art.

In accordance with certain examples, patients with dominant loss-of-function mutations may be treated by gene replacement therapy. A copy of the normal HF gene or a part of the gene that directs the production of a normal HF protein with the function of the HF protein may be inserted into cells, e.g., cardiac cells, using viral or non-viral vectors which include, but are not limited to vectors derived from, for example, retroviruses, vaccinia virus, adenoviruses, adeno-associated virus, CMV, lentiviruses, herpes viruses, bovine papilloma virus or additional, non-viral vectors, such as plasmids. In addition, techniques frequently employed by those skilled in the art for introducing DNA into mammalian cells may be utilized. For example, methods including but not limited to electroporation, DEAE-dextran mediated DNA transfer, DNA guns, liposomes, direct injection, pressure delivery through a catheter and the like may be used to transfer recombinant vectors into host cells. Alternatively, the DNA may be transferred into cells through conjugation to proteins that are normally targeted to the inside of a cell. For example, the DNA may be conjugated to viral proteins that normally target viral particles into the targeted host cell. Additional techniques for the introduction of normal HF gene sequences into mammalian cells, e.g., human cells, will be readily selected by the person of ordinary skill in the art, given the benefit of this disclosure.

In accordance with certain examples, patients with dominant gain-of-function mutations may be treated by gene replacement therapy. A copy of the gene that can down-regulate a HF gene may be inserted into cells, e.g., cardiac cells, using viral or non-viral vectors which include, but are not limited to vectors derived from, for example, retroviruses, vaccinia virus, adenoviruses, adeno-associated virus, CMV, lentiviruses, herpes viruses, bovine papilloma virus or additional, non-viral vectors, such as plasmids. In addition, techniques frequently employed by those skilled in the art for introducing DNA into mammalian cells may be utilized. For example, methods including but not limited to electroporation, DEAE-dextran mediated DNA transfer, DNA guns, liposomes, direct injection, pressure delivery through a catheter and the like may be used to transfer recombinant vectors into host cells. Alternatively, the DNA may be transferred into cells through conjugation to proteins that are normally targeted to the inside of a cell. For example, the DNA may be conjugated to viral proteins that normally target viral particles into the targeted host cell. Additional techniques for the introduction of a gene into mammalian cells, e.g., human cells, to down-regulate a HF gene will be readily selected by the person of ordinary skill in the art, given the benefit of this disclosure.

In accordance with certain examples, in instances where a gene or HF gene is very large, e.g., 12 kbp or greater, the introduction of the entire gene coding region (or HF coding region) may be cumbersome and potentially inefficient as a gene therapy approach. However, because the entire gene product may not be necessary to avoid the appearance of HF symptoms, or treat HF symptoms, the use of a “minigene” therapy approach (see, e.g., Ragot, T. et al., 1993, Nature 3:647; Dunckley, M. G. et al., 1993, Hum. Mol. Genet. 2:717-723) may serve to ameliorate such HF symptoms. Such a minigene system comprises the use of a portion of a gene coding region which encodes a partial, yet active or substantially active gene product. As used herein, “substantially active” signifies that the gene product serves to ameliorate HF symptoms at least to some degree. Thus, the minigene system uses only that portion of a gene which encodes a portion of the gene product capable of ameliorating HF symptoms, and may, therefore represent an effective and even more efficient gene therapy than full-length gene therapy approaches. Such a minigene can be inserted into cells and utilized via the procedures described herein for full-length gene replacement. The cells into which the minigene is to be introduced are, preferably, those cells that are affected by HF gene up-regulation and/or down-regulation. Alternatively, any suitable cell can be transfected with a minigene as long as the minigene is expressed in a sustained, stable fashion and produces a gene product that ameliorates HF symptoms. Regulatory sequences by which such a minigene can be successfully expressed will vary depending upon the cell into which the minigene is introduced. The person of ordinary skill in the art, given the benefit of this disclosure, will be aware of appropriate regulatory sequences for a selected cell to be used. Techniques for such introduction and sustained expression are routine and are well known to those of ordinary skill in the art.

In accordance with certain examples, a therapeutic minigene for the amelioration of HF symptoms may include a nucleotide sequence which encodes at least one HF gene product peptide domain derived from the HF sequences (SEQ. ID NOS.: 1-1143 or SEQ. ID NOS: 1144-1233) disclosed herein. Among the ways whereby the HF minigene product activity can be assayed involves the use of HF knockout animal models, such as those described herein. The production of such animal models may be as described above, and involves methods well known to those of ordinary skill in the art. HF minigenes can be introduced into the HF knockout animal models as, for example, described above. The activity of the minigene can then be assessed by assaying for the amelioration of HF-like symptoms. Thus, the relative importance of each of the HF peptide domains, individually and/or in combination, with respect to HF gene activity can be determined. Cells, preferably, autologous cells, containing normal HF expressing gene sequences may then be introduced or reintroduced into the patient at positions which allow for the amelioration of HF symptoms. Such cell replacement techniques may be preferred, for example, when the HF gene product is a secreted, extracellular gene product.

In accordance with certain examples, a kit comprising one or more of the polynucleotides disclosed herein, or some portion thereof, may be used to diagnose patients with heart diseases or evaluate response to therapies, such as DCM. For example, the kit may include one or more polynucleotides selected from SEQ. ID NOS.: 1-1143 or SEQ. ID NOS.: 1144-1233. The kit may also include primers, enzymes (e.g., polymerases) and the like to provide for amplification of any DNA sequences in a patient sample. Additional components for inclusion in kits will be readily selected by the person of ordinary skill in the art, given the benefit of this disclosure.

In accordance with certain examples, one or more primers may be provided that is complementary to, or is the same as, the polynucleotide sequences disclosed herein. In certain examples, the primer comprises an effective amount of contiguous nucleotides from an oligonucleotide selected from the group consisting of SEQ. ID NOS.: 1-1143 or SEQ. ID NOS.: 1144-1233. As used herein, “an effective amount of contiguous nucleotides” refers to the number of nucleotides that are capable of providing a working primer to amplify a particular gene or nucleotide sequence. In certain examples, the effective amount of contiguous nucleotides is at least about 10, 15, 20, 25, 30, 35, 40 or 50 nucleotides, though fewer nucleotides may be used depending on the exact makeup of the gene. The primer may be the same as the polynucleotide sequences disclosed herein or may be complementary to the polynucleotide sequences disclosed herein. It will be within the ability of the person of ordinary skill in the art, given the benefit of this disclosure, to select suitable primers for use with the technology disclosed herein.

In accordance with certain examples, the identified compounds that inhibit HF expression, synthesis and/or activity can be administered to a patient at therapeutically effective doses to treat heart diseases, such as dilated idiopathic cardiomyopathy. A therapeutically effective dose refers to that amount of the compound sufficient to result in amelioration of symptoms of the heart disease. It will be recognized by the person of ordinary skill in the art, given the benefit of this disclosure, that the therapeutically effective dose may vary with patient age, sex, weight, metabolism, physical condition, overall health, disease stage, the presence of other compounds or drugs, etc.

In accordance with certain examples, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any selected compound, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography or other suitable analytical techniques. Additional factors that may be utilized to optimize dosage can include, for example, such factors as the severity of the HF symptoms as well as the age, weight and possible additional disorders which the patient may also exhibit. Those skilled in the art, given the benefit of this disclosure, will be able to determine the appropriate dose based on the above factors.

In accordance with certain examples, pharmaceutical compositions for use in accordance with the instant disclosure may be formulated in conventional manner using one or more pharmaceutically acceptable carriers or excipients. Thus, the compounds and their pharmaceutically acceptable salts and solvates may be formulated for administration by inhalation or insufflation (either through the mouth or the nose) or oral, buccal, parenteral or rectal administration or other selected methods commonly used to administer compounds in solid, liquid, aerosol or other form, e.g., direct cardiac injection, assist devices, stents, delivery devices such as nets that surround the heart, etc. For oral administration, the pharmaceutical compositions may take the form of, for example, tablets or capsules prepared by conventional means with pharmaceutically acceptable excipients such as, for example, binding agents (e.g., pregelatinized maize starch, polyvinylpyrrolidone or hydroxypropyl methylcellulose), fillers (e.g., lactose, microcrystalline cellulose or calcium hydrogen phosphate), lubricants (e.g., magnesium stearate, talc or silica), disintegrants (e.g., potato starch or sodium starch glycolate), or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled or sustained release of the active compound. For buccal administration the compositions may take the form of tablets or lozenges formulated in conventional manner. For administration by inhalation, compounds may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In accordance with certain examples, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

In accordance with certain examples, a variety of methods may be employed, utilizing reagents such as the HF polynucleotide sequences described herein, and antibodies directed against a HF gene product, as also described herein. Specifically, such reagents may be used for the detection of the presence of HF mutations, down-regulation of HF genes, up-regulation of HF genes levels, etc. The methods described herein may be performed, for example, by utilizing pre-packaged diagnostic kits, e.g., kits with cDNA chips, comprising at least one specific HF nucleic acid or anti-HF antibody reagent described herein, which may be conveniently used, e.g., in clinical settings, to diagnose patients exhibiting HF abnormalities or evaluating response to therapeutic interventions. Any tissue in which a HF gene product is expressed may be utilized in the diagnostics described herein.

In accordance with certain examples, RNA from a selected tissue to be analyzed may be isolated using procedures which are well known to those in the art. Diagnostic procedures may also be performed in situ directly upon tissue sections or biological samples (fresh, fixed and/or frozen) of patient tissue obtained from biopsies or resections, such that no RNA purification is necessary. Nucleic acid reagents such as those described herein, may be used as probes and/or primers for such in situ procedures (Nuovo, G. J., 1992, PCR in situ hybridization: protocols and applications, Raven Press, N.Y.). HF nucleotide sequences, either RNA or DNA, may, for example, be used in hybridization or amplification assays of biological samples to detect abnormalities of HF gene product expression; e.g., Southern or Northern analysis, single stranded conformational polymorphism (SSCP) analysis including in situ hybridization assays, alternatively, polymerase chain reaction analyses. Such analyses may reveal both quantitative abnormalities in the expression pattern of the HF gene, and, if the HF gene mutation is, for example, an extensive deletion, or the result of a chromosomal rearrangement, may reveal more qualitative aspects of the HF gene abnormality.

In accordance with certain examples, preferred diagnostic methods for the detection of HF specific nucleic acid molecules may involve for example, contacting and incubating nucleic acids, derived from the target tissue being analyzed, with one or more labeled nucleic acid reagents under conditions favorable for the specific annealing of these reagents to their complementary sequences within the target molecule. Preferably, the lengths of these nucleic acid reagents are at least about 15 to 30 nucleotides. After incubation, all non-annealed nucleic acids may be removed. The presence of nucleic acids from the target tissue which have hybridized, if any such molecules exist, is then detected. Using such a detection scheme, the target tissue nucleic acid may be immobilized, for example, to a solid support such as a membrane, or a plastic surface such as that on a microtiter plate or polystyrene beads. In this case, after incubation, non-annealed, labeled nucleic acid reagents are easily removed. Detection of the remaining, annealed, labeled nucleic acid reagents is accomplished using standard techniques well known to those or ordinary skill in the art. Alternative diagnostic methods for the detection of HF specific nucleic acid molecules may involve their amplification, e.g., by PCR (the experimental embodiment set forth in Mullis, K. B., 1987, U.S. Pat. No. 4,683,202), ligase chain reaction (Barany, F., 1991, Proc. Natl. Acad. Sci. USA 88:189-193), self sustained sequence replication (Guatelli, J. C. et al., 1990, Proc. Natl. Acad. Sci. USA 87:1874-1878), transcriptional amplification system (Kwoh, D. Y et al., 1989, Proc. Natl. Acad. Sci. USA 86:1173-1177), Q-Beta Replicase (Lizardi, P. M. et al., 1988, Bio/Technology 6:1197), or any other RNA amplification method, followed by the detection of the amplified molecules using techniques well known to those of skill in the art. These detection schemes are especially useful for the detection of RNA molecules if such molecules are present in very low numbers.

In accordance with certain examples, a cDNA molecule may be obtained from the target RNA molecule (e.g., by reverse transcription of the RNA molecule into cDNA). Tissues from which such RNA may be isolated include any tissue in which a wild type HF gene product is known to be expressed, including, but not limited, to cardiac tissue. A target sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the HF nucleic acid reagents described herein or primers suitable to anneal to one or more of the sequences disclosed herein (SEQ. ID NOS.: 1-1143 or SEQ. ID NOS.: 1144-1233). The preferred lengths of such nucleic acid reagents are at least 15-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

In accordance with certain examples, antibodies directed against a wild type, mutant HF gene product or aberrant HF gene product (e.g., misfolded gene product) or peptides may also be used as HF diagnostics, as described, for example, herein. Such diagnostic methods may be used to detect abnormalities in the level of HF protein expression, abnormalities in the location of the HF tissue, extracellular, cellular, nuclear, or subcellular location of HF protein, inoperative HF protein or HF protein with aberrant activity. For example, in addition, differences in the size, electronegativity, or antigenicity of a mutant HF protein relative to the normal HF protein may also be detected. Protein from the tissue to be analyzed may easily be isolated using techniques which are well known to those of ordinary skill in the art. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

In accordance with certain examples, preferred diagnostic methods for the detection of a wild type, aberrant or mutant HF gene product or peptide molecules may involve, for example, immunoassays wherein HF peptides are detected by their interaction with an anti-HF specific peptide antibody. For example, antibodies, or fragments of antibodies, such as those described above, may be used to quantitatively or qualitatively detect the presence of a wild type, aberrant or a mutant HF peptide. This detection can be accomplished, for example, by immunofluorescence techniques employing a fluorescently labeled antibody (see below) coupled with light microscopic, flow cytometric, or fluorimetric detection. Such techniques are especially preferred if a HF gene product or peptides are expressed on the cell surface. The antibodies (or fragments thereof) may additionally be employed histologically, as in immunofluorescence or immunoelectron microscopy, for in situ detection of HF gene product or peptides. In situ detection may be accomplished by removing a histological specimen from a patient, and applying thereto a labeled antibody. The histological sample may be taken, for example, from cardiac tissue suspected of exhibiting heart failure or heart disease symptoms. The antibody (or fragment) is preferably applied by overlaying the labeled antibody (or fragment) onto a biological sample. Through the use of such a procedure, it is possible to determine not only the presence of the HF peptides, but also their distribution in the examined tissue. The person of ordinary skill in the art, given the benefit of this disclosure, will readily perceive that any of a wide variety of histological methods (such as staining procedures) can be modified in order to achieve such in situ detection.

In accordance with certain examples, immunoassays for a wild type, aberrant or a mutant HF gene product or peptide typically comprises incubating a biological sample, such as a biological fluid, a tissue extract, freshly harvested cells, or cells which have been incubated in tissue culture, in the presence of a detectably labeled antibody capable of identifying HF peptides, and detecting the bound antibody by any of a number of techniques well-known in the art. The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled HF specific antibody. The solid phase support may then be washed with the buffer a second time to remove unbound antibody. The amount of bound label on solid support may then be detected by conventional means. By “solid phase support or carrier” is intended any support capable of binding an antigen or an antibody, e.g., wells of a microtiter plate, beads and the like. The term “solid phase support or carrier” may be used interchangeably herein with the term substrate. Well-known substrates include, but are not limited to, glass, polystyrene, polypropylene, polyethylene, polydimethylsiloxane, dextran, nylon, amylases, natural and modified celluloses, polyacrylamides, gabbros, and magnetite. The nature of the carrier can be either soluble to some extent or insoluble in water or a selected buffer or solvent. The support material may have virtually any possible structural configuration so long as the support material is capable of binding to an antigen or antibody or interacting with an antigen or antibody, e.g., through hydrophobic interactions. Thus, the support configuration may be spherical, as in a bead, or cylindrical, as in the inside surface of a test tube or well, or the external surface of a rod. Alternatively, the surface may be flat such as a sheet, test strip, chip, array, microarray, etc. The person of ordinary skill in the art, given the benefit of this disclosure, will select many other suitable carriers for binding antibody or antigen, or will be able to ascertain the same using the instant disclosure.

In accordance with certain examples, the binding activity of a given lot of anti-wild type or mutant HF peptide antibody may be determined according to well known methods. The person of ordinary skill in the art, given the benefit of this disclosure, will be able to determine operative and optimal assay conditions for each determination by employing routine experimentation. For example, one of the ways in which the HF peptide-specific antibody can be detectably labeled is by linking the same to an enzyme and use in an enzyme immunoassay (EIA) (Voller, A., “The Enzyme Linked Immunosorbent Assay (ELISA)”, Diagnostic Horizons 2:1-7, 1978) (Microbiological Associates Quarterly Publication, Walkersville, Md.); Voller, A. et al., J. Clin. Pathol. 31:507-520 (1978); Butler, J. E., Meth. Enzymol. 73:482-523 (1981); Maggio, E. (ed.), ENZYME IMMUNOASSAY, CRC Press, Boca Raton, Fla., 1980; Ishikawa, E. et al., (eds.) ENZYME IMMUNOASSAY, Kgaku Shoin, Tokyo, 1981). The enzyme which is bound to the antibody will react with an appropriate substrate, preferably a chromogenic substrate, in such a manner as to produce a reaction product that can be detected, for example, by spectrophotometric, fluorimetric or by visual techniques. Enzymes which can be used to detectably label the antibody include, but are not limited to, malate dehydrogenase, staphylococcal nuclease, delta-5-steroid isomerase, yeast alcohol dehydrogenase, alphaglycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection may be accomplished by colorimetric methods which employ a chromogenic substrate for the enzyme. Detection may also be accomplished by visual comparison of the extent of enzymatic reaction of a substrate in comparison with similarly prepared standards. Detection may also be accomplished using any of a variety of immunoassays. In some examples, an ELISA on a microchip with electrochemical detection may be used. In other examples, a paramagnetic ion, e.g., for NMR or ESR spectroscopy, may be used. In yet other examples, quantum dots or radioisotopes may be used. For example, by radioactively labeling the antibodies or antibody fragments it is possible to detect HF wild type or mutant peptides through the use of a ELISA, bispecific enzyme linked signal enhanced immunoassay (BiELSIA) radioimmunoassay (RIA) (see, for example, Weintraub, B., Principles of Radioimmunoassays, Seventh Training Course on Radioligand Assay Techniques, The Endocrine Society, March, 1986, which is incorporated by reference herein) or the like. The radioactive isotope can be detected by such means as the use of a gamma counter or a scintillation counter or by autoradiography.

In accordance with certain examples, it is also possible to label the antibody with a luminescent compound. When the luminescently labeled antibody is exposed to light of the proper wavelength, its presence can then be detected due to luminescence, e.g., fluorescence or phosphorescence. Among the most commonly used luminescent labeling compounds are fluorescein isothiocyanate, rhodamine, phycoerythrin, phycocyanin, allophycocyanin, o-phthaldehyde, fluorescent beads, and fluorescamine. The antibody can also be detectably labeled using fluorescence emitting metals such as ¹⁵²Eu or other species in the lanthanide or actinide series or species that are transition metals. These metals can be attached to the antibody using such metal chelating groups as, for example, diethylenetriaminepentacetic acid (DTPA) or ethylenediaminetetraacetic acid (EDTA). The antibody also can be detectably labeled by coupling it to a chemiluminescent compound or an electrochemiluminescent compound, e.g., dinitrophenyl (DNP). The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound may be used to label the antibody. The presence of a bioluminescent protein may be determined by detecting the presence of luminescence. Illustrative bioluminescent compounds for purposes of labeling are luciferin, luciferase, aequorin and quantum dots.

Certain specific examples are described below to further illustrate some of the features, aspects and embodiments of the technology described herein.

### EXAMPLE 1

In accordance with certain examples, findings in the avian model were compared to studies of human myocardium from patients with heart failure as well as non-failing donor hearts. These studies revealed several key factors associated with heart failure. This example describes some results to demonstrate (1) that Fz-treatment of turkey poults leads to the development of DCM, and (2) that Fz-induced DCM in turkey poults shares several key features with human DCM. One of the characteristics of human DCM is decreased energy metabolism, marked by a decrease in energy markers, such as citrate synthase, lactate dehydrogenase, creatine kinase, and creatine (Nacimben at al., 1991, Hammer et al. 1989). Furthermore, intracellular cAMP levels are decreased due to a down-regulation of β1-receptors in the sarcolemmal membrane (Bristow et al., 1986, Feldman at al., 1981). Other hallmarks of human DCM are reduced sarcoplasmic (SR) ATPase (Limnas et al., 1987), reduced myofibrillar ATPase (Pagani et al., 1988), negative force interval relationship, slowed time course of the calcium transient, and overall reduced myofibrillar protein content (Gwathmey et at. 1987, 1988).

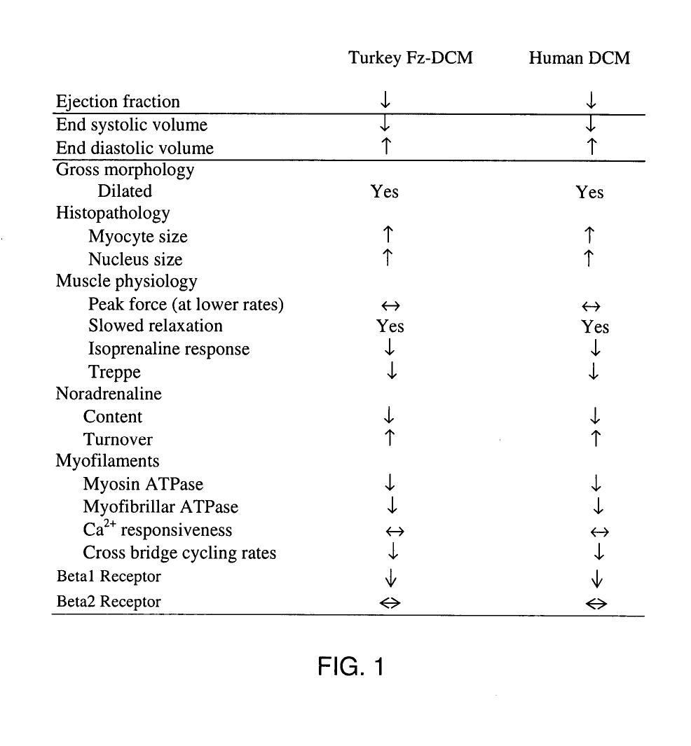

It has been observed that the peak force in isolated muscle strips stimulated at lower frequencies is similar if not greater in normal and diseased human myocardium (Boehm et al., 1991, Feldman et al., 1987, Gwathmey and Hajjar, 1990, Gwathmey at al., 1992). However, with higher rates of stimulation there is a decrease in peak twitch force (i.e., negative treppe). Studies in the avian model of DCM have shown that in all these features in Fz-DCM and human DCM correlate. A marked decrease in energy markers, such as citrate synthase, lactate dehydrogenase, creatine kinase and creatine, was observed. After three weeks of Fz treatment, at a time of acute heart failure, all energy markers were decreased, but additionally there was a decrease in SR-Ca²⁺-ATPase activity and myofibrillar ATPase activity, suggesting that a decrease in energy supply may contribute to heart failure. A summary of the similarities of human DCM and avian DCM is shown in FIG. 1.

### EXAMPLE 2

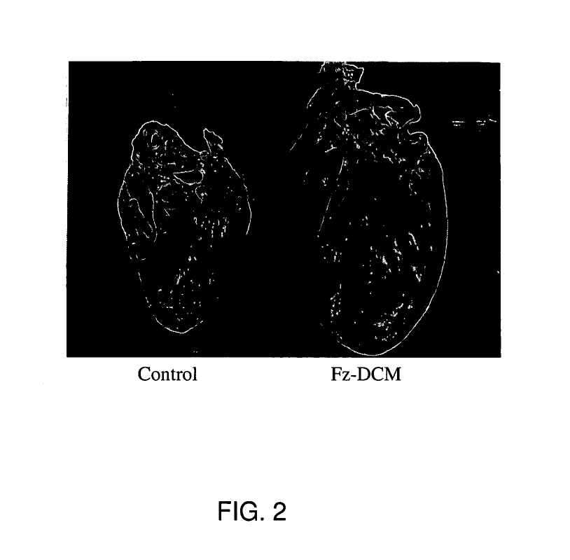

In accordance with certain examples, Fz treatment leads to the development of DCM in turkey poults. FIG. 2 shows a typical control heart and a Fz-DCM heart. There is marked dilation with wall thinning (Hajjar et al., 1993). Hearts from Fz-DCM animals are also enlarged with increased weight, left ventricular wall thinning and have increased left ventricular volume, as listed in Table 3 (Hajjar et al., 1993). In Table 3, HW=Heart Weight; BW=Body Weight; LV=Left Ventricle; *P<0.05 compared to control. An increase in HW/BW ratio indicative of heart failure and heart enlargement is demonstrated in the DCM group.

TABLE 3

| | | LV volume | LV width | LV thickness | |
|---|---|---|---|---|---|
| Group | HW/BW (%) | (ml) | (mm) | (mm) | N |
| Control | 0.67 ± 0.13 | 0.4 ± 0.2 | 29 ± 2.3 | 4.3 ± 0.5 | 9 |
| DCM | 0.88 ± 0.23* | 2.7 ± 1.8* | 35.3 ± 6.6* | 3.8 ± 0.9 | 10 |

### EXAMPLE 3

In accordance with certain examples, an extensive analysis of energy marker levels in DCM animals versus normal animals is shown in Table 4 below. In addition, SR-Ca²⁺-ATPase and myofibrillar ATPase activities were reduced, and as described above, the levels correlate with observations made in human DCM hearts. In Fz-DCM hearts, the myofibrillar protein content was reduced when compared to control animals (average±standard error of the mean)−46.3±3.2 mg/g in control animals vs. 34.6±2.5 mg/g in Fz-DCM animals (p<0.01). The values shown in Table 4 are the average values±the standard error. The values in parentheses indicate the number of hearts. CK is creatine kinase, LDH is lactate dehydrogenase, and AST is aspartate transaminase. Ca²⁺-ATPase activity was normalized per gram of protein. * represents p<0.05.

TABLE 4

| Metabolic Marker | Control | DCM |
|---|---|---|
| Total ATPase, IU/g | 35.5 ± 1.9 (7) | 16.8 ± 0.9* (4) |
| CK, IU/g | 2,450 ± 94 (18) | 1,400 ± 129* (9) |
| LDH, IU/g | 275 ± 8 (18) | 219 ± 12* (9) |
| AST, IU/g | 274 ± 8.8 (17) | 187 ± 8.2* (9) |
| ATP synthase, IU/g | 145 ± 4.2 (8) | 87 ± 4* (4) |
| Myoglobin, μg/g | 50.9 ± 6.7 (10 | 27.2 ± 3.1* (5) |
| Total protein mg/g | 128 ± 2 (36) | 111 ± 3.0* (8) |
| SR Ca²⁺ cycling | | |
| Ca²⁺-ATPase, IU/g | 11.4 ± 0.7 (8) | 3.4 ± 0.6* (4) |
| Ca²⁺-ATPase pump, nM/s | 41.8 ± 2.1 (23) | 24.4 ± 6.3* (9) |

### EXAMPLE 4

In accordance with certain examples, to document the progression of Fz-DCM development, gross morphological studies of the turkey heart may be performed. Criteria for DCM in turkey poults are typically: (1) larger heart weight, (2) larger heart-to-body weight ratio, (3) left ventricle wall thinning, (4) septum wall thinning, and (5) increased left ventricle volume. Animals may be wing-banded for easy identification at age 1 day and housed in heated brooders. The animals may be fed a commercial starter mash and water. Birds may be randomized into control or Fz group at 7 days of age. For example, animal groups may be as shown in Table 5.

TABLE 5

| | | | | 2 weeks |
|---|---|---|---|---|
| | Treatment Time (weeks) | | | off Fz |
| | 1 | 2 | 3 | 5 |
| Untreated | 6 | 6 | 6 | 6 |
| Lower dose Fz (500 ppm) - control | 6 | 6 | 6 | 6 |
| Higher does Fz (700 ppm) - DCM | 6 | 6 | 6 | 6 |

Each group of six animals typically includes three males and three females to account for gender-specific gene expression. Untreated animals are generally not used for subtractive screening. However, gross morphological measurements from untreated animals may be used to confirm the absence of DCM development in the animals treated with a lower dose of Fz. Animals taken off Fz for two weeks may undergo gross morphological studies to confirm the presence of DCM in the higher dose animals and the absence of DCM in the lower dose animals. Tissues are typically stored at 80° C.

DCM animals may receive a high (700 ppm) dose of Fz. The control animals may receive a lower dose of Fz (500 ppm) that has been shown to not induce DCM (unpublished data), in order to subtract gene expression that might be related to the effects of Fz-treatment rather than to the development of DCM. The concentration of 300-500 ppm has been previously established in pilot studies.

Six animals (three males and three females) may be randomly euthanized from each group (control, low dose and high dose) on week one, two and three of Fz treatment. It is expected that after three weeks of Fz treatment, 100% of the birds receiving the high dose of Fz have DCM. Fz may then be removed from the feed of all remaining animals for an additional two weeks prior to euthanasia with pentobarbital. After two weeks off Fz, another group of six animals may be euthanized from each group (control, low dose and high dose) for comparison. We have shown that animals receiving 700 ppm Fz remain myopathic, and that animals receiving 500 ppm Fz do not develop DCM after Fz removal from the feed for three weeks. Furthermore, no Fz can be detected in feces or blood after two weeks (unpublished data). The gross morphological studies on these animals may serve as further proof that a dose of 500 ppm Fz does not induce the development of DCM, and that lower dose animals are a valid control for high dose Fz-DCM animals.

Before sacrificing, the animals may be weighed. The hearts may then be excised quickly and weighed to establish the heart to body weight ratios. The following gross morphological studies may be then performed on all animals. The atria may be excised and the left ventricle arrested in diastole and filled with normal saline and a LV heart volume recorded. Measurements of left ventricle and septum walls may be taken at the level of the mitral valve as previously described (Gwathmey 1991). The diameter of the left ventricular lumen may be measured just apical to the mitral orifice and just basilar to the apex of the posterior papillary muscle. The means of each measurement may be calculated for each group. The left ventricle walls may be dissected and used for further studies. The LV may be placed in liquid nitrogen and stored at −80° C. for later use. The right ventricle, left and right atria, and septum wall may also be placed in liquid nitrogen and stored at −80° C.

### EXAMPLE 5

In accordance with certain examples, the expected results of a subtraction experiment are discussed now. The result of a subtraction experiment should be six subtracted cDNA pools (see Table 6 below): 1) genes that are differentially expressed during early DCM development (one week after 700 ppm Fz treatment) versus 2) genes that are exclusively expressed in normal tissues and turned off during early DCM development. (These cDNA pools may be referred to as “Forward 1” versus “Reverse 1”, respectively) versus 3) genes that are differentially expressed two weeks after 700 ppm Fz treatment versus 4) genes that are exclusively expressed in normal tissues and turned off two weeks after 700 ppm Fz treatment. (These cDNA pools may be referred to as “Forward 2” versus “Reverse 2”, respectively) versus 5) genes that are differentially expressed during heart failure (three weeks after 700 ppm Fz treatment) versus 6) genes that are exclusively expressed in normal tissues and turned off during heart failure. (These cDNA pools may be referred to as “Forward 3” versus “Reverse 3”, respectively). Pools 1, 3, and 5 may contain very similar expression profiles, as may pools 2, 4, and 6. These cDNAs may be used to construct stage-specific cDNA libraries that can be used in a differential screening step to reduce further a background of genes expressed in both, the tester and the driver samples. In Table 6 below, high Fz=700 ppm Fz in feed, which we have shown to result in DCM after three weeks in 100% of animals, lower Fz=500 ppm Fz which was shown to not induce DCM. Hearts of animals that have been taken off the drug for two weeks after 3 weeks of treatment with the high and lower doses of Fz may be used for gross morphological studies and stored for potential later use.

TABLE 6

| Subtracted cDNA libraries | |
|---|---|
| 1. High Fz (1 week)-lower Fz (1 week) | Forward 1 |
| 2. Lower Fz (1 week)-High Fz (1 week) | Reverse 1 |
| 3. High Fz (2 weeks)-lower Fz (2 weeks) | Forward 2 |
| 4. Lower Fz (2 weeks)-High Fz (1 week) | Reverse 2 |
| 5. High Fz (3 weeks)-Lower Fz (3 weeks) | Forward 3 |
| 6. Lower Fz (3 weeks)-High Fz (3 weeks) | Reverse 3 |

### EXAMPLE 6

Two subtracted pools of cDNA were produced and cloned. Because furazolidone (Fz) at 700 ppm leads to idiopathic dilated cardiomyopathy (DCM) in the turkey model, the first subtracted cDNA pool was produced using cDNA derived from a group of untreated turkey hearts subtracted from cDNA isolated from a group of furazolidone (Fz-700 ppm) treated turkey hearts. The resulting subtracted cDNA pool was enriched for differentially expressed sequences unique to the DCM turkey heart tissue.

In order to identify genes that are differentially expressed in turkey heart failure due to induction of DCM and not due to a Fz drug effect, a second subtracted cDNA pool was produced. It has been previously reported that lower doses of Fz (500 ppm) do not lead to heart failure in the turkey model. The second cDNA pool was produced using cDNA isolated from turkey hearts that had been treated with a low dose of Fz (500 ppm). This pool of cDNA was subtracted from cDNA derived from DCM turkey hearts. The resulting subtracted cDNA pool was enriched for differentially expressed sequences unique to the DCM turkey heart tissue.

The subtractive hybridization produced an enrichment of differentially expressed sequences in the subtracted population, but this cDNA population still contained some cDNA sequences that are common to both populations. In some instances, the number of genes that are differentially expressed are few. Therefore, a differential screening method was used to efficiently identify those genes that were truly unique to the subtracted cDNA population and thus, unique to the DCM (high dose Fz-treated) turkey heart tissue.

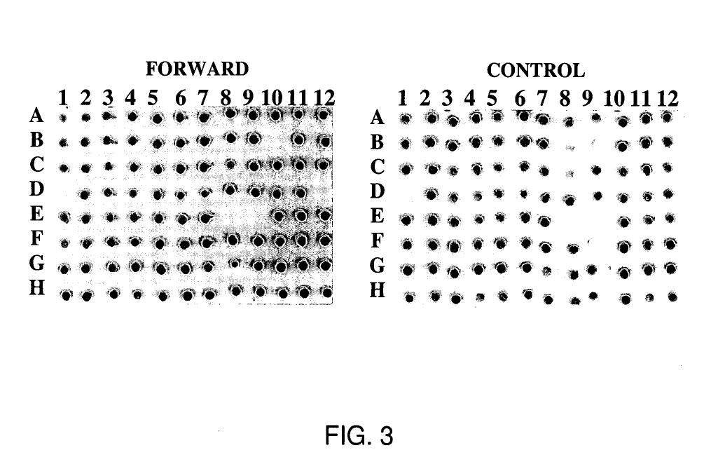

This method of differentially screening the subtracted cDNA libraries involved hybridizing clones of the subtracted library with labeled forward subtracted, reverse subtracted, and unsubtracted pools of cDNA. An example of a pair of hybridized blots is shown in FIG. 3. The left panel of FIG. 3 shows the forward subtracted sample. Compared to the control (right panel), the darker the spot the higher degree of overexpression of the gene. 10 μL of each purified PCR product was combined with 10 μL 0.6 N NaOH in a 96-well microtiter dish format. Using a multi-channel pipette, 2 μL of this PCR mixture was spotted onto a gridded nylon membrane (Hybond N+, Amersham). Three replicate spotted membranes were produced for each microtiter dish. The membranes were neutralized with 0.5M Tris pH. 7.5, equilibrated with 5×SSC, UV linked and baked at 70° C. Each membrane was hybridized with a labeled probe produced from the purified secondary PCR products (using conventional PCR purification methods) of forward subtracted cDNA, reverse subtracted cDNA, and unsubtracted cDNA.

### EXAMPLE 7

Clones produced using the methods of Example 6 were selected for subsequent sequencing and identification based on the following criteria: (1) Clones that hybridized to the forward-subtracted and unsubtracted probes but not to the reverse-subtracted probe were identified as putative differentially expressed genes; (2) Clones that hybridized only to the forward-subtracted probe and not to the reverse-subtracted and unsubtracted probes were identified as strong candidates for differentially expressed genes—these clones may correspond to low-abundance transcripts that were enriched during the subtraction procedure; (3) Clones that hybridized to both the forward and reverse subtracted probes but hybridized with an increased intensity (greater than five-fold) to the forward-subtracted probe were also identified as possible differentially expressed genes.

Clones from the forward subtracted cDNA library were sequenced and analyzed by similarity searches. Based on Basic Local Alignment Search Tool (BLAST) searches of the Genbank nucleotide, protein, and EST databases, the identified sequences represented genes of both known and unknown function. Table 7 summarizes the data on the differentially expressed genes. About 305 differentially expressed genes were identified, and the exact function of about 102 of these genes remains unknown. Unknown genes are defined as those showing no meaningful similarity to genes of known function by BLASTX (amino acid blast search) and BLASTN (nucleotide blast search) analyses.

TABLE 7

| | Number of Genes differentially | | |
|---|---|---|---|
| | expressed | | |
| Number of | | | 10 fold |
| Genes | | 2.5-3 fold | difference |
| unknown | Total | difference | or greater |
| 102 | 305 | 273 | 32 |

A selection of certain genes identified is shown below in Table 8.

TABLE 8

| | | Blast | |
|---|---|---|---|
| Putative Identification | Organism | Search | E Value |
| Titin | Chicken | Blast X | 6E−67 |
| Phosphodiesterase interacting protein | Homo sapiens | Blast X | 3E−26 |
| Troponin T | Homo sapiens | Blast X | 4E−46 |
| Titin Isoform | Homo sapiens | Blast X | 2E−17 |
| Myosin regulatory light chain cardiac | Chicken | Blast X | 1E−84 |
| muscle isoform | | | |
| Phospholamban gene | Chicken | Blast N | 2E−45 |
| Calmodulin | Chicken | Blast N | 0 |
| ATPase Ca⁺⁺ transporting cardiac | Rat | Blast X | 8E−71 |
| muscle | | | |
| Phosphorylase Kinase (muscle) | Homo Sapiens | Blast X | 1E−35 |
| Heart alpha kinase | Mus Musculus | Blast X | 2E−59 |
| Adenovirus | Homo Sapiens | Blast X | 1E−97 |
| receptor protein | | | |

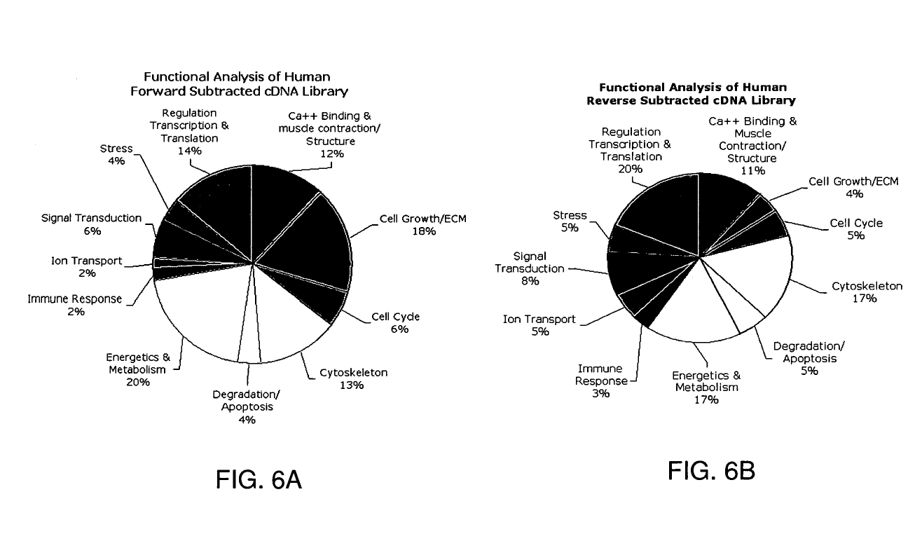

A sequence listing of some of the sequences that were used may be found at SEQ. ID NOS.: 1144-1233 in the Sequence Listing appended hereto. A gene representation based on functional groups for each subtracted library is shown in the pie charts in FIGS. 6A and 6B.

### EXAMPLE 8

Using the technique of subtractive suppression hybridization (SSH) both a forward (DCM minus non-failing) and reverse (non-failing minus DCM) subtracted cDNA library was constructed following the manufacturers instructions (Clontech, Mountain View, Calif.). Each library was constructed using pooled mRNA from left ventricle tissue of 5 male, 6 female (group a), and 6 female (group b) DCM transplant patients and pooled mRNA from 10 non-failing donors.

Patient consent was obtained from all transplant patients. Family consent was provided for brain dead organ donors. Hearts from donors were due to cardiac arrest with resuscitation, blood transfusion, or lack of a suitable recipient. The clinical characteristics of the DCM transplant patients are summarized in Table 9. In Table 9, ND refers to no data, FS (%) refers to percent fractional shortening, LVEF refers to left ventricular ejection fraction, PCW refers to pulmonary capillary wedge pressure, M refers to male, and F refers to female. Each male and female patient shown was diagnosed with idiopathic dilated cardiomyopathy (DCM) and underwent cardiac transplantation.

TABLE 9
Patient data

| | | | FS | | |
|---|---|---|---|---|---|
| Sex | Age | LVEF | (%) | PCW | Medications |
| M | 65 | 76 | 15 | 25 | Lasix, Digoxin, Captopril, Coumadin, Cozaar, KCL |
| M | 57 | ND | 20 | 16 | Furosemide, Spironolactone, Milrinone, Amiodarone, |
| | | | | | Allopurinol, Isosorbide, Celexa, Tapazole, Lipitor, Nexium, |
| | | | | | Warfarin, Cozaar, KCl, Teroxalene, Vitamin D |
| M | 64 | 75 | 20 | 19 | Lasix, Aldactone, Lisinopril, Coumadin, Lipitor, Flomax, |
| | | | | | Azmacort, Albuterol |
| M | 47 | N/D | 20 | 23 | Lasix, Spironolactone, Digoxin, Captopril, Isodinitrate |
| M | 56 | 51 | 22 | 26 | None |
| F | 55 | 67 | 22 | 29 | Aldactone digoxin, Captopril, Atrovent, Nexium, |
| | | | | | Glyburide, Singulair, Theophilline, Tapazole, Cozaar, Prev |
| | | | | | Amiod |
| F | 63 | 64 | 10 | 35 | Diuretic, Aspirin |
| F | 43 | 69 | 15 | 34 | Hydrochlorothiazide, Metoprolol, Amlodipine, Prazosin, |
| | | | | | Folate, Valacyte |
| F | 65 | 71 | 10 | 28 | Spironolactone, Furosemide, Digoxin, Captopril, |
| | | | | | Amiodarone, Atorvastatine, Paroxetine, Levothyroxine |
| F | 31 | 80 | 10 | 14 | Lasix, Spironolactone, Coreg, Digoxin, Captopril, MMF, |
| | | | | | Heparin, Nexium, Folate, Iron, Paxil |
| F | 33 | 65 | 23 | 20 | Torsemide, Spironolactone, Digoxin, Captopril, Folic acid, |
| | | | | | Thiamine, Amiodarone, Levothyroxine, Allopurinol, |
| | | | | | Esomeprazole, Warfarin, Acetaminophen |
| F | 39 | ND | ND | ND | Carvedilol, Digoxin, Losartan, Coumadin, Nipride, L- |
| | | | | | Thyroxine, Ranitidine, Spironolactone, K-dur, Mg |
| | | | | | gluconate |

Each male and female patient shown was diagnosed with dilated cardiomyopathy (DCM) prior to transplantation. FS (%)=Percent fractional shortening, LVEF=Isolated left ventricular ejection fraction, PCW=Pulmonary capillary wedge pressure, M=male, F=female. The average age of the 5 male and 6 female patients was 57+/−6.5 yrs. and 48+/−14.8 yrs. (p>0.05), respectively, and the average age of the pooled non-failing male and female donor samples are 58+/−5.5 yrs and 57+/−4.5 yrs (p>0.05). All patients presented idiopathic dilated cardiomyopathy at the time of transplantation. All female and male patients were classified as non-ischemic (no evidence of coronary artery disease). At the time of transplantation, the majority of patients were on diuretics, digoxin, angiotensin converting enzyme inhibitors (ACE-I), and anticoagulants.

Left ventricle tissue was pulverized in liquid nitrogen, placed in TRIZOL® reagent and immediately homogenized using a rotor-stator homogenizer. Total RNA was isolated according to the manufacturer's instructions (Invitrogen, Carlsbad, Calif.) with the following exceptions. An additional extraction with phenol (pH 4.3)/chloroform was performed as well as an additional isopropanol precipitation to purify further the RNA.

Messenger RNA (mRNA) was purified from each total RNA sample using the Poly(A) Pure mRNA isolation Kit (Ambion, Inc., Austin, Tex.). 700 μg of total RNA was used for each sample and mRNA isolation was performed according to the manufacturer's instructions. The eluted mRNA was ethanol precipitated and washed once with 70% ethanol for purification and concentration.

The forward subtracted DCM cDNA library is enriched for genes that are increased in expression levels or turned on during DCM. Conversely, the reverse subtracted cDNA is enriched for genes that are decreased or turned off during DCM. Over one thousand clones were randomly chosen from each library, PCR amplified, and sequenced on a single pass basis to produce an expressed sequence tag (EST) for each clone. Sequences were identified through NCBI database queries.

Genes that showed expression differences in human heart failure tissues by means of subtractive suppression hybridization were used to make a human heart failure microarray. All contigs (consensus sequence of clustered EST's representing one gene) representing a gene derived from both forward and reverse human subtracted cDNA libraries, identified through NCBI database queries, were chosen for production of a heart failure oligo microarray. GenBank accession numbers were obtained for each contig representing a gene of known function and the full-length database sequence of these known genes were used for oligo design. Contig sequences representing genes of unknown function were also used for oligo design. A total number of 1,143 genes (SEQ. ID NOS.: 1-1143) were represented on the heart specific microarray along with 8 control oligonucleotides representing sequences that do not hybridize to mammalian sequences (Ambion, Austin, Tex.). Microarray oligos (70 nucleotides in length) were designed for each contig representing a human gene using ArrayOligoSelector software and the oligonucleotides were synthesized by Illumina (San Diego, Calif.). These oligonucleotides were spotted in triplicate onto epoxy-coated slides obtained from MWG (Germany) and stored at −20° C.

cDNA was synthesized from 2 μg of total RNA isolated from left ventricle tissue of each patient (DCM) or non-failing left ventricles were pooled as controls (n=10). cDNA construction and microarray hybridization were performed using the 3DNA Array 900 Detection System (Genisphere, Inc., Hatfield, Pa.) following the manufacturers instructions except cDNA hybridization was performed over night at 62° C. Seventy four additional sequences not identified through SSH but thought to play a role in heart failure according to recent published microarray data (Barrans D J et al. American Journal of Pathology, 2002; 160 (6):2035-2043; Hwang J J et al. Physiol Genomics. 2002; 10: 31-44; Grzeskowiak R et al., Cardiovascular Research. 2003; 59: 400-41; Tan F-L et al. PNAS. 2002; 99(17):11387-11392; Steenman M et al. Physiol Genomics. 2003; 12:97-112) were added to the microarray as additional oligonucleotide probes as a means to verify previous array studies. Oligonucleotides representing 18S rRNA and GADPH were added to the microarray as control sequences.

Based on gene expression differences in turkey heart failure tissues obtained from the subtractive hybridization screening as well as the validation of genes specific for heart failure in the avian model and human, gene were selected for printing on an avian heart failure specific microarray. All contigs (consensus sequence of all EST's resulting from one gene) derived from both forward and reverse turkey subtracted cDNA libraries and identified through NCBI database queries, were chosen for production of a first heart failure oligo microarray. GenBank accession numbers were obtained for each contig representing a gene of known function and the full-length database sequence of these known genes were used for oligo design. Contig sequences representing genes of unknown function were also used for oligo design. A total number of 1,143 genes were printed in a custom human heart failure microarray. These genes represent three categories, heart failure specific genes (1061 genes), control genes (8) (sequences that do not hybridize to mammalian sequences) and 74 additional sequences not identified through SSH were added to the microarray as additional oligonucleotide probes as a means to verify previous array studies. Control RNA transcripts corresponding to the oligonucleotide controls were used in the hybridization process for the normalization and validation of gene array data. Microarray oligos (70 nucleotides in length) were designed for each contig representing a turkey gene using ArrayOligoSelector software and the oligos were synthesized by Illumina (San Diego, Calif.). These oligos were spotted in triplicate onto epoxy-coated slides obtained from MWG (Germany) and stored at −200° C.

Hybridizations were performed as follows: cDNA was synthesized from 2 μg of total RNA isolated from left ventricle tissue of turkeys with heart failure or normal left ventricles were pooled as controls. cDNA construction and microarray hybridization were performed using the 3DNA Array 900 Detection System (Genisphere) following the manufacturers instructions. A total of two technical replicates for each patient were performed (dye swap). cDNA hybridization was performed over night at 62° C. The slides were washed and then hybridized to fluorescent dendrimers. The microarray slides were scanned twice in a Perkin Elmer HT scanner. Photomultiplier (PMT) values were set at 69 and 60 volts for Cy3 and Cy5, respectively. An additional scan was done for each slide with the PMT set at 54 and 46 volts.

The Cy3 and Cy 5 scans for each slide were superimposed onto each other, and values corresponding to the fluorescence intensity for each oligonucleotide spot were obtained, exported to an Excel spreadsheet, and imported into GeneSpring 7.1 software (Agilent, Redwood city, Calif.). Local background fluorescence intensity was subtracted from individual spot fluorescence intensities. The mean signal and control intensities of the on-slide duplicate spots were calculated. A Lowess curve was fit to the log-intensity versus log-ratio plot. Twenty percent of the data was used to calculate the Lowess fit at each point. This curve was used to adjust the control value for each measurement. If the control channel was lower than 10 relative fluorescence units (RFUs) then 10 was used instead. Mean signal to Lowess adjusted controlled ratios were calculated. The cross-chip averages were derived from the antilog of the mean of the natural log ratios across the 2 microarrays (technical replicates-dye swaps). Oligonucleotide elements that received a “present” call (intensity>200 RFUs or local signal-to-background>2) by the ScanArray software in one of the on-slide replicates in at least half the transplant recipients in either the Cy3 or Cy5 were identified (1037 genes), and all others were excluded from the analysis.

Data were filtered using the coefficient of variation (CV) function in GeneSpring software. The genes with a CV<30% between the dye swaps for each patient were selected. A list of genes which appeared in 70% of the CV<30% lists was compiled. Genes were selected from the 70% list, which were at least 1.8-fold up or down-regulated in 3 of 5 male and 3 of 6 female transplant recipients as compared to the pooled male or female non-failing control samples respectively.

Clustering analysis produced 535 contigs (consensus sequence of clustered EST's representing one gene) unique to the forward subtracted library and 495 contigs uniquely represented in the reverse subtracted library. Sequences identified by means of BLAST alignment to the Genbank databases showed 95%-100% homology at the nucleic acid level. Seventy five percent of those contigs were identified and assigned a function. All contig sequences with both known and unknown function were used to produce an oligonucleotide based human heart failure microarray. As a result, the heart failure gene array contained 1,143 heart specific oligonucleotide probes (SEQ. ID NOS.: 1-1143).

To address the question of gender-specific gene expression in end-stage DCM left ventricle tissue, individual DCM RNA was hybridized to the heart failure microarray against pooled samples from non-failing female donors (n=10) or non-failing male donor (n=10) RNA samples.

Microarray data filtering analysis was performed to identify genes that are differentially expressed in female and male DCM left ventricle tissue. A gene was considered significantly up or down-regulated if the average normalized fluorescence showed a fold difference of at least 1.8 (compared to non-failing female or male samples from non-failing hearts n=10) in at least 3 of the 5 male or 3 of 6 female patients (P<0.05).

Tables 10 and 11 lists 80 genes determined by means of statistical analysis to be differentially expressed in female end-stage heart failure consequent to DCM (53 up-regulated (Table 9); 27 down-regulated (Table 10)). In Tables 9 and 10, bolded and italicized rows represent genes that were found to be coordinately up or down-regulated (at least 1.8×) in at least 3 of 5 male and 3 of 6 female transplant recipient samples. Rows that include a “*” represent genes that were found to be coordinately up or down-regulated (at least 1.8×) in at least 3 of 6 transplant recipients. Fold change represents mean fold change in 6 female transplant recipients and 5 male transplant recipients.

TABLE 10
Up-Regulated Genes

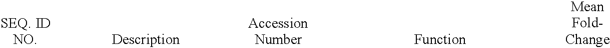

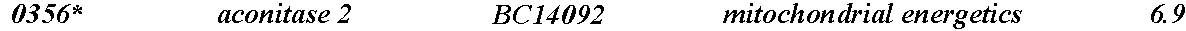

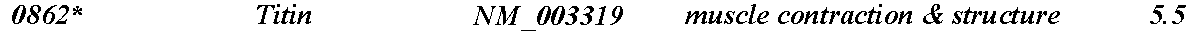

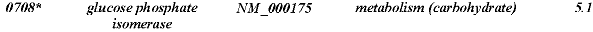

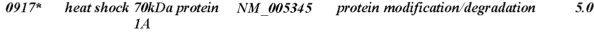

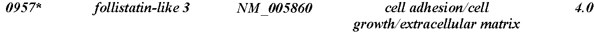

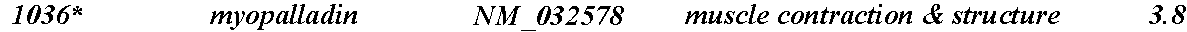

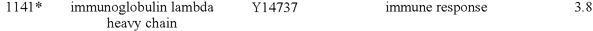

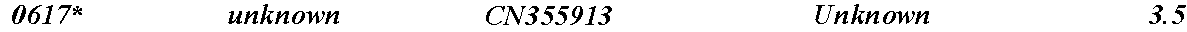

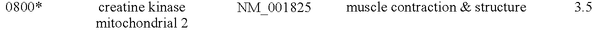

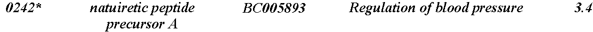

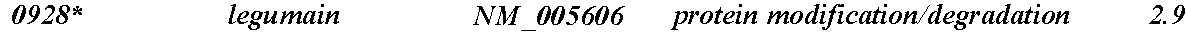

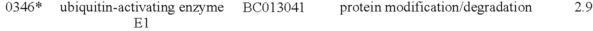

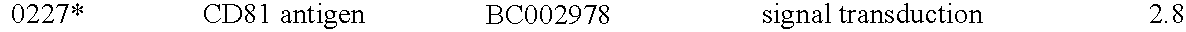

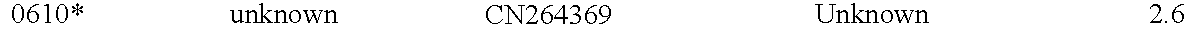

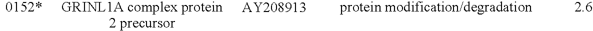

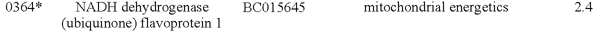

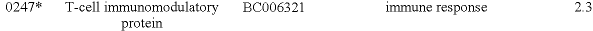

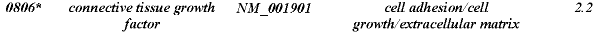

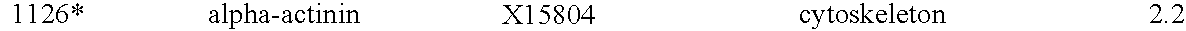

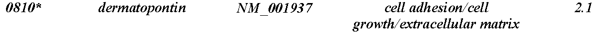

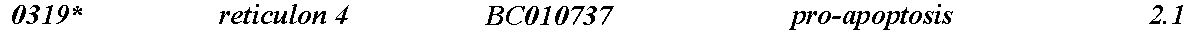

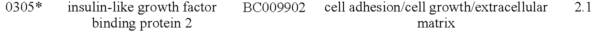

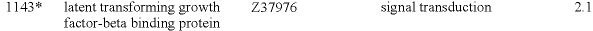

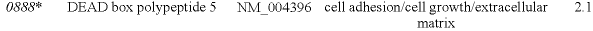

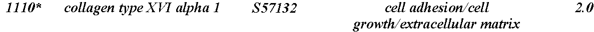

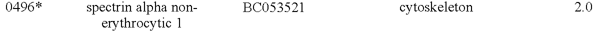

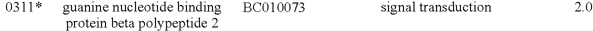

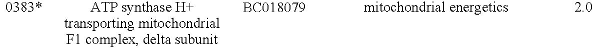

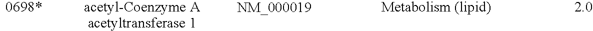

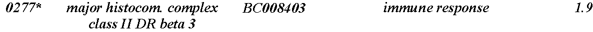

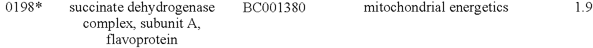

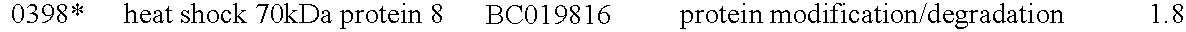

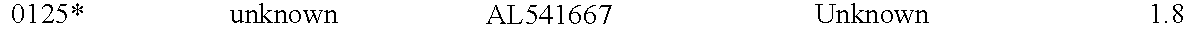

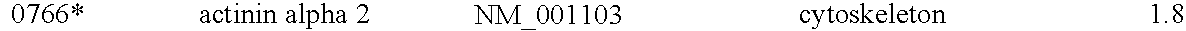

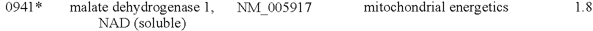

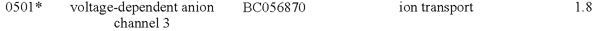

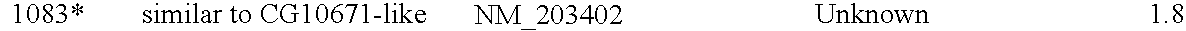

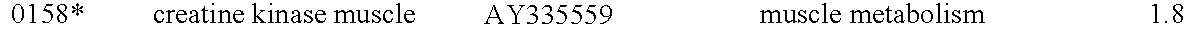

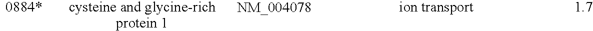

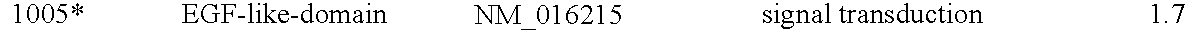

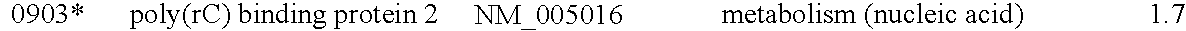

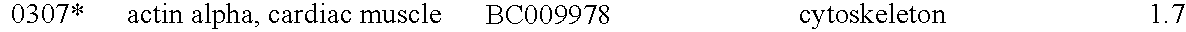

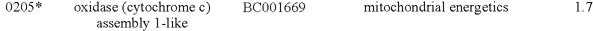

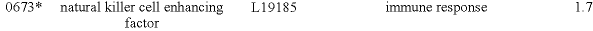

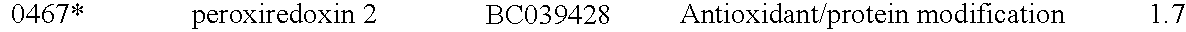

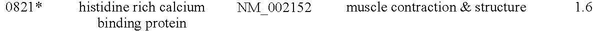

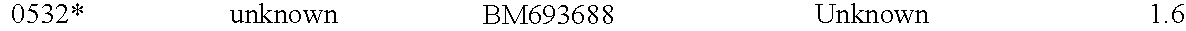

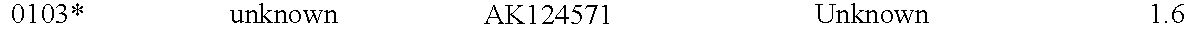

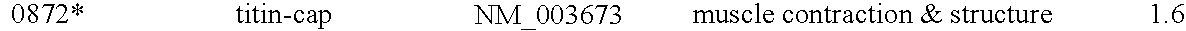

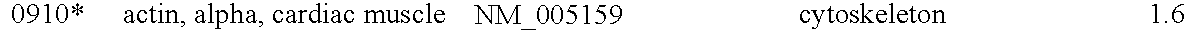

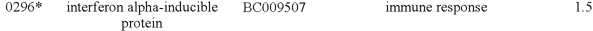

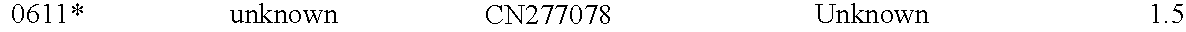

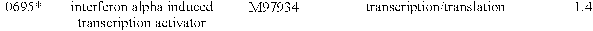

TABLE 11
Down-Regulated Genes

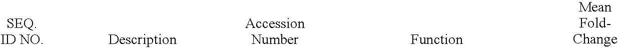

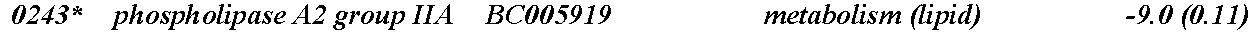

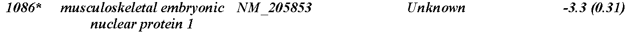

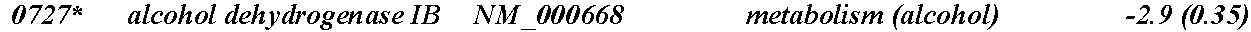

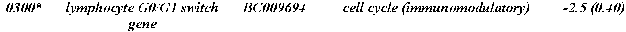

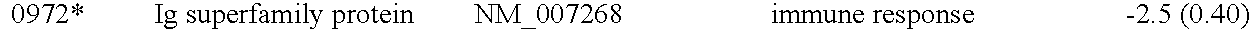

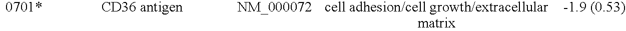

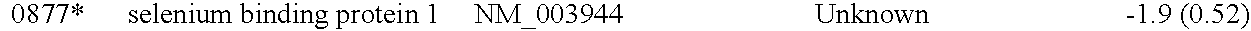

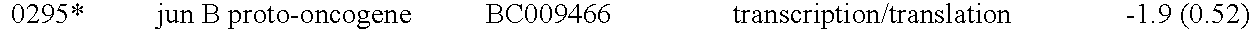

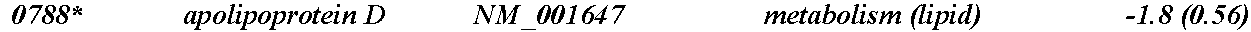

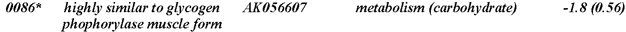

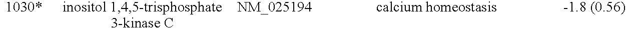

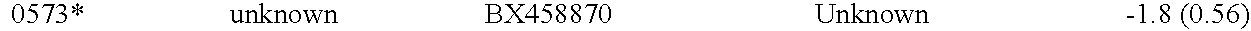

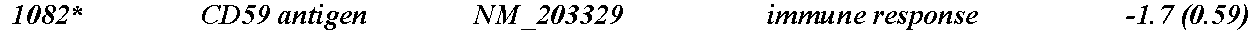

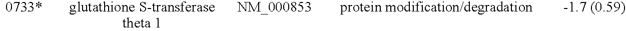

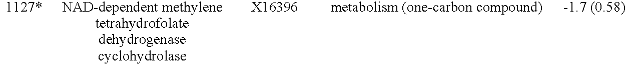

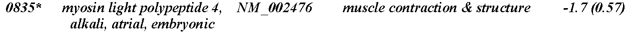

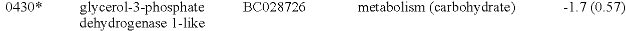

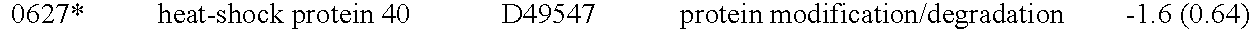

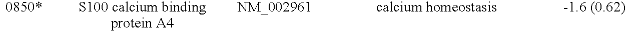

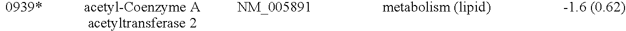

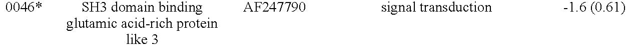

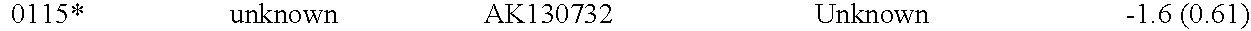

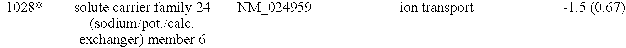

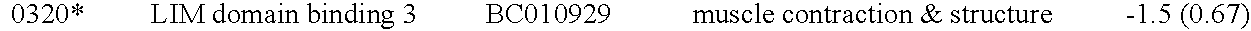

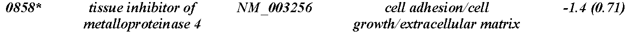

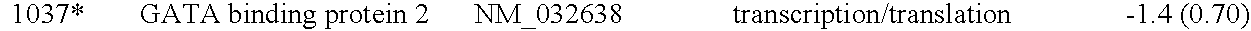

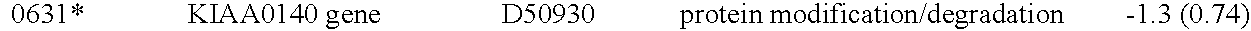

Only 23 of those genes were found to be significantly up (14 genes) or down (9 genes) regulated coordinately in the male patient samples (listed in bold and italicized in Tables 10 and 11). In females, 17 genes were found to be up-regulated and 8 genes were down-regulated.

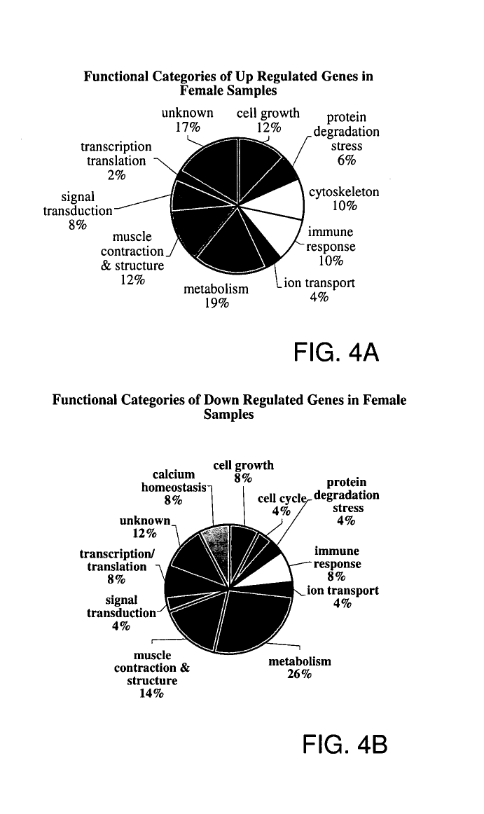

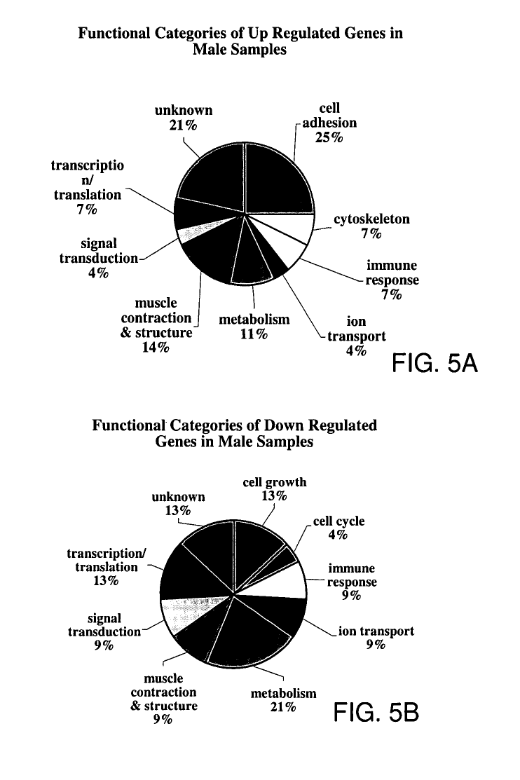

Many of the genes that displayed differentially expression encode proteins with known functions, whereas others correspond to genes of unknown function (these genes include novel and previously unidentified EST's). Genes of known function were classified of the basis of biological function according to a modified version of the NCBI Gene Ontology (GO) classification scheme. The functional classification scheme consisted of 9 categories and subgroups within each category. Functional classifications within the expression clusters of the female cohort are illustrated in FIGS. 4A and 4B. Functional classifications within the expression clusters of the male cohort are illustrated in FIGS. 5A and 5B.

Overall analysis of differentially expressed genes in the female (a) cohort based on functional category shows a female specific expression pattern. Genes encoding metabolic proteins made up a majority (19%) of the female-specific up-regulated expression pattern. In general, a majority of this functional category included proteins involved in oxidative phosphorylation such as ATP synthase, NADH dehydrogenase, malate dehydrogenase 1, cytochrome C oxidase, and succinate dehydrogenase as well as acetyl-Coenzyme A acetyltransferase (lipid metabolism), and poly (rC) binding protein 2 (regulation of nucleic acid metabolism). As shown in Table 10, a majority of genes found to be significantly up-regulated coordinately in both male and female cohorts represented proteins involved in cell growth, cell adhesion, and the extracellular matrix. This observation is indicative of ventricular remodeling that occurs at end-stage IDCM irrespective of sex. Insulin-like growth factor binding protein 2 and latent transforming growth factor-beta binding protein were uniquely up-regulated only in the female cohort.

Transcripts down-regulated in the female cohorts were those involved with lipid and carbohydrate metabolism. Apolipoprotein D and phospholipase A2 (both involved in lipid metabolism) were found to be coordinately down-regulated in both the male and female cohorts with Acetyl-Coenzyme A acetyltransferase 2 uniquely down-regulated only in the female cohorts. Likewise, glycogen phosphorylase (carbohydrate metabolism) was coordinately down-regulated in both male and female cohorts whereas glycerol-3-phosphate dehydrogenase 1 was uniquely down-regulated only in the female cohort. Transcripts involved in ion transport, calcium signaling and homeostasis including S100 calcium binding protein A4, inositol 1,4,5-trisphosphate 3-kinase C, and solute carrier family 24 showed significantly lower levels of expression unique to the female dataset.

Because DNA microarrays are not available for the turkey genome, the technique of subtractive suppression hybridization (SSH) represents a large scale, unbiased method of detecting differentially expressed genes between healthy and diseased tissue. The avian sequences obtained from the subtractive hybridization libraries were queried in the NCBI databases using BLASTN. Homology searches were based on sequence similarity of at least 55% at the nucleotide level. The process revealed that 60 genes identified in the turkey subtracted cDNA libraries had homologues in our human subtracted cDNA library dataset (Table 12). Forty-four out of 56 human and turkey homologues were identified in the same subtracted cDNA library either forward (F) or reverse (R), seven genes were identified in opposite libraries and 5 turkey homologues were not contained in the human cDNA libraries. These data further support the usefulness of the avian DCM model. In Table 12 below, F represent a gene that was identified in the forward subtracted library, R represents a gene that was identified in the reverse subtracted library, and N represents a gene not identified in reverse or forward libraries.

TABLE 12

| Turkey | | | Human |
|---|---|---|---|
| Sub. | | Accession | Sub. |
| cDNA | | Number for | cDNA |
| library | Gene Name | Human Gene | library |
| F/R | Atrial natriuretic peptide precursor | BC005893.1 | F |
| F | Arg/Abl-interacting protein ArgBP2a | BC011883.1 | F |
| F | Cysteine-rich protein precursor | AF167706.1 | F |
| F/R | Lactate dehydrogenase B | BC002362.1 | R |
| F/R | Cardiac LIM protein | U20324 | N |
| | (cysteine/glycine-rich protein 3) | | |
| F | Titin | NM_003319.2 | F |
| F/R | Myosin, 6 heavy chain, cardiac | NM_002471.1 | F/R |
| F | Serine/threonine kinase | AJ303380.2 | F |
| F | Nuclease-sensitive binding protein | BC013838.1 | R |
| F | Tropomyosin 3 | NM_152263.1 | F |
| F | Gelsolin | NM_198252.1 | F |
| F | Reticulon 4 | BC010737.1 | F |
| F | Actin beta | NM_001101.2 | F/R |
| F | Adducin 2 beta | NM_017488.1 | N |
| F/R | Tropomodulin | NM_003275 | N |
| F | Ubiquitin-conjugating enzyme | BC000848.1 | R |
| F | DNAJ homolog (HSP40) | D49547 | F |
| F/R | Ribosomal protein L4 | BC005817.2 | F |
| F | Complement component c7 | J03507 | R |
| F | Decorin | NM_133506.2 | F/R |
| F | Actin, alpha 2, smooth muscle, aorta | NM_001613.1 | F |
| F/R | Myosin binding protein C | NM_000256.2 | F/R |
| F/R | NADH dehydrogenase subunit 2 gene | AAL48387.1 | R |
| F/R | Aldose reductase | BC007024.1 | R |
| F | Ryanodine receptor 2 | NM_001035.1 | R |
| F | Proteasome subunit 26S | NM_002805.4 | R |
| F | Ribosomal protein L36 | NM_033643.1 | F |
| F | Insulin-like growth factor-binding | BC018962.2 | R |
| | protein 3 | | |
| F | Calnexin | BC003552.1 | F |
| F | SH3 domain-binding glutamic | BC030135.2 | R |
| | acid-rich protein | | |
| F | Voltage-dependent anion channel | L06132.1 | F |
| F | Lumican | BC007038.1 | F |
| F | Fatty acid binding protein 4 | BC003672.1 | F |
| F | Protein phosphatase 1, beta | BC002697.2 | R |
| R | Gamma-sarcoglycan | U63395.1 | F |
| F | Heat shock protein 90 | BC009206.2 | F/R |
| F | Fibulin 1 | NM_006486.2 | F |
| F | Annexin 1 | NM_000700.1 | F |
| F | Supervillin | AF051850.1 | F |
| F | Tubulin-specific chaperone a | NM_004607.1 | F |
| R | ATPase Ca++ transporting (SERCA) | NM_001681.2 | R |
| R | Translation elongation factor 2 | NM_001961.2 | F |
| R | Integrin beta 1 | NM_002211.2 | R |
| R | Myosin heavy chain 7 | NM_000257.1 | F/R |
| F/R | Cysteine and glycine-rich protein 2 | BC000992.2 | F |
| F/R | Glyceraldehyde-3-phosphate | BC023632.2 | F/R |
| | dehydrogenase | | |
| F/R | Translation elongation factor 1 | BC018641.2 | F/R |
| | alpha 1 | | |
| R | Translation elongation factor 1 | NM_001958.2 | F/R |
| | alpha 2 | | |
| R | Myosin light chain 3 | NM_000258.1 | R |
| R | Troponin C | NM_003280.1 | F/R |
| R | CD36 antigen | NM_000072.1 | R |
| R | RAB18 | NM_181070.2 | R |
| R | LIM domain binding 3 | BC010929.2 | F/R |
| R | Casein kinase 1, epsilon | L37043 | N |
| R | Na+/K+ ATPase alpha | 1309271B | F |
| R | DEAD box protein 5 | NM_004396.2 | R |
| R | Transferrin receptor | NP_003225.1 | N |
| F/R | Actinin alpha 2 | NM_001103.1 | F/R |
| R | Cyclin I | D50310.1 | F |
| R | Malate dehydrogenase 1 (NAD) | NM_005917.2 | R |
| | soluble | | |

### EXAMPLE 9

Real time RT-PCR was used to confirm the relative expression patterns of 46 transcripts from Example 8 identified as differentially expressed in DCM by means of microarray analysis. For each gene of interest real time RT-PCR was performed using RNA derived from pooled male DCM (n=5) and pooled female IDCM (n=6), and non-failing pooled RNA male or female samples (n=10) was used as a reference. Two-step real-time RT-PCR was performed using 10 ng of total RNA per reaction. Triplicate aliquots of each RNA sample were used in the same reactions. All samples were normalized to 18S rRNA as an internal control. For all experimental samples, the relative fold difference of each gene was determined as it compares to the pooled non-failing male or female reference sample (n=10) by means of the ΔΔCt (threshold cycle) method (Applied Biosystems, Foster City, Calif.).

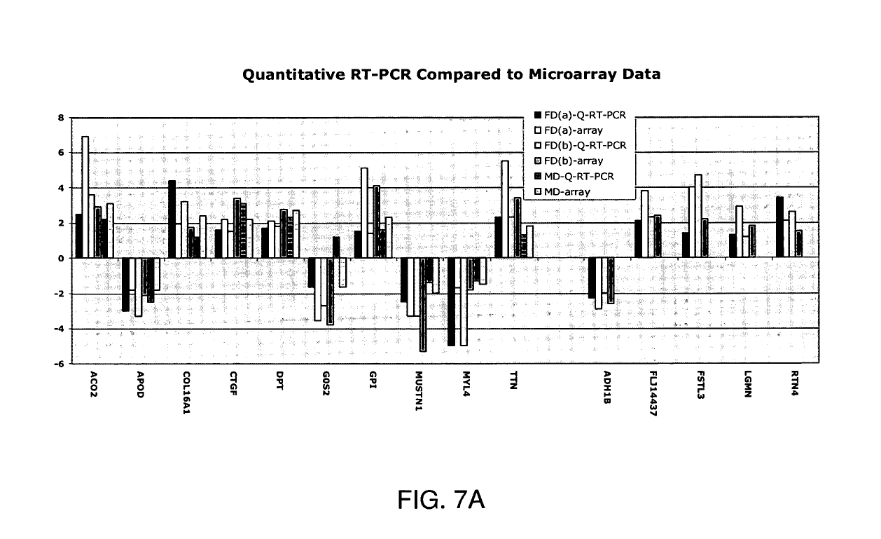

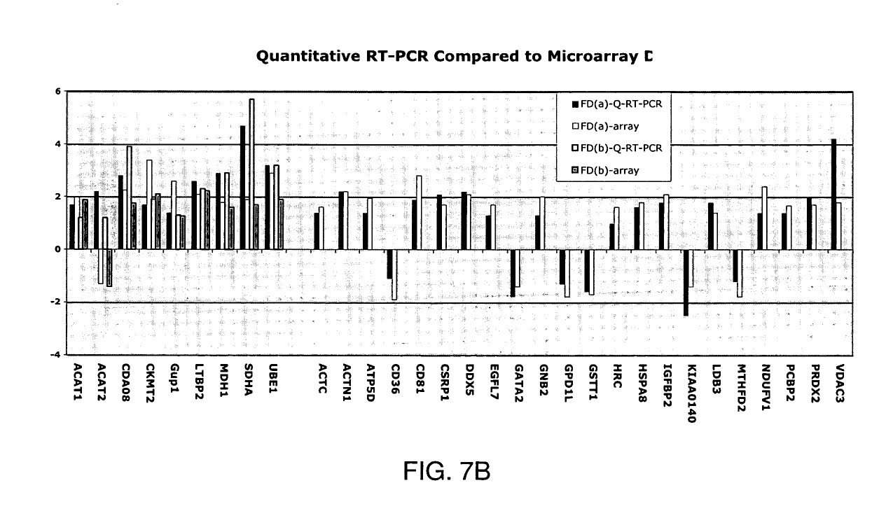

Expression fold change was determined for each pooled DCM RNA sample (male n=5; female n=6; as it relates to the pooled non-failing male or non-failing female sample (n=10). Expression of 45 of the 46 genes tested by means of Quantitative RT-PCR paralleled our results obtained with microarray analysis (see FIGS. 7A and 7B). Positive numbers represent fold up-regulated and negative numbers represent fold down-regulated.

NPPA (natriuretic peptide precursor) showed a high level of up regulation across all IDCM female and male patients confirming NPPA's role as a powerful marker of heart stress. Although, despite NPPA's significant average up regulation, the level of NPPA was highly variable among individuals in both the male and female patient groups. Table 13 shows the results of the RT PCR studies.

TABLE 13

| | Turkey | Human | |
|---|---|---|---|
| | Sub. | Sub. | Turkey |
| | cDNA | cDNA | qPCR Fold |
| Gene Identification | library | library | Change |
| Atrial natriuretic peptide precursor | F/R | F | 2.1* |
| Arg/Abl-interacting protein ArgBP2a | F | F | 2.0* |
| Titin | F | F | 7.2* |
| Myosin, 6 heavy chain, cardiac | F/R | F/R | 0.3* |
| Tropomyosin 3 | F | F | 1.5* |
| Complement component c7 | F | R | 0.6* |
| Decorin | F | F/R | 1.6* |
| Lumican | F | F | 1.3* |
| Fatty acid binding protein 4 | F | F | 1.0 |
| Fibulin 1 | F | F | 0.9 |
| Myosin heavy chain 7 | R | F/R | 1.2 |
| CD36 antigen | R | R | 0.4* |

The largest group of genes consistently up-regulated in DCM female patients were those involved in general cell growth/extracellular matrix, metabolism (e.g. mitochondrial oxidative phosphorylation and ATP synthesis), and muscle contraction and structure. Most of those genes coordinately up-regulated in both the female and male groups are those involved in general cell growth and extracellular matrix indicative of myocyte and ventricular remodeling.

Although the female pattern of 54 genes that are up-regulated consequent to end stage DCM shares 14 genes in common with male samples, unique to female DCM samples in this gene list are several genes involved in mitochondrial oxidative phosphorylation. In the stressed heart, metabolic remodeling precedes, triggers, and sustains functional and structural remodeling (Taegtmeyer H. Ann, Biomed. Eng. 2000; 28:871-876.) Adaptations to sustained heart stress induce changes of the metabolic machinery at a transcriptional and/or translations level of the enzymes of metabolic pathways. Concomitant with an increase of gene expression of enzymes associated with oxidative phosphorylation is an increase in peroxidoxin 2 in female specific analysis, an enzyme involved in the reduction of the oxygen radical hydrogen peroxide (a destructive by-product of oxidative phosphorylation). This observation of deregulation of genes associated with energy transduction and antioxidant activity unique to female patients at end stage heart failure may suggest a higher level of metabolic adaptation in female hearts due to stress resulting in increased myocyte survival.

There is striking evidence found in cancer cells that implicates a link between cell survival and metabolism. Cancer cells are shown to possess an increase rate of glucose metabolism and oxidative phosphorylation accompanied by a reduction in cell death when stressed (Warburg O, Science. 1956; 123:309-314; Hanahan D and Weinberg R A. Cell. 2000; 100:57-70). Perhaps female hearts possess a greater ability, due heightened metabolic adaptation, to maintain energy for contractile function of the stressed heart leading to less cardiac dysfunction, cell death, and remodeling.

The transition from compensated cardiac hypertrophy to decompensated heart failure is accompanied by marked changes in the array of genes in the heart. These observed gender-specific differences in the gene expression pattern consequent to end-stage DCM could indicate a diverse compensation mechanism in female heart failure. This data hints that increased compensation mechanisms in female heart failure may lie in increased or prolonged efficiency of metabolic adaptation to pressure overload. The modulation of energy metabolism to improve performance of dysfunctional myocardium has been intensely studied (Stanley W C et al. Cardiovasc Res. 1997; 33:243-257). Further study is needed to assess any increased beneficial effects of metabolic modulation in female heart failure that may improve metabolic/mechanical coupling.

### EXAMPLE 10

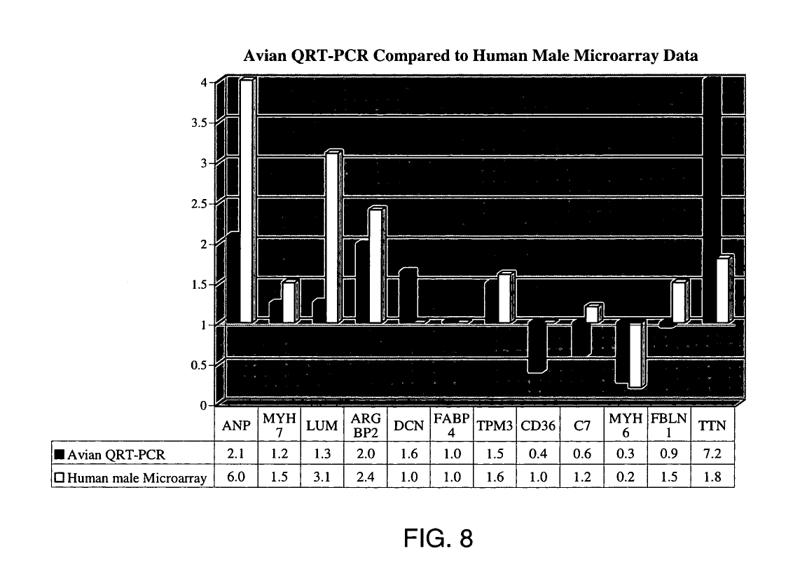

To determine if changes in the gene expression pattern that we have identified in human male patients with end-stage heart failure were also observed in our male turkey heart failure model, Q-RT-TPCR analysis using avian heart failure genes chosen from our avian forward and reverse subtracted libraries was compared to microarray gene expression data obtained from samples from male transplant recipients with idiopathic dilated cardiomyopathy. A human homologue was identified for each of the 12 avian genes and was available on the constructed human heart failure microarray (see FIG. 8). These data are consistent with a majority (8 of 12) of the selected genes being coordinately regulated in the turkey model as compared to human samples. Six of the randomly selected genes known to be up regulated due to idiopathic dilated cardiomyopathy were shown to be coordinately up regulated in the in our avian model. These genes include atrial natriuretic peptide precursor (regulator of blood pressure), myosin heavy chain 7, Arg/Abl-binding protein 2, and titin (muscle structure proteins), lumican (extracellular matrix constituent), and tropomyosin 3 (muscle contraction). Myosin heavy chain 6 (muscle structure) shows a down-regulation in both avian Q-RT-PCR and human male microarray analysis. Fatty acid binding protein 4 both shows no change in both avian Q-RT-PCR and human microarray analyses. These results are a powerful validation of the avian model for heart failure on the molecular or gene expression level.

### EXAMPLE 11

To determine if protein levels change in parallel with gene expression in heart failure, randomly selected proteins that were found to be differentially expressed in both the human and turkey samples at the gene expression level were selected. Protein expression levels using were studies using semi-quantitative Western blot analysis. Total protein was extracted from the left ventricle samples of female patients undergoing cardiac transplantation for idiopathic dilated cardiomyopathy. Similarly, total protein was extracted from avian control and heart failure left ventricle samples. The protein was quantified using a standard Bradford protein assay. Equal amounts of protein were pooled for female samples and control samples. Two hundred micrograms (200 μg) of each protein sample was run on a 10-20% gradient Tris-glycine polyacrylamide gel and transferred to a PVDF membrane in a standard electro-blotter system (Owl Separation Systems). A Horseradish peroxidase (HRP) conjugate and the SuperSignal West Femto Chemiluminescent substrate were used for detection (Pierce). Quantitative data (DCM relative to donor/control samples) were obtained using the NIH densitometry software (NIH Image).

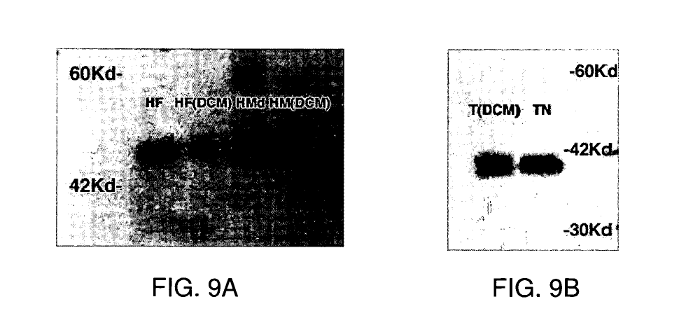

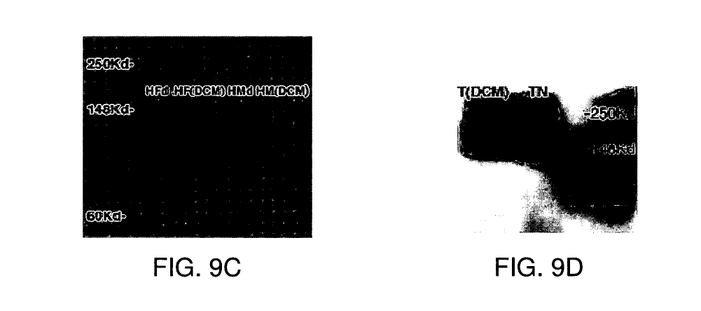

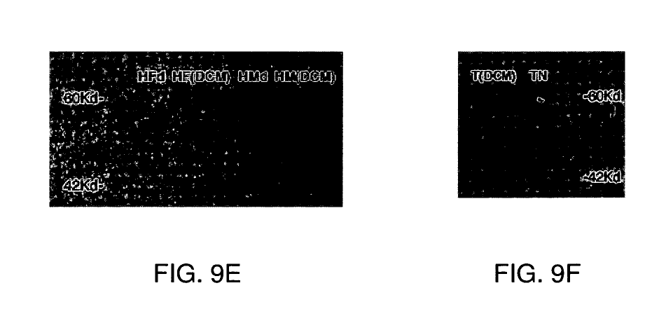

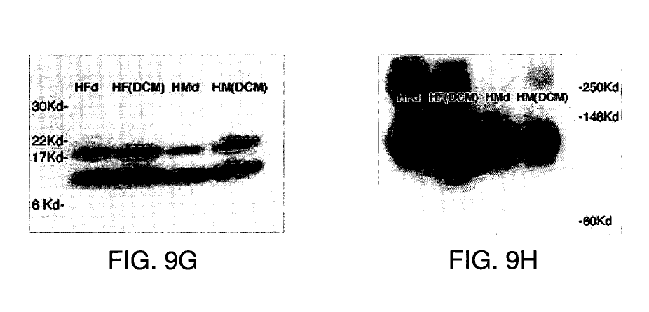

Tropomyosin 3 (TPM3) gene expression was consistently up regulated in heart samples from male transplant recipients with idiopathic dilated cardiomyopathy (DCM) as assessed by microarray analysis. Differential gene expression of the TPM3 gene in turkey heart failure samples mimicked expression in the DCM samples and was found to be up regulated (1.5 fold) consequent to heart failure as assessed by Q-RT-PCR (see FIG. 8). FIGS. 9A and 9B show Western blots of tropomyosin 3 (TPM3) for human and turkey, respectively. FIGS. 9C and 9D show Western blots of Myosin Heavy Chain alpha 6 (MYH6) for human and turkey, respectively. FIGS. 9E and 9F show Western blots of alpha Tubulin (ATUB) for human and turkey, respectively. FIG. 9G shows a Western blot of Fatty Acid Binding Protein 4 (FABP4) for human. FIG. 9H shows a Western blot of Sarcoplasmic Reticulum Ca²⁺ ATPase (SERCA) for human. 200 μg of total protein was used for each sample. Relative specific protein levels between idiopathic dilated cardiomyopathy and non-failing donor samples were obtained using the NIH densitometry software (NIH Image). HMd (Human Male donor)=200 μg total protein isolated from human male donor left ventricle tissue (normal). HMDCM (Human Male heart failure)=200 kg total protein isolated from human male patients undergoing cardiac transplantation (IDCM). TN (Turkey Normal)=200 μg total protein isolated from turkey (male) normal left ventricle tissue. TDCM (Turkey heart failure)=200 μg total protein isolated from turkey heart failure (male) left ventricle tissue.

Quantitative western blot data suggests little correlation of tropomyosin 3 protein levels and gene expression data in the human samples. Quantitative data (as assessed by means of densitometry measurements) demonstrated no change in TPM3 protein levels. In contrast, the turkey western blot data showed a 40% increase in TPM3 as a consequence of heart failure correlating with the avian gene expression data as assessed by Q-RT-PCR

Gene expression of Myosin heavy chain 6 (MYH6) was observed to be consistently down regulated in human male heart failure samples as assessed by microarray analysis. Conversely, quantitative western blot analysis suggests an increase in MYH6 protein. Q-RT-PCR data shows the MYH6 gene to be down regulated in turkey heart failure samples. Western blot analysis of the turkey samples detects a 150 Kd protein (MYH6). Quantitative analysis shows a 27% decrease of MYH6 protein in the heart failure samples as compared to the normal control samples correlating with the gene expression data. Alpha Tubulin (ATUB) gene expression was consistently down regulated in male human samples as assessed by microarray analysis. Quantitative Western blot analysis of the ATUB protein suggests a 75% decrease of ATUB protein in the male human samples. Corresponding to these data in the human male sample microarray similarly indicated a decrease in the turkey heart. A 22% decrease in ATUB protein content was found in the turkey heart failure sample by means of western blot analysis.

Gene expression analysis of Fatty Acid Binding Protein 4 (FABP4) showed no significant deregulation in male human samples as assessed by microarray analysis. No change in expression levels was found in turkey samples by means of Q-RT-PCR concurring with the human microarray gene expression data. Western blot analysis of the human FABP4 showed two bands at approximately 17 kDa and 12 kDa. In contrast to gene expression data, densitometry analysis indicates a 77% increase in FABP4 protein content in human male heart failure samples.

Gene expression analysis of Sarcoplasmic Reticulum Ca²⁺ ATPase (SERCA) shows this gene to be down regulated in male human transplant recipients as assessed by microarray analysis. Western blot analysis corresponds to the male gene expression pattern and indicated an 88% decrease in protein content in the male human heart failure samples. As expected, protein expression analysis of TPM3, MYH6, and ATUB in our avian heart failure model correlated well with gene expression data in human male heart failure samples as well as avian Q-RT-PCR analysis.

### EXAMPLE 12

### Alcohol Induced Heart Failure Studies

Excessive alcohol consumption is recognized as a cause of left ventricular dysfunction and often leads to alcohol-induced heart failure. It is thought that 36% of all cases of dilated cardiomyopathy are due to excessive alcohol intake.

The DCM array noted above was used to screen RNA samples from transplant recipients and organ donors with alcohol associated heart failure. In brief and more fully described above, a unique human heart failure microarray for idiopathic dilated cardiomyopathy (as discussed in Example 8 above) was developed by means of subtractive suppression hybridization of left ventricles from transplant recipients undergoing cardiac transplantation and normal tissue obtained from brain-dead organ donors. All samples obtained from transplant recipients were with patient consent and family consent was obtained for brain dead organ donors. A total number of 1,143 genes are represented on our human heart specific microarray along with 8 control oligonucleotides representing sequences that do not hybridize to mammalian sequences. Control RNA transcripts corresponding to the oligonucleotide controls were used in the hybridization process for the normalization and validation of gene array data. Microarray oligos (70 nucleotides in length) were designed for each human gene using ArrayOligoSelector software and the oligos were synthesized by Illumina (San Diego). These oligos were spotted in triplicate onto epoxy-coated slides obtained from MWG (Germany) and stored at −20° C.

RNA samples from left ventricle tissue of a male with confirmed alcohol-induced heart failure and two males with heart failure with alcohol as a complication were hybridized to the heart failure microarray and compared to a pooled RNA normal samples (20 normal left ventricle samples from male and female donors). cDNA was synthesized from 2 μg of total RNA from normal samples. cDNA was synthesized from 2 μg of total RNA isolated from left ventricle tissue of the alcohol-induced heart failure or normal left ventricle pooled control samples. cDNA construction and microarray hybridization were performed using the 3DNA Array 900 Detection System (Genisphere) following the manufacturers instructions. A total of two technical replicates for each sample were performed (dye swap). cDNA hybridization was done over night at 62° C. The slides were washed and then hybridized to fluorescent dendrimers. The microarray slides were scanned twice in a Perkin Elmer HT scanner. Photomultiplier tube (PMT) values were set at 69 and 60 volts for Cy3 and Cy5, respectively. An additional scan was done for each slide with the PMT set at 54 and 46 volts.

Data were filtered using the coefficient of variation (CV) function in GeneSpring Software. The Genes with a CV<30% between the dye swaps were selected. We compiled a list of genes which appeared in 70% of the CV<30% list. Genes were selected from the 70% list which were at least 1.8-fold up or down-regulated in the alcohol-induced samples as compared to the pooled normal controls.

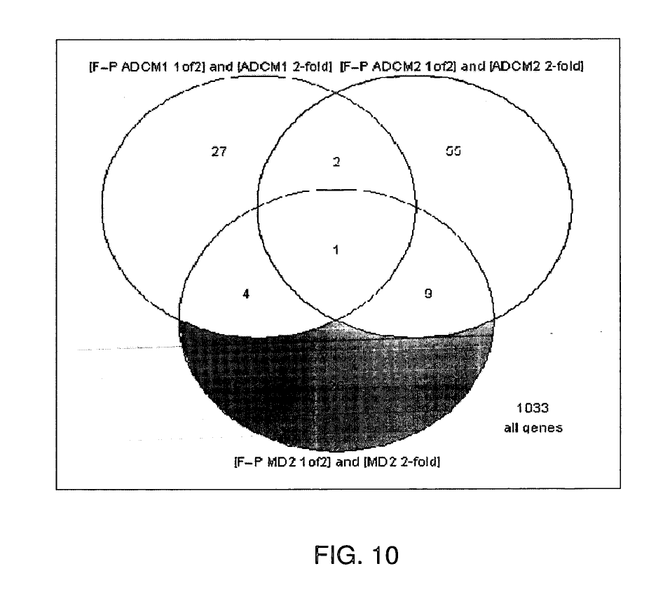

Using stringent analysis criteria, 32 differentially regulated genes were identified with 14 of the identified genes being significantly up-regulated and 18 of the identified genes being significantly down-regulated in the confirmed alcohol-induced heart failure sample. FIG. 10 shows an overlap diagram of genes found to be differentially expressed >2 fold up or down (compared to normal hearts) in the 3 alcohol DCM Hearts. In FIG. 10, ADCM1-refers to confirmed Alcohol induced DCM heart, ADCM2 refers to putative Alcohol induced DCM heart and MD2 refers to putative Alcohol induced DCM heart. There is a clear lack of overlap among the alcohol-induced heart failure sample and the samples from patients with idiopathic dilated cardiomyopathy with excessive alcohol consumption as a complication.

Dermatopontin and tropomyosin are both up-regulated in the confirmed alcohol-induced heart failure (AHF1) male sample and putative alcohol-induced heart failure (AHF3) sample and represent the extracellular matrix and muscle contraction respectively. Alternatively, a muscle contraction gene, myosin heavy chain, was down-regulated in AHF1 and AHF3. Collagen type III (associated with the extracellular matrix) was common to all three samples (AHF1, putative alcohol-induced heart failure sample 2 (AHF2), AHF3), but was down-regulated in AHF1 and up-regulated in AHF2 and AHF3. This difference in expression of collagen type III could be specific to the alcohol induced etiology of heart failure.

A differential gene expression in two male idiopathic dilated cardiomyopathy transplant recipients with the additional disease of alcoholism at the time of diagnosis (putative alcohol-induced heart failure) was also investigated. Among the 32 genes found to be up or down-regulated (at least 1.8-fold) in the confirmed alcohol-induced heart failure (AHF1) male sample, only five of these genes were significantly deregulated (at least 1.8-fold) in putative alcohol-induced heart failure sample 2 (AHF2) and only two of those genes were deregulated (at least 1.8-fold) in putative alcohol-induced heart failure 3 (AHF3).

These gene expression data suggest that putative alcohol-induced heart failure (AHF 2, AHF 3) is the result of an alternative etiology and heart failure was not induced solely by chronic excessive alcohol consumption.

### EXAMPLE 13

A comparison of gene expression profiles obtained from alcohol-induced heart failure and heart failure due to idiopathic dilated cardiomyopathy was performed. A unique pattern of gene expression in left ventricles from transplant recipients with idiopathic dilated cardiomyopathy has been previously identified and is shown in Table 14 below. In Table 14, representative list of genes found to be differentially regulated (at least 1.8-fold) up or down in our alcohol-induced heart failure samples that were also differentially regulated in 7 male or 6 female transplant recipients with heart failure due to idiopathic dilated cardiomyopathy are shown. Up-regulated and down-regulated genes are presented as a relative fold change compared to the pooled normal samples. Fold change above 1 denotes up-regulated, and fold change below 1 denotes down-regulated. Asterisk indicates a discrepancy in fold change between the alcohol-induced heart failure sample and idiopathic dilated cardiomyopathic heart failure samples. Randomly selected calponin and tropomyosin were validated with QT-PCR.

TABLE 14

| | Average | Fold change in |
|---|---|---|
| | (DCM/Normal) | Alcohol-induced |
| | fold change in | DCM |
| Gene Identification | 7 male patients | DCM/Normal |
| Lipocalin (LCN6) | 0.17↓ | 2.2↑* |
| Dermatopontin | 2.3↑ | 1.9↑ |
| (DPT) | | |
| Phospholipase A2 | 0.30↓ | 0.46↓ |
| (PLA2G2A) | | |
| Tropomyosin 3 | No significant | 3.2↑* |
| (TPM3) | Change | |
| Titin N2B (TTN) | No significant | 0.41↓* |
| | Change | |
| Collagen Type III | No significant | 0.40↓* |
| (COL3A1) | Change | |
| Calponin 1 (CNN1) | No significant | 0.35↓ |
| | Change | |
| Musculoskeletal | No significant | 0.31↓ |
| Embryonic Protein | Change | |
| (MUSTN1) | | |

Of particular note in Table 14 were six genes that were deregulated in opposite directions in alcohol-induced heart failure samples as compared to idiopathic dilated cardiomyopathy heart failure samples. Tropomyosin, a muscle development gene, was shown by means of microarray analysis, as well as QRT-PCR to be up-regulated in alcohol-induced heart failure samples. Titin, a structural muscle gene, and collagen type III, a structural cellular matrix gene, by array analysis were down-regulated in alcohol-induced heart failure samples. Similarly, calponin and musculoskeletal genes were significantly down-regulated in alcohol induced heart failure samples. These data indicate that etiology and pathogenesis of heart failure appears to be relevant at the gene level.

Alcohol-induced heart failure was associated with a significantly higher percentage of changes in matrix/structural proteins. These proteins tended to be turned off with alcohol-induced heart failure. A striking difference in the functional patterns was the presence of proapoptotic genes that were up-regulated in the alcohol-induced heart failure gene group, but were not present in the idiopathic dilated cardiomyopathy heart failure gene group. Also evident was a greater proportion of up-regulation of cell adhesion/extracellular matrix genes in the idiopathic dilated cardiomyopathy group (27%) compared to the alcohol-induced heart failure gene group (9%). A final important difference was evident in the muscle structure/muscle contraction category. Most genes in this functional category due to alcohol-induced heart failure that were involved in the regulation of muscle contraction were down-regulated. On the contrary, most genes in this functional category due to idiopathic dilated cardiomyopathy induced heart failure involved in muscle structure were up-regulated. These differences may lead to a better understanding of the development of alcohol-induced heart failure.

The results listed above were consistent with alcohol-induced heart failure having a “specific fingerprint” profile of de-regulated genes. This profile may differentiate patients with pure alcohol-induced heart failure from patients with heart failure from idiopathic dilated cardiomyopathy or other unknown etiologies with alcohol as a complicating or contributing factor. Furthermore, the pattern of gene de-regulation may suggest a role for changes in matrix, cytoskeletal, and basement membrane proteins that are likely involved in the development of heart failure resulting from excessive alcohol consumption. The results also demonstrate that the human heart failure array can be used to generate fingerprint profiles for other forms of heart failure, e.g., non DCM or alcohol induced heart failure.

Each of the citations listed above is hereby incorporated herein by reference for all purposes, and the full citation of certain citations listed above is provided below.
Adams M D, Kerlavage A R, Fleischmann R D, Fuldner B A, Bull C J, Lee N H, Kirkness E F, Weinstock K G, Gocayne J D, White O. (1995) Initial assessment of human gene diversity and expression patterns based upon 83 million nucleotides of cDNA sequence. Nature 377:3-174;
Akopian A N, Wood J N (1995) Peripheral nervous system-specific genes identified by subtractive cDNA cloning. J Biol. Chem. 270: 21264-21270;
Barrans D J, Allen P D, Stamatiou D, Dzai V J, and Liew C C. Global gene expression profiling of end-stage dilated cardiomyopathy using a human cardiovascular-based cDNA microarray. American Journal of Pathology, 2002; 160 (6):2035-2043;
Boehm M, Morano I, Pieske B, Ruegg J C, Wankerl M, Zimmermann R, Erdman E (1991). Contribution of cAMP-phosphodiesterase inhibition and sensitization of the contractile proteins for calcium to the inotropic effect of pimbobendan in the failing human myocardium. Circ. Res. 68:689-701;
Bristow M R, Ginsburg R et al. (1986) β1- and β2-adrenergic-receptor subpopulations in non-failing and failing human ventricular myocardium: coupling of both receptor subtypes to muscle contraction and select β1-receptor down-regulation in heart failure. Circ. Res. 59: 297-309;
Carroll J D, Carroll E P, Feldman T, Ward D M, Lang R M, McGaughey D, Karp R B. Sex-associated differences in left ventricular function in aortic stenosis of the elderly. Circulation. 1992; 86(4):1099-1107;
Chapados R A, Gruver E J, Ingwall J S, Marsh J D, Gwathmey J K. (1992) Chronic administration of cardiovascular drugs: altered energetics and transmembrane signaling. Am J Physiol 263: H1576-H1586;
Davidoff A J, Gwathmey J K. (1994) Pathophysiology of cardiomyopathies: Part I. Animal models and humans. Current Opinion in Cardiology. 9:357-368;
Deleersnjider W, Hong G, Cortvrindt R, Poirier C, Tylzanowski P, Pittois K, van Marck E, Merregaert J. (1996) Isolation of markers for chondro-osteogenic differentiation using cDNA library subtraction. Molecular cloning and characterization of a gene belonging to a novel multigene family of integral membrane proteins. J Biol Chem 271: 19475-19482;
Diatchenko L, Lau Y-F C, Campell A P, Chenchik A, Moqadam F, Huang B, Lukyanov S, Lukyanov K, Gurskaya N, Sverdlov E D, Siebert P D. (1996) Suppression subtractive hybridization: A method for generating differentially regulated or tissue-specific cDNA probes. Proc Natl Acad Sci USA 93: 6025-6030;
Du X-J, Samuel C S, Gao X-M, Zhao L, Parry U, Tregear G W. Increased myocardial collagen and ventricular diastolic dysfunction in relaxin deficient mice: a gender-specific phenotype. Cardiovascular Research. 2003; 57:395-404;
Felman M D, Copelas L, Gwathmey J K, Phillips P J, Schoen F, Grossman W, Morgan J P. (1987) Deficient production of cyclic AMP: pharmacologic evidence of an important cause of contractile dysfunction in patients with end-stage heart failure. Circulation 75:331-339;
Gao X M, Agrotis A, Autelitano D J, Percy E, Woodcock E A, Jennings G L, Dart A M, and Du X J. Sex Hormones and Cardiomyopathic Phenotype Induced by Cardiac B₂-Adrenergic Receptor Overexpression. Endocrinology. 2003; 144(9):4097-4105;
Glass M G, Fuleihan F, Liao R, Lincoff A M, Chapados R, Hamlin R, Apstein C S, Allen P D, Ingwall J S, Hajjar R J, Cory C R, O'Brien P J, Gwathmey J K. (1993) Differences in cardioprotective efficacy of adrenergic receptor antagonists and calcium channel antagonists in an animal model of dilate cardiomyopathy: Effects on gross morphology, global cardiac function, and twitch force. Circ Res 73: 1077-1089;
Grzeskowiak R, Witt H, Drungowski M, Thermann R, Hennig S, Perrot A, Osterziel K J, Klingbiel D, Scheid S, Spang R, Lehrach H, Ruiz P. Expression profiling of human idiopathic dilated cardiomyopathy. Cardiovascular Research. 2003; 59: 400-41;
Gwathmey J K, Hamlin R L. (1983) Protection of turkeys against furazolidone-induced cardiomyopathy. Am J Cardiol 52: 626-628;
Gwathmey J K, Copelas L, Mackinnon R, Schoen F J, Feldman M D, Grossman W, Morgan J P. (1987) Abnormal intracellular calcium handling in myocardium from patients with end-stage heart failure. Cir Res 61:70-76;
Gwathmey J K, Slawsky M T, Hajjar R J, Briggs G M, Morgan J P (1988) Role of intracellular sodium in the regulation of intracellular calcium and contractility. Effects of DPI 201-106 on excitation-contraction coupling in human ventricular myocardium. J. Clin. Invest. 82:1592-1605;
Gwathmey J K and Hajjar R J. (1990) Effect of protein kinase C activation on sarcoplasmic reticulum function and apparent myofibrillar Ca²⁺ sensitivity in intact and skinned muscles from normal and diseased myocardium. Circ Res 67: 744-752;
Gwathmey J K. (1991) Morphological changes associated with furazolidone-induced cardiomyopathy: effects of digoxin and propranolol. J Comp Pathol 104: 33-45;
Gwathmey J K, Warren S, Briggs G M, Copelas L, Feldman M D, Phillips P J, Callahan M, Schoen F J, Grossman W, Morgan J P. (1991) Diastolic dysfunction in hypertrophic cardiomyopathy. Effect on active force generation during systole. J Clin Invest 87: 1023-1031;
Gwathmey J K and Hajjar R J. (1993) The complexity of Simplicity: the pathophysiology of heart failure—hype or hope? Res & Staff Phys 39:45-59;
Gwathmey J K, Davidoff A J. (1993) Experimental Aspects of cardiomyopathy. Curr Opin Cardiol 8:480-495;
Gwathmey J K, Liao R, Hajjar R J. (1994) Intracellular free calcium in hypertrophy and failure. In Lorell B H, Grossman W eds. Diastolic Relaxation of the Heart, Second Edition. Norwell M A, Kluwer Academic Publishers, pp 55-64;
Gwathmey J K, Davidoff A J. (1994) Pathophysiology of Cardiomyopathies: Part II. Drug-induced and other interventions. Current Opinion in Cardiology 9:369-378;
Gwathmey J K, Kim C S, Hajjar R J, Khan F, DiSalvo T, Matsumori A, Bristow M R. (1999) Cellular and molecular remodeling in a heart failure model treated with the β-blocker carteolol. Am J Physiol 276: H1678-H1690;
Haghighi K, Schmidt A G, Hoit B D, Brittsan A G, Yatani A, Lester J W, Zhai J, Kimura Y, Dorn II G W, MacLennan D H, and Kranias E G. Superinhibition of Sarcoplasmic Reticulum Function by Phospholamban Induces Cardiac Contractile Failure. J Biol. Chem. 2001; 276 (26):24145-24152;
Hajjar R J, Liao R, Young J B, Fuleihan F, Glass M G, Gwathmey J K. (1993) Physiological and biochemical characterization of an animal model of dilated cardiomyopathy: comparison to findings in human dilated cardiomyopathy. Cardiovasc Res 27:2212-2221;
Hanahan D, and Weinberg R A. The hallmarks of cancer. Cell. 2000; 100:57-70;
Hammer D R, Starling R C et al. (1989) ATP and total adenine nucleotide depletion in failing human myocardium. J. Mol. Cell. Cardiol. 21 (Suppl. II): S28;
Hanatani A, Yoshiyama M, Kim S, Omura T, Ikuno Y, Takeuchi K, Iwao H, Yoshikawa J. (1998) Assessment of cardiac function and gene expression at an early phase after myocardial infarction, Jpn Heart J 39:375-388;
Hwang J J, Allen P D, Tseng G C, Lam C W, Fananapazir L, Dzau V J, and Liew C C. Microarray gene expression profiles in dilated and hypertrophic cardiomyopathic end-stage heart failure. Physiol Genomics. 2002; 10: 31-44;
James J F, Hewett T E, Robbins J. (1998) Cardiac physiology in transgenic mice. Circ. Res. 82:407-415;
Jankus E F, Noren G R, Staley, N A (1972) Furazolidone-induced cardiac dilatation in turkeys, Avian Dis. 16:958-961;
Jasmin G, Proschek L. (1984) Calcium and myocardial cell injury. An appraisal in the cardiomyopathic hamster. Can J Physiol Pharmacol. 62(7):891-898;
Jensen L S, Chang C H, Washburn K W (1975) Differential response in cardiomyopathy of chicks and turkeys to furazolidone toxicity. Avian Dis. 19: 596-602;
Kadokami T, McTiernan C F, Kubota T, Frye C S, and Feldman A M. Sex-related survival differences in murine cardiomyopathy are associated with differences in TNF-receptor expression. J. Clin. Invest. 2000; 106:589-597;
Kass D A, Hare J M, Georgakopoulos D (1998). Murine cardiac physiology: A cautionary tail. Cir. Res. 82:519-522;
Kim C S, Matsumori A, Goldberg L, Doye A A, McCoy Q, Gwathmey J K. (1999) Effects of pranidipine, a calcium channel antagonist. Cardiovascular Drugs and Therapy 13:455-463;
Kirkwood F. Adams, Jr, M D; Carla A. Sueta; Mihai Gheorghiade; Christopher M. O'Connor, Todd A. Schwartz; Gary G. Koch, Barry Uretsky; Karl Swedberg, William McKenna, Jordi Soler-Soler, Robert M. Califf, Gender Differences in Survival in Advanced Heart Failure Insights From the FIRST Study. Circulation. 1999; 99:1816-1821.
Kuang W W, Thompson D A, Hoch R V, Weigel R J (1998) Differential screening and suppression subtractive hybridization identified genes differentially expressed in an estrogen receptor-positive breast carcinoma cell line. Nucleic Acid Res. 26: 1116-1123;
Li, Y, Kishimoto I, Saito Y, Harada M, Kuwahara K, Izumi T, Hamanaka I, Takahashi N, Kawakami R, Tanimoto K, Nakagawa Y, Nakanishi M, Adachi Y, Garbers D L, Fukamizu A, and Nakao K. Androgen Contributes to Gender-Related Cardiac Hypertrophy and Fibrosis in Mice Lacking the Gene Encoding Guanylyl Cyclase-A. Endocrinology. 2004; 145(2):951-958;
Liang P, Pardee A B. (1992) Differential display of eukaryotic messenger RNA by means of the polymerase chain reaction. Science 257:967-970;
Liao R L, Nascimben J, Friedrich J, Gwathmey J K, Ingwall J S (1996) Decreased energy reserve in an animal model of dilated cardiomyopathy: relationship to contractile performance. Circ. Res. 78:893-902;
Lisitsyn N, Lisitsyn N and Wigler M. (1993) Cloning the differences between two complex genomes. Science 259: 946-951;
Limas C J, Olivari M T et al., (1981) Calcium uptake by cardia sarcoplasmic reticulum in human dilated cardiomyopathy. Cardiovasc. Res. 21:601-605;
Lowes B D, Minobe W, Abraham W T, Rizeq M N, Bohlmeyer T J, Quaife R A, Roden R L, Dutcher D L, Robertson A D, Voelkel N F, Badesch D B, Groves B M, Gilbert E M, Bristow M R. (1997) Changes in gene expression in the intact human heart. J Clin Invest 100: 2315;
Mendez R E, Pfeffer J M, Ortola F V, Bloch K D, Anderson S, Seidman J G, Brenner B M. (1987). Atrial natriuretic peptide transcription, storage, and release in rats with myocardial infarction. Am J Physiol 253:H1449-H1455;
Murakami M, Kusachi S, Nakahama M, Naito I, Murakami T, Doi M, Kondo J, Higashi T, Ninomiya Y, Tsuji T. (1998) Expression of the β1 and β2 chains of type IV collagen in the infarct zone of rat myocardial infarction. J. Mol. Cell. Cardiol. 30:1191-1202;
Nascimben L, Pauletto P et al., (1991) Decreased energy reserve may cause pump failure in human dilated cardiomyopahty. Circulation 84:II 560-563;
Pagani E D, Alousi A A, Grant A M, Older T M, Dziuben S W jr, Allen P D (1988) Changes in myofibrillar content and Mg-ATPase activity in ventricular tissues from patients with heart failure caused by coronary artery disease, cardiomyopathy or mitral valve insufficiency. Circ. Res. 63:380-385;
Sehl P D, Tai J T N, Hillan K J, Brown L A, Goddard A, Yang R, Jin H, Lowe D G. (1999) Application of cDNA microarrays in determining molecular phenotype in cardiac growth, development, and response to injury. Circulation 101:1990-1999;
Sokolov B P, Prockop D J. (1994) A rapid and simple PCR-based method for isolation of cDNAs from differentially expressed genes. Nucleic Acid Res 22: 4009-4015;
Stanley W C, Lopaschuk C D, Hall J L, McCormack J G. Regulation of myocardial carbohydrate metabolism under normal and ischaemic conditions: potential for pharmacological interventions. Cardiovasc Res. 1997; 33:243-257;
Stanton L W, Garard L J, Damm D, Garrick B L, Lam A, Kapoun A M, Zheng Q, Protter A A, Schreiner G F, White R T. (2000) Altered patterns of gene expression in response to myocardial infarction. Cir. Res. 86:939-945;
Steenman M, Chen Y W, LeCunff M, Lamirault G, Varro A, Hoffman E, and Leger J J. Transcriptomal analysis of failing and nonfailing human hearts. Physiol Genomics. 2003; 12:97-112;
Tan F-L, Moravec C S, Li J, Apperson-Hansen C, McCarthy P M, Young J B, and Bond M. The gene expression fingerprint of human heart failure. PNAS. 2002; 99(17): 11387-11392;
Tamura T, Said S, Gerdes A M. Gender-Related Differences in Myocyte Remodeling in Progression to Heart Failure. Hypertension. 1999; 33:676-680;
Taegtmeyer H. Genetics of energetics: transcriptional responses in cardiac metabolism. Ann, Biomed. Eng. 2000; 28:871-876;
Von Stein O D, Thies W G, Hofman M (1997) A high-throughput screening for rarely transcribed differentially expressed genes. Nucleic Acid Res., 25:2598-2602;
Warburg O, On the origin of cancer cells. Science. 1956; 123:309-314.
White M, Wiechmann R J, Roden R L, Hagan M B, Wollmering J D, Port E, Hammond W T, Abraham E E, Wolfel E E, Lindenfeld J, Fullerton D, Bristow M R. (1995) Cardiac beta adrenergic neuroeffector systems in acute myocardial dysfunction related to brain injury: evidence for catecholamine-mediated myocardial damage. Circulation 92: 2183-2189;
Wong B R, Park C G, Lee Y S, Choi Y (1996) Identifying T cell signaling molecules with the Clontech PCR-Select cDNA subtraction kit. Clontechniques XI: 32-33;
Wong B R, Rho J, Arron J, Robinson E., Orlinick J, Chao, M Kalachikov S, Cayani E, Bartlett F S, Frankel W N, Lee S Y, Choi Y (1997) TRANCE is a novel ligand of the tumor necrosis factor receptor family that activates c-Jun N-terminal kinase in T cells. J. Biol. Chem. 272: 25190-25194;
Xiao-Jun Du. Gender modulates cardiac phenotype development in genetically modified mice. Cardiovascular Research. 2004; 63:510-519;
Yang M, Sytowski A J. (1996) Cloning differentially expressed genes by linker capture subtraction. Anal Biochem 237: 109-114;
Yokomizo T, Izumi T, Chang K, Takuwa Y, Shimizu 1 (1997) A 0-protein' coupled receptor for leukotriene B4 that mediates chemotaxis, Nature 387: 620-624; and
Yue P, Long C S, Austin R, Chang K C, Simpson P C, Massie B M. (1998) Post-infarction heart failure in the rat associated with distinct alterations in cardiac myocyte molecular phenotype. J Mol Cell Cardiol 30:1615-1630.

When introducing elements of the examples disclosed herein, the articles “a,” “an,” “the” and “said” are intended to mean that there are one or more of the elements. The terms “comprising,” “including” and “having” are intended to be open ended and mean that there may be additional elements other than the listed elements. It will be recognized by the person of ordinary skill in the art, given the benefit of this disclosure, that various components of the examples can be interchanged or substituted with various components in other examples. Should the meaning of the terms of any of the patents, patent applications or publications incorporated herein by reference conflict with the meaning of the terms used in this disclosure, the meaning of the terms in this disclosure are intended to be controlling.

Although certain aspects, features, examples and embodiments have been described above, it will be recognized by the person of ordinary skill in the art, given the benefit of this disclosure, that additions, substitutions, modifications, and alterations of the disclosed illustrative aspects, features, examples and embodiments are possible.

In the sequences provided in the attached Appendices A and B, certain nucleotides are represented as nucleotides other than A, C, T or G. In particular, each of the symbols Y, R, H, K, M, W and S, as listed for example, in SEQ. ID. NOS. 129, 554, 556, 558 and 570, may represent any nucleotide including A, C, T, G, hypoxanthine, xanthine, uric acid or other known nucleotides. Also, the letter “N” indicates the nucleotide may be any of A, C, T or G. SEQ. ID. NOS. 1-1143 (Appendix A) are from Homo sapiens and SEQ ID. NOS. 1144-1233 (Appendix B) are from Gallus gallus. All the sequences shown are deoxyribonucleic acid (DNA) sequences.

APPENDIX A

| | |
|---|---|
| | SEQ. |
| | ID. |
| Sequence | NO.: |

| | |
|---|---|
| GGAAAACAGCCGGTGATCTTCTACCAATAAAGCCAGTGGAAATTGCCATAGAGGCATGGTGGGTGGTGCA | 1 |
| TTTGCAAATATGTGTATAACCACATTGGTGGGGAGCATTCCGCTGTGATCCCAGAGCTGGCAGCCACAGT | 2 |
| CCTGGCTTGGAGACCCCTCTCTGCCATCTGTTGACTGGCTCTGTAATTCTGGAAAACACCCTTTCTAAAC | 3 |
| ACTGGTAAGACCATCACTCTCGAGGTGGAGCCGAGTGACACCATTGAGAATGTCAAGGCAAAGATCCAAG | 4 |
| TACTAAAAATACAAAAATTAGCCCGGTGTGGTGGCAGGTGACTGTAGTCCCAGCTACTCGGCAGGCTGAG | 5 |
| AAATTGTCCTAATAATATGTGGTGCTCATGAGTGCGGGACCTGACTGGGCTCAGCTAGGCGGTTCTCACT | 6 |
| ATACAAAAAATTAGCCCAGTGTGGTGGCACATGCCTGTAGTCCCTCAGACCTGTAAGCTACTCAGGAGGC | 7 |
| GTATGTAGAAGACTTCAAAGCCCTAGAGGATGGCAGAGCCACCAGCTGGACAAAAACTGGGCCCAGAATT | 8 |
| CATCCCACTCCATCCCTTCTGGGATGTGAATCATCCGTTTGTCCAGCGTATTCACGCTATATATGCTCCC | 9 |
| AGATATCGAGCTCAGGACTATTAAGCACGCCTGTCTACCCACAGCACAGTACTGATCATTACAGGGCGCA | 10 |
| ACTCATACCTCCCATCTTCCAGCTGAAGGGCTCTCAAGCCCGCTAAGCAAGCTTCTTTATTTACTCGGCT | 11 |
| GTACCGCCCCATGTATAAGGCTTTCCGGAGTGACAGTTCATTCAATTTCTTCGTTTTCTTCTTCATTTTC | 12 |
| TTTAGCCACAGACGTAGGCTACAAGACAGCGGAACATCACTTTACGGCTTTGCCCACAGACATGAAGGTG | 13 |
| CAGGGCGTAGGGCCTGGGCCGGGGTCGGCGGCGCCCCCGGGGCTGGAGGCGGCCCGGCAGAAGCTGGCGC | 14 |
| GTTGGGGTCCATCCCTCTCTGATGTGCTTTTTCCACAACACATATCTGGTCCTCTGGCAGGATTGTGGAT | 15 |
| CTGTGGTTGGAGTCCGTGCGGCTGGAGTACCGTGCGGGGCTGAAGAACATCGCAAATACACTCATGGCCA | 16 |
| GTCCCTGGGCAGCCCTCCATTTGAGAAACCTAATATTGAGCAGGGTGTGCTGAACTTTGTGCAGTACAAG | 17 |
| ACCTCGCCCATCTTCACTTAGCCTTCGTATTTGTGAAGGATTCAGCCACCTTCCTTCTTCACCCCATGCT | 18 |
| CAATGAAGATATTTTAGAGTACAAAAGAAGAAATGGGCTGGAATAAACTTTTGAAACACTAATGTAGTAT | 19 |
| CGCGTCGCTAGCTAGTCGTTCTGAAGCGGCGGCCAGAGAAGAGTCAAGGGCACGAGCATCGGGTAGCCAT | 20 |
| CGCAAGCTTGGCAGCCTTTGGTAGAGGGTAGCGAGAACAAGGGAATGTTGAGAGAATATGGAGAGACAGA | 21 |
| ATTTACCAACCTGGGGGATTGATACGACCGGGGAAAATGTTCCTAAACCAGGAAGCTGCGTTAGCCGATC | 22 |
| GCAGTGTGGGACAAAGTCCTTAGACAAGAAGCAGCCCAGGGTATCCAATAATTGAAAAAGGAGGCTGGGG | 23 |
| TGTGGGAGTATACATCGGTGCAGGCTTCCTGGATGACAGTTGGGTGATATGTGTCATGTGGCCTAAAAGC | 24 |
| CCCCTCTCCCAGGTGTCCCCTTGTAGCATATGCATTATGTCATCTGAATTGAGGCCTTTCTGTGAACAGC | 25 |
| ATTTTACACTTTGTTACTAATTTGCAGAACTCTATTAATTGGGTAGGATTTCACCCATTCCTAGCTAAGT | 26 |
| TGCCACGTATAGCTGGAATTAAGTGTTGTCTTGGAGCTGTTGTACATTTAAGAATAAACTTTTGTAAAAA | 27 |
| TGGGTCGGTAGTAGCGATGGCGGGTCTGACTGACTTGCAGCGGCTACAGGCCCGAGTGGAAGAGCTGGAG | 28 |
| GTCATCCAGCCCTGCTGTAAAATATGAAGCTGCTGGGACATTAGTGACACTCTCTAGTGCACCAACTGCA | 29 |
| CCTCTGAGGAGCCCTCCTGGATGAATGGAGGGAGGCACTCGGCTAACAAATTAGGGCTTCTCGACGTCCT | 30 |
| CAGGAAGCAGCGTCTCATCAGGACAGAAGGTAGGATGAAGACATGGGGTAATGTGAGAGAGTAGAACACC | 31 |
| TGAATCCCACTCCCACCAGAGAATTAGCGCGGGCGGACGAGCAAAGTGAAACTTAGTAGCCCGGAACTTC | 32 |
| CCCTGGAGCTGAGCACAAAGAGTCATGTGACSGAAGAGGAGGAGGAGGAAGAGGAAGAAGAATCAGATTC | 33 |
| GCGTGAGACACATCACATTTGTGGACAATGCCAAGATCTCCTACTCCAATCCTGTGAGGCAGCCTCTCTA | 34 |
| AGGGCCTGCTCCATCCCACCTTCCTTTCTGCTGCCTGATGTCTCAATGGCTTCTGAATGACTGTTCTAAT | 35 |
| GCGGACGCTATCTACGACCACATCAACGAGGGGAAGCTGTGGAAACACATCAAGCACAAGTATGAGAACA | 36 |
| TATGGGACCACACTGTGCTGAGAAGCTTCCTGAGGCCCCTCAACCTGAAGGCCCTGCTACAAGCAGTTCA | 37 |
| GCACTCCCTTGGTGTAGACAAATACCAGTTCCCATTGGTGTTGTTGCCTATAATAAACACTTTTTCTTTT | 38 |
| GCACCATTGAATTCTGCAGTTCCTAGTGCTGGTGCTTCCGTGATACAGCCCAGCTCATCACCATTAGAAG | 39 |
| ATCTGTGCGGAAGTAGCTTGCCTCACTTCTGCTTAGGAAAGCGGCTGTTGCTCCATAACTCTAACCAGCA | 40 |
| GCGCGCACGCACGCCTTGAGCAGTCAGCATTGCACCTGCTATGGAGAAGGGTATTCCTTTATTAAAATCT | 41 |
| GACCTACTGTATTAGACAGTAACCTCTAACCTCACCTCCAAGCCCAAGTATATGGCCCTGCTGGGTTACC | 42 |
| CATATCTGTTTCCTCCATCGGAGCAAAACCACTGAGATCATCCATTCAACCCTGAATCCCACGTGGGACC | 43 |
| TTACATCCATCTATGAGTGGAAAGGGAAGATCGAGGAAGACAGTGAGGTGCTGATGATGATTAAAACCCA | 44 |
| GTATTCAGCCTTTAGGATGATCAGAAAAGCAGAAAGAGAGAGTGGCCGGATGGGGCTGAGGGGAGAAAGA | 45 |
| AGGCCCCGCAGTCCCTCTCCCAGGAGGACCCTAGAGGCAATTAAATGATGTCCTGTTCCATTGGCAAAAA | 46 |
| TACTAATAATTATTAGCTACAGGCGGGCGCAGTGGCTCACAACCGTAATCCCAGCAGTTTGGGAGGCTGA | 47 |
| GCCGCCACTCCAGCCTAATCCCAACCCCAGGGCGAACGTTTTCTTATTTATTTCCGTTTTCTCGCCACTA | 48 |
| GGATCTGGGCAGTCAGCACTCTTTTTAGATCTTTGTGTGGCTCCTATTTTTATAGAAGTGGAGGGATGCA | 49 |
| TCGCCCACTAAGCCAATCACTTTATTGACTCCTAGCCGCAGACCTCCTCATTCTAACCTGAATCGGAGGA | 50 |
| GCCAATGGTGGCAGCAGAAGTAGGCGTATGGGATAACTATTGTGTAAAGAAACAGCTTCTTCACTCCTGC | 51 |
| CTTATGACATTATCTCTAGGCTGCCACTTAAAGTATGGTTTGAAGACAGGGAGAACGGGGCGGCGGAGTG | 52 |
| ACTAGCCGTGTTTTCTCAGACTCCACCTTTGTTTGCACTCTGTTGCCTGTGAGGAGCTTTCTGGCATGTG | 53 |
| GAGGAGCTCTCGACTTAGAGGTAATATGAACAGATGAACAGACACTGTGGCTGGAGCCCCAAAGTGTGGA | 54 |
| TGCCCCACTGAGAAGGGTCTAGCGGAGCACAGGTCACCAGCTGGGCAACATTCAGAAAGTTAGTCTTCCT | 55 |
| CGGGAGGACAACCAGACCAACCGCCTGCAGGAGGCTCTGAACCTCTTCAAGAGCATCTGGAACAACAGAT | 56 |
| TGAGGCATGTACTCCCCATGAGGCCACACAAGAGCTGTGCTTTCTTAGATCTGGATCCCACTACCACATA | 57 |
| TGAGCCAGGCCTACTCGTCCAGCCAGCGCGTGTCCTCCTACCGCCGCACCTTCGGCGGGGCCCCGGGCTT | 58 |
| CGGCTTCGACCCTATATCCCCCGCCCGCGTCCCTTTCTCCATAAAATTCTTCTTAGTAGCTATTACCTTC | 59 |
| GAACAGCCAAGCTTTGTGCTACTATGGGATTTCGTTTTCTGCGGTTCCAAGTCTTGATCCACGTCCTGCC | 60 |
| CATGTCATGCAGCTCAGCTGGGAGCTGCTTAGGTGGAAAACTCCAAATAAAGTGCGCCTGTCGCAGAAAA | 61 |
| CCCAGAAGCAGTTAAGTCTCCAAAACGAGTGAAATCTCCAGAACCTTCTCACCCGAAAGCCGTATCACCC | 62 |
| CCAGGAAAGATTTGCCCTCAAGAACCTCAAATGTAGAGAGAAAAGCATCTCAGCAACAATGGGGTCGGGG | 63 |
| CTGTGGCCAGGGGTCCAAACAGAAAATAACCGGAGAAGACAAGGAGGTCAAAGGATCAGGGAACTAAGCA | 64 |
| AACCCTGGGGATTGGGTGCCATCTCTCTAGGGGTAACACAAAGGGCAAGAGGTTGCTATGGTATTTGGAA | 65 |
| AAGAGCGTCAAGCAGACCTGTGACAAGTGTAACACCATCATCTGGGGGCTCATTCAGACCTGGTACACCT | 66 |
| CCTCTGACCGTTTCAGCACCCTGGGTTGTTACCACGTCCTACAACTCTGACATTTCTTGTTCTCAAGCGT | 67 |
| ATTGGTGAGCTGAAGTCTGTCCTTGCACCATGTTATCATCTGTTTCTCGTGTCCGCCTGGTTGAGGAGGA | 68 |
| AAGCTCACCTGGGCAGGTCTCTGCCACCTCCTTGCTCTGTGAGCTGTCAGTCTAGGTTATTCTCTTTTTT | 69 |
| GAGAATGATTTACAACCCCTGCTAGCCTGGCTTAAGGTCATGGAGAAAGCCCACATCAACCTGGTGAGGT | 70 |
| CATTTTCTGTTGCAGGAAGCCACTCCACCACAGAATGCTAATATGCCAGTGGTACCCAGTACCTCTTGTA | 71 |
| TCCCTTGATCATTATCTCTGAAGTCCCTACCTGCACTTCCCTGATTGCCCTGTAGCAACACCAGCATGGT | 72 |
| TTCTGACAGGAAAGGGGCTCCGGAAAATCATAAAACAAGCAGGTGAACAAGACCAGGTGTGTCGGCACCT | 73 |
| TTTTCTCACAAGAACCCAGTTAGCTGATGTTTTATTGTAATTGTCTTAATTTGCTAAGAACAAGTAATAA | 74 |
| GGGTTTGTGAAAAGTGTATGTATTTAAATTTGCTGTAAAACATAATCACTAATAATATGCAATAAATATT | 75 |
| TTTGGGAGAGACTTGTTTTGGATGCCCCCTAATCCCCTTCTCCCCTGCACTGTAAAATGTGGGATTATGG | 76 |
| CTCCGTGAGAGCAAGGATCCTCCTGTTTACCCTGTACCTCCAATGTCTGGCACTTGTAGGTGCTCAAATA | 77 |
| CTCGGAGCAGAACCCAACCTCCGAGCAGTACATGCTAAGACTTCACCAGTCAAAGCGAACTACTATACTC | 78 |
| GGGACTGCCAGCCCCTAACTGAAATCTGAAGCTTTTTATCGCTTATTTTTCCTCGCCCTGTCTCCTCCCT | 79 |
| ACCACGGCTGTGCTACTCACGGTCATGCTGGAGGGATGCAGAAACTAAATGAATCCACAGCTACTTACTC | 80 |
| CTCCCCAGCCACAAGGAGTAGAACCAGTAGCTCAAGGAATTGTTTCACAGCAGTTGCCTGCAGTTAGTTC | 81 |
| ACGAGGAGACAGGGAAAGTGAAGGCCCACTCACAGACTGACCGAGAGAACCTGCGGATCGCGCTCCGCTA | 82 |
| CGAAATGAAGTTTATCATAGGAAAATCATCTCTTGGTTTGGTGATTCCCCCTTGGCTCTTTTTGGCTTAC | 83 |
| GCTGAAAGATGTACTGCAGTCAGCTTCAGGGCAGCTTCCTGCCACAGGAGCATTAAATGAAGTTGGAATT | 84 |
| GTACTTGGAGTTGGGACCTCACCTGGCTCTCCCTTATCTTTCCGGCTGCCATTTTTTCCCCTTTCTAACT | 85 |
| TAGATCTCTAAGCCCCTCCTGGAACCCTCATTTTCCCCACTCTCAATGTCCCAGTGTCCAGCGTGACTAA | 86 |
| CCAGGGACTGCCCCAGCTGTCCTGGGCACAAGTCTCTCCAGCATCTTTGTTCATTGATTCAACAAAGTAT | 87 |
| AATAATGCCTGGTCATTGGGTGACCTGCGATTGTCAGAAAGAGGGGAAGGAAGCCAGGTTGATACAGCTG | 88 |
| TACGGCCGCGCCTTTGTGTTCCTGTCTTCTCTCCACCACCAAAAGCAAAAGATGATTTCCCATTCACTGC | 89 |
| ATCATCATGTCCTAGCACAGATGGCCCCAAGCAGGGGAAGTACAATACTGCAGGCTGCAAATCCATGTCA | 90 |
| CCCAGACTCACCGGACAGGATAACTGTGGCCTCTTCATTAAACTGCACCGTGTTCACCTTCTGAGAAAGT | 91 |
| ATTTGCATCTGAAAGGTCCCAAGGTGAAGGGCGATGTGGATGTTTCTCTGCCCAAAGTGGAAGGTGACCT | 92 |
| GATGGAAAGAGTCTCACTTGCAGTTGCTTCAGTCACAACCCAGGCGTCTGCCTTAATAGCATCACCTGTG | 93 |
| AATACAGCAATTTTGGCAATAACTCTTATCACTCCTCAGGGCTTAGGGTGGTCCCAGGTACCCAGGGGTC | 94 |
| GAAATGGTGCGTTGGTGGTCATACTTAGTGTTCTAGGCTGTGAAATCATGGAGTTCTTCCACTTCCAAGA | 95 |
| TGTTGGGCCCTGAAAAATTAGTCCGATTTTGTGGTGGTAATGGGAGAAGGACATCCCAGGAGCAGGGTCT | 96 |
| CCATGACCCTGAAACTAGAACAACACGTCTCCTCCCTAAGTCTGCAGCTTCCAGATCCTCGAATTGCAAC | 97 |
| CCCTCTGCCAGGCGCTAGACATGTACAGAGGTTTTTCTGTGGTTGTAAATGGTCCTATTTCACCCCCTTC | 98 |
| GGGGGAGTTGAGCAGGCGCCAGGGCTGTCATCAACATGGATATGACATTTCACAACAGTGACTAGTTGAA | 99 |
| CCCATCCCTAATAGGCTGGGCTTTGCAGGAAATGGCATGAAATCAGCTCTTCTGAGTGCACAGAAGAACC | 100 |
| GGGGGTTCCTTCCTGTTGCTAAGGTTTGGAGGTGTTCTGTTATTTACCTGAAGTGCTGCAGCTGGGAATC | 101 |
| ATCATTGAAAGGTCCTCTCTGCCAGCAGTGGTGCCACCCTTTGGTTTGCTGTGGTACTTTGCTGTGTACT | 102 |
| GCCGGTTTTTCCATGTCATACAAAAAAGTCCTGGCTGTTTCTCCGAACTGGCTGCCTGCATTCCCGTCTT | 103 |
| CTGAGAGGAACCTGGACATGGTCCCGGGCATCTGAATGATCTGTAGGGGAGGGAGTTCAAATAAAGCTTT | 104 |
| TCAAAACCCTGAGCCCTGTGCATGCTTTCTCAGTCTTGTGGTGGGACTGGATACAATGACTAACTTCCCC | 105 |
| GGTTGCACTGGGGAGGTCTGGGAAGATAGCTGTTTCTGAAGACTTGCCGCTGTGGACACAGTTAACTAAA | 106 |
| GGAGAAAGAAGAGCTCGCTGTGAAAAACGCTCCACAATGCTGCAGAGCCTTGTGAAGGTGGAAGAGTACT | 107 |
| ATAAAGTTGTTACAAAGTGACCTTGAGTGTCTTCCTTGGTGCACCCGAAACCCCGCCTTCTTCATCCGGG | 108 |
| AGCCAGTCCTGTTGGTGGAGGGGATCACCGAGAGTGTCTGTATCATTTTGTAGCCCTTTTCTCTGACGTT | 109 |
| GGGCATCTGAGGGCAGTAAGGAACAGGTGTCCAAAGGAGGAATGTTGGTGCCTATGAGTATGTTTTCCAG | 110 |
| AAATGGCCACCACCATTCTCCTTCCCCACCCCACCACAAAAAGAGAAGCTGTGTCTTTAGACAACCCTGA | 111 |
| GCCCGCAGTTGGAGTTGGACTGTCTTAACAGTAGCGTGGCACACAGAAGGCACTCAGTAAATACTTGTTG | 112 |
| CTCTGAAGCGAGCTGGTTTAGTTGTAGAAGATGCTCTGTTTGAAACTCTGCCTTCTGACGTCCGGGAGCA | 113 |
| CCCACCTGTAGATCCATAGCAACAGTGGATCAGGGCAGGAAGCAAGCACATAAAGTGGAGTTTCCCTTCT | 114 |
| CTGAGCCTAGAGCAGGGAGTCCCGAACTTCTGCATTCACAGACCACCTCCACAATTGTTATAACCAAAGG | 115 |
| GCCTGGGGAACGTGGTTGGCTCAGGGTTTGACAGAGAAAAGACAAATAAATACTGTATTAATAAGATGTT | 116 |
| GCACCGTTAGGTTTCAGATCTCCCGTGTGGTGTTTGATGTCGGCTTTTGTTCCTACCTTGGGAGTTTGGA | 117 |
| GTAAGTAACTTGTGCTAGTCACTGGGGGACCTGGGTTTCAGACTGGGCAATCTGGCTGATCATTTTCCAG | 118 |
| CACCTTGGCCTCTGAAAGTGCTAGAATTACGGGCATGAGCCACCGCATCCAGCCAGAAAGATACATATCT | 119 |
| ATCCATTCTCACATTTAAACTACTGTCCAGGGCCGGGCGCAGTGGGTCACGTCTGTAATTCCAGCACTTT | 120 |
| GTTTGGACTATAGAAATGCGGCTGTTCGCTGCAACCAATCAAAACCCTCTGTGGTTTAGGCTAGCGGGCT | 121 |
| GGCCAAAGAGAACACCAGAAGACCCTTAATTTTACAGGCAGAGTTGCCTCAGGCCAATGACTGGCTCCAA | 122 |
| CATTCTCCTAAAGGTGACTCCAGTCCTGTGCTGAGTCCTGTGCATTCTCCTAAAGGTGACTCTAGTCCTG | 123 |
| GCGTCAGGAGCCGGCTGTGTCCTTCCTGCCACACTCGGGGATTCATTCCTTAGAAACTGAAATAAATTCT | 124 |
| GGGCACTAATGGAGATACTCATCTGGGGTGGAGAAGACTTTGACCAGCGTGTCATTGGACACTTCATCAA | 125 |
| CCAGGTCTCTGTAGTACTTGGCAAACCTGAAATTGTAGCCAGGAGATACGTTGTGCTCAACGTCCCGTGT | 126 |
| CCATAAAATGTTTCTCTTCTGAACAAGCCCCATCATTTGGTGAACCTCCACCCTAACAAAGTAGGATGGG | 127 |
| TCAAGTGGAGCTTCATGAATAAGCCCTCAGATGGCAGGCCCAAGTATCTGGTGGTGAACGCAGACGAGGG | 128 |
| GCTATAGGTTGGAGCTTGGCTCTATCTGCTGTCTCAATAACAGCCTTTGAACTGTCCACGTATCTYAAAA | 129 |
| TCCTCCAGGTTTTTCAATTAAACGGATTATTTTTTCAGACCGAAAAGAGATGGTCTGAGTTTGTCTTAGA | 130 |
| CTGCTGCCATGTGAGTATGTGGGCCCAGTGTTGCCAGATCACCTGCTTTTATACGAAGACCCTAAACTCT | 131 |
| TATAAAAATTAGCCAGTACTAGGAAGGCTGAGGCAGGATAATCGCTGGAACCCGGGAGGTGGAGGTTGCA | 132 |
| AAAAGAAATTAGCTGCACATTGTGGTGAGCGCCTGTAATCCCAGCTACTCAGGAGGCTGAGGCAGGAGAA | 133 |
| GCACTCTCAAATTTCTACGCTCAAACAATCCTTCCACCTCAGGCTCCTGAGTAGCTGGGACTACAGGCAT | 134 |
| AAAAAAGAGCCCGCATCGCCAAGTCAATCTTAAGCCAAAAGAACAAAGCCAGAGGCATCACACTACCTGA | 135 |
| AATTCCCGGTTCTCAGAATTGTTATCACTCTGGTGCATGCTGTCACAGGGGCCGTTGCGTTTGGCTTTGT | 136 |
| GTGGATGGATCACAAGGTCAGGAGATCGAGACCATCCTGGCTAATACGGTGAAACCCCGTCTCTACTAAA | 137 |
| CTATCAAAGGGTGGGGTGGTGCCACCTCCGTGCTGTGCAGGAGTCAAAAAGTTGAACGGTATGGCTCAAA | 138 |
| TTAGTGCCTGCACCTCACCACGATATTGAGGAAGCACAGGACATCCAAGGGTACTCTCCAGTTTGGCTGT | 139 |
| TCATCTTTTAGAGCAGCTGCCATCACATCGGACATATTGGAGGCCCTTGGAAGAGACGGTCACTTCACAC | 140 |
| CAGGCCACCTACTCATGCACCTAATTGGAAGCGCCACCCTAGCAATATCAACCATTAACCTTGCCTCTAC | 141 |
| AAGCCCCTATTTTTTCCAAGCACGAAGCCACCAGTCTTCCCCAGGGAGCNATCAGNAGGGACATGGATGT | 142 |
| CTGCGCCTGAGGGGTGGCTGTTAATTCTTCAGTCATGGCATTCGCAGTGCCCAGTGATGGCATTACTCTG | 143 |
| TAGGCTTAAAAACAGATGCAATTCCCGGACGTCTAAACCAAACCACTTTCACCGCTACACGACCGGGGGT | 144 |
| CTGGAAAAGGGACAGACTATCAGAGAGTTGCACTGTTGCGGTATGGGCCAAATCCAACATAATACCCGCT | 145 |
| TCGCACCACTGCACTCCAGCCTGGGCAACAGAGCAAGACTGTGTCTTGACAGCAACAAAAAAAGAAGATA | 146 |
| CGCTGATTTCCTGAAATAGAGATACCCCTTTGAGTGATAAATTTGCAAAATGCTGTCTTCATTTTCTGTA | 147 |
| CCGCTGGGGAGGTCCTCCATGCGCAGTCATGAGTCGCTTCAAGTTTATCGTTTATGATTACAGGTGGAAA | 148 |
| CAAATACTTTTCCTGCCTCCACCAAACCCCTACAGAACATCACCTGGAATTGCCACTCACACTGGGTTGG | 149 |
| TTTAGCCACAGACGTAGGCTACAAGACAGCGGAACATCACTTTACGGCTTTGCCCACAGACATGAAGGTG | 150 |
| GGATATTCTGTTGGTGATACCAAACACCAAGGGGCCTCCAGCTGGGTTTCAGTAGTACGATGAGTCACTG | 151 |
| CATCCAAACCCAGTCGTCTGCCCTGGATCGTTTTAATGCCATGAACTCAGCCTTGGCGTCAGATTCCATT | 152 |
| TTTCTTTAGACCCATACTTACTGTTCCTCAAATGCCTGCAGTTTGCCCGGGAGTCGTCTCTGCAACTGGC | 153 |
| TCGCCCACTAAGCCAATCACTTTATTGACTCCTAGCCGCAGACCTCCTCATTCTAACCTGAATCGGAGGA | 154 |
| GGGTACCACCCAAGTATTGACTCACCCATCAACAACCGCCATGTATTTCGTACATTACTGCCAGCCACCA | 155 |
| TCGCCCACTAAGCCAATCACTTTATTGACTCCTAGCCGCAGACCTCCTCATTCTAACCTGAATCGGAGGA | 156 |
| TAAGCAGTGATCTTTGCTGCTGCTTTCCCCCTTTGTCTGCCCTTAGGTCACTAAGGATTGTAGGGCCTTC | 157 |
| AAGCACAAGACTGACCTCAACCATGAAAACCTCAAGGGTGGAGACGACCTGGACCCCAACTACGTGCTCA | 158 |
| TGTGAGGTTTTACAGTATTCTGCAAGGGAAGCTCAAGATTCAAAAAAGGTGGTAGAGGACATTGAATACC | 159 |
| AGAAATGGATGTGGGAACAGATGAAGAAGAAACAGCAAAGGAATCTACAGCTGAAAAAGATGAATTGTTG | 160 |
| CCTACCTGCCAACCTCTCCTCTGCTGGCAGATTGTATCATCCCCATTACTGATATCAGGGCTTTCACAAC | 161 |
| AAGTGTGACAACTTGATCTACTAGCGAGGCTGCATGGGGAAACAGGCACTTTCATAGGTAGCTGGTGGGA | 162 |
| TTGTAATCCAGGACATCTGATCTCCTACATCAAAAACTCCAATGGGGCCAGGTGTGGTGGCACTTGCCTG | 163 |
| GCCATGAAGGCACTGAGTCTGTCTGGTTTCCTGAGGGTTAAAAGATTAGGGCTGGGATCACCACAGCATT | 164 |
| GACCTCCCCAGCATCCCTGAGGTGTGGCTGCTTAGTTTTCGATACTTACCTTGTTACCAGATGTCAGACT | 165 |
| GAATTGCCCAGTGCTGCCAGAGTGAGTGAGTGTAATTCTCCTTTCAGGTAAAGATAGGCTATCTCAACAC | 166 |
| AAATAGGGCTGGATCTTATCACTGCCCTGTCTCCCCTTGTTTCTCTGTGCCAGATCTTCAGTGCCCCTTT | 167 |
| ATGTCATAACTTCTGTTACTCCTTTGGCCCATAGCTAAGGTCATCCTTCCCCACAGGGGTGGCTTTGGGA | 168 |
| CTTGCCAGAAGATGATCTTAGAGTTGTTTTCTAAGGTGCCATCCTTGGTAGGAAGCTTTATTAGAAGCCA | 169 |
| CTGGTCACCGTTTCACCATCATGCTTTGATGTTCCCCTGTCTTTCCCTCTTCTGCTCTCAAGAGCAAAGG | 170 |
| AGAGTGTTGTCCAGATGTTTCTGTACTGGCATAGAAAAACCAAATAAAAGGCCTTTATTTTTAAACAAAA | 171 |
| AATGGAAACATCTGCCCCACGTGCCGGAAGCCAAGTGGTGGCGACAACTGCGCGCCACTCCGCGGCCTAC | 172 |
| TAGTGCCACTAACGGTTGAGTTTTGACTGCTTGGAACTGGAATCCTTTCAGCAAGACTTCTCTTTGCCTC | 173 |
| TAATCCTGCCAGTCTTTCTCTTCAAGCCAGGGTGCATCCTCAGAAACCTACTCAACACAGCACTCTAGGC | 174 |
| TTTCACATATGTTGTGAATTTTCCTTGGTTCTTTTTAAAGGAATGATAATAAAGTTACTTGCTTTAGGAA | 175 |
| GGGTGTCCGCTGCTGCTTTCCTTCGGAATCCAGTGCTTCCACAGAGATTAGCCTGTAGCTTATATTTGAC | 176 |
| CTGTTGCAACTCGGCTGTTCTGGACTCTGATGTGTGTGGAGGGATGGGGAATAGAACATTGACTGTGTTG | 177 |
| GATTGCTGTGTACCCTGCCTTTGAAGCACCTCCTCAGTACGTTTTGCCAACCTATGAAATGGCCGTGAAA | 178 |
| CACACGTAGTGGCTTAAAGCAACGAACATTCACTCTCTCACAGTCTGTGTCAGTCGGGGATTTGGGAGTG | 179 |
| TCCTCTAACTAGGACTCCCTCATTCCTAGAAATTTAACCTTAATGAAATCCCTAATAAAACTCAGTGCTG | 180 |
| GAAATGGGTCCCTGGGTGACATGTCAGATCTTTGTACGTAATTAAAAATATTGTGGCAGGATTAATAGCA | 181 |
| ATTATTGCAAATACTATGGGTACCGCAATCCTTCCTGTGAGGATGGGCGCCTTCGGGTGTTGAAGCCTGA | 182 |
| AAGCTACACTCAAAGACACTCCCACCAGGCTCTTTCTCCCTTTTCCTCTTGCTCACTGCCCTGGAATCAA | 183 |
| ACTTGAAAAATTACACCTGGCAGCTGCGTTTAAGCCTTCCCCCATCGTGTACTGCAGAGTTGAGCTGGCA | 184 |
| CCAATCTTTTACAAAGCATGGGAGTGCAGCTGCCTGACAACACCGATCACAGACCAACAAGTAAGCCAAC | 185 |
| CCCAGCTCATCCAGGGAGGGCGGCTTATCAAACACGAGATGACTAAAACGGCATCTGCATAACAATGGAA | 186 |
| ATTTGGATCTCACGCTGCCTCTGTGGTTCCCTCCCTCATTTTTCCTGGACGTGATAGCTCTGCCTATTAC | 187 |
| TGGAGGCCTGTGGTTTCCGCACCCGCTGCCACCCCCGCCCCTAGCGTGGACATTTATCCTCTAGCGCTCA | 188 |
| GGACAAGAAAGAAATGGCCATCAATGACTGCAGCAAAGCAATTCAATTAAACCCCAGCTATATCAGGGCA | 189 |
| GTCCCCAACCTAGCTTGGTGAGGGCTGTAACTGTTTCCAAGTACTTGTACATTGGAAGTCTGAATGTGTA | 190 |
| GGCTGGCTAACTCGTAGGAAGAGAGCACTGTATGGTATCCTTTTGCTTTATTCACCAGCATTTTGGGGGA | 191 |
| GCGGCCGGCATCATGACCCTGTTTCACTTCGGGAACTGCTTCGCTCTTGCCTACTTCCCCTACTTCATCA | 192 |
| ATCATGCATGAAGCGCCAAAGATGCACCATGTAGAATTTTCACTTTGTACTGGCAGGCTCGTTTTACCTC | 193 |
| TCTCCTCTAGACCAAGGCAGGCAGCCCCGACATCTGCTTCCTCTATCGCCCAATGCAAAATCGATGAAAT | 194 |
| GGTCCGGTGACCCCCTGGCCCCAGATGGCACTGAGTTTTTCATTCATTGAAGATTTGATTTCCTTGAAAA | 195 |
| CAAGGTACTCTGGTGAGTCACCACTTCAGGGCTTTACTCCGTAACAGATTTTGTTGGCATAGCTCTGGGG | 196 |
| GAAATTAGGGCCTCCTCTGATCTCTCGCTATCTGCGGGTCCTGTCCTTTTCTCAAGACCTTCACCATTAC | 197 |
| CCTTCCTTGCCAGGACCTAGAGTTTGTTCAGTTCCACCCCACAGGCATATATGGTGCTGGTTGTCTCATT | 198 |
| GAAGATGGAGACACCCTCTGGGGGTCCTCTCTGAGTCAAATCCAGTGGTGGGTAATTGTACAATAAATTT | 199 |
| GTGTAGGGAAAAGGATCCACTGGGTGAATCCTCCCTCTCAGAACCAATAAAATAGAATTGACCTTTTAAA | 200 |
| TCAGGCTTTCTGTGCATGTACTAAAAAAGGAGAAATTATAATAAATTAGCCGTCTTGCGGCCCCTAGGCC | 201 |
| GGTGCTAGGAGAGGATGGTCTCCACCCATCTTTCTATTTCCAGTACACGTCACATTATTTTACCGGTGAG | 202 |
| GGCCAAAAACATACAGAGGTGCATGGCTGGCAGTCTTGAAATTGTCACTCGCTTACTGGATCCAAGCGTC | 203 |
| TTTCCCCTGCTCGGAAGGGTTGGCCTGCCTGGCTGGGGAGGTCAGTAAACTTTGAATAGTAAGCCAAAAA | 204 |
| AACATGGTATTAAACTCTATAAACCTCTCATTCTCCCTGTGACTCAGGCCCCAATCTTCATCTCCTTCTT | 205 |
| GGCACTGTGCATATTTTCAACCAGATCACCAGGAGCTGAGATCTTCTTCAGTCCCTAGCCAGGAATACCC | 206 |
| AATTCGGCACGAGGCCCGACGCTGTGGTTGCTGTAAGGGGTCCTCCCTGCGCCACACGGCCGTCGCCATG | 207 |
| AGATGGACGTGCACATTACTCCGGGGACCCATGCCTCAGAGCATGCAGTGAACAAGCAACTTGCAGATAA | 208 |
| ACTGAGGGGCAAGATTAGCGAGCAGGACAAAAACAAGATCCTCGACAAGTGTCAGGAGGTGATCAACTGG | 209 |
| GCCACCAGAGACTGAGTGGAAATCGCCCCTTTTGAAGGTGCCATTCTTATGAGCCAAAAGTTTGTCATTT | 210 |
| CAGTATGAGAAAAATATTCAAGTAACACTTTAAAACCAGTTACCCAAAATCTGATTAGAAGTATAAGGTG | 211 |
| CGGCCATGCCTTTCTTGGACATCCAGAAAAGGTTCGGCCTTAACATAGATCGATGGTTGACAATCCAGAG | 212 |
| GAGGTTGCTCAGCTCAAGAAAAGTGCAGATACCCTGTGGGACATCCAGAAGGACCTAAAAGACCTGTGAC | 213 |
| CAAAGGAAATCAGCAGTGATAGATGAAGGGTTCGCAGCGAGAGTCCCGGACTTGTCTAGAAATGAGCAGG | 214 |
| TATCAGAGGTGTGGAAGAAGAGGAAGAAGATGGGGAAATGAGAGAATAGCATCTTTTGTGGGGGATTTTT | 215 |
| GAACAAGTGGTTCTTCCAGAAACTGCGGTTTTAGATGCTTTGTTTTGATCATTAAAAATTATAAAGAAAA | 216 |
| AGGGATCCACTGTGCGGTGCCAAAAAAGAGGCGGAGGCTCGCGGCACAGCTCTCCCGGCGCAGCTCTCGG | 217 |
| AATGTTCTCCGAAACAGGATCAACGATAACCAGAAAGTCTCCAAGACCCGCGGGAAGGCTAAAGTCACCG | 218 |
| AGTTCCTTCTTGAACCCTGGTGCCTCCTACCCTATGGCCCTGAATGGTGCACTGGTTTAATTGTGTTGGT | 219 |
| AGGTTTTCATTCGCACGGAACACCTTTTGGCATGCTTAACTTCCTGGTAACACCTTCACCTGCATTGGTT | 220 |
| CCAGCCCTTAAAATGAAATTAACTTCCTACTCAGGCACCCTGCTTAGGTGCACAGCTGTTCAATATACAC | 221 |
| AGGACAGTCATCAGAGGCTCTCAGGCTGAGCTCAAGTGCCCCGTGTGTCTTTTGGAATTTGAGGAGGAGG | 222 |
| TGACGACTTCGCCGCGCGTTGGTCAGCCATGGCCACCGCTCTCGCGCTACGTAGCTTGTACCGAGCGCGA | 223 |
| GCCTAGAGCCTTCAGTCACTGGGGAAAGCAGGGAAGCAGTGTGAACTCTTTATTCACTCCCAGCCTGTCC | 224 |
| ATTATATCCCCATTAAGGCAACTGCTACACCCTGCTTTGTATTCTGGGCTAAGATTCATTAAAAACTAGC | 225 |
| CCTTCTGTGACATGTGTTTATAAAAAATGGTTAAGTATATAATAAATTGAACATCTTTGAGATTGGAGAA | 226 |
| GCCGCCATGGGAGTGGAGGGCTGCACCAAGTGCATCAAGTACCTGCTCTTCGTCTTCAATTTCGTCTTCT | 227 |
| CCCTTGGGGAGGGGCCACCTGTAGTATTTGCCTTGATTTGGTGGGGTACAGTGGATGTGAATACTGTAAA | 228 |
| CTATGGTTGGATCTCAGCTGGAAGTTCTGTTTGGAGCCCATTTCTGTGAGACCCTGTATTTCAAATTTGC | 229 |
| GGGACCCTGTTACAGACATACCCTATGCCACTGCTCGAGCCTTCAAGATCATTCGTGAGGCTTACAAGAA | 230 |
| TTAAGGAACGCTAGCAGGGCATGGCACGTGAGCTCCGGAATAGATGTCTTCATCACTTCTTCCACTGTGT | 231 |
| AATGTCTGTCAGTAACGAGGCTTTTGATGTGTTGAGCTGGAGGTGAGTGGACCGGGGGCTGTGTTTTAAG | 232 |
| GCGCCGCTGAGTTGTCTGGCCCCGCCGACCCACGGCCCACGACCCACCGACCCACGAATCGGCCCGGCCG | 233 |
| CCGATACTCCCAGATCTGTGCAAAAGCAGTGAGAGATGCACTGAAGACAGAATTCAAAGCAAATGCTGAG | 234 |
| GCACCCTCCTGAAAACTGCAGCTTCCTTCTCACCTTGAAGAATAATCCTAGAAAACTCACAAAATGTGTG | 235 |
| GGAGTTTCTGACTAATCAAAGCTGGTATTTCCCCGCATGTCTTATTCTTGCCCTTCCCCCAACCAGTTTG | 236 |
| ATTTACAAGACAGGTTTTAACTCAGCCGAGGTGGGAAATGGTGTCCCTGTCCCTCCCAAAGCACAGAGCA | 237 |
| CCTTGCTTCTGACTTTCGCCTCTGGGACAAGTAAGTCAATGTGGGCAGTTCAGTCGTCTGGGTTTTTTCC | 238 |
| TGAAGCAGATGATGAAAACTCTCAACAACGACCTGGGCCCCAACTGGCGGGACAAGTTGGAATACTTCGA | 239 |
| TCATTTCCTCAATGGGACGGAGCGAGTGTGGAACCTGATCAGATACATCTATAACCAAGAGGAGTACGCG | 240 |
| CTGCAGAGAAGAAACCTACTACAGAGGAGAAGAAGCCTGCTGCATAAACTCTTAAATTTGATTATTCCAT | 241 |
| GACCATTTGGAAGAAAAGATGCCTTTAGAAGATGAGGTCGTGCCCCCACAAGTGCTCAGTGAGCCGAATG | 242 |
| GAATGAGACATCCAGCAGATTTCCAGCCTTCTACTGCTCTCCTCCACCTCAACTCCGTGCTTAACCAAAG | 243 |
| TAGTTCTTCACCTTTTAAATTATGTCACTAAACTTTGTATGAGTTCAAATAAATATTTGACTAAATGAAA | 244 |
| CCGAGGAAGATACTGAGGGAGCACAGGAGCAGTCACCGCTGCCACTGCTACTGCCGCTACTGCTGCCGGC | 245 |
| GGGGCAGCACTGGGCCTGGCCCCCCGGGTATTTATTGCTGTACATAGTGTATGTTTGTGATATATAAGGT | 246 |
| CAAACATTAGATCCTAACAATATGACCATACTCAATAGGACTTTTCAAGATGAGCCACTAATTATGGATT | 247 |
| GAGGGGAAGCCACTTAATAAGGAGTCAGACCTAAAAGGGGGTGGGGGACATTTTCTTACCTCACCCAAGA | 248 |
| AATCCACTCACGTTCATAAAGAGAATGTTGATGGCGCCGTGTAGAAGCCGCTCTGTATCCATCCACGCGT | 249 |
| GCCATCCTAAGATTAGGACTTCTTCTTGACTGCCCGAGACTCGCCATTTCTGCCCGTGAATTTGTGTCTG | 250 |
| TAAAGCAAGGGGACCTTGGCACTCTCAGCTTTCCCTGCCACATCCAGCTTGTTGTCCCAATGAAATACTG | 251 |
| GAGGGCTCACTGAGAACCATCCCAGTAACCCGACCGCCGCTGGTCTTCGCTGGACACCATGAATCACACT | 252 |
| CTATGAATCTTTGTGAGCAATTATGCTCCCAAATCTAAGCAAGTAAAATACACATTTTGTCTTTCTTAAA | 253 |
| ACAACAGGCATTTAAGCAATGAAGATATGTTTAGAGAAGTGGATGAAATAGATGAGATAAGGAGAGTCAG | 254 |
| TAACCAGGCCAGTGACAGAAATGGATTCGAAATACCAGTGTGTGAAGCTGAATGATGGTCACTTCATGCC | 255 |
| AATCTGGCAGCCAGTTCCGTCCTGACAGAGTTCACAGCATATATTGGTGGATTCTTGTCCATAGTGCATC | 256 |
| CTGCCCCCTGAAACTTATTTTTTTCTGATTGTAACGTTGCTGTGGGAACGAGAGGGGAAGAGTGTACTGG | 257 |
| AACAAATGGTACAGTCATAAGAGCCATCTGTCACGGACCCACGCCCAGAGGAACGTGCAGAAAAAAGCAG | 258 |
| AGGATAGTTGGCTTCCTGCCTCTCTCCTCTAAAATAGCAAGTCTGGGAAATCCTGGGGTGAGTGGAGTCA | 259 |
| TGCGATTGGTTCTTCTGCCATGGCTTCAACAAGTGGCCTAGTAATCACCTCTCCTTCCAACCTCAGTGAC | 260 |
| GCTCCCAGCACACTCGGAGCTTGTGCTTTGTCTCCACGCAAAGCGATAAATAAAAGCATTGGTGGCCTTA | 261 |
| CCACATATATGCGAATCTATAAGAAAGGTGATATTGTAGACATCAAGGGAATGGGTACTGTTCAAAAAGG | 262 |
| TTCAGTCAGCCTCAGAGGTTGACTTCTACATTGATAAGGACATGATCCACATCGCGGACACCAAGGTCGC | 263 |
| CCTTCCATTTTCCCCCACTACTGCAGCACCTCCAGGCCTGTTGCTATAGAGCCTACCTGTATGTCAATAA | 264 |
| GCACCTCTAGTGCTACTGCTAGATATCACTTACTCAGTTAGAATTTTCCTAAAAATAAGCTTTATTTATT | 265 |
| ACGCTCACTGCCTGGCTTGGAAAAGTTAAGAAGCCCCTCAGGAAGAGAATCGAGGCCAAGTTCCTCTGCG | 266 |
| GGCTTTTGAATCGTAATAGCAATGTGAGGGTGAGGTACACCTACAGACATTAAATAATTTGCTGTGAAAA | 267 |
| TCGCCTACACAATTCTCCGATCCGTCCCTAACAAACTAGGAGGCGTCCTTGCCCTATTACTATCCATCCT | 268 |
| TTCATCTCTGGATGACAAGCCCCAGTTCCCAGGGGCCTCGGCGGAGTTTATAGATAAGTTGGAATTCATC | 269 |
| GCACTGCTCTCAGACTATGTTCTCCACAACAGCAACACCATGAGACTTGGTTCCATCTTTGGGCTAGGCT | 270 |
| AAGTGGTGGAATCGGCTATCCATACCCTCGTGCCCCTGTTTTTCCTGGCCGTGGTAGTTACTCAAACAGA | 271 |
| GTTTAACACTAAACCAAGGTCATGAGCATTCGTGCTAAGATAACAGACTCCAGCTCCTGGTCCACCCGGC | 272 |
| TAGTGTCAGTCACCAAAGAAGGCCTGGAACTTCCAGAGGATGAAGAAGAGAAAAAGAAGCAGGAAGAGAA | 273 |
| CCCACTGTCTGGGGCAGGGGGAGAAGGTATTTTCGAGATAAAGCACAGGCACCACAAATAAAAGTCGTGA | 274 |
| GAGGTAATCTGGGTGCACAGAATTTATCTGAGTCTGCTGCTGTGAAGGAGATACTGAAGGAGCAGGAAAA | 275 |
| ACAGTCATGCGCAGGGACGATCCTTGTTCTCTGCTGTAAACTGTAAAAAGTTTATGGAGACTTAAAGTCT | 276 |
| TACTGGAACAGCCAGAAGGACATCCTGGAAGACAAGCGGGCCGCGGTGGACACCTACTGCAGACACAACT | 277 |
| CACCTGAGGTCGGGAGTTCGCGACCAGCCTGACCAACATGGAAAAACCCCGTCTCTACTAAAAATACAAA | 278 |
| AGTCGGGCTACCCACTGATTTTCCTTCCCTTACTTCCCCTGAGCCCTTGGGCCCACTTCCCAGCCTACCG | 279 |
| CAGAGAAACGGCAGGAAGACCCTTACTACTGTCCAAGGGATCGCTGATGATTACGATAAAAAGAAACTAG | 280 |
| CCCCATCTTAACTGATTTAACCCCTGAAACAACCCGACGCTGGAAGTTGGGTTCTCATCCCCACTCTACA | 281 |
| TGGGCTACCATCTGCATGGGGCTGGGGTCCTCCTGTGCTATTTGTACAAATAAACCTGAGGCAGGAAAAA | 282 |
| GGGCCCAATTCTTCTCCACGACAATGCCCGACCGCATGTTGCACAACCCACACTTCAAAAGTTGAATGAA | 283 |
| CACGTCTGACAGCCATGTCCACCTGTGCCCACAGCTTCCGCCCACAGACCTCCAGGGACAGGAGCAAATT | 284 |
| TTAAAAAAGTTGGGTTTTCTCCATTCAGGATTCTGTTCCTTAGGATTTTTTCCTTCTGAAGTGTTTCACG | 285 |
| GAGGGGAGGGGCCTAGGGAGCCGCACCTTGTCATGTACCATCAATAAAGTACCCTGTGCTCAACCAAAAA | 286 |
| GGATACTGCGAGTATGGCGGCGTCAAAGGTGAAGCAGGACATGCCTCCGCCGGGGGGCTATGGGCCCATC | 287 |
| TTAGGTTAGGAGTTCATAGTTGGAAAACTTGTGCCCTTGTATAGTGTCCCCATGGGCTCCCACTGCAGCC | 288 |
| GGCCTCAAGAGGTTTGGAGCAGGTATGTTAAGAAGTTAGGGGATTTTGCTAAGCCGGAGAATATTGACTT | 289 |
| GATCTTCCCTGTCTCACACTTCTTTTCTCCCATCCCGGTTGCAATCTCACTCAGACATCACAGTACCACC | 290 |
| ACAGATTGTTCCTCCCATTCCCCTTGCCGCTTTTTGCCTATCGATGGGTAGCAAGAGTCTTTGAAATAAG | 291 |
| GGGCCCCCAGCCTCATCTCCGGCTCCAGCCCCTAAGTTTTCTCCAGTGAGTCCTAAGTTTACTCCTGTGG | 292 |
| CCTTCAGCTAATTTCTGCTCCCCTGAGATTCGTCCTTCAGCCCCATCATGTGCTTTGGGATGAGTGTAAA | 293 |
| AGTGGCCCATCTTTGTTGGCCTACGAACTTTGGTTTGATGCCAGTCAGGTGCCACATGAGAACCTTTGCT | 294 |
| CCCCCTGCCCTCCCCTCTCTGCACCGTACTGTGGAAAAGAAACACGCACTTAGTCTCTAAAGAGTTTATT | 295 |
| ACAAATGCGACGAACCTCTGAACATCCTGGTGAGGAATAACAAGGGCCGCAGCAGCACCTACGAGGTGCG | 296 |
| TAGCCCAGGCTGTGGAGGGGCCCAGTGAGAATGTCAGGAAGCTGTCTCGTGCAGACTTGACCGAGTACCT | 297 |
| CTCAATTTTGTGAGGCTGTGTTGGAAATAACCCGCCTCTAGTGCTGTTGGTATGCAAGGCAGCGGTGCTT | 298 |
| TGCTCAAATTACCCTCCAAAAGCAAGTAGCCAAAGCCGTTGCCAAACCCCACCCATAAATCAATGGGCCC | 299 |
| GACTCCGCTGGGAGAGTGCAGGAGCACGTGCTGTTTTTTATTTGGACTTAACTTCAGAGAAACCGCTGAC | 300 |
| CGCAGCTTAGAGAGACTCACCAGCGAGCGTCATTGTTGTCTTTCTGGGAACTCATTCCCATGAGATCAGA | 301 |
| CAGTGGAACTGTCCCACAAGAATTCACAGGTCTCAAAGCAGGAACAGTGGGTTTGTGTCTCACCTGAGTA | 302 |
| AGCTAGTGCCGACTCCCGCCTAGCTCTTTTGACTCTGTTCGCGGGAAGAATGGGGAAACAGTAAGGTTGC | 303 |
| ACACTGTTTGGAAGAAAGCTAAACCCTGAAGATCAGTAGCCCCTAATCACATGTGCTGCAAATAGCCTTC | 304 |
| TTGTGGTCGGGGAGCTGGGGTACAGGTTTGGGGAGGGGGAAGAGAAATTTTTATTTTTGAACCCCTGTGT | 305 |
| GATCTGGTTACCTGTGCAGTTGTGAATACCCAGAGGTTGGGCAGATCAGTGTCTCTAGTCCTACCCAGTT | 306 |
| TGCTCCAACTGACCCTGTCCATCAGCGTTCTATAAAGCGGCCCTCCTGGAGCCAGCCACCCAGAGCCCGC | 307 |
| CCCCGCTTCCCCAGTCTTTAAACATTGGACGCTATTTACTCAGCTACCCAGTAGAGCTTGAAGCTGACCT | 308 |
| CTTTCAGTCTTTATGTCACCTCAGGAGACTTATTTGAGAGGAAGCCTTCTGTACTTGAAGTTGATTTGAA | 309 |
| AGGCCCCTGCTGGATTGGCAGGCCCTGTCCGAGGAGTTGGGGGACCATCCCAGCAGGTAATGACTCCACA | 310 |
| AAGACAGCGCCGCCCGCGCACCGCCAGCGACCCCCGCCGCAGAGTCCCACCGCCACAGGCCTCGGGCCAG | 311 |
| TCTGTTCTGTTTGTACATGGCTGACGGAAATCTCTTTGGTACAACCGAATAAAGCCTGGTGGCAGTGCTG | 312 |
| CCAAGTACCATAGGACAGTCACATAGGAGCGTGTAGTCGTGACTGAATAAAGAAAGCAAAAGCCTGAAAA | 313 |
| AGTGGCTAAATTGCAGTAGCAGCATATCTTTTTTTCTTTGCACAAATAAACAGTGAATTCTCGTTTAAAA | 314 |
| AAGCATCTTGCTTGGTTGCTACATTCTGGTGTGATGGGTGCAGTGGTGCCTCCTCTGACAATATTAGGGG | 315 |
| TTTTAATTGGAGAAGGGTATAGAGGTAGTCCAGGTGGGAACGCCAGAAGTGCTGATTGCCCAGCCATTGG | 316 |
| AAGCCTGTGAGATCTTGTGTTGCAGCGTGGTTTGGCCCTAGCGTTCTTGCATGCTAACCTAAGGTAGAAG | 317 |
| AAAAGCGTACAAAAGATACTTAAAAGGGCTCCTGGGGTACACAAGCCCAGCAGGTCCTGAGTGAAGCCGT | 318 |
| AAGACCTGGACCAGTCTCCTCTGGTCTCGTCCTCGGACAGCCCACCCCGGCCGCAGCCCGCGTTCAAGTA | 319 |
| GGGGCATGCACCCTCCTTTCTGTACCGTGTGTGCTGGCTCCATAGTTCTCTCTTCTGTACATATAAGCAT | 320 |
| GAAGGCTCAGCCTCAAGATTCACAGCATCTCAGACACAGCCTAGGCCGCACCAGGATGTCGGACACCGAG | 321 |
| GGGGGAGTTGAGCAGGCGCCAGGGCTGTCATCAACATGGATATGACATTTCACAACAGTGACTAGTTGAA | 322 |
| TCAGCCAGCACCAAGCCTTGTTGGGCACTATCAGGGCTGAGGGAAAGATCTCAGAACAATCAGATGCAAA | 323 |
| GGCCACGGGAACAGGACCATGGTTAAGCAACCATATAGAAAGCTTTGTTGAAAGAAAGTATGGCATCTTG | 324 |
| ACAACTTGGAGAAATTTGGAAAACTCAGTGCGTTCCCCGAACCTCCTGAGGATGGGACGCTGCTATCGGA | 325 |
| CCCATGGGGGGTGGATGATTTGCACTTTGGTTCCCTGTGTTTTGATTTCTCATTAAAGTTCCTTTCCTTC | 326 |
| TGGGTCCTGGGAATGCTGCTGCTTCAACCCCAGAGCCTAAGAATGGCAGCCGTTTCTTAACATGTTGAGA | 327 |
| TGGCAAAAACGGCCAGGTACAACACCTTTTTCATACAAGGCCCAGGAGGCTTAGTCCAGTCTGTGCTCCT | 328 |
| GCCCACTGTAGTATCCACAGTGCCCGAGTTCTCGCTGGTTTTGGCAATTAAACCTCCTTCCTACTGGTTT | 329 |
| GAGCAAAAGACCGTGAGTCCCCTAGAAGTTACTCATCCACTTTGACTGACATGGGGAGAAGTGCACCAAG | 330 |
| GGGGTGAGTGTAGTTCTGGCCTAGCAGCACCCTCTTGTGGCTTGTTCTAGCGTGTATTAAAACTTGACAC | 331 |
| GTGTGAGAGTGTGAATGCACAGGTGGGTATTTAATCTGTATTATTCCCCGTTCTTGGAATTTTCTTCCCC | 332 |
| GCCTTCCCTCAGTGATGGGTTCAGTTCCGGAAGGTGTCTTAGAGGACATTAAAGCGCGTACTTGCTTTGT | 333 |
| CCATATGTCACTGGGGGAAAGGCTGCCTGTACCTCTCAAGCTTTGCATTTTACTGGAAACTGAGGCGTCA | 334 |
| AGAATACAGTTGTCTAGCCAAGCCATCAAGTGTCTGAAATTCAATATTGGTTTATGCAAATACAGCAAAC | 335 |
| CGGCTCTGGTTGTTGGCAGCTTTGGGGCTGTTTTTGAGCTTCTCATTGTGTAGAATTTCTAGATCCCCCG | 336 |
| TGGTGGCATAATTGGAGCCTTGCTGGGCACTCCTGTAGGAGGCCTGCTGATGGCATTTCAGAAGTACTCT | 337 |
| ATACGGTGTTTTCTGTCCCTCCTACTTTCCTTCACACCAGACAGCCCCTCATGTCTCCAGGACAGGACAG | 338 |
| CCCTTATCTGCTACCCTGAATCACCTGTCCTGGTCTTGCTGTGTGATGGGAACATGCTTGTAAACTGCGT | 339 |
| CCTAGCGCGCGGGGGGCGCCCCCCAGCCCGGAGGCTGGCTTTGCTACAGCTGACCACTCCGGTCAGGAGA | 340 |
| GTGAAGTGTTGGAGGTTGTGAACTCTGTAGACATCTTTATTGCTTGGCTAAGAGTAGATTTAATAAATGT | 341 |
| CCCCATAGTCAGGTGTACCAGCCAGCCAAACCAACACCACTTCCTAGAAAAAGATCAGAAGCTAGTCCTC | 342 |
| GTTGCTGCCATCGTAAACTGACACAGTGTTTATAACGTGTACATACATTAACTTATTACCTCATTTTGTT | 343 |
| GGGGACCAGCAGATAAATCCCACCCTTCCTTGAGCTGTCGCTGTACTCTGAAGTTCAGCCAGCTCAGATT | 344 |
| GAGAAGGACAAAATCACCACCAGGACACTGAAGGCCCGAATGGACTAACCCTGTTCCCAGAGCCCACTTT | 345 |
| ATCTATGATGACGATTTTTTCCAAAACCTAGATGGCGTGGCCAATGCCCTGGACAACGTGGATGCCCGCA | 346 |
| GCTTGGAGTGAAAGTGACTCTCAGGTGGTGGGGTGGGGAATGTGAATAAACATGATTTCTTGCCGGGCAA | 347 |
| GCGTTTAAAATAAAATATGCAACAAAATGGATGACTTAGTGGAGATGGAAGCCCATTAATTGGGTTCCCC | 348 |
| CACTCCCTAATCCCCTACCCCTGTCTCCCCTTCAAGGACTTCTCCCTTGTGGTTTTGTAAAGTGCAAACT | 349 |
| GTGAATTTTTGCACATTCTACACACAGTGCCTGTAAATCTCATTTGTATTTTCAGTTTGCCCTTAATTTT | 350 |
| TTTTTACTCCCCTTCAGCCCCCCGGCTGATGCCATCTCTGGTTCTGGACAATTATCAAATATATCAGTGG | 351 |
| GGATATAGACCACGATTCCGCAGGGGCCCTCCTCGCCAAAGACAGCCTAGAGAGGACGGCAATGAAGAAG | 352 |
| CAGGAGGGCAGTGGTGGAGCTGGACCTGCCTGCTGCAGTCACGTGTAAACAGGATTATTATTAGTGTTTT | 353 |
| TATTTGACAGTGTAGGAAATTGTCTATTCCTGATATAATTACTGTAGTACTCTTGCTTAAGGCAAGAGTT | 354 |
| CGAAGGAGTTGCGGTTGCTCCATGTTCTGACTTAGGGCAATTTGATTCTGCACTTGGGGTCTGTCTGTAC | 355 |
| CATTATGACCTGCTAGAGAAGAACATTAACATTGTTCGCAAACGACTGAACCGGCCGCTGACCCTCTCGG | 356 |
| TAATTTGTAAGTTATGTTAGCGGGATCCTCAAGGCCTTGCTTTGCCCCGTGGAGACGCTTGCTCGGATGA | 357 |
| GCACAGATGAAACTGAGCTGGGACTGGAAAGGACAGCCCTTGACCTGGGTTCTGGGTATAATTTGCACTT | 358 |
| GAGACAGAGTAATTTGCAGTTTGTTTGATTTATACTTTTGTTTATCTACAACCCAATAACAGACATGAGG | 359 |
| CTGGGGAAGCATTTGACTATCTGGAACTTGTGTGTGCCTCCTCAGGTATGGCAGTGACTCACCTGGTTTT | 360 |
| GCCAAGGGGCCAGCTGCCCCTCATTTATCACTCTGACCTTCACAGGGACAGATCTGATTTATTTATTTTG | 361 |
| GTGGGAGCAGCAGAGATGTCCAGGGTACAGATGCAAGTCTTGATGAGGAACTTGATCGAGTCAAGATGAG | 362 |
| TAAAGGCCCGGGAGCGGCTAGAGCTCTGTGATGAGCGTGTATCCTCTCGATCACATACAGAAGAGGATTG | 363 |
| TCAAGTGGAGCTTCATGAATAAGCCCTCAGATGGCAGGCCCAAGTATCTGGTGGTGAACGCAGACGAGGG | 364 |
| TAAAAACACCTTGGGGGCAGGCAGGGGCATTTAAAAATGTAGGACCTATCGTCCAGACTCACAGAGTGGG | 365 |
| GTGGCTTTCCTTACTGCGAAGAATGCTAAGACCCCTCAGCAGGAGGAGACAACTTACTACCAAACAGCAC | 366 |
| GCGGACGCTATCTACGACCACATCAACGAGGGGAAGCTGTGGAAACACATCAAGCACAAGTATGAGAACA | 367 |
| TCCCTTCTGGGTTCCGAGGCCCAAGCCCTTGGCAGTGTTTGTGAGTGGAAGGGAGGTCACGCTATCGTCC | 368 |
| TTATTTCCCTTCCACAGTGTGGTTTCTTCCTCTGCGGTAAAGGACTTGGTCTGTTCTACCCCCTGCTCCA | 369 |
| GAGCATTCATCGTGAGGGGTCTTTGTCCTCTGTACTGTCTCTCTCCTTGCCCCTAACCCAAAAAGCTTCA | 370 |
| TTCATCAAGAACCACGCCTTTCGCCTGCTGAAGCCGGGGGGCGTCCTCACCTACTGCAACCTCACCTCCT | 371 |
| CTGTGAAAATACCCCCTTTCTCCATTAGTGGCATGCTCATTCAGCTCTTATCTTTATATTCCAGTAAGTT | 372 |
| CGTCCACGGACTCTCCGTTATTTTAGGAGGTCCCTGGCCAAAGATTTATTTCTCTTGACAACCAAGGGCC | 373 |
| CGATGAGAAGGTTTACTACACTGGAGGCTACAACAGTCCTGTCAAATTGCTTAATAGAAATAATGAAGTG | 374 |
| TGGGTGATCTCTTTGCTGAATTAATGAGTTCTTAACATGTGGACCCAACTGCCTGTGTGAGATCTGTGTC | 375 |
| CTCACAGCGGCCCGCGGGCCGGGCGTCATGGGCGGCCTCTTCTGGCGCTCCGCGCTGCGGGGGCTGCGCT | 376 |
| TGATCCCGCACGGCACATCACTGGGGAGAAGCTCGGAGAGCTGTATAAGAGCTTTATCAAGAACTATCCT | 377 |
| AATACACATTTGAAAATTTCCAGTATCAATCTAGAGCGCAAATAAATCACAGTATTGCTATGCAGAATGG | 378 |
| CTCATCCACAGAAAGGGAGGATGGGCGATGACAGTTGTTTCTATGCCTTCTGACCCAGTTTCCCAGTTTA | 379 |
| ACGTCTGGTAGGAAGATTGTTAGTGCCTCAAGTTACACCTGTGCAGCTTGGGTCTGAGTTTTGATAGAAC | 380 |
| GAATGTTTAGGGGCCTGTGTGAACGCACCAATGGTTCAAATAAATGACAATTACTATGAGGATTTGACAG | 381 |
| GGGGCTGTTAAGTCTGACCATACATCACTGTGATAGAATGTGGGCTTTTTCAAGGGTGAAGATACAAGTC | 382 |
| CGCGCTGCTCCGCCGCCCGGGACTTGGCCGCCTCGTCCGCCACGCCCGTGCCTATGCCGAGGCCGCCGCC | 383 |
| CTTTGTTGGGAGGCGGTTTGGGAGAACACATTTCTAATTTGAATGAAATGAAATCTATTTTCAGTGAAAA | 384 |
| GGTGACCTCTGCCCCAGATAGGTGGTGCCAGTGGCTTATTAATTCCGATACTAGTTTGCTTTGCTGACCA | 385 |
| GTTTTTAAAATCAGTACTTTTTAATGGAAACAACTTGACCAAAAATTTGTCACAGAATTTTGAGACCCAT | 386 |
| GCCCCTGGCTTCACCCTGTCAGGCCAGCTCCACTCCAGGACTGAATAAAGGTCTTTGACAGCTCTAAAAA | 387 |
| ATTGGCAGATCAAGCGCCAGAATGGAGATGATCCCTTGCTGACTTACCGGTTCCCACCAAAGTTCACCCT | 388 |
| GCCAGGAGGCCCTGGGTTCCATTCCTAACTCTGCCTCAAACTGTACATTTGGATAAGCCCTAGTAGTTCC | 389 |
| TTGTGGACTTCCTCATTGGCTCCGGCCTCAAGACCATGTCCATCGTGAGTTACAACCACCTGGGCAACAA | 390 |
| TGTTAGAGATGCTATTTGATACAACTGTGGCCATGACTGAGGAAAGGAGCTCACGCCCAGAGACTGGGCT | 391 |
| ACAAAGTGAAAAACAGCCTTTTGAGTCTTTCTGATACCTGAGTTTTTATGCTTATAATTTTTGTTCTTTG | 392 |
| CCGCAATGTTGGTTTCACTGAGAGCTGCCTCCTGGTCTCTTCACCACTGTAGTTCTCTCATTTCCAAACC | 393 |
| GGGAGGAAGCATGTGTTCTGTGAGGTTGTTCGGCTATGTCCAAGTGTCGTTTACTAATGTACCCCTGCTG | 394 |
| CAAGGAAGGGGTAGTAATTGGCCCACTCTCTTCTTACTGGAGGCTATTTAAATAAAATGTAAGACTTCAA | 395 |
| GTTGGTGAGGTAACATACGTGGAGCTCTTAATGGACGCTGAAGGAAAGTCAAGGGGATGTGCTGTTGTTG | 396 |
| GAAAGCACCTGCTCCAAAGGCATCTGGCAAGAAAGCATAAGTGGCAATCATAAAAAGTAATAAAGGTTCT | 397 |
| TGCTTGTGAACGTGCTAAGCGTACCCTCTCTTCCAGCACCCAGGCCAGTATTGAGATCGATTCTCTCTAT | 398 |
| CTGCCTTGTTTTGCGACATTGTCCCATTCACACAGATATTTTGGGATAATAAAGGAAAATAAGCTACAAA | 399 |
| GATATTTAAAGTTTTGGCAGTAAAATACTCTGTTTTTAAGTATGAATGTATTTCATTCATATTTCCTCTC | 400 |
| TGGTTGATTTTGTACTTTGGAACTGTACCTTGGATGGTTTTGTTTATTAAAAGAGAAACCTGAACCAAAA | 401 |
| GGAGGCAGAACCAGCAACAACTCTGGGCGTGCCTGTGTCTGCACATGTGGATGTACATATGTCTGTATAT | 402 |
| AGGCGGCGAGCGGGGCCCGGCGCCGACCCTGAGTGCAGCCTGACCCGCCCTCGCGCGCGCGCCCTCCCGG | 403 |
| GTGAAAAGCCTAAATGACATCACAGCAAAAGAGAGGTTCTCTCCCCTCACTACCAACCTGATCAATTTGC | 404 |
| CGCCACCCTTGACGCTTGCAGCTTCGGAGTCACGGGTTTGAAACTTCAAGGGGCCACGTGCAACAACAAC | 405 |
| GCCCAGGGAAGACACATGATTAATGATTTAGCTCCCTCCATACCTCGAACATCAGTTGGGATCCCTCCTC | 406 |
| CGATTCCACTGGTGGTAGTTTGCTAGTGCTTCTAAAAGTTGCTCCCTAGCACTGAGAGGTGTGGGTAGGT | 407 |
| ATGGGCCGACCTGGCTGGGACTCGTGAATCTGGAGAAGAGCTGGAGAATGGATAGTATTGTCTGTATTTG | 408 |
| GAGGACCCCTACACATCTTTTGTGAAGTTGCTACCTCTGAATGATTGCCGATATGCTTTGTACGATGCCA | 409 |
| GCGAGCGCGCCTGCGCGCTGGGTGATTTTTTCACGTGTCGCCAGGGCCGGACTGCGAGTCTCTTTGCGGC | 410 |
| GACTACAAATGGACGAGAGAGGCGGCCGTCCATTAGTTAGCGGCTCCGGAGCAACGCAGCCGTTGTCCTT | 411 |
| GAGGGGAGGGGCCTAGGGAGCCGCACCTTGTCATGTACCATCAATAAAGTACCCTGTGCTCAACCAAAAA | 412 |
| TTTAGGCTGGAAGCGCCTTAGAGGAGCCATTTTTCCAGGTGGGGCCCCAGGCAGAGGCTCCGACAGGGAG | 413 |
| CACTACCGTGGAGATCCCAACTGGTTTATGAAGAAAGCGCAGGAGCATAAGAGGGAATTCACAGAGAGCC | 414 |
| CGCTTAAATCATGTGAAAGGGTTGCTGCTGTCAGCCTTGCCCACTGTGACTTCAAACCCAAGGAGGAACT | 415 |
| GAGTTCGAGACCAGCCTGAGCAACATGGCGAAACCCCGTCTCTACTAAAAATACAAAAATCACCCGGGTG | 416 |
| TGAGGATGGCTTGACCCGAGTCGGCTTCOGCACAGTGTTGCTGAGAATACGAGAACAGTGGAAACAGAAC | 417 |
| ATGTTGGGCGAGTCACTGCGTCTCGGGCATTGGTGTCCTGTCAGTAAAGAGATAATAATGGCTGTACCTC | 418 |
| GTGCACGTGTGAAGCCCCCTCACTCTTCCGCTAGGGATAAAGCAGATGTGGATGCCCTTTAAGAGATATT | 419 |
| CAGGAACCTGCTTCACTGTATTAACTAGTCCATGGGCTGAGACCGGGGCATCTCTTTTCTTCATACTGCA | 420 |
| CAGCATACCCCCGATTCCGCTACGACCAACTCATACACCTCCTATGAAAAAACTTCCTACCACTCACCCT | 421 |
| GCTGCCTGCCCTCCTCCTCTCACCCGATGTCCAGGTGGGATTTTAAAGTCTGCATTGGTTATAATAACAG | 422 |
| ATAAGGTTTCCAGTAAGCGGGAGGGCAGATCCAACTCAGAACCATGCAGATAAGGAGCCTCTGGCAAATG | 423 |
| CTAGTTATTAAGCCCAGCATGCATTAGCTCTTTTTCCTGATGCTCTCCCTCCCTTCATCATCCGCCCTCC | 424 |
| CTACTTCTAAGTCTGAATCCAGTCAGAAATAAGATTTTTTGAGTAACAAATAAATAAGATCAGACTCCAA | 425 |
| CCCACGCGCACTTACACGAGAAGACATTCATGGCTTTGGGCAGAAGGATTGTGCAGATTGTCAACTCCAA | 426 |
| GAACCCCTGTGGCGGAGGACTGGCCTGTGTCTGTTATTTTGGTTGTAAATCATTCTCCTGTGGAATTGGC | 427 |
| CCTGAATTCACTCGGGTATATTGATTGGCTGGATGATCTTGGTGCCGCCCACTTGACGTTTCCAGAAGAG | 428 |
| GCACAAAGGAGGCTTTTTCTGTGCTTTGACATTCTAGCACTTCAGGGATGAGAGGGAGGGAGAATCCTGG | 429 |
| GCATCCACACCAAGAGGGTGTTGTGATGAGGTGCCGGTGTGCAAAGGGAACTTTAGTTTTTCCACTGGTT | 430 |
| GTGTGAAACTTGCTCTACTCTCTGAAATGATTCAAATACACTAATTTTCCATACTTTATACTTTTGTTAG | 431 |
| TAAGCGCTGACGCATGCGCATAGCTAACCGCACCCGGTTCAGCTCGCCTTTCTTGGCCAGAGGCGCCGGT | 432 |
| ATACTTTGGACTTCCTCTCGCCAAAGACCTTCCAGCAGATTCTGGAGTATGCATATACAGCCACGCTGCA | 433 |
| TGTACACTTGACAAGTGCTTACTCAGCAAGTCCCAGACCCACGGCCTTTTATCTCCCAAGACTGGCTTTG | 434 |
| GCGCCGCCCATTGGTCCCGAGCGCGATGACTTGGCGGGCGGAGCAGGAAGGAAACCGCTCCCGAGCACGG | 435 |
| CGTGGCCGCACATCCTACAGTTGGAAATCCATCCAGAGGCCATGTTCCAATAAACAGGAGGTCGTGTAAA | 436 |
| TCTACGCCCCAGGGCTGTCGCCAGACACTATCATGGAGTGTGCAATGGGGGACCGCGGCATGCAGCTCAT | 437 |
| CCTTAAGTCTAATAAGGTCATGGCTGAGTCTCTCAGAGTGTGGACCTGCCCCCTTCTACTCTGGGCGGTT | 438 |
| CTGAGAGGAACCTGGACATGGTCCCGGGCATCTGAATGATCTGTAGGGGAGGGAGTTCAAATAAAGCTTT | 439 |
| GGCGGGGGCCTTGGGGCAGTCCGAGGGTGCGGTGAAGAGGTGACGGAGGGCTGGCTATGGGCGGCCGGCC | 440 |
| GTGGTGGGAGGTGTTTAATGACGACCTTACCAAGCCAATCATTGATAATATTGTGTCTGATCTCATTCAG | 441 |
| CAACCCTGACCCGTTTGCTACATCTTTTTTTCTATGAAATATGTGAATGGCAATAAATTCATCTAGACTA | 442 |
| ACTTTGCAGTGGATCCTGACCAGCCGCTGAGCGCCAAGAGGAACCCCATTGACGTGGACCCCTTCACCTA | 443 |
| CTTCTTCTTCTCTCCCAGCTGAACCCGAGGCTAAAGAAGATGAGGCAAGAGAAAATGTACCCCAAGGTGA | 444 |
| CACATGGCTGGGCTGACAGCATCCCCTACACCCCCTTCTTCAAGCATAATTACTTACTGACTTTCCTCCA | 445 |
| CCACCCTGGAGCCAAGGGTCTTTCACATCACCTATCCCTACATACATACCAAATGGAAAAGTGGCCATCC | 446 |
| TTAAGACTTTCCAAAGATGAGGTCCCTGGTTTTTCATGGCAACTTGATCAGTAAGGATTTCACCTCTGTT | 447 |
| AGGAGCAGTAAACATAGCCAAGGCCTAAGGGATCAAGGAAACCAAGAGCAGGATCCAAATATTTCCAATG | 448 |
| AGAAGGGCCCCAATGCCAACTCTTAAGTCTTTTGTAATTCTGGCTTTCTCTAATAAAAAAGCCACTTAGT | 449 |
| ACATTCCAGATGGCTATCCTGCTTCAGTACAACACGGAAGATGCCTACACTGTGCAGCAGCTGACCGACA | 450 |
| GGGGCATCAGAGTCTTGGCTGGGCTGAATCTGCTGCTTGTTGGTTCAGTGTTTCTTATGAACAAGAGCCA | 451 |
| GAGAGTTCGATATGATTCTTGGGAAACTAGAGAATGACGGAAGTAGAAAGCCTGGAGTCATAGATAAGTT | 452 |
| GAGAGTTGCTGCCTTTGATAGACCCATGCTGACCACAGCCTGATATTCCAGAACCTGGAACAGGGACTTT | 453 |
| GTCTGAGCAAGGGGTGTACACCTGCACAGCACAGGGCATTTGGAAGAATGAACAGAAGGGAGAGAAGATT | 454 |
| AGAGACCGCTGGCAGCACCAGTATTCCCAAGAGGAAGAAGTCTACACCCAAGGAGGAAACAGTTAATGAC | 455 |
| CTAAGACTCGCGGGAGGTTCTCTTTGAGTCAATAGCTTGTCTTCGTCCATCTGTTGACAAATGACAGATC | 456 |
| GCCAGATAGCTAGGTTTCTGGTTCCCCCACAGTAGGTGTTTTCACATAAGATTAGGGTCCTTTTGGAAAG | 457 |
| AAGCACGTTGCCCAAGGTTGCACAGCAAGAAAAGGGAGAAGTTGAGATTCAAACCCAGGCTGTCTAGCTC | 458 |
| CTGCAAAGAGGCCAACACACTAGAAATCAGAAATCTTGACTCCTAGCCCACCGTCCCCTAAAACATGGGC | 459 |
| CGCGGTTTGGTTTGCAGCGACTGGCATACTATGTGGATGTGACAGTGGCGTTTGTAATGAGAGCACTTTC | 460 |
| TCGGACTCCTGCCTCACTCATTTACACGAACCACCCAACTATCTATAAACCTAGCCATGGCCATCCCCTT | 461 |
| AGGAGCAGCCCATGGAGACGACGGGCGCCACCGAGAACGGACATGAGGCCGTCCCCGAAGGCGAGTCGCC | 462 |
| GCTTCTTGCTGCCGCCATATGAAGAAGGACGTGTTCGCTTCCCCTTCCTCCATGATTGTAAGTTTCCTGA | 463 |
| CATGTTAAAATGGGGAAGGATGATAGCTACATGTATGCCGGTCCTACTCACGCGACACCCGTGTGCTCAA | 464 |
| ACATGACCCCAGCAACTGTGGTGGTATCTAGAGGTGAAACAGGCAAGTGAAATGGACACCTCTGCTGTGA | 465 |
| GCCCCTGGCAAATGCACAGACCTCATGCTAGCCTCACGAAACTGGAATAAGCCTTCGAAAAGAAATTGTC | 466 |
| GTGGTTGATGGCGCCTTCAAAGAGGTGAAGCTGTCGGACTACAAAGGGAAGTACGTGGTCCTCTTTTTCT | 467 |
| TCCTTCCTAGTAATACTTTGCCTTTTTCACTGTGTATGGAATGAAACATGTAAAGCTGTCACAATCAATG | 468 |
| GCAAGACTCTTACGCCCCACACTGCAATTTGGTCTTGTTGCCGTATCCATTTATGTGGGCCTTTCTCGAG | 469 |
| CCACAGAAGACACGTGTTTTTGTATCTTTAAAGACTTGATGAATAAACACTTTTTCTGGTCAATGTCAAA | 470 |
| GCAGCTTTGAACTAGGGCTGGGGTTGTGGGTGCCTCTTCTGAAAGGTCTAACCATTATTGGATAACTGGC | 471 |
| CCCAGGCTTTGTCCCAGGCTTTCTGGTGTGTGCCCTCCTGGTAACAGTGAAATTGAAGCTACTTACTCAT | 472 |
| CAGGTGCCTAGTCTTGAGTGAATTGTTAGATGTGCACTGAACTCGGGATGTTGGGGATTGGAGAGAGAGA | 473 |
| AAAAGTATTTTGTGGTGACCATAAGAATGTCCCTCCCCAAACAAGTAAACTTGTGAAAGTTTAATTTGGA | 474 |
| ATGATCCTGTTAGCTCTTCCAGCTCTCCAGGCGCCAACAACCATATGGTCTCGGTAACGACTGCTCCCCA | 475 |
| GAGAATACAAGATATTATGTATAAAATGTAACACTGATGATAGGTTAATAAAGATGATTGAATCCAAAAA | 476 |
| TACCCCTTCCACTGCTCACTTTGTGGATGGTAGCATGAGCTGTCTACCAAGAAGAAACCTGCTGCTCTCT | 477 |
| CAACTGGATGAAAAGGAAAAGGATTTGGTGGGCCTGGCTCAGATCGCAGAGGTCCTCGAGATGTTCGATT | 478 |
| CTAATCCCCTTGATGAGCTTTCACGAAGTCTCACGGCTTCTCTAGGGACTCCATGGTCTTCAGAGTCGTT | 479 |
| AGATGGGATAGTTTACTGACTAGTTGGAGCATTTGTAAGCACATGGTGAAATCAGCCCCTGCCCACCAAA | 480 |
| CCTGGGATTCTTTTTCTAGGGATGTAATACATATATTTACAAATAAAATGCCTCATGGACTCTGGTGAAA | 481 |
| TTTAATCGCTTTGAATAAATACTCCCTTAAGTAGTTAAATATAGGAGGAGAAAGAATACATCGGTTGTTA | 482 |
| GGCAATGCCTACCCCCAGCGTTATTTTTGGGGAGGGAGGGCTGTGCATAGGGACATATTCTTTAGAATCT | 483 |
| TGGAATAAAAGGAGAGAAGGGTTTCCCCGGATTCCCTGGACTGGACATGCCGGGCCCTAAAGGAGATAAA | 484 |
| GGCCAACCGAGCGCCATGAACCAGATAGAGCCCGGCGTGCAGTACAACTACGTGTACGACGAGGATGAGT | 485 |
| GCCAAAGTGCTCAGAGACCTTCTATGACACATTAGTGTCACATGGTTGCGTGTCCAGCCGAAGCAGTGTA | 486 |
| ATACAAAAGTGGCACATGCCTGTAATGCCAGCTACTGGGGAGGCTGAGGTAGGAGAATTGCTTGAACCTG | 487 |
| CCGGCAGTTCTTGGGTCAAATGACACAATTAAACCAACTCCTGGGAGAGGTGAAGGACCTTCTGAGACAG | 488 |
| GTGGCTGGCCCGGCCTCCACAGCACCCCACCCCATATCTTCTTTCCATTTATTTCGTACCAAAAACAATT | 489 |
| CATTTTTTGTAATTTTTGTAAAACAAAAAGTACCAATCTGTTTGTAAATAAAAATCATCCTAAAATTCGA | 490 |
| TGATCTTTCTGGCTCCACTCAGTGTCTAAGGCACCCTGCTTCCTTTGCTTGCATCCCACAGACTATTTCC | 491 |
| TCATAACTGGCTTCTGCTTGTCATCCACACAACACCAGGACTTAAGACAAATGGGACTGATGTCATCTTG | 492 |
| AGCCAGGATTTCCCTCAGTGCAACACCATTGAGAATACAGGAACTAAACAGTCCACCTGTAGTCCAGGGG | 493 |
| CGTAGACTCGCTCATCTCGCCTGGGTTTGTCCGCATGTTGTAATCGTGCAAATAAACGCTCACTCCGAAT | 494 |
| GACACTGGCCCCTCTCAGGTCAGAAGACATGCCTGGAGGGATGTCTGGCTGCAAAGACTATTTTTATCCT | 495 |
| TTTGCCCAGCACGCCAACGCCTTCCACCAGTGGATCCAAGAGACCAGGACATACCTCCTCGATGGGTCCT | 496 |
| CACAACATGAAAGAAATGGTGCTACCCAGCTCAAGCCTGGGCCTTTGAATCCGGACACAAAACCCTCTAG | 497 |
| ATCCCCATGCCCTTGACCTCTTCTGGCATTCTCCTGTGCTCTGACAAACTGAGCCAGCCTTTTAGATCTA | 498 |
| AAGTTTCCGACCCTGGCTTATAGGCACCACACCTCATGTACTCCTCATGGCTTGGATCTCTGTATTCAGC | 499 |
| AAGGTCTGACGCCACCTCAAGGTGACAGCTCATCTCCAGCACAGCACAGGCGTGTGCACACAGAGGTGTT | 500 |
| CGGAGCAGAGACAGGCCCTCGGGGTGGAGGTCTTTGGTTTCATAAGAGCCTGAGAGAGATTTTTCTAAGA | 501 |
| ATAAGTCACATTGGTTCCATGGCCACAAACCATTCAGATCAGCCACTTGCTGACCCTGGTTCTTAAGGAC | 502 |
| CTACTTCGGAGTCTATGATACTGCCAAGGGGATGCTGCCTGACCCCAAGAACGTGCACATTTTTGTGAGC | 503 |
| ACTGTTGCTTGCTGGTCGCAGACTCCCTGACCCCTCCCTCACCCCTCCCTAACCTCGGTGCCACCGGATT | 504 |
| GACAGGGCCAGTGCAGTTTGGTGTGTCCTCCGCCTTTCCAGGAGAAGAACCTGAAGAACTATTTTTCGTT | 505 |
| GGTCAGGGACTGAATCTTGCCCGTTTATGTATGCTCCATGTCTAGCCCATCATCCTGCTTGGAGCAAGTA | 506 |
| GGGAGAGTGCCGGGCGGTCGGCGGGTCAGGGCAGCCCGGGGCCTGACGCCATGTCCCGGAACCTGCGCAC | 507 |
| TGCTCTAAGGGACCTTGGAGACAGGCCTTTCAGGTGGATGTTCATGTTTCTGACCTTGCACTACCCCAAT | 508 |
| ATTCGCCGTTCGAAAGCAGGGACTAAAAGCCCCACTTCGTCTTACGTTCCGAAAGGAAGGCGTCTGTTGA | 509 |
| GGTGGAGTTGTTAGTGTCCTATGGCAACACCTTCTTTGTGGTTCTCATTGTCATCCTTGTGCTGTTGGTC | 510 |
| TTGCCTCATCACCTTGTCCAAATGAGCTAGACCTCCCTGTCCCGGAGGGAAAAACATCTGAAAAGCAGAC | 511 |
| TTTTTAAGCTCAAGCAAATGTTTGGTAATGCAGACATGAATACATTTCACACCTTCAAATTTGAAGATCC | 512 |
| TTTGGGAGAGACTTGTTTTGGATGCCCCCTAATCCCCTTCTCCCCTGCACTGTAAAATGTGGGATTATGG | 513 |
| GAACTGTGGCCACCTAGAAAGGGGCCCATTCAGCCTCGTCTCTTTACAGAAGTAGTTTTGTTCATGAAAT | 514 |
| ACTCCAAGAAGTACATTGCCTTCTGCATCAGCATCTTCACGGCCATCCTGGTGACCATCGTGATCCTCTA | 515 |
| AAGACCTGAACCAGAGATCCATCATGGAGAGCCCAGCCAACAGTATTGAGATGCTTCTGTCCAACTTCGG | 516 |
| TATTTTTCTTAACATGTTAGTACTTCTACGACTTTGGAGCCACTGATGGGTCCACTCATGGCCTCAGCTG | 517 |
| GGTCAGCAAAGGAAAGTGGAAGTTGGATTCTGAAAGATCGAGGTGCCCACAGGAATTTTATGGTCGTCGG | 518 |
| AGCACACCCGTCTATGTAGCAAAATAGTGGGAAGATTTATAGGTAGAGGCGACAAACCTACCGAGCCTGG | 519 |
| AAGGAAAACCGGCCCCAGAAACAGGGGTGTGCTTTCCCACCAATAAAAGGCCGTGGAACCCGAGGGCTTT | 520 |
| GGGATATAGGGTCGAAGCCGCACTCGTAAGGGGTGGATTTTTCTATGTAGCCGTTGAGTTGTGGTAGTCA | 521 |
| GCTCCTTTGTTTTACAGAGCAGGGTCACTTGATTTGCTAGCTGGTGGCAGAATTGGCACCATTACCCAGG | 522 |
| TGAACAAAAGAAGCCACGAGGTGGAACAAGGTCTCTGTCAGTCACAGGCACCCCTGAGAACCGGGAACAT | 523 |
| GCTACTGAGGGTCTAAGTCCGGGCAGCCGAAGAGTGTGGTAGGTAACGGTCCTCAGCGCAAGGGTCATTT | 524 |
| TCTACAAAGGGTTCATGCCCTCCTTTCTCCGCTTGGGTTCCTGGAACGTGGTGATGTTCGTCACCTATGA | 525 |
| TTTATCCCCAGACCAGGCATCACCTATGAGCCACCCAACTATAAGGCCCTGGACTTCTCCGAGGCCCCAA | 526 |
| ATGCCGTCGGAAATGGTGAAGGGAGACTCGAAGTACTCTGAGGCTTGTAGGAGGGTAAAATAGAGACCCA | 527 |
| TTTTTAAGTAGCCTCCTTTCCACTATTTAGTAATTGGCTGTGAGCTGGGCTGGGGGAGAAATGGGGCGGG | 528 |
| TTTTTGAGACAGAGTTTTGCTCTCGTTGCCCAAGCTTGAGTGTAATGGCATGGTCTTGGCTCACTGCACT | 529 |
| ACCCTGGNNAGATAGACTTCCCTGTTTCCAAGGGGCGTGGGACTTTCTACCACGTCCATCAACTCGTGGC | 530 |
| ATAGTGTTTGGCTTATTTTCCATCCCAGTTCTGGGAGGTCTTTTAAGTCTCCTTCCTTTGGTTGCCCCAC | 531 |
| AACTATTTGCGCAATCTGTGGGTCTGTGGATTCACGGGGCTTTCTGTGTGGGTGCTGCAGTTGCTTTTGT | 532 |
| TGGGCCTTGTGACATTGTCTACCTGTGGTCATTCCTTAACTGCTTTGGCCTCAACTTTGAGCTCTGGATG | 533 |
| TTTTAAAAATCCACTTATGGCTGGGCACAGAAGCTCACGCCTGTAATCCCAGCACTTTGGGAGGCTGAGG | 534 |
| AAACCCCATCTCTACTAAAAATACAAAAAATTAGCCGGGCGTGGTAGCGGGCGCCTGTAGTCCCAGCTAC | 535 |
| CTGGATCTTGGCCTTTACATTTTCTATCGTATCCGAGGGTTCAACCTCGAGGGTGATGGTCTTCCCCGTA | 536 |
| GGACAAGAACACAGTCAACTTTGGCTTTGCTTGGAAAGCTGCTTCAGATACATAACTCCCGGCCCCTCCT | 537 |
| CTTCTTCTTCTCTCCCAGCTGAACCCGAGGCTAAAGAAGATGAGGCAAGAGAAAATGTACCCCAAGGTGA | 538 |
| ATCCAACCCTTTAAGATGAGTGCCACTGGTTGCCCATTTTACAGATGAGAAACTGGGCTCACAGACACAC | 539 |
| GGACATTTGGGTTGGTTCCAAGTCTTTGCTATTGTGAATAGTGCCGCAATAAACATACGTGTGCATGTGT | 540 |
| ATCAGCCGTAAGCCTAGAAGCAGAGCGGGATCGAGGCGTTTTTAATAATTCGAGTTGGGAAGACCCGGAT | 541 |
| GCTCTAGCTACTfGGACTATTCAGGGAGCTGCAAATGCCCTCTCTGGTGACGTTTGGGACATTGACAATG | 542 |
| GCGGAGATTCAAGGACCTAAGCTTCCAGGAGGAGTACAGCACACTGTTCCCTGCCTCGGCACAGCCGTAG | 543 |
| CAGTGTTCGAATCATCGACAAAAATGGCATCCATGACCTGGATAACATTTCCTTCCCCAAACAGGGCTCC | 544 |
| TATCTTGCTGGTCAAAATATACAAGATGTGAGCCTGGAAAGCCTTCGGAGGGCAGTGGGAGTGGTACCTC | 545 |
| GGCTTCTGGNAAGCTGTTGNAGCCCAATTGAACCANAAAGTTTGGTGGCCTATCAGNTGGACCTTGTATG | 546 |
| ACCTTCACTGTCAGCGCCTGGAAAACTTGGCTCACGAAACCAGGGAACGAAGAAAAACCTCCAGGGGAAC | 547 |
| CCCTCTGGTCCAGCCCCTCACGCCTCCTCTCAGTCTACTCAATTGTGACTGTCCCTCCTGATGTATTTTT | 548 |
| AATTCCCGGTTCTCAGAATTGTTATCACTCTGGTGCATGCTGTCACAGGGGCCGTTGCGTTTGGCTTTGT | 549 |
| CTCTGTGTTTCATGTGTCCCAGGTCCCCCAAAAAACAGGTGGTGGTGGATTATACATGGCTTTCAGTAGT | 550 |
| AGTACCTGCACAACCAGCACATCCTGCACCTGGACCTGAGGTCCGAGAACATGATCATCACCGAATACAA | 551 |
| CACCACTCTGAACAGCTNCTTGATGGTGTCATTCAAGTTATTGGGCTTTCTCTCCCGCTGGAGCCTCAGC | 552 |
| GGACGTGTAAACAGACGGTACCCTACTCTTGTGGCAATCACTAAGTTTCAGCCAACCAAAGACAGCGAAC | 553 |
| RATCATAAGTGAGAHTCYKCCCAGTYTTMTTTGTGCTTYTCTTTTGGGRAGAWTTAGTAAYTGTGCCACT | 554 |
| CTACAATAAGGGCAACTGCAGTCTCATATGTCCAACATCGAGCAACATTACGGATTGTGTAGCCACCTCC | 555 |
| KGYACCACAGGRTTGAGCCGTCGAGGGGKGAGTGCTGTTATTATWTCTTAAAAAATCTGATGACCCGGGV | 556 |
| CACTGACAGGGATCAAGTTTGTGGTTCTAGCAGATCCTAGGCAAGCTGGAATAGATTCTCTTCTCCGAAA | 557 |
| AAATGGACAAGGCCAGGTATAGCGAATGGCTTTGCTCCTGTAGAGAACCGTCACTCGGTCAGAMAARCCT | 558 |
| ATGCCGTCGGAAATGGTGAAGGGAGACTCGAAGTACTCTGAGGCTTGTAGGAGGGTAAAATAGAGACCCA | 559 |
| ATCACCTGCTGTATGCCGATCATCTCAGAAAGGGCTGTGTAGAGTAGGGCCCTGTTCTCCTTAGGATGTT | 560 |
| CGACATCATTGTTGGCGATGGTGATGACCACATCTGGGACATTGTAGGGAGTGTCTAGGTGACTCTCCAT | 561 |
| CTCTGTATGAGAACTCCCCAGAGTTCACACCTTACCTGGAGACAAACCTCGGACAGCCAACAATTCAGAG | 562 |
| AGCCTGGGGTGCTTCGTGGGCTCCCGCTTTGTCCACGGCGAGGGTCTCCGCTGGTACGCCGGCCTGCAGA | 563 |
| TCATCGACATGCTCATGGAGAACATCTCCACCAAGGGCCTGGACTGTGACATTGACATCCAGAAGACATC | 564 |
| TGAGTCCCGGGTAGTTGGAGCCTGTCAGTCGCCGGGTCAGTAGGTCGCGGAGTCTGCGAGAAGCCACTAT | 565 |
| AAGTTACGCAGATCCCATAAAGCTACGGTCTTATCCGCAGAGCCGGTGGCTAGAATAAAATCGCCTGTAG | 566 |
| AAGATTATTTTTTAAATCCTGAGGACTAGCATTAATTGACAGCTGACCCAGGTGCTACACAGAAGTGGAT | 567 |
| GAAGCCAGACTACACTGCTTACGTTGCCATGATCCCTCAGTGCATAAAGGAGGAAGACACCCCTTCAGAT | 568 |
| ACCCACAGGTCCTAAACTACCAAACCTGCATTAAAAATTTCGGTTGGGGCGACCTCGGAGCAGAACCCAA | 569 |
| GCATGAAAAACTCCAAATAAGAGATCYCTCAGGATTATAAAAGTTGTAAATGCACTGTWTKCTGGSAAAA | 570 |
| CCTTCTGCACATCTAAACTTAGATGGAGTTGGTCAAATGAGGGAACATCTGGGTTATGCCTTTTTTAAAG | 571 |
| AGGGTCTTCTCGTCTTGCTGTGTCATGCCCGCCTCTTCACGGGCAGGTCAATTTCACTGGTTAAAAGTAA | 572 |
| CCTCTTCCGGAGATGTAGCAAAACGCATGGAGTGTGTATTGTTCCCAGTGACACTTCAGAGAGCTGGTAG | 573 |
| ATGTGTACCTTGGAGTCATCCTCTTGGTCTTGTATTCATATTGTGGGACAGTGGGAATAGCAGCTTGTAG | 574 |
| ACCTTTTCTGGCAAGACTGCTCTGCATTTCTGCTGCCCTCATACCTCACCCAGCCAACCTACCAAACATT | 575 |
| CCATAAAGACTCCGTGTAACTGTGTGAACACTTGGGATTTTTCTCCTCTGTCCCGAGGTCGTCGTCTGCT | 576 |
| ACCTGTTGTTACAGGGCAGGATCGGATGATGGACACTGAAGTCCTCAGCTTGCTAAGTTCAGTTGCTCTC | 577 |
| TGTTTCTACCAACACTGCACCTTATCCCAGGAACCTGCCCTAGACCTCCAGAGACCATATTTTCTCTCCC | 578 |
| AACTTGAACCTAAAAATTAGCCCCTCATAGTGTAGCCGCCGGACTTTGCTCATAGCTGGCAGGCTGGACT | 579 |
| GTAGGAGCTCGTCACTCTTTTGACAAAAAGGGGGTGATTGTGGTTGAAGTGGAGGACAGAGAGAAGAAGG | 580 |
| GACAGTGTGGGTATCAAGAGCCAATGTGATCCAGCGCCGGGGCCGGGCGGGCCGCTGCCAGTCCGGCTTT | 581 |
| ATAAGGTTTCCAGTAAGCGGGAGGGCAGATCCAACTCAGAACCATGCAGATAAGGAGCCTCTGGCAAATG | 582 |
| CTGCAGTCCTCACTNGAGAAAATCACTCCCTCTGGGAGATTGGAAGTTGCTGGAAAGAAAACAGGTCCAA | 583 |
| AATCTGGCCAAAAGAGTTCGCGCTTTCCCCCATGGATGTTTTCTACCACAAGAATATAAGTGCTGAAAAT | 584 |
| CAGAGTACTTCGAGTCTCCCTTCACCATTTCCGACGGCATCTACGGCTCAACATTTTTTGTAGCCACAGG | 585 |
| CGCCCACGGACTTACATCCTCATTACTATTCTGCCTAGCAAACTCAAACTACGAACGCACTCACAGTCGC | 586 |
| TGCGACAGGCACGCAGCCTACTAGGTGTGGCGGCGACCCTGGCCCCGGGTTCCCGTGGCTACCGGGGGCG | 587 |
| TGAATGGTCAGCTTGTCCACAGGGTGAATCTTGTTGTAGTCAGCCGGGTCAGCGAAGGTCAGAGGCAGCA | 588 |
| CCCATCATACTCTTTCACCCACAGCACCAATCCTACCTCCATCGCTAACCCCACTAAAACACTCACCAAG | 589 |
| GCACCCAATACAGGAGCAGCCAGATTCATAAAGCAAGTCCTGAGTGACCTACAAAGAGACTTAGACTCCC | 590 |
| AGCTCTCTGCTCTCCCAGCGCAGCGCCGCCGCCCGGCCCCTCCAGCTTCCCGGACCATGGCCAACCTGGA | 591 |
| TTCAGCGTGGGGCGCCCACAATTTGCGCGCTCTCTTTCTGCTGCTCCCCAGCTCTCGGATACAGCCGACA | 592 |
| CCCAACCCGTCATCTACTCTACCATCTTTGCAGGCACACTCATCACAGCGCTAAGCTCGCACTGATTTTT | 593 |
| GAGTACACCGACTACGGCGGACTAATCTTCAACTCCTACATACTTCCCCCATTATTCCTAGAACCAGGCG | 594 |
| CTGGAGCCGGAGCACCCTATGTCGCAGTATCTGTCTTTGATTCCTGCCTCATCCTATTATTTATCGCACC | 595 |
| CTTCGAATGTGTGGTAGGGGTGGGGGGCATCCATATAGTCACTCCAGGTTTATGGAGGGTTCTTCTACTA | 596 |
| TCTCAACTTAGTATTATGCCCACACCCACCCAAGAACAGGGTTTGTTAAGATGGCAGAGCCCGGTAATCG | 597 |
| AGCATTCCTGCACATCTGTACCCACGCCTTCTTCAAGCCATACTATTTATGTGCTCCGGGGTCATCATCC | 598 |
| CCCAACCCGTCATCTACTCTACCATCTTTGCAGGCACACTCATCACAGCGCTAAGCTCGCACTGATTTTT | 599 |
| CCGCCATCTTCAGCAAACCCTGATGAAGGCTACAAAGTAAGCGCAAGTACCCACGTAAAGACGTTAGGTC | 600 |
| CCGGGATCGTCATCTACTCTACCATCTTTGCAGGCACACTCATCACAGCGCTAAGCTCGCACTGATTTTT | 601 |
| ACTTTTGAAATTCACACATTGTGAAGCCTGCCAGTCCCCGCCAGGTGAAGAGCTCATGGTATCCACCTTC | 602 |
| CTGGTGAAGCCCCAGCTATCATGGCAGTGAAGGGCTCTGGCTAGATTTGGATGTCAACTGCTGAGTTCTA | 603 |
| CGCTGGACCGGTCCGGATTCCCGGGATGTCCACACAGGCAGACTTGACCTTGACAGATAGTCTTCAAGAT | 604 |
| ACCCACAGGTCCTAAACTACCAAACCTGCATTAAAAATTTCGGTTGGGGCGACCTCGGAGCAGAACCCAA | 605 |
| CACCCTAGTAGGCTCCCTTCCCCTACTCATCGCACTAATTTACACTCACAACACCCTAGGCTCACTAAAC | 606 |
| ACAGAGCTCCTTCAAACTTCAGAACGGCCTATGAAGGAGTCCCGTGGAAACATCTGGGAGGACTTTCAAG | 607 |
| TAGTTAGGGCCCTCGGCCACACTCAAGTTCTGCTCCTCCAACAGGGCCTGAAAGTTTTTTCGGAAGCGAA | 608 |
| TGGTCGTGGGAGGGCTGAACACACATTACCGCTACATTGGCAAGACCATGGATTACCGGGGAACCATGAT | 609 |
| ACGCGTCCGCTCTGACTTCTTGGACTACATGGGGATCAAAGGCCCCAGGATGCCTCTGGGCTTCACGTTC | 610 |
| ACAGAATATCCTGTAGAAAAACTAATGAGGGATGCCAAAATCTATCAGATTTATGAAGGTACTTCACAAA | 611 |
| CCCACGCGTCCGAGCAAGTTGAAAATGGATTGAGACTGCATGGTGGCATAAATGAGAAATTGCCTGTAGC | 612 |
| CAAAGTAGTGATGGATTCAGTACTCCTCAACCACTCTCCTAATGATTGGAACAAAAGCAAACAAAAAAGA | 613 |
| TACCCAGCACATCCCACTATACCAGATGAGTGGCTTCTATGGCAAGGGTCCCTCCATTAAGCAGTTCATG | 614 |
| AAGAACAGTACAAAGAACATCCGTGTACCCAGTACCCTGACTACCGACTACCTACAACCCGTCCCTGCCC | 615 |
| CCTTACCACCAAACATACCAAAATGCACCTCTTTCATAAGTGAGTTACTAAGATTTCTATACCTGGAATA | 616 |
| CCTATTTGGACCAGAAACCCTGATGACATCACCCAAGAGGAGTATGGAGAATTCTACAAGAGCCTCACTA | 617 |
| ACGGGAGAGGTACTGAGGACAAATCAGTTCTCTGTGACCAGACATGAAAAGGTTGCCAATGGGCTGTTGG | 618 |
| TACCCTAGCCAACCCCTTAAACACCCCTCCCCACATCAAGCCCGAATGATATTTCCTATTCGCCTACACA | 619 |
| TACAGAGTCACACTCAATCCTCCGGGCACCTTCCTTGAAGGAGTGGCTAAGGTTGGACAATACACGTTCA | 620 |
| ACCCTTGGCCATAATATGATTTATCTCCACACTAGCAGAGACCAACCGAACCCCCTTCGACCTTGCCGAA | 621 |
| GCCCACTTCTTACCACAAGGCACACCTACACCCCTTATCCCCATACTAGTTATTATCGAAACCATCAGCC | 622 |
| CACTTCTGGTTGCCAGGAGACAGCAAGCAAAGCCAGCAGGACATGAAGTTGCTATTAAATGGACTTCGTG | 623 |
| CAAAGGAAATCAGCAGTGATAGATGAAGGGTTCGCAGCGAGAGTCCCGGACTTGTCTAGAAATGAGCAGG | 624 |
| TGACCTGGCCTCTCCCCCACAGGAACAAAACACTGCCTCCAGAGTCTTTAAATTCTCAGTTATCAACGCC | 625 |
| GAGAAGGTAAGCACATTTGAGGCCACCTAGCCTTTGCTTCTCTGTTCAAATCAATTATATTTCAAAAGCT | 626 |
| GGTGTACACTCAAAACCTGTCCCCGGCAGCCAGTGCTCTCTGTATAGGGCCATAATGGAATTCTGAAGAA | 627 |
| GGGACATGCTTCCCCTTGTCCACCTTTGCAGCCTGTTTCTGTCATGTAGTTTCAACAAGTGCTACCTTTG | 628 |
| ACGCTCTTCGCTGTCGTTTGTGGTCTCGCGCAGGGCGGCCCCGGTTCTGGTGTTTGGCGTCGGAATTAAA | 629 |
| TAAGAGACGACAGGGACCGAAGAGGACCTCCACTCAGATCAGAACGTGAAGAAGTAAGTTCTTGGAGACG | 630 |
| GCACAGGCTGTGGCTTGCACTCCAGCCGCTCTAGTCTCTCAGGAATTTGCTTGTTACTTGTACTGTGTAA | 631 |
| CCAAACCAACTCTTTGCCAGCAGCCACAACATGCATTGACAGCGGCACAGTGAGATATAACTGATGGGCT | 632 |
| TATATATTGTGCATCAACTCTGTTGGATACGAGAACACTGTAGAAGTGGACGATTTGTTCTAGCACCTTT | 633 |
| TCAGAAATGAGGTGTAATTCCCCAACCCCTGCCCGCAAGAGCTAAGTAGGATCTTACTGTAAGTTGAAGG | 634 |
| AAATGGCCACCACCATTCTCCTTCCCCACCCCACCACAAAAAGAGAAGCTGTGTCTTTAGACAACCCTGA | 635 |
| GCATTTCTTCTATGCACTTATCAGAAAGATCAAAGNCTTTAATACTTTCACTAATTTTGCTACTGCTATC | 636 |
| ACCGGCGTCAAAGTATTTAGCTGACTCGCCACACTCCACGGAAGCAATATGAAATGATCTGCTGCAGTGC | 637 |
| TTGAGAAGTATCCTGAGGCATGGGGGGTTCATAGTAGAAGAGCGATGGTGAGAGCTAAGGTCGGGGCGGT | 638 |
| TCCCCCTACACCTGTGTCAGCTGCGGTGCCCGGGCAGGTACATCTACCTTCGAGGCAGGAGCCCTCTCAT | 639 |
| TTTCCATCAATTAGCTCCCGCACAAGTGTGGTCTCTTGCCCGTCCCATTTCTGCAGGTGAACAAGTTTCC | 640 |
| GGAAATGTGAGCCCTGCATTCTGAATGAGTTTTAGGATTATATTCTGATTCACTAATTCTCCTTTCAACC | 641 |
| GGGAGTGTAAAATGCTTCAGCCACTTTAGAAAATAGTTTTGCAGTTTCTTACAACATTAAAAATATATTT | 642 |
| TTCTGCTCCACGGGAGGTTTCTGTCCTCCCTGAGCTCGCCTTAGGACACCTGCGTTACCGTTTGACAGGT | 643 |
| ACATTCTGTGGGAATAAACACAACTTGCTTGCCCTGATGCTCAATAGCAGTGTAATCACCATGTTTAAAC | 644 |
| CTTTGATGCCTTGACCTATGATTTCAATACAGCGCATTACTTTGGCTGCTAATTTTTCTGGGAGGGCACA | 645 |
| GTTTCTAAAAATAGTGTTATAGGCTGGACTGTGTCCCCTTCCTGTGCCCCGCCGCCATGCATATATGGAC | 646 |
| GTCTTATTATTTTTTGTCGATCAATGCTTATCCTCGTGTTCGTTTTGATATATTAATGTATATGTGTTGG | 647 |
| CATCTTTAGTGAAAGAGTAAATGGTGGCCGAGGGCTCCTTTTGTGAGGGATGTGCCTTGGTGAAGAAGGC | 648 |
| CGGATCACTTGAGGTCAGGAGTTCGAGACCAGCCTCCAACATGGCAAAGCCCTGTCTCTACTAAAAATAC | 649 |
| CATCTCTCCAATCTACCCAAGAGGAACCCAGTTACCCGAAGGCAGGTTCCACAGCCCACTCCCAGCAGCA | 650 |
| TTCCCTGACCCCCATACCCTCACCCTTAAAATTCTCCTGTAACTCAACTAACAAAATCAAGCCTGATTCA | 651 |
| GGTCTTCTAAGCCAGGCAGGTGAGGCAATTTCATGTCTGTGATGTGCATCCGCTCCACTTTATCCCTTGT | 652 |
| CACGACGGTCTAAACCCAGCTCACGTTCCCTATTAGTGGGTGAACAATCCAACGCTTGGTGAATTCTGCT | 653 |
| GCATCAGCAGGCAGTTGGTTGAAGTCAGCGGAGGGGTGTTCCATTCTTTGTTTTTCCAGGGCTTGTTTTC | 654 |
| TCTATCCAACTTTGCCATCTTAGACTAGCCTTCTTTACCCTACTGACCCATACATTGGTCTCTGTATCCT | 655 |
| CTCCACCCCGGTGGTGCTGGTCCGGAAGGACGACCTGCACAGAAAGAGACTGCACAACACGATAGCACTG | 656 |
| CTAACCATTCGTGATTATTAAGATAGGGTTGGGTCAGGGCTTAGGGAGGGGGCAGAAATATTGGGGATAG | 657 |
| GACTACTTCCCAATTAACTCCAACTCACAGTGATCCTTTCAACTCATGCGGCATCTATTTTTGCCACCAC | 658 |
| AGCCCTCAGTAGACACGTCTAGGGCAGGCTTGAGAGATCAGATGGCGTGAAAGGCTTGTGATCTGTTCGT | 659 |
| GGGCCTGGAATTTCCTTTCCACTTGATAGAAGTATATATTAGGAAGTCCAGTTAATAGTATTTTTATTTA | 660 |
| CGTCCATGCCCTGAGTCCACCCCGGGGAAGGTGACAGCATTGCTTCTGTGTAAATTATGTACTGCAAAAA | 661 |
| TTGGGATCTGAGGGGTCCTCTCTGTGCCCATCACAGTTTGAGCTTCAGGGAAAAGAAGAAGAGGTCTTTG | 662 |
| CGCAGGCAACCAAAACTAAAGCACCCGACGACTTAGTTGCTCCGGTCGTGAAGAAACCACACATCTATTA | 663 |
| AAACAGATAGCCACAAGAGGTTGGGACAGAGGAGGGTAAAGGCTCAGAAGGAGGTTCAACCTCTGACTCA | 664 |
| CCACGTGGTCTCACGTTTTCATGTTGACAGCCAGTCAGAGTCAAGAGCTCAGCTGTATCGACAGATCGTC | 665 |
| TGTGAAGCCAGGTGTGGGTTCTACTCAGTGCGATAGATAGACTGAGTCTTCTCTCGTAGGTTACCATTAC | 666 |
| GTCCAACAGAGGAGGGATGTGGAGAGCGTTTCAGGTGCTTTTCAGGTCAGTGCATCAGCAAATCATTGGT | 667 |
| GAAAACATAACCAGCCATTGGCTATTTAAACTTGTATTTTTTTATTTACAAAATATAAATATGAAGACAT | 668 |
| AGGGTGTGGGTGGCTCCCCTCCAGGGATGGCTGCTCCACGGTTTGCATTAAAGGTTCTGTATAAGGCCAA | 669 |
| AGCATGGAAACAAGATGAAATTCCATTTGTAGGTAGTGAGACAAAATTGATGATCCATTAAGTAAACAAT | 670 |
| CCTTGGTTCCCTAACCCTAATTGATGAGAGGCTCGCTGCTTGATGGTGTGTACAAACTCACCTGAATGGG | 671 |
| CAATCTGAAATAAAAGTGGGATGGGAGAGCGTGTCCTTCAGATCAAGGGTACTAAAGTCCCTTTCGCTGC | 672 |
| TGATGGCGCCTTCAAAGAGGTGAAGCTGTCGGACTACAAAGGGAAGTACGTGGTCCTCTTTTTCTACCCT | 673 |
| GGAGTAGCTGAGATCTTAGAAGCCGTCACCTACACTCAAGCCTCGCCCAAAGAAGCAAAAGTTGAACCCA | 674 |
| AGGGAATAGAAATGAAACAAATTATCTCTCATCTTTTGACTATTTCAAGTCTAATAAATTCTTAATTAAC | 675 |
| CCCAGAAAACAGAAGTTTCTACTGTCTCGTCTACCCAAGTTGGCCCCAACTGAGGACCCAATATTGGCCT | 676 |
| CGTGTGATTGGTGCAGGAGAATTCGGTGAAGTCTGCAGTGGCCGTTTGAAACTTCCAGGGAAAAGAGATG | 677 |
| CAAAACTGGATGGCATCCGAATTGTCTGGAAGTTTTGTCTTGGGCATGATGGGCTGGGCCAAATGAAATG | 678 |
| CACCCTTCAGGGGATGAGAAGTTTTCAAGGGGTATTACTCAGGCACTAACCCCAGGTTAGATGACAGCAC | 679 |
| TGGAGGACCGAACCGTAGTACGCTAAAAAGTGCCCGGATGGACTTGTGGATAGTGGTGAAATTCCAATCG | 680 |
| TAAGCTTGCGTTGATTAAGTCCCTGCCCTTTGTACACACCGCCCGTCGCTACTACCGATTGGATGGTTTA | 681 |
| CATCATTCAGATGGCTTTCCAGATGACCAGGACGAGTGGGATATTTTGCCCCCAACTTGGCTCGGCATGT | 682 |
| CTGACTATTACTGTAACTCCCGGGACAGCAGTGGTAACCGTCTGGTATTCGGCGGAGGGACCAAGCTGAC | 683 |
| GCGCGCTCGCCCCGCCGCTCCTGCTGCAGCCCCAGGCCCCTCGCCGCCGCCACCATGGACGCCATCAAGA | 684 |
| AGCGAGTTCTACATCCTAACGGCAGCCCACTGTCTCTACCAAGCCAAGAGATTCGAAGGGGACCGGAACA | 685 |
| GATCTCGGATGACCAAACCAGCCTTCGGAGCGTTCTCTGTCCTACTTCTGACTTTACTTGTGGTGTGACA | 686 |
| TGGTCCTGCGCTTGAGGGGGGGTGTCTAAGTTTCCCCTTTTAAGGTTTCAACAAATTTCATTGCACTTTC | 687 |
| AAGACGAATAGTCAAAAGGGAGCCTCTTCTACCTGGATGAAGGCAATTGTGTCATCGGGGACACTAGGTG | 688 |
| CCTACATTCCCTCTCCTGCCCAGATGCCCTTTGGAAAGCCATTGACCACCCACCATATTGTTTGATCTAC | 689 |
| AACATGACAAGGAATTCTTCCACCCACGCTACCACCATCGAGAGTTCCGGTTTGATCTTTCCAAGATCCC | 690 |
| ACCGTGACAATTGGCCTCCGGGGGCCACTTTCGGCGGAGGGACCAAGGTGGAGATCAAACATACCACCGG | 691 |
| CCAACTACCGCGCTTATGCCACGGAGCCGCACGCCAAGAAAAAATCTAAGATCTCCGCCTCGAGAAAATT | 692 |
| AGATTCCAGGCGGTGCAACCCTGGTGTTCGAGGTGGAGCTGCTCAAAATAGAGCGACGAACTGAGCTGTA | 693 |
| GCTTCGAGGGTGTGAAGGGAAAGAAGAAGATGTCAGCAGCAGAGGCAGTGAAAGAAAAATGGCTCCCGTA | 694 |
| ATAGGAAGTAGACCTCTTTTTCTTACCAGTCTCCTCCCCTACTCTGCCCCCTAAGCTGGCTGTACCTGTT | 695 |
| AGCGCCGCTGTGACTCGGGGTGACCTCCGCATCCTGCCTGAGGCCCATCAGCGCACATGGCATGCCTGGA | 696 |
| GCAAGTGGTCAACAGCAGGTGGCTGTCGAGACGTCTAATGACCATTCTCCATATACCTTTCAACCTAATA | 697 |
| CCGCTAGGGGTGCGGGGTTGGGGAGGAGGCCGCTAGTCTACGCCTGTGGAGCCGATACTCAGCCCTCTGC | 698 |
| GCGCAGATAGCACTTCAGCTCGGCCATCTCAGATCCCAACTCCAGTGAATAACAACACAAAGAAGCGAGA | 699 |
| TAGCTGGACAGGCCCTGCCCCTCACCAGCAAGAGGCATGATTGGATGGAGCTTCTAATGTCATTCAAAAA | 700 |
| GTATATCTTTAATTCTGGGAGAAATGAGATAAAAGATGTACTTGTGACCATTGTAACAATAGCACAAATA | 701 |
| TCAAGACTTTACCACTGGTTGACTCAAAAGATTCAATGATCCTGCTGGGCTCGGTGGAGCGGTCGGAACT | 702 |
| ATGGGGACTTGTGAATTTTTCTAAAGGTGCTATTTAACATGGGAGGAGAGCGTGTGCGCTCCAGCCCAGC | 703 |
| CATGTCTCCCATCAGAAAGATTCATTGGCATGCCACAGGGATTCTCCTCCTTCATCCTGTAAAGGTCAAC | 704 |
| AGCCGCCGCGTCCCCTCGCCGAGTCCCCTCGCCAGATTCCCTCCGTCGCCGCCAAGATGATGTGCGGGGC | 705 |
| CATAAATCAACTGTCCATCAGGTGAGGTGTGCTCCATACCCAGCGGTTCTTCATGAGTAGTGGGCTATGC | 706 |
| TATCTATATTTTACATAAATTTAGTATTTTGTTTCAGTGCACTAATATGTAAGACAAAAAGGACTACTTA | 707 |
| CTCGCGTCTCACTCAGTGTACCTTCTAGTCCCGCCATGGCCGCTCTCACCCGGGACCCCCAGTTCCAGAA | 708 |
| CGAAATATCAATGCAAACTAGGATATGTAACAGCAGATGGTGAAACATCAGGATCAATTAGATGTGGGAA | 709 |
| TTTACTAAGTAAAAGGGTGGAGAGGTTCCTGGGGTGGATTCCTAAGCAGTGCTTGTAAACCATCGCGTGC | 710 |
| ATAGATCTTGGCCCTGTTAAGGCATCCACTTCACAGTTCTGAAGGCTGAGTCAGCCCCACTCCACAGTTA | 711 |
| GCGGATCAGTGATAGCCATGAGGACACTGGGATTCTGGACTTCAGCTCACTGCTGAAAAAGAGAGACAGT | 712 |
| AAAAAAGACTTTGAGCTGAATGCTCTCAACGCAAGGATTGAGGATGAACAGGCCCTCGGCAGCCAGCTGC | 713 |
| CATCAACTATGAAGCATTTGTGAAGCACATCATGTCCAGCTAAACCTCGTGCCCAGAAGCCAGGAAGGCT | 714 |
| GAAATTCTTGGAAACTTCCATTAAGTGTGTAGATTGAGCAGGTAGTAATTGCATGCAGTTTGTACATTAG | 715 |
| AGCCCTTGCAAAAACACGGCTTGTGGCATTGGCATACTTGCCCTTACAGGTGGAGTATCTTCGTCACACA | 716 |
| TTTACTTGGTATAATATACATGGTTAAAATGCTTATGTGACTTCGAGTAGGTGAATCTTAAAGAAATAAA | 717 |
| CTGCTATAGCGGTGTCATGTTGGATCGCTTTGTGACTGTTCATCTGTCCTTGACAGTGGCTGTCATCTTG | 718 |
| ACAAGGAGTCAGACATTTTAAGATGGTGGCAGTAGAGGCTATGGACAGGGCATGCCACGTGGGCTCATAT | 719 |
| CAAAGCCAGACAAGCCAACGACACAGCTAAAGATGTACTGGCACAGATTACAGAGCTCCACCAGAACCTC | 720 |
| AGGCGCGGAGGTCTGGCCTATAAAGTAGTCGCGGAGACGGGGTGCTGGTTTGCGTCGTAGTCTCCTGCAG | 721 |
| TTGGAGGCATTCCTACTTACGGGGTTGGAGCTGGGGGCTTTCCCGGCTTTGGTGTCGGAGTCGGAGGTAT | 722 |
| CCGTCTGTCCTTTGTCCACAAGGAATTTCCCTGGGCGCTAATTATGAGGGAGGCGTGTAGCTTCTTATCA | 723 |
| CTGGTATTATCTCTCTATCAGATAAGATTTTGTTAATGTACTATTTTACTCTTCAATAAATAAAACAGTT | 724 |
| AGATGGGTGCTGGTCCTGTTGATCCCAGTCTCTGCCAGACCAAGGCGAGTTTCCCCACTAATAAAGTGCC | 725 |
| GTGCTACACCCTTTTCCAGCTGGATGAGAATTTGAGTGCTCTGATCCCTCTACAGAGCTTCCCTGACTCA | 726 |
| ATCCCAGTGGAGGGGACCCTTTTACTTGCCCTGAACATACACATGCTGGGCCATTGTGATTGAAGTCTTC | 727 |
| AAAAGCCACGGACCGTTGCACAAAAAGGAAAGTTTGGGAAGGGATGGGAGAGTGGCTTGCTGATGTTCCT | 728 |
| ATGCCGGCCTCCCTGTTGTCCACTGCCCCAGCCACATCATCCCTGTGCGGGTTGCAGATGCTGCTAAAAA | 729 |
| CCCCAAACCATAAAACCCTATACAAGTTGTTCTAGTAACAATACATGAGAAAGATGTCTATGTAGCTGAA | 730 |
| TGCACTCCAGCCGGGGTGACAGAAGAGACCTTGTCTCGAAAACGAATCTGAAAACAATGGAACCATGCCT | 731 |
| GAGGACCTCCGCTGCAAATACATCTCCCTCATCTACACCAACTATGAGGCGGGCAAGGATGACTATGTGA | 732 |
| GAGGCCTTGTGTCCTTTAATCACTGCATTTCATTTTGATTTTGGATAATAAACCTGGCTCAGCCTGAGCC | 733 |
| TCCAAGGCAGGTCATCCTGACACTGCAACCCACTTTGGTGGCTGTGGGCAAGTCCTTCACCATTGAGTGC | 734 |
| TAATGCTCTGGGAGGATGGGGAGAACTACAGAATTCGGTAAAGACATTTGGGGAGACACATCCTTTCACC | 735 |
| TTCCCCAATTATCCTCCTTCACTCCCTGTCATAGTTACCGATGGTGTCCCGTTGTGTGGGTTTACTCTGT | 736 |
| CGTAAGGGCTACAGTCGAAAAGGGTTTGACCGGCTTAGCACTGAGGGCAGTGACCAAGAGAAAGAGGATG | 737 |
| CTGCAGAGAAGAAACCTACTACAGAGGAGAAGAAGCCTGCTGCATAAACTCTTAAATTTGATTATTCCAT | 738 |
| TGAGAGCTAAACCCAGCAATTTTCTATGATTTTTTCAGATATAGATAATAAACTTATGAACAGCAACTAA | 739 |
| GGCTGGAACCATGGAGGGTGTAGAAGAGAAGAAGAAGGAGGTTCCTGCTGTGCCAGAAACCCTTAAGAAA | 740 |
| AAGAACTTGCCACTAAACTGGGTTAAATGTACACTGTTGAGTTTTCTGTACATAAAAATAATTGAAATAA | 741 |
| GGTGCTGTGGAATGCCCAGCCAGTTAAGCACAAAGGAAAACATTTCAATAAAGGATCATTTGACAAGTGG | 742 |
| CGGGCCAGCCGAGGCTACAAAAACTAACCCTGGATCCTACTCTCTTATTAAAAAGATTTTTGCTGACAAA | 743 |
| TAATCATGTCGTCGCCAAGTCCCGCTTCTGGTACTTTGTATCTCAGTTAAAGAAGATGAAGAAGTCTTCA | 744 |
| GAGGAGATCATCAAGACTTTATCCAAGGAGGAAGAGACCAAGAAATAAAACCTCCCACTTTGTCTGTACA | 745 |
| TTCTCGTGGTAATACCAGAGTAGAAGGAGAGGGTGACTTTACCGAACTGACAGCCATTGGGGAGGCAGAT | 746 |
| TCTTGCTGATATAATGGCCAAGAGGAATCAGAAACCTGAAGTTAGAAAGGCTCAACGAGAACAAGCTATC | 747 |
| TGAGGAAATCTGAAATAGAGTACTATGCTATGTTGGCTAAAACTGGTGTCCATCACTACAGTGGCAATAA | 748 |
| CCCAGGCTGTTTGGCGCTGCCCAGGAATGGTATCAATTCCCCTGTTTCTCTTGTAGCCAGTTACTAGAAT | 749 |
| CTGTCCAATAGAAAAAGTTGGTGTGCTGGAGCTACCTCACCTCAGCTTGAGAGAGCCAGTTGTGTGCATC | 750 |
| GCCAAGGAAGAGTCGGAGGAGTCGGACGAGGATATGGGATTTGGTCTCTTTGACTAATCACCAAAAAGCA | 751 |
| GAGCGCGGCGGCAAGATGGCAGTGCAAATATCCAAGAAGAGGAAGTTTGTCGCTGATGGCATCTTCAAAG | 752 |
| TCGGACGCCGGATTTTGACGTGCTCTCGCGAGATTTGGGTCTCTTCCTAAGCCGGCGCTCGGCAAGTTCT | 753 |
| GCCAAGCTGACTCCTGAGGAAGAAGAGATTTTAAACAAAAAACGATCTAAAAAAATTCAGAAGAAATATG | 754 |
| TTCCTCTCCAGCCCCTGCGTAATCGATAAGGAAACCCGGACGCTGCTGCCCCTTTCTTTTTTTCAGGCGG | 755 |
| TTTCGTTGCCTGATCGCCGCCATCATGGGTCGCATGCATGCTCCCGGGAAGGGCCTGTCCCAGTCGGCTT | 756 |
| TCTTTTACCAAGGACCCGCCAACATGGGCCGCGTTCGCACCAAAACCGTGAAGAAGGCGGCCCGGGTCAT | 757 |
| AACGACGCAAACGAAGCCAAGTTCCCCCAGCTCCGAACAGGAGCTCTCTATCCTCTCTCTATTACACTCC | 758 |
| GTTGAGGTGGAAGTCACCATTGCAGATGCTTAAGTCAACTATTTTAATAAATTGATGACCAGTTGTTAAA | 759 |
| GCTGGTGAAGATGCATGAATAGGTCCAACCAGCTGTACATTTGGAAAAATAAAACTTTATTAAATCAAAA | 760 |
| GCCTCGTCGAAGGTGCTAAAAAGATCAAAGTTGCAGAACTGTTAGCCAACATGCCAGACCCCACTCAGGA | 761 |
| CTATTCCCTCAAATCTGAGGGAGCTGAGTAACACCATCGATCATGATGTAGAGTGTGGTTATGAACTTTA | 762 |
| TATTTGTATGTGGGGAGTAGGTGTTTGAGGTTCCCGTTCTTTCCCTTCCCAAGTCTCTGGGGGTGGAAAG | 763 |
| CGGAGAAGAATCGGATCAATAAGGCCGTATCTGAGGAACAGCAGCCTGCACTCAAGGGCAAAAAGGGAAA | 764 |
| AGTGGGTGGAGGCAGCCAGGGCTTACCTGTACACTGACTTGAGACCAGTTGAATAAAAGTGCACACCTTA | 765 |
| GACCGGTTAAGGAGAAGCCAGAGTTAGAGTAGGAGAGGACTAATTCTCAGCAGCAGTGGAGGTGAGTTCT | 766 |
| TATTGATGGGCCCAAGCGTAACCAGGCTCTTCTGATTGGCCGGTGTACTTCAGTTTCCGTCCAAGGTCCG | 767 |
| TCTTTTGTGGTTGTTGCTGGCCCAATGAGTCCCTAGTCACATCCCCTGCCAGAGGGAGTTCTTCTTTTGT | 768 |
| GAGGGCAGGGACCGTATCTTATTTACTGTTAGTATCCGTTGCATCTAGTGTGGTGCACCTGGCACACAGT | 769 |
| TGGCAAGAGAGCCTCACACCTCACTAGGTGCAGAGAGCCCAGGCCTTATGTTAAAATCATGCACTTGAAA | 770 |
| GGAGCCTCTTTGTAGGGACTGTGCCTAGGTAGCATGTCCTAACATTTGTTCTGGTCTTGCATAACTTCAG | 771 |
| GTATCCCGCGGGTGGAGGCCGGGGTGGCGCCGGCCGGGGCGGGGGAGCCCAAAAGACCGGCTGCCGCCTG | 772 |
| GTAGGGATGGGGCTGTGGGGATAGTGAGGCATCGCAATGTAAGACTCGGGATTAGTACACACTTGTTGAT | 773 |
| CGCGGTTTGGTTTGCAGCGACTGGCATACTATGTGGATGTGACAGTGGCGTTTGTAATGAGAGCACTTTC | 774 |
| GCCTGCACCAGTGCCGTCCTGCTGATGTGGTAGGCTAGCAATATTTTGGTTAAAATCATGTTTGTGGCCG | 775 |
| TCACTCCTTAAATTCACACTTTGCCACTTAACTCCAGTGTGGATGACAGAGCGAGACCCTGCCTCAAAAA | 776 |
| GCCCTGGGCAGCCAGCATTCATTGTAAGTTCCCTCTTTGAAAACTGGTGTGTGGGTGTTCAGTTCTGTGT | 777 |
| AGAAAAAAGTCACGTTAAATGGTTTCTTGGACACGCTTATGTCAGATCCTCCCCCGCAGTGTCTGGTCTG | 778 |
| CATGTGGGCAAAGCCTTCAATCAGGGCAAGATCTTCAAGTGAACATCTCTTGCCATCACCTAGCTGCCTG | 779 |
| ATGAAGCCAGGATTCAGTCCCCGTGGGGGTGGCTTTGGCGGCCGAGGGGGCTTTGGTGACCGTGGTGGTC | 780 |
| GCAGCTATTTCAAAGTGTGTTGGATTAATTAGGATCATCCCTTTGGTTAATAAATAAATGTGTTTGTGCT | 781 |
| GACCAGTTGTTATTTACAGCTCTGTAACCTCCCGTTGCGTCAAGTCTAAACCAAGATTATGTGACTTGCA | 782 |
| CACTTCACAGTAAATGCCAAAGCTGCTGGCAAAGGCAAGCTGGACGTCCAGTTCTCAGGACTCACCAAGG | 783 |
| AGGAAGTTATGGGAATACCTGTGGTGGTTGTGATCCCTAGGTCTTGGGAGCTCTTGGAGGTGTCTGTATC | 784 |
| CTCACTGGGTGGCTTTGCCTATGTGGAGATCAGCTCCAAAGAGATGACTGTCACTTACATCGAGGCCTCG | 785 |
| AGCGTGAGATTGTCCGGGACATCAAGGAGAAACTGTGTTATGTAGCTCTGGACTTTGAAAATGAGATGGC | 786 |
| TAGAATCCTCAACCGTGCGGACCATCAACCTTCGAGAAATTCCAGTTGTCTTTTTCCCAGCCGCATCCTG | 787 |
| CAACCACGACAAAGGAAGTTGACCTAAACATGTAACCATGCCCTACCCTGTTACCTTGCTAGCTGCAAAA | 788 |
| GAGGCTCTGTAACCTTATCTAAGAACTTGGAAGCCGTCAGCCAAGTCGCCACATTTCTCTGCAAAATGTC | 789 |
| TAGGCGGAGCCTCGGCCGCGGGCCGCCTTGGTATATCTGCGTGCGCGCGTCTGCTGGGCCAGTCGGGACA | 790 |
| CTAGCGGTTACGCCAACGCGCGCGTGCGCCCTTGCGCGTTTCTCTCTTCCCACTCGGGTTTGACCTACAG | 791 |
| CGCAAGGAGGGGCTGCTTCTGAGGTCGGTGGCTGTCTTTCCATTAAAGAAACACCGTGCAACGTGAAAAA | 792 |
| GGAGTTGGTCAAATGAGGGAACATCTGGGTTATGCCTTTTTTAAAGTAGTTTTCTTTAGGAACTGTCAGC | 793 |
| AGGCATCTGGAGAGTCCAGGAGAGGAGACTCACCTCTGTCGCTTGGGTTAAACAAGAGACAGGTTTTGTA | 794 |
| AACTAATCCATCACCGGGGTGGTTTAGTGGCTCAACATTGTGTTCCCATTTCAGCTGATCAGTGGGCCTC | 795 |
| ACAATTTGTTTCAGAGAAGAGAGTTGAACAGTGGTGAGCTGGGCTCACAGCTCCATCCATGGGCCCCATT | 796 |
| TGTTGACACAGGTCTTTCCTAAGGCTGCAAGGTTTAGGCTGGTGGCCCAGGACCATCATCCTACTGTAAT | 797 |
| CTGGGGGAGTGGAATAGTATCCTCCAGGTTTTTCAATTAAACGGATTATTTTTTCAGACCGAAAAGAAAA | 798 |
| GCTCCCAGCACACTCGGAGCTTGTGCTTTGTCTCCACGCAAAGCGATAAATAAAAGCATTGGTGGCCTTA | 799 |
| GGCCACTTTTCACTAACAGAAGTCACAAGCCAAGTGAGACACTCATCCAAGAGGAAGGATGGCCAGTATC | 800 |
| AGACCAGAGATAGTGGGGAGACTTCTTGGCTTGGTGAGGAAAAGCGGACATCAGCTGGTCAAACAAACTC | 801 |
| CCATGGATGAGAAAGTCGAGTTGTATCGCATTAAGTTCAAGGAGAGCTTTGCTGAGATGAACAGGGGCTC | 802 |
| GTTTTTCAGCTCACTTCAAGGGTACCTGAAGCGAATTGGCACCAAAGCAGCAGCTGTATTGCCGCAGTTC | 803 |
| GGATAGATAATTTTATTTGAAATTTTACACACTGAAAGCTCTAAATAAACAGATACATTCACATTCAAAA | 804 |
| AGTTTCCCCACCAGTGAATGAAAGTCTTGTGACTAGTGCTGAAGCTTATTAATGCTAAGGGCAGGCCCAA | 805 |
| GGGGAGGCATCAGTGTCCTTGGCAGGCTGATTTCTAGGTAGGAAATGTGGTAGCTCACGCTCACTTTTAA | 806 |
| GACGCGGCTCAAAAGGAAACCAAGTGGTCAGGAGTTGTTTCTGACCCACTGATCTCTACTACCACAAGGA | 807 |
| CGGCCGAACCCAGACCCGAGGTTTTAGAAGCAGAGTCAGGCGAAGCTGGGCCAGAACCGCGACCTCCGCA | 808 |
| CTTGAAATTGTCCCCGTGGTCTCTTACTTTCCTTTCCCCAGCCCAGGGTGGACTTAGAAAGCAGGGGCTA | 809 |
| CAGGGGCCAGGGGAACCCGTGAGGATCACTCTCAAATGAGATTAAAAACAAGGAAGCAGAGAATGGTCAG | 810 |
| CCAAGTCCCTGAAGTCTGGAGACGCGGCCATCGTGGAGATGGTGCCGGGAAAGCCCATGTGTGTGGAGAG | 811 |
| ACAAGGTGGGGACAGACTTGCTGGAGGAGGAGATCACCAAGTTTGAGGAGCACGTGCAGAGTGTCGATAT | 812 |
| AACGCATTAAGAGGTTTATTTGGGTACATGGCCCGCAGTGGCTTTTGCCCCAGAAAGGGGAAAGGAACAC | 813 |
| CCTGCCCTGCACCCTTGTACAGTGTCTGTGCCATGGATTTCGTTTTTCTTGGGGTACTCTTGATGTGAAG | 814 |
| GAAGAAGGGCCCCAATGCCAACTCTTAAGTCTTTTGTAATTCTGGCTTTCTCTAATAAAAAAGCCACTTA | 815 |
| AAAGTGTGAATGTGGGTGTCGGCTGCGGCATTAAATTCATCATCTCAACCCAGAGTGTCTGGTCTCCCTG | 816 |
| CTTTTCCCTATCCACAGGGGTGTTTGTGTGTGTGCGCGTGTGCGTTTCAATAAAGTTTGTACACTTTCAA | 817 |
| ATGCGCAGCAGCGGCGCCGACGCGGGGCGGTGCGTGGTGACCGCGCGCGCTCCCGGAAGTGTGCCGGCGT | 818 |
| GGGGTCAAAAGGTACCTAAGTATATGATTGCGAGTGGAAAAATAGGGGACAGAAATCAGGTATTGGCAGT | 819 |
| ATCAGTTCTTAATTTAATTTTTAAGTATTGTTTTACTCCTTTTTATTCATACGTAAAATTTTGGATTAAT | 820 |
| AAAGAGGGTCCATCAAAGAGATGAGCCATCACCCCCCAGGACACACAGTGGTCAAGGATAGAAGCCATTT | 821 |
| GCACGGCATGGATTAACACGGCAGAGGAACAAAGGTGTGCTCTGAGCTTCTTCATATTTCACCTTCACCC | 822 |
| GGCTATGCAACAGCTCTCACCTACGCGAGTCTTACTTTGAGTTAGTGCCATAACAGACCACTGTATGTTT | 823 |
| GTACAGTCGCCGCGTGCGGAGCTTGTTACTGGTTACTTGGCCTCATGGCGGTCCGAGCTTCGTTCGAGAA | 824 |
| AGCATATTGTCTGGGGATTGTTGGGACAGGTTTTGGTGACTCTGTGCCCTTGCTCTCTAACTTCTGAGCC | 825 |
| AGACACATGGAACAAAGAAGCTGTGACCCCAGCAGGATGTCTAATATGTGAGGAAATGAGATGTCCACCT | 826 |
| AGTTCGTTGTGCTGTTTCTGACTCCTAATGAGAGTTCCTTCCAGACCGTTAGCTGTCTCCTTGCCAAGCG | 827 |
| GCTCCAGGTTGGGTGCTCACAGAACCCTTTTCCTGACTCTCATGGAAGATGGTGGAAGGAAAATAGACTG | 828 |
| AAAAAATCTTACACATCTGCCACCGGAAATACCATGCACAGAGTCCTTAAAAAATAGAGTGCAGTATTTA | 829 |
| GTTGAAGGGGCTGGTGCCACTGGGACCCGAATCAAGTCGACACACTACGTTGAGTTTATTAACAAAAGCC | 830 |
| AGAAGACAAAGAGCAAGGGGCCCTACATCTGCGCTCTGTGCGCCAAGGAGTTCAAGAACGGCTACAATCT | 831 |
| CCTACCCCGAACTCCAAAAATTACACCTGGAGTCAGGTGCAGAAGGGAACCTTGTATTTCACAGGCCTCA | 832 |
| ACCACAGTGGTGTCCGAGAAGTCAGGCACGTAGCTCAGCGGCGGCCGCGGCGCGTGCGTCTGTGCCTCTG | 833 |
| ACAGTAAGATTGAGGATGAGCAGGCGCTGGCCCTTCAACTACAGAAGAAACTGAAGGAAAACCAGGCACG | 834 |
| TGAGGCTCCCAAGGAACCTGCCTTTGACCCCAAGAGTGTAAAGATAGACTTCACTGCCGACCAGATTGAA | 835 |
| CCAAAATACTTGCATCCAAGGTTCTAGTCTCTGTTGCTGTGCTGGTCTTTAGCCCCACTGCTGGCACTGA | 836 |
| GAGTGTGTCTCATGCTTTCAGATGTGCATATGAGCAGAATTAATTAAACATTTGCCTATGACTCCAACAA | 837 |
| ATATTGCAAAAGGATGTGTGTCTTTCTCCCCGAGCTCCCCTGTTCCCCTTCATTGAAAACCACCACGGTG | 838 |
| CACTTCTGGTTGCCAGGAGACAGCAAGCAAAGCCAGCAGGACATGAAGTTGCTATTAAATGGACTTCGTG | 839 |
| TAATCATTTTCTAGAAAGTATGGGTATCTATACTAATGTTTTTATATGAAGAACATAGGTGTCTTTGTGG | 840 |
| AATGTAACTATTTAGCCCTGGATTATACATACTGTCCAATTTTCATTAAATTTTTGTCTTATAACTATAA | 841 |
| TTGGCTGCCGGTGAGTTGGGTGCCGGTGGAGTCGTGTTGGTCCTCAGAATCCCCGCGTAGCCGCTGCCTC | 842 |
| TTACTACTGTGGGTTTAAAGCCACTGCAGCGGGAGTTAAACAAACTGAGTCAACCAGCTTCCTTGAAAAA | 843 |
| GCAGCCATCTCGCCGTGAGACAGCAAGTGTCGCGCAGCCGTGCGATGTTGTCCTCTACAGCCATGTATTC | 844 |
| GGAAGTGAGTGGACAGCCTTTGTGTGTATCTCTCCAATAAAGCTCTGTGGGCCAAGTCCTCTAGGAAAAA | 845 |
| AAATCTGGGTTCAACCAGCCCCTGCCATTTCTTAAGACTTTCTGCTGCACTCACAGGATCCTGAGCTGCA | 846 |
| TAAGGTAGCAGGCAGTCCAGCCCTGATGTGGAGACACATGGGATTTTGGAAATCAGCTTCTGGAGGAATG | 847 |
| AGCTAGTGCCGACTCCCGCCTAGCTCTTTTGACTCTGTTCGCGGGAAGAATGGGGAAACAGTAAGGTTGC | 848 |
| CATCTTGGGTTACCCACTCTGTCCACTCCCATAGGCTACAGAAAAAGTCACAAGCGCATGGTTTCCAACC | 849 |
| TTTTTCCACCCTGGCTCCTTCAGACACGTGCTTGATGCTGAGCAAGTTCAATAAAGATTCTTGGAAGTTT | 850 |
| TGCCATGTACTATTTTACCTATGACCCGTGGATTGGCAAGTTATTGTATCTTGAGGACTTCTTCGTGATG | 851 |
| GCCCTGCCACCGTGGGGAGTCTGGTTTTTCTCTTCATCCTGTCTCTCTCCTCCTTACTCTTGGATAAATA | 852 |
| AGGCCGAGCTCTGCAGAGCTTACAATTGAGACTGCTAACCCCTACCTTTGAAGGGATCAACGGATTGTTG | 853 |
| CCATCTCTAGGATGTCGTCTTTGGTGAGATCTCTATTATATCTTGTATGGTTTGCAAAAGGGCTTCCTAA | 854 |
| TGTTGGTTTATTGCTGGCAACGTGAATTCTCTCAGGGGTCTAGGAGGGGCATTTTGGAGACTGCCTGACA | 855 |
| CACTACCGTGGAGATCCCAACTGGTTTATGAAGAAAGCGCAGGAGCATAAGAGGGAATTCACAGAGAGCC | 856 |
| ATGGTTCCAGGACTACAATGTCTTTATTTTTAACTGTTTGCCACTGCTGCCCTCACCCCTGCCCGGCTCT | 857 |
| GACCATCACATCCCTTCAAGAGTCCTGAAGATCAAGCCAGTTCTCCTTCCCTGCAGAGCTTTGGCCATTA | 858 |
| AGGAGGGTCTTCGAGGGGCCTGGGGGCGGGGGACTAAGATGGACGCCTGGGAAGGGAACTGGGAGGCAGC | 859 |
| TGTCCTCAACCCCAAATCCCCCGACTCCCTCCCCAGATCTGTCCTGGGGGATGCAAATAAAGCCTGCTCT | 860 |
| GCCGTGCTTCTGCCCCTACAAGGTTTGGGCCGAGGTGGGGGAGGGTCCTGGTTGCCGGCCCCGCCCGGTC | 861 |
| CCCAGAAGCAGTTAAGTCTCCAAAACGAGTGAAATCTCCAGAACCTTCTCACCCGAAAGCCGTATCACCC | 862 |
| GTGCTTGTGGACATCAGGCCTCCTGCCAGCAGTTCTTGAAGCTTCTTTTTCATTCCTGCTACTCTACCTG | 863 |
| GGCGGGAGGATCACTTGAGGCCAGGACTTTGAGACCAGCCAGGGCAACATAATAAGACTTTTCTCTACTT | 864 |
| CCCAAGTGCACTCATCCAGGTCAGTGCTCAGATGTGTTTAAGGAGACCCTATATTCAGGGAAGTTGCGTG | 865 |
| CCTAGGTTCAGAGCATGGGTGCTCTGAGGGACAAAGTTGGATTAGTATAAGGGAGCTGGAGCAGCTGATA | 866 |
| GGCAGGACCTGTGGCCAAGTTCTTAGTTGCTGTATGTCTCGTGGTAGGACTGTAGAAAAGGGAACTGAAC | 867 |
| GGTGCCTGATACCTCTCAGCATTTGAGGGCCTTTTCTCTTCCTGCTTCATCTCTAAAGGTCCTTCTAGGA | 868 |
| TCACCACGTCTGGTCGAAAGATGGCAGAGCTGCCGGTGGACCCCATGCTGTCCAAAATGATCTTAGCCTC | 869 |
| AAAGGATAAACCCCGATATTGGGACCTCACAGTGGGTGTCTGAAAGGACAGATCACTCCGGAGTATCAGG | 870 |
| AAGAGAAATACACACTTCTGAGAAACTGAAACGACAGGGGAAAGGAGGTCTCACTGAGCACCGTCCCAGC | 871 |
| AATGAGGAGTGATCATGGCTACCTCAGAGCTGAGCTGCGAGGTGTCGGAGGAGAACTGTGAGCGCCGGGA | 872 |
| GAATTCTCAGCTCTTGGGAACCCCCTTGCTCCCAGGGGAGGGGAAACCTTTTTCATTCAACATTGTAGGG | 873 |
| AAATTCCTAAAACTGTGGAATGGATCACGTAGACATGTAACCCAGCAGCAGTTTGCTTCTGTTGTCCACT | 874 |
| GTACCATTCAGAATGGACTGTTTGTACGAAGCATGTATAATGCAGTTATCTTCTTTCTTTCGTCGCAGCC | 875 |
| CTTCTCCTCGACCAGCCATCATGACATTTACCATGAATTTACTTCCTCCCAAGAGTTTGGACTGCCCGTC | 876 |
| CCTGGCTTCATTCTGCTCTCTCTTGGCACCCGACCCTTGGCAGCATGTACCACACAGCCAAGCTGAGACT | 877 |
| AGTTATCATTACCATGTTGGTGACCTGTTCAGTTTGCTGCTATCTCTTTTGGCTGATTGCAATTCTGGCC | 878 |
| CCGCGAGATCTAGCATCTCTGAAATCCTGGCTGTCGAGGCTTTGAAGCATGTGTTACCTGGTTAAGCTTG | 879 |
| ACGAGGAAAATGGCGCTAGCTCGGAAGCTACCGAGGTGCTAGGAGTTGCCGAAGCAAGTCCGGAAGCTAC | 880 |
| TTGAAAATTAAACGTGCTTGGGGTTCAGCTGGTGAGGCTGTCCCTGTAGGAAGAAAGCTCTGGGACTGAG | 881 |
| TGAAGCTGGTGGTGTCTCGGGGCGGCCTGTTGGGAGATCTTGCATCCAGCGACGTGGCCGTGGAACTGCC | 882 |
| TCCATGTTTGATGTATCTGAGCAGGTTGCTCCACAGGTAGCTCTAGGAGGGCTGGCAACTTAGAGGTGGG | 883 |
| GCCATTCCATTCCCAGCAGCTTTGGAGACCTCCAGGATTATTTCTCTGTCAGCCCTGCCACATATCACTA | 884 |
| GATAAAAGGGGGAGACAAAAGATGTACAGAAATGATTTCCTGGCTGGCCAACTGGTGGCCAGTGGGAGGT | 885 |
| CAATAATCAGTGGTGCTTTTGTACCTAGGTTTTATGTGATTTTAATGAAACATGGATAGTTGTGGCCACC | 886 |
| TACACTGCTGTACCCAGATGCCTACAACCATCCCTGCCACATACAGGTGCTCAATAAACACTTGTAGAGC | 887 |
| TCGGGAACTGGCCCAACAGGTGCAGCAAGTAGCTGCTGAATATTGTAGAGCATGTCGCTTGAAGTCTACT | 888 |
| TAACTCTGGGAGGGGCTCGAGAGGGCTGGTCCTTATTTATTTAACTTCACCCGAGTTCCTCTGGGTTTCT | 889 |
| GATTAAGCTGAAGATGTTTATTACAATCACTCTCTGTGGGGGGTGGCCCTGCTGCTCCTCAGAATCCTGG | 890 |
| CATCTACCCCTGCTAGAAGGTTACAGTGTATTATGTAGCATGCAAATGTGTTTATGTAGTGGCTTAATAA | 891 |
| CCGCTGTCGCCGCCGCGGAGACAAAGATGGCTGCGAGAGTCGGCGCCTTCCTCAAGAATGCCTGGGACAA | 892 |
| TTCCATGGGAGATGACTCTTAAGCCATAGGGGCTGGTTTTCCGTACTCCAAACCATCAGGTGGACACAGT | 893 |
| ATTGTTTTTATCTGGTTACATATATATTTCTTTGTCTAATTTAATATGTCAAATAAATGAGTTCATCTAA | 894 |
| TCTGCGTGGGTGGTGATGGGGGTTCACCTGAACACAGAGTGTATTTTCTTATTGAGGCCCTGTACCTTCT | 895 |
| GAATACATTTCTGCCTGATAATCATGCTGGGTTCTAATAAGCCCTACTTCCACCTAATCTGTTTACAGTC | 896 |
| GGCCCAGAAGAAATTTAAGCGTCTTATGCTGCATCGGATAAAGTGGGATGAACAGACATCTAACACAAAG | 897 |
| GCCGAGTGTATTATAAAATCGTGGGGGAGATGCCCGGCCTGGGATGCTGTTTGGAGACGGAATAAATGTT | 898 |
| GCAGCGCCTCCCTTGTCTCAGATGGTGTGTCCAGCACTCGATTGTTGTAAACTGTTGTTTTGTATGAGCG | 899 |
| GCATACAGGTTATTGGAGAAATTTTCCTTTTGTTGCATTTGTGGAAGTTAGTTTTCTGGCCCGTGGCCTT | 900 |
| TTGGCGTAGCCATGGCGTCTCGTGTCCTTTCAGCCTATGTCAGCCGCCTGCCCGCGGCCTTTGCGCCGCT | 901 |
| CTTCAAATATGGCCGCCAAGCTCCGTTCTCTTTTACCGCCTGATCTACGGCTACAATTCTGGCTTCATGC | 902 |
| AGTGTGTCAAACAGATCTGCGTGGTCATGTTGGAGACTCTGTCCCAGTCCCCCCCGAAGGGCGTGACCAT | 903 |
| GGGCCAGGGCTGGATGGACAGACACCTCCCCCTACCCATATCCCTCCCGTGTGTGGTTGGAAAACTTTTG | 904 |
| CCTACTTCTTCAGCTGACACCCCGTGAGCCTTGTCAGTGTGTAAATAAAGCTCTTTTGCCACCCCCCAAA | 905 |
| GGCCCAACACAATTCTTCTTCCAACGTGGCCCAGAGAAGCCAAAAGATTGGATACGCATCAGACAGATGG | 906 |
| ATCCCAACGATGACAAGGACAGTGGCTTCTTTCCCCGAAACCCATCGAGCTCCAGCATGAACTCGGTTCT | 907 |
| TTCCTCGGGCATCGACGTGCTCATTTCCAAAGATGATGGTGCAGGTGACCTTTTCCATCGTGAGCTAAGA | 908 |
| AAAGGTTTTCACACCAGACACTGCAGCAGACACCCATGATAAGTACCATGACTCCAATGAGTGCCCAGGG | 909 |
| TGCTCCAACTGACCCTGTCCATCAGCGTTCTATAAAGCGGCCCTCCTGGAGCCAGCCACCCAGAGCCCGC | 910 |
| GACCATAGGATGGGAGGATAGGGAGCCCCTCATGACTGAGGGCAGAAGAAATTGCTAGAAGTCAGAACAG | 911 |
| ACTACTCTCTGAAGGAGTCCACCACTAGTGAGCAGAGTGCCAGGATGACAGCCATGGACAATGCCAGCAA | 912 |
| AGCCGGGCGAGCGCTGTGGGCCAAGCAGGGGTTGCAGGGTAGTAGGAGTGCAGACTGAAAAAATGCAGAC | 913 |
| GCCCCAGCGGTAACCACCAATCTTCTTTTGCCAATAGACCTCGAAAATCATCAGTAAATGGGTCATCAGC | 914 |
| CTAGTTATGATCAGAGCAGTTACTCTCAGCAGAACACCTATGGGCAACCGAGCAGCTATGGACAGCAGAG | 915 |
| AAAAATGTATAATATAAAATTGTAATACACTCAAATGATTATAAAAGTAAAAGTTGGTAATTTAGGCAAA | 916 |
| ACTACCTTTTTCGAGAGTGACTCCCGTTGTCCCAAGGCTTCCCAGAGCGAACCTGTGCGGCTGCAGGCAC | 917 |
| GGTGAACCTATGGGTCGTGGAACAAAAGTTATCCTACACCTGAAAGAAGACCAAACTGAGTACTTGGAGG | 918 |
| GGGGAAGCATTTGACTATCTGGAACTTGTGTGTGCCTCCTCAGGTATGGCAGTGACTCACCTGGTTTTAA | 919 |
| AGCAGGCTGTGCAGAGCGCGTTGACCAAGACTCATACCAGAGGGCCACACTTTTCAAGTGTATATGGTAA | 920 |
| CTCGGACGGGACTTTCTTGGTGCGGCAGAGGGTGAAGGATGCAGCAGAATTTGCCATCAGCATTAAATAT | 921 |
| CTTCAGGTTCCTCTTACTATGATAATGTCCGGCCTCTGGCCTATCCTGATTCTGATGCTGTGCTCATCTG | 922 |
| CACTGTGTACCCCGAGCAACATTCTAAGGGTGTGCTTTCGCCTTGGCTAACTCCTTTGACCTCATTCTTC | 923 |
| GAATCTAAGTTACCATCCCTTGGAAATTCTGGAGAAGGAGTCTCATGCACCACCTATCACACTCCCTCAC | 924 |
| GCCAGGATTGCTACAGTTGTGATTGGAGGAGTTGTGGCCATGGCGGCTGTGCCCATGGTGCTCAGTGCCA | 925 |
| GTCTTCAACTGGTTAGTGTGAAATAGTTCTGCCACCTCTGACGCACCACTGCCAATGCTGTACGTACTGC | 926 |
| CAAGAGGAGAGTGAAGAGGAAGAGGTCGATGAAACAGGTGTAGAAGTTAAGGACATAGAATTGGTCATGT | 927 |
| CAAGGTGCAGAATGGTTTGGAAAGTAGCTGTATTCCTCAGTGTGGCCCTGGGCATTGGTGCCATTCCTAT | 928 |
| CCTCGTCAGCAGCGAGGAAGGAAACAGCGGCGACAGCCCTGTACTGTGTCTGAAATTTTCCATTTTTGTT | 929 |
| ATGTACACACGTGCACGTACACACATGCATGCTCGCTAAGCGGAAGGAAGTTGTAGATTGCTTCCTTCAT | 930 |
| AACAAACCCTCATCTCATGAAGGACGGGGTGTGTGTGTGGCGTTGATCTTTAGCCTGTCTCACACCAGTT | 931 |
| AATTTTCTGCAGCATTAAAGCTGGCGCTTAATAAGAATAAGTAATAATAAAGAAATTTCTAACATTCCAA | 932 |
| GCCTGGAACAAGGACCGCACCCAGATTGCCATCTGCCCCAACAACCATGAGGTGCATATCTATGAAAAGA | 933 |
| GGAGTGCTTCCATCCCTCTCCACCCCTTCCCCCCAAAAGGTTTTCTTTGCAAGTGCTTTTGGAACTAAGA | 934 |
| AGCAGCTGCCTCACCGCCCAGACATTGATTTGTTCAGATGTTTCAATGCCTCATGATACAATAAAACCAC | 935 |
| AGAACAGGTTTTCAAAGTGGCCTCCTCAGACCTGGTCAACATGGGCATCAGTGTGGTTAGCTACACTCTG | 936 |
| TTCTCTGCTGGTAATTCCTGAAGAGGCATGACTGCTTTTCTCAGCCCCAAGCCTCTAGTCTGGGTGTGTA | 937 |
| GAGACCAGCCTGGAGCCTAGATCTGGTGCTTCTTCTGTGCTGTGGTTTACCCCAAACCTTTAGGTTGTTT | 938 |
| GGATGGGAATAGCAATGTGTGTTCAGAGAGAATGACAATGTGTGTTCAGAGAGAATGAATTGCTTAAACT | 939 |
| ACGCATTTGAGCGATTGCTCTGTGAAGAGTTGTACACTGAACACTTTCAGGGGAGGCTGTTTACCCAGGC | 940 |
| TGACTCTCTGAGGCTCATTTTGCAGTTGTTGAAATTGTCCCCGCAGTTTTCAATCATGTCTGAACCAATC | 941 |
| CGGAGGTGGTCAAGGCTAAAGCCGGAGCAGGCTCTGCCACCCTCTCCATGGCGTATGCCGGCGCCCGCTT | 942 |
| CTGGGTCCTGGGGCAGGGCGAGTCCAAGTGTGAGGCTGTTGATTTGTTTTCAATATTTCTTTTCGTGCTG | 943 |
| CTTAAGCCTTCCAGGACACTAAGGTCGTGGGAGCGGGACTGCAACAAGCAATGCCAGATAACTGAGAAAT | 944 |
| TATTTATCCCTTCTTGCCTGTGAGGACTGCGGCTTTTCGCTGTGGCTCGTCCTTAACGTTTCTGAACCAC | 945 |
| GGGACCCTGTTACAGACATACCCTATGCCACTGCTCGAGCCTTCAAGATCATTCGTGAGGCTTACAAGAA | 946 |
| GCAGCCCCTTTCCGGGACACCTGGGTTCACACAGCTTTTTAGCTTACATAACTGGTGCAGATTTTCTGTG | 947 |
| GCAAAATGAATTCCTGGCTTCAGTTAGCTATTATTTTTTTAATGACAACATAGACTGTGCTCTAAGTTTA | 948 |
| AATGCAAGCTCACCAAGGTCCCCTCTCAGTCCCCTTCCCTACACCCTGACCGGCCACTGCCGCACACCCA | 949 |
| TATGATGTATTTCTGAGCTAAAACTCAACTATAGAAGACATTAAAAGAAATCGTATTCTTGCCAAGTAAC | 950 |
| ATTTTACCTCTTTACCCTGTCGCTCATAATGAGGCATCATATATCCTCTCACTCTCTGGGACACCATAGC | 951 |
| GACACCTATCTAAGCCATTTTAACCCTCGGGATTACCTAGAAAAATATTACAAGTTTGGTTCTAGGCACT | 952 |
| ATTGAAAGCTAAGTGAGAGAGCCAGAGGGCCTCCTTGGTGGTAAAAGAGGGTTGCATTTCTTGCAGCCAG | 953 |
| ACATTCACATCTAGTCAAGGGCATAGGAACGGTGTCATGGAGTCCAAATAAAGTGGATATTCCTGCTCGG | 954 |
| CAAGGGCGCAAGAGTAGCGGTCCAAGCCTGCAACTCATCTTTCATTAAAGGCTTCTCTCTCACCAGCAAA | 955 |
| AGCACCGCCGCGGAGAACAAGGCCAGCCCCGCGGGGACAGCGGGGGGACCTGGGGCTGGAGCAGCTGCTG | 956 |
| CTAGAAGACTGCAGGCTGGATCATGCTTTATATGCACTGCCTGGGCCAACCATCGTGGACCTGAGGAAAA | 957 |
| GAGAAATCGAATATTCTGGAGCACTGATTGCAGCAGGGTGGCTCCTTTGTGTGCAGCAGGTGTAGTAGTC | 958 |
| CACTGCTGTTGTCATTGCTCCGTTTGTGTTTGTACTAATCAGTAATAAAGGTTTAGAAGTTTGACCCTAA | 959 |
| CTCGGACAATTTCTGGGTGGTGACTGAGTACCCCTTTAGTGAGTACCCCTTTAGTGCTATATTTGTGCCA | 960 |
| CGCTTAAATCATGTGAAAGGGTTGCTGCTGTCAGCCTTGCCCACTGTGACTTCAAACCCAAGGAGGAACT | 961 |
| GTATGTTCACCAGGGGAATGGCTGGGATTTCTCGGCACTCTGCATCATCCATCTTTTCTTATAGGTGGGA | 962 |
| CCTCATTCCCTTTTTTCTTTACCCAGGATTGGTTTCTTCAATAAATAGATAAGATCGAATCCATTTAAAA | 963 |
| CAGTGGCCATCATCCTCCCGCCAGGAGCTTCTTCGTTCCTGCGCATATAGACTGTACGTTATGAAGAATA | 964 |
| AGCACAAGCAGTTGGAGCTTCCACCCCTACGACCAGTAGCCCAGCACCTGCAGTATCCACTTCAACATCA | 965 |
| GCCTATCACCTCCAGCACAATCCCAGCGAAAAAGGTGTGAAGCACCCACCATGTTCTTGAACAATCAGGT | 966 |
| GGGAACAGTGGTACTAACCCACGATTCTGAGCCCTGAGTATGCCTGGACATTGATGCTAACATGACATGC | 967 |
| AACAGAAGCCGCAGTCCCGTGGGGTCTGGAGACGCAGTTTCCTTGTTAATGACAATAAATCCCTGCTCCC | 968 |
| CTGCCACAGGGCCCTTCCTACCTTTGGATCTGTGAGAAGGTGAATACAAAGCAGCAGGCAGAGTAAAATC | 969 |
| TTCCCACATGCCGTGACTCTGGACTATATCAGTTTTTGGAAAGCAGGGTTCCTCTGCCTGCTAACAAGCC | 970 |
| CTTCCTCTTTCCCTCGGAGCGGGCGGCGGCGTTGGCGGCTTGTGCAGCAATGGCCAAGATCAAGGCTCGA | 971 |
| TCCTCCACTATAAGTCTAATGTTCTGACTCTCTCCTGGTGCTCAATAAATATCTAATCATAACAGCAAAA | 972 |
| GAATCGACGTCTCAAGAGGTTCTCCATGGTGGTACAGGATGGCATAGTGAAGGCCCTGAATGTGGAACCA | 973 |
| CCCCTGTCCCCACTCGCGTTCCGCATGGAGGATACTGAGGCCTTACCCCTAACCCCGATCCTCTACCCAA | 974 |
| ATCACTGTAAATGGTAATCAGTTGGAATTCTCCTAAATGTCTTCCAGACACTAGTAAAAAACGACCTGAA | 975 |
| GCAGGAAAACTAGCATGAAATATTGTTTCAGGCCCTGGGTTCTATGTGACACTACATTAGGAATTGGATT | 976 |
| GAAAATCGGGTTCACAGGCTCCACAGAGGTGGGCAAGCACATCATGAAAAGCTGTGCCATAAGTAACGTG | 977 |
| GAGTGATTCTGATATATGTACTTGTCACATTGGTGTTGGACACATTTGCGCCAAAAGTATGGTAATTCTA | 978 |
| GGCATGGCAGTACCCATGTTGATTTGACATCTCTCTAGCCCATCCATTGCTTACAGTAGAAGAGTGGGGC | 979 |
| GCCTCTCAGTCTTAGGGGACATGGCAGAGATGAAAGAAAGAAAGAGTGGGTTTCAGAAGTGTCAGGGTGG | 980 |
| GATGCGGGGCCTGGCGGTCTTCATCTCGGATATCCGCAACTGTAAAAGTAAAGAAGCAGAAATAAAAAGG | 981 |
| CCACCTGGTCATATACTCTGCAGCTGTTAGAATGTGCAAGCACTTGGGGACAGCATGAGCTTGCTGTTGT | 982 |
| ATTGAACATGGTCTTGTGGATGAGCAGCAGAAAGTTCGGACCATCAGTGCTTTGGCCATTGCTGCCTTGG | 983 |
| GAGGGCAGTAGGCCATCCCCCAGGAGAATGACAGAAGCAAAGGACTTGTTACTAAGCAGATTTAAGGGTC | 984 |
| CCCCCCTCTGAATTTTACTGATGAAGAAACTGAGGCCACAGAGCTAAAGTGACTTTTCCCAAGGTCGCCC | 985 |
| AATGTTGCTGATAGGGATAAATCTTGAGGCTGAGGGCGGGTGGTACAGATGTGTATGGGAAACCCCAACC | 986 |
| CGTGCTGCCTCTCTTCTGTGTCGTTTTGTTGCCAAGGCAGAATGAAAAGTCCTTAACCGTGGACTCTTCC | 987 |
| GGCCAGGCGCGCTCTGCCCAGCCCAGCCTACAGTGCGGATAAAGGTGCGGATGCTGCTGGCCCTGAAAAA | 988 |
| AGATCTGCTGCCTCGCCTCTAGATATGGTGCCCTGGTCTTCATGGATGAATGCCATGCCACTGGCTTCCT | 989 |
| CCCAAGTGAAGAGAACGTCATGAGTGTAAGTGCAAATCAGTGGAAGGAGCGGCAAACTGGGACATGCAGA | 990 |
| TGTGGAGGGCGAGCTGAGCCCTGGCCGCCGCCACAATGGGCCGCGAGTTTGGGAATCTGACGCGGATGCG | 991 |
| CCCCCTGAAGTCAGGACCAGTGCCTGTGATCTCCATTACTTTATTTTCCTGGAGGTATTAGCCAACACAG | 992 |
| TGGGAGGCGGGCGCAGGGTAGCTGTTGGCGCCGCCGCGTTTCTGGGCCTGGCCAACTCACGTGACCGACG | 993 |
| GGAAAGACCTGCCCCCGTGATTAAATTATTTCCCACCAGGCCCCTCCCACAACATGGAATAATGGGAGAT | 994 |
| GGTGTGGATTATTGGGCCAAAAGAGGAAGAGGTCGTGGTACTTTTCAACGTGGCAGAGGGCGCTTTAACT | 995 |
| CTGCCCTTGGTGCATTAGCAAGGGTCCTGAGAGAAGACTGGAAGCAAAGTGTCGAGTTAGCTACAAACAT | 996 |
| ATCGAGATGCCAAGAAGGGCTATGGAACTATGCAGGTGGCTAGTGGTCAGACTGAAGTCACCAGCTGAAT | 997 |
| CATCATACAAACCACATTACTTCTGTCACTTCAGGGCATCGGGACTGGCTGGCGCCCTTGTTATGTGCTA | 998 |
| TCACTCGCCCAGTCTTCAGTCTCCTGACTTAGAGATACAATCACGTCACAGGTCTCTTGGCCTCAATCTG | 999 |
| AGGGGCTGCTGTCCACAGCTTGGGGCTGAAGACTCCCAGGCCATTAACCCCTTAGCTTTTAGGAAGATTA | 1000 |
| CTCACGCTGATGGCTTGGCAGAGCACCTTCGGTTAACTTGCATCTCCAGATTGATTACTCAAGCAGACAG | 1001 |
| CGAGTGGTCTGTGTTCCTATTGCTGGTGGGGTGATAGGGTGGGCTAAAAACCATGCACTCTGGAATTTGT | 1002 |
| GAGGTGCTCAATAAGCAAAAGTGGTCGGTGGCTGCTGTATTGGACAGCACAGAAAAAGATTTCCATCACC | 1003 |
| ATTACTGTGGAGCAGCTTTCATTCCTACCCACTTGCAAACCTTGGCGCTGTTGTCTGAGATTGCTGCAGC | 1004 |
| TGGACAGTGCAATGAAGGAAGAAGTGCAGAGGCTGCAGTCCAGGGTGGACCTGCTGGAGGAGAAGCTGCA | 1005 |
| TCGCTCAAGCTTTCGAAGACACATGATGGCACACACTGGAGATGGCCCTCATAAATGCACAGTATGTGGG | 1006 |
| ACCCAATGTGGACTTCTTTTAAACCTTTCTAATGCCCATAACCCAGCCTCAGACCCATGGAGCCCACGAG | 1007 |
| AGGTCCTCTGAGGATCAGATCATGCATGCGCCATTTTTTACTTAATGCAGCTGTTAAATTGGCAAAGCTC | 1008 |
| CAGCCCATAAGAGACATTCTCAGATGAAACTCTGTTTTCTTGCCCCAGTCAGGCTCAAGCCCTGTGGTTG | 1009 |
| GCTCTGTATGTCCTCAGGGGACTGACAACATCCTCCAGATTCCAGCCATAAACCAATAACTAGGCTGGAC | 1010 |
| AATTCCAGTGGCAAAAATTCGAACAGAACAGGAAAGCAAAGGCCCTATGACCCGCCGACTGCTGCTGCAT | 1011 |
| CCAATACTTTAGAAGTTTGGTCGTGTCGTTTGTATGAAAATCTGAGGCTTTGGTTTAAATCTTTCCTTGT | 1012 |
| TTTTCTAGAGCAAAGCAAAGTAGCTTCGGGTCTTGATGCTTGAGTAGAGTGAAGAGGGGAGCACGTGCCC | 1013 |
| GCTCTAGGCCCTCACCTCAAACCTTGCCATTGGTTGCCGTATTTCAAGGTCAATATAGTTTCCCTCACTT | 1014 |
| GCTCCATTAAATAGCCGTAGACGGAACTTCGCCTTTCTCTCGGCCTTAGCGCCATTTTTTTGGAAACCTC | 1015 |
| TGACAACGAAGGCCGCGCCTGCCTTTCCCATCTGTCTATCTATCTGGCTGGCAGGGAAGGAAAGAACTTG | 1016 |
| GGGGAGCACATATTGGATGTATATGTTACCATATGTTAGGAAATAAAATTATTTTGCTGAAACTTGGAAA | 1017 |
| CTTTGGATCCATTTCATGCAGGATTGTGTTGTTTTAACTGTTGTTGAGGAAGCTAATAAATAATTAAATT | 1018 |
| GAACCCAATGGTAGTCTTAAAGAGTTTTGTGCCCTGGCTCTATGGCGGGGAAAGCCCTAGTCTATGGAGT | 1019 |
| CCTTCTCCAACATACATCCTGCATTACATGAATGGATTATTCCTAATAATTAATAAAAAGGTATTTTTTC | 1020 |
| GACAACACAAAACTAGAGCCAGGGGCCTCCGTGAACTCCCAGAGCATGCCTGATAGAAACTCATTTCTAC | 1021 |
| GCACAGAGTCAGGATCTCACATTTCACCCCAGGCTCAACTGAGGATGTGGCTTATTAAACACGGAAGTGC | 1022 |
| TCCCGTGCAACAGCAGAATCAAATTGGATATCCCCAACCTTATGGCCAGTGGGGCCAGTGGTATGGAAAT | 1023 |
| GCTCCCACGGAGGGGAGCAGGAATGCTGCACTGTTTACACCCTGACTGTGCTTAAAAACACTTTCACTAA | 1024 |
| TAAAAATACAAAAATTAGCCGGGCGTGGTGGCTTACGCCTGTAATCCCAGCACTTTGGGAGGCCAAGGTG | 1025 |
| TGGCTGTGCTTATTGCCCTCACCATTTATGACGAAGATGTGTTGGCTGTGGAACATGTGCTGACCACCGT | 1026 |
| GGAAAAGCATTGGCACGCAACGCAGCATGTGGCTTCATTGAGGCAGTTGATGGAGTTAAACCATCTGCTC | 1027 |
| CGTGCCTTCTTGCTGTCATGCAATGACCCCGCCTTATGTTGCCGAAATAAGCAACTCTTAGGTTTGCCTG | 1028 |
| GAGTTTTCCTCGGAAACACTCTTGAATGTCTGAGTGAGGGTCCTGCTTAGCTCTTTGGCCTGTGAGATGC | 1029 |
| GAGGCAGAATGGCTGTGCTGAGCCTCCTACCCATGACAACACCCCAATAAACAGAACATTCAGAGCCAAA | 1030 |
| GGGTTTTCCTGGGAGCGAATATCAAGTGCCTGAGAGCAACTACAGGACTAACTGTGTTTGGGTTGGGTGT | 1031 |
| GGAACCCCAGGTTCGCGGCCCGTGTTTCCGACCGGCGGAGGGGGCTCAGCGGCCCGATCCCACGGAAGCG | 1032 |
| TCTCCAGATGAGGTTGCAAGGACCAACCAGTGCCTACCCGCCCATGCTCCCCCGAAACTGGGAACTGACA | 1033 |
| CCCTGCTATTAGACCACCCCCTCATGGCACAACTGCCCCTCACAAGAATTCAGCTTCAGTGCAAAATTCA | 1034 |
| ACATTCTTCCTTTGCATTTGCTGGTCTGGCCTTTGCGTCCTTCTACCTGGCAGGGAAGTTACACTGCTTC | 1035 |
| ACAGGGGAAGATCCCGAGTGCAAGAAAGAGACAAAGAGCCCCTACAGGAACGCTTTTTCCGACCACATTT | 1036 |
| GGGCAGTCGCTGCAGGGAGCACCACGGCCAGAAGTAACTTATTTTGTACTAGTGTCCGCATAAGAAAAAG | 1037 |
| GCTGCAATGATGTTAGCTGTGGCCACTGTGGATTTTTCGCAAGAACATTAATAAACTAAAAACTTCATGT | 1038 |
| AATTCATGACCCACAAACTTAAACATACTGAGAATACTTTCAGCCGCCCTGGAGGGAGGGCCAGCGTGGA | 1039 |
| AGTTGGTCGGGATCCTGCTCAGCGCCCTGCTAGGGGTTGCCCTGGGACACCGCAGGCGGTGCTATGACTG | 1040 |
| GATAATATCTCTCACCCGGATCCCTCCTCACTTGCCCTGCCACTTTGCATGGTTTGATTTTGACCTGGTC | 1041 |
| TGGCCGCCATGAGGAAAGCTGCTGCCAAGAAAGACTGAGCCCCTCCCCTGCCCTCTCCCTGAAATAAAGA | 1042 |
| CAAGGCCAGTAGAAAGCTATGGCTGCAAAACCCTGGGGTGGACGATGTTTGATGATTAGACGGTCATCTC | 1043 |
| CCCAGGAGTTTGAGGCCAGCCTGGGCAACATGGTGAAACCCGGTGTCTACCAAAAATACAAAATGTATCC | 1044 |
| CTGTTTTTCTGTATGCTCTGTGCTAGTAGGGTGGATTCAGTAATAAATATGTGAAAGCTTTTGTTTCCAA | 1045 |
| TGAATTCTACAACCGGTTCAAGGGCCGCAATGACCTGATGGAGTACGCAAAGCAACACGGGATTCCCATC | 1046 |
| CGCTGTAAAACTCCGAAATGTGGCACAAACCCAACACGGAGCTACGCAATACTGCTGGAGAGCATTTGCT | 1047 |
| ACTTCACCGAAGACCAGACCGCAGATCTGATCCCAAGCACTGAGTTCAAGGAGGCCTTCCAGCTGTTTGA | 1048 |
| CGAGGCCTGGGGAGATGTTGTTTTCATGCTGCTTCCACCATCACACTGGGGTTTCTGGATGGGAAATAAA | 1049 |
| CACGCAGCCATGGTTGTGCCTGCCGTTCATGGTGGTCTTTCAGGTTATCTTGGCAACATGTACATTGCTT | 1050 |
| GTGTGGCTGCGGTTGGGTATGGATCAAGCAAGGGTTCAGATTACATCATTGTGAAGAATTCTTGGGGACC | 1051 |
| TGCCTTCTAAATGTGGTGTCGATCTCCCTTACAAGTTCAGCCCTTCCACTGACTGCGACAGTATCCAGTG | 1052 |
| ACGGGCTTATGATCCCTCGAGCACTATTTATCCGTGATTTGATGTGGCTCACTGGTTCGCTATGGGCAAC | 1053 |
| CGCCCTGAAGGAGTACATCGTCTAGTGAGGGACAGACCAAGCACGCAAAACAAATTGCAATATAATGTGA | 1054 |
| GCTCATTTGAGATAAAGTCAAATGCCAAACACTAGCTCTGTATTAATCCCCATCATTACTGGTAAAGCCT | 1055 |
| TCTTTCCTTCTGATCTGAGAAGACATGAACGTTTTCTCTTCACCGCCGTGGGGTGTATTGACTGGTCCCC | 1056 |
| CTTTCCCAGAAGATGGAGGAGAGTATATGTGTAAAGCAGTCAACAATAAAGGATCTGCAGCTAGTACCTG | 1057 |
| TCACATTTTCCCAAAAAAAGTTGATCTCTCCCAGTGGGCTGTAGGCAGGGTCCTCCATGGGTTTCCAACC | 1058 |
| AAAATTCCAGAGTGACCGTGGCACTTGGGTGTACAGGTAATTCCTCCAGAGCTGTTTGCTGGCTTCAGGA | 1059 |
| GTGGGGAAGAGCTATTGTAGGCTCCCCCTCCTCTGACTTATGTAATCAAAGCCACTTTTGTGTGTGTCTA | 1060 |
| TCATCTTGCTTGGGCTTACCAAATGCATTAGTCTTTGTGTTTGGGTCGACAGCGAGTGTGCCTGTGCTGG | 1061 |
| CTGTTCTTGTTTCAAAGCACCACTTGGAGGCTGCGGAAGATACCCGTGTAAAGGAACCACTGTCTTCAGC | 1062 |
| CCTCTGCAGTCCGTGGGCTGGCAGTTTGTTGATCTTTTAAGTTTCCTTCCCTACCCAGTCCCCATTTTCT | 1063 |
| GTGCCACTTCATGGTGCGAAGTGAACACTGTAGTCTTGTTGTTTTCCCAAAGAGAACTCCGTATGTTCTC | 1064 |
| AACCCTCCATAAACCTGGAGTGACTATATGGATGCCCCCCACCCTACCACACATTCGAAGAACCCGTATA | 1065 |
| GAGTACACCGACTACGGCGGACTAATCTTCAACTCCTACATACTTCCCCCATTATTCCTAGAACCAGGCG | 1066 |
| ACCCTTGGCCATAATATGATTTATCTCCACACTAGCAGAGACCAACCGAACCCCCTTCGACCTTGCCGAA | 1067 |
| TCGCCCACGGGCTTACATCCTCATTACTATTCTGCCTAGCAAACTCAAACTACGAACGCACTCACAGTCG | 1068 |
| CTTCCCACCTCAGCCTCCTGAATAGCTGGGACTACCAGCACGCTCCACCATGCCTTGCTAATTATTTTTT | 1069 |
| TTGCTACTTTGGCAAAAACTAGCGAGGGGTAGCAGAAACCTGCACCAAGGATTGTCCCTATGTCTTGGCC | 1070 |
| TATCAATAAAGTTGCTCACTTGTTGCCGGCCCGCTAGCCCGAAAGGTTGCGCGCGCAGACCGAGAAGTCT | 1071 |
| AAGATTGATGGAAGCCTCGGGCCTAAAGAATCACAGAGTTATGGAGAAGGTAGCTCGGAGAGCCTCCTGA | 1072 |
| ACTAACCCTATGTTGCACACGCTGGGTTCCTGATCTTGGTGCGATGTTTTGGTTACATGGCATCTGGCAG | 1073 |
| TAAAGATAAAGCCAGAAGCTAAGCTGCAGTGAGGCTGTGATTGGGCGTAGAAGTGGGAGCATTGGGACCT | 1074 |
| ACAACCAGAAGGCCCTTAACTATCACCAGTGCATCACATCTGCACACTCTCTTCTCCATTCCCTAGCAGG | 1075 |
| CAGTATCCAAAAATAGCCCTGCAAAAATTCAGAGTCCTTGCAAAATTGTCTAAAATGTCAGTGTTTGGGA | 1076 |
| GCACGGCATGGATTAACACGGCAGAGGAACAAAGGTGTGCTCTGAGCTTGTTCATATTTCACCTTCACCC | 1077 |
| AGAGTGTCATGGACCTGATAAAGCGAAACTCCGGATGGGTGTTTGAGAATCCCTCAATAGGCGTGCTGGA | 1078 |
| CAGAGTGTTGGGTCTGTAGCCAGCAAATTACTTCATCATCTAGATTATCCATTCAGTTGATCCTAATTAG | 1079 |
| TTGCTCACCCTCGGTAAAGAGAGAGAGGGCTGGGAGGAAAAGTAGTTCATCTAGGAAACTGTCCTGGGAA | 1080 |
| CCGCTCCCACCTCCCTGCTGGGAAACCACAGCATTATCACAGCATTATTGTGACAGCCACGAACCCATTG | 1081 |
| GGAGAGGTAGGTGACATAGTGCTTTGGAGCCCAGGGAGGGAAAGGTTCTGCTGAAGTTGAATTCAAGACT | 1082 |
| GCCGGGGCCCGAATCCAGGCACTGCTGGGCTGCCTGCTCAAGGTGCTGCTCTGGGTGGCCTCTGCCTTGC | 1083 |
| CAGGAAGCAGCGTCTCATCAGGACAGAAGGTAGGATGAAGACATGGGGTAATGTGAGAGAGTAGAACACC | 1084 |
| GAGGCGTCCAGCGAGCCGCCGCTGGATGCTAAGTCCGATGTCACCAACCAGCTTGTAGATTTTCAGTGGA | 1085 |
| AAACAGGAGCCTTACCCAGGAACTCTTTTTTATGCCAGAACGCTTCCTCTCCCCTGCTGTCTCTGGGGCT | 1086 |
| GAGCGCGGCTGCGCCGGCGCGTCGAGGGGAGAGGCAGCAGCCGCGATGGACGTGTTCCTCATGATCCGGC | 1087 |
| CGGAAAAAATTGTATTGAAAACACTTAGTATGCAGTTGATAAGAGGAATTTGGTATAATTATGGTGGGTG | 1088 |
| CACGGACCAGGTTCCCGCAAAACATTGCCAGCTAGTGAGGCATAATTTGCTCAAAGTATAGAAACAGCCC | 1089 |
| TCAGGTTCCAGGACCTTGGCTGGCTGGTAATTGCTGACTCTCCTTGTTTCTGTGCCGCACCACAGGCAGG | 1090 |
| GGTAGCGGCCGAGGTACACTCGGCTTGGCTGTTGGAGTTGCTTGTGGCATGTGCCTGGGCTGGAGCCTTC | 1091 |
| TCAAAGTATATGTAGAGATGACTATTTTATATTACATGACCCAATCCTGTATTTATTTCTACCCCCTTTT | 1092 |
| ATTAAAGTTCTTTTTATTGCAGTTTGGAAAGCATTTGTGAAACTTTCTGTTTGGCACAGAAACAGTCAAA | 1093 |
| TCATTAAGAACTTTTCAAAAGTGAATTAGTGAGGATTCAGCTTAATACCTGTATCAAATGAGGAAGTGGT | 1094 |
| CCCCGATCATCGTGCTTATCTAATACCTCACGACCTTCTCTCGGCGGGCCCTGGTTTCCTGCTGAACGAT | 1095 |
| ACATGATGAGTTGGCATTAGCTTCTCCAGGCATGGGAACTTAACAGATGAGGTTAAGAACCGTAGACAGT | 1096 |
| CATCAGAAGTGTTTCTTATTATTAYTTTATATTGAGTTGAATATTGAACTCTAACAGTTTTCTACATACA | 1097 |
| AGACATAATGTAGACATAGAGGAGGAACAGCTGAGAGTCTCTGCATCACAGAAAGAGAAACCTGAGCAAA | 1098 |
| AATGTCCTAGAAACAAATATAGAAAAATATATTCATGAGCTTAGGAGAATGTAGGCAAAGTTTTCCTGGC | 1099 |
| CGGCCCTGTGTGCCTCAGGGCAGATATAGCAAGCTCTTTCGACCATAGTTGATGGTAGGACATTTTAGAC | 1100 |
| GGACATTGTATTTGATGGCATCGCTCAGATCCGTGGTGAGATCTTCTTCTTCAAGGACCGGTTCATTTGG | 1101 |
| GGGATGAGGGATCATGCATGATCAGTTAAGTCACTCTGCCACTTTTTAAAATAATACGATTCACATTTGC | 1102 |
| AACATCATTCTCACCACCAGTCTCTTCTCTGTGCCTTTCTTCCTGACGTGGAGTGTGGTGAACTCAGTGC | 1103 |
| CCGCACCTGGCCTTCCCTGCTTCCTCTCTAGAATCCAATTAGGGATGTTTGTTACTACTCATATTGATTA | 1104 |
| AAGGAGATTGAGTACGAGGTGGTGAGAGACGCCTATGGCAACTGTGTCACGGTGTGTAACATGGAGAACT | 1105 |
| ATGTTCAAGTTCCACATTGGTCTTCAACTCTCTGGCGGGGTCAGAGGACCATCTGTGCTCGCTCAGATAT | 1106 |
| ACAGCGGCAGTCGGGCCCACACGTCCATGACTGGTCGTCCTAGATTTTAGGTGTCGATGAATACGGCCCA | 1107 |
| CCTTCTGTGACTCCCTGCAGCCACTGCTTCTTGAAGCCTTTGTCTCTAAGCTTCTGTCCAGCTCAAACCC | 1108 |
| CGGAAACGGGAAGGCCTGCTGCATTCCAGCCACATCTCGGAGGAGCTGACCACAACTACAGAGATGATGA | 1109 |
| CATGAAAACCATGAAGGGGCCTTTTGGCTGAAATTCCCCACCTGCCTTTGGATGAAAGACTCCGTTGGGA | 1110 |
| AGAGGAGCCCACGTCGCCTGTCACCCAATATCTCCAGCCGCGCAGTCCCGAAGAGTGTAAGATGTTCGCC | 1111 |
| GAGTATGAAGGAGAGAAGAGGGTACTGACCATGCGTTTCAACATACCAACTGGGACCAATTTACCCCCTG | 1112 |
| TAAATACATCCAAACATGATGATCGTTGGAGCCGGAGGTGGCAGGAGTCGAGGCGCTGATCCCTAAAATG | 1113 |
| GATTCCTCCTTTCCCCCCCAAATATTAACTCCAGAAACTAGGCCTGACTGGGGACACCCTGAGAGTAGTA | 1114 |
| TACTAAACATAAAAAAATTAGCCTGGCATGGTGGTGTACGCCTGTAATCCCAGTGACTTGGGAGGCTGAG | 1115 |
| GGTGGAGAGGAATTGCCGGAGCTCTGAAAATCCTAATGAAGTGTTCCGCTTCTTGGTGGAGGAAAGGATC | 1116 |
| AGCTGCTCTATAGCAATGTTTCTAACTTTGCCCGCCTGGCTTCCACCTTGGTTCACCTCGGTGAGTATCA | 1117 |
| TCCCAACAGATTGGGCTGGGTGGGGGTTGACAATGGGGTCAGATACTAAAGGGTCAGAATTTCTAAGCAG | 1118 |
| AGCCACATCTGCCTCTGAGCTGCCTGCGTCCTCTCGGTGAGCTGTGCAGTGCCGGCCCCAGATCCTCACA | 1119 |
| TGGCCTGCTTGGCAAGGCAAGTAGCGGCGGCGCTTCAAGATGCGCTGCCTGACCACGCCTATGCTGCTGC | 1120 |
| GAGAGCATTCCGCAAAGCTGCTTGTTTTCCAATTTCTTCATTCTTCCCCTTAGCACTGGTGCAGCTGAAT | 1121 |
| CTGGTGGCCATTAGTCACTCTTCATTTGGCTGGAACTACCGCACGGACCCTTTGAAGATATGTGTGGATG | 1122 |
| GCCCTGGGCCTTAAGAGCCAGCTCTTCCTATCCTGTAGCGTGTAGAAAACGTGGACTCATTTCACTATGT | 1123 |
| AGATTCATATGGGCTGGTGTTCCTGTGCGCTGTGGGTGTGGTGATTCAGCCTGGCATTTCTACCATAAGT | 1124 |
| CGGAAAAAATTGTATTGAAAACACTTAGTATGCAGTTGATAAGAGGAATTTGGTATAATTATGGTGGGTG | 1125 |
| CGAGCCCGGCCCCGCCAGCCCAGCCCAGCCCAGCCCTACTCCCTCCCCACGCCAGGGCAGCAGCCGTTGC | 1126 |
| GCTTGGGTAAGTACGCAACTTACTTTTCCACCAAAGAACTGTCACCACCTGCCTGCTTTTCTGTGATGTA | 1127 |
| TTCCCTGAGGAGGCGAATCCGGCGGGTATCAGAGCCATCAGAACCGCCACCATGACGGTGGGCAAGAGCA | 1128 |
| CGACAAGGAAGATTTGCATGATATGCTTGCTTCATTGGGGAAGAATCCAACTGATGAGTATCTAGATGCC | 1129 |
| TGATTATTACTGTGCAGCATGGGATGACAGCCTGAATGGTGTGGTATTCGGCGGAGGGACCAAGCTGACC | 1130 |
| CTACAGTTGGAAATCCATCCAGAGGCCATGTTCCAATAAACAGGAGGTCGTGTATTTGGTCACGACATTT | 1131 |
| CTACCGCCCAGTCACTCAAATCCGTGGACTACGAGGTGTTCGGAAGAGTGCAGGGTGTTTGCTTCAGAAT | 1132 |
| CTCTGAAAAAAATGATTTCAAGGCATGGAAGTYCTCTGTGATACAACAATACGTATTCTTCAAATGCGCC | 1133 |
| GCCCATCTCAGCAAGTTCCATGTCAGCCTTGGCAGAAGCCTCTTTCTTTCCTCTTCCCCATAAGAGACAT | 1134 |
| GGGGACAGCGCTTGCCTTGGTCAGACCTTCCCACATCTACATACTCTCAAATACATGACCAGGTGATCAA | 1135 |
| GCATCTGTAGGAGACAGAGTCACCATCACTTGCCGGGCAAGTCAGAGCATTAGCAGCTATTTAAATTGGT | 1136 |
| GGCAGCGAACTGAGTGAAGGGGAATTGGAAAAGCGCAGAAGAACCCTTTTGGAGCAACTGGATGATGATC | 1137 |
| ATGGCCTATTCACACAGATCCATCAGCGCACTGCCAGCAAGCTTCTCGGTCACTAGAATGAGATTAAAAA | 1138 |
| AAGCTCGCAACTGTGTAGGATGAATTCTGTACACTTTTATTTCCCTCTGTTCTCCTTTCCTATTTGAAAG | 1139 |
| AGGAGCGTCGGTAGTTCTTGCAGTAGGCACTTTATCAGGACCTGACCTGTTGCTGGGTGATTTTAGTCTC | 1140 |
| TCGCACGAGGATGCTTGGCACGTACCGCGTCTACATACTTCCCAGGCACCCAGCATGGAAATAAAGCACC | 1141 |
| TAGGAGATTTTCATTTTGTGTGACTCCCATGGGGAGGAACAGACTGGCAGGAAGCACACCGGGGTTAACA | 1142 |
| CACTCCAACCCAAACTAGCTGGGAGTTCAGAACCATGGTGGAATAAAGAAATGTGCATCTGCTCGTGCCG | 1143 |

APPENDIX B

| | SEQ. |
|---|---|
| | ID. |
| SEQUENCE | NO.: |
| AGGTACCAATAAAACGTAGGCTTTTGACTTTAGGAAAATACAAGA | 1144 |
| AATTTTAATGCAACAGACAGGAGAGAGGTCCAGCATAGATATCTA | |
| ACGTGTTAGTTTTATTTAAAGATGGTCTCACGGTGCYAGAGTAAG | |
| AAATGTTATTAAGAAAACGAGAGAGAGAGGGAGAGAGATCAAATA | |
| AATAAATAAATAAATAAATAAAAATAGGAATAACTTTCTGTTGAC | |
| GAGCTTTCATCTTGGGAGGAACGGGTTCTGTGAAGCATTCTTCAG | |
| AGTGAAGTGGTCCTAATTCTTCCTGGAACCATTGCAACCCATTCC | |
| ACTCAGGGAGCCAATCCTATCAATTCTTCTGCCGAAGCAGCCAGA | |
| ATCTCTCATCATCCGGGGCATCTGCACCCCCCTCAGTCTCTTGAG | |
| GAAGGGGTTCCTGTAGGACAGAGGAGTGTTGGATGCTAGCTTGGG | |
| TTCAGCCTTCTGCTCATCGCTGTCATCTATGAGTTCTGGTGGAAT | |
| CTCCTCTCGGGTTTGGGGCTCTTGTAGGTCAGGATTGGACTCCAG | |
| GGCTTCAATCAGTGCGAATTTGTCCTCCAGTCTCTCCAGAAGAGC | |
| CTCCATGCTGGCCAGTTCTTTGGCAGGGCTGAGGTTGTAGATGGG | |
| GTTGGCCCTGCTGGGCTGCAGCTGGACAAGAAGCAGCAAGAGGAA | |
| ACCACAGGAAAATGAGCCTCTAGTGTCCATGGCGCTGGGTTCGTT | |
| GGGAATATGGGAAGTTCAAGCTGTTTCTTCTGAGATGGCTCTTCA | |
| GGTCTCTCTCTTSGCKGGGACCASGCTAATCAS | |
| CTGATTAGCCTGGTCCCCGCCCAGGTACTCCAAGGGACAGAAGAG | 1145 |
| GAGCTACAGATAAAGAGAAACTGCCAGCTAAAGCTGTATATGACT | |
| TTAAAGCTCA AACTTCTAAA GAGCTGTCCT TTAAGAAAGG | |
| AGATACTGTC TATATCCTCA GGAAAATTGA TCAAAACTGG | |
| TATGAAGGTG AGCACCATGG AAGAGTTGGA ATATTCCCAA | |
| TTTCTTATGT TGAGAAACTT TCTCCTCCAG AAAAAGCACA | |
| GYCTGCCAGG CCGCCTCCCC CTGCACAGAT TGGAGAGATT | |
| GGAGAAGCTA TTGCCAAATA TAATTTCAGT GCAGACACAA | |
| ATGTGGAGTT ATCACTTAGA AAGGGAGACA GAGTCGT | |
| ACAGTAAAGT CTTTATTAAA GTTATTGTTG GGTGATCACA | 1146 |
| TAACTTTCTT TTTAAAAAAA AAAAACAACT GCTGTCTGAA | |
| TTGGAAACCA GATCATACCT TTTTGTATTG GGATTTTGGA | |
| CCATATATCT TATGTTTCTG TACCTC | |
| ACATGCTGTT TTTCTACTGC TGCAATCTAA CATGTGAGTT | 1147 |
| ACAGATCCTT AAGATCTTTC TGGATGCTCC ACAATGTGTC | |
| TGCACTCTTC TTCAGCTGAG CCACTTCATC ATCCTTCAAC | |
| TTTTGGTTGA TGACACTTGT CAATCCAGAG GCACTCAGGA | |
| CACAAGGCAG GCTCAGGAAG ACATCGTTCT CAATGCCATA | |
| CATGCCCTTT ACCAGTGTTG ACACAGAATG AACTCTGCAC | |
| AAGTTCTTCA GCATCGTCTC ACAGAGCTCA GCAACGCTAA | |
| GGCCAATGGC CCAGTTTGTA TAACCCTTGA GTCTGATTAC | |
| CTCATAGGCA CTTTCAACAA CCTGCTTGTG GACTTCCTTC | |
| CAGTTCTCAC TGTCTTTGTC AGTTCCCATG GCAGGATTCA | |
| GCTCCTGGAG AGAAACACCT GCCACATTAA CTCCGCTCCA | |
| AACAGCCACA CTAGAATCAC CATGTTCTCC TAAAATCCAG | |
| CCATCGCAGC TGGTTGGGTG GATATCAAGT CTCTCAGCCA | |
| TCAGGTAGCG GAATCTAGCT GTGTCTAGAT TGCAGCCACT | |
| TCCAATCACA CGGTGCTTTG GCAGGCC | |
| AGGTACCATG CTGAGGAAAT TCAGTGCAAT GGAAGGAGTT | 1148 |
| TTCACAAGAC GTGCTTCCTC TGCATGGCTT GCAGGAAGGC | |
| TCTGGACAGC ACCACAGTGG CAGCTCACGA ATCTGAAATC | |
| TACTGCAAAA CTTGCTACGG GAGAAAATAC GGCCCCAAAG | |
| GTGTTGGCTT TGGACAAGGG GCCGGATGTC TCAGCACCGA | |
| CACTGGGGAC CATCTGGGCC TAAACCTGCA ACAGGGATCA | |
| CCAAAGTCTG CTCGCCCTTC TACACCAACT AATCCTTCAA | |
| AGTTTGCCAA AAAGATCGTT GATGTGGATA AATGTCCCCG | |
| GTGTGGCAAA TCGGTGTATG CTGCAGAGAA GATAATGGGA | |
| GGAGGAAAAC CTTGGCATAA AACATGCTTC CGCTGTGCTA | |
| TCTGTGGAAA GAGTTTAGAG TCTACAAATG TTACAGACAA | |
| AGATGGAGAG CTCTACTGTA AAGTTTGCTA CGCAAAGAAT | |
| TTTGGTCCCA AAGGAATTGG TTTTGGTGGC CTCACTCAAG | |
| TGGAAAAGAA AGAGTGTGAG TGAAAAGGAG TGGATGCAAC | |
| AGAGTCAACC TGCTGCTGAT GTCAGCAGAT AATAGTTGTC | |
| AAGTAAAACC AAATCACCAC CTACTGCTCA TAACCTAGGG | |
| CATTCATTAA ATATTTTCCA TCTTGCAGGA AGCCTTCTGA | |
| AGCCTTCTGA AGAAAAAGCA AGTTTTCTTA GAATATAGTG | |
| TTTCAGTTTT GTTATTGT | |
| ACCTAAATGG CTTTGTTAAT TATGGGGATG GCCAGGTGAT | 1149 |
| GTTATTTTTT TTAAAGCCGT TCAGGCAGTC AGTGTGTCAG | |
| AAGCAGCACC CAAACAGTGA GCGAACAAAG TGCTGGCCGC | |
| TTGCACTTTG TTCAAAAATT GTCAGTCAGG TATGGAAATT | |
| ACGACATAAT CAGATCCAAT GTAAAACATT AAAGTAATAC | |
| TTACAGGAGG GTAATTGTAA ATATCACCAG TGCCTTACAT | |
| TTCTGATTCA ACATAGTTAT TTGTGCATGT ATGAAATACT | |
| ATGCACAGTA TCCTCTCTTG GTGGTAGGTA ATCTTCAACA | |
| GGGAGCCTCT CTACCTGTTG AGGCATTCTT AAACATCAAC | |
| AATGAGTTGA GGCACAAAAA TTAGTTAAAT GTTGAGCAGG | |
| ATAGTCGTTT GCCAGGAAAC TTTCTCCTAC CAACTGTTAA | |
| TTCCAAAAGT TACATTTCAA AATGTCATAA AACAAATGGT | |
| ACCTCGGCC | |
| CAGGTACAAA ACCTGTTCAA CACTTTGACC TTTGGTCACA | 1150 |
| TACTCATTGC CAACTTTCAC TCTAGGGTGT AATAAACCCT | |
| TAACTAAATC AGAGGAGTTG ATTCCCATTA GGTAGGCAGC | |
| TTTGTCAGCA CTTTCAGTGC CATCAGCCTC TGCCTGCTCT | |
| TCTCTGGGTC GCTGTTTGAA TTTCATGTTC CCAAAGTGCA | |
| TAATGGCACC TGTGAGCTTG TAGGCACCAG ACTTCTCATC | |
| TTGCACAAAT CCCAAAATGT CCATGGCTTG ATCTGTTGCC | |
| ATCAATTCTT CTCCGTCATC CAAGTTGTCC ACTGTAACTA | |
| CTCCTTGGGA GCAAAAGTGG TAGTCATATG GGTTGGTGGA | |
| GACCAATAGC ATATCCAGTA ACTCTGGCTT CTTTCCTGAT | |
| AAGATCTGGT AGAAGATGTG ATAGTCTCTC TCACCCGGTT | |
| GCTGAAAAAT CACTCGGGAT TTCTCCAGTA AATAGATCTC | |
| AATATCAGCA GATGACAGCT TGCCCGTGGT TCCAAAATGG | |
| ATTCGAATAA ATTTACCAAA ACGTGAGGAG TTGTCATTTC | |
| TCAGGGKTTT GGCGTTTCCA AAAGCTTCTA GGGCTGGGTT | |
| TGCTTGAATG ATTTGATCTT CCAAGGTTCC CCCAG | |
| GGGTAGGTAC CTCGTTGAGT GGATAGGAGT ATATAAAGGG | 1151 |
| TGTAGGAAGC AGTTAAGAGC GTTGCAGTTC CGGTTAAGAT | |
| AATTGTGGGA GGGGATCAGT TAAATAAGGC AATTATAATT | |
| GTTAATTCTG CCATTAGATT GGTTGTTGGA GGGAGGGCTA | |
| TGTTGGTTAG GTTAGCTAGG AGTCATCATG TGGCTATTAG | |
| GGGTAGGAGG GGTTGTAGGC CTCGTGTAAG AATGAGAATG | |
| CGGCTATGCG TTCGTTCGTA GTTCGTGTTA GCTAAGCAGA | |
| ATAGGAGTGA TGATGTAAGT CCGTGGGAGA TTATTAGGAT | |
| TATTGCCCCT GAAAATGATC ATTGAGTTTG GATCATGCTT | |
| GCGGCGATTA CGAGTCCTAT GTGGCTTACG GATGAGTAGG | |
| CAATGAGAGA TTTTAAGTCT GTTTGGCGTA AGCAGATGGA | |
| GCTGGTCATT AGGGCCCCTC ATAGGGCTAG GGTGAGGAAA | |
| GGGAAGTGTA GATAGTTGCA TACGGGCTGT ATGAGTAGTG | |
| TTACTCGTAT AATACCGTAG CCGCCTAGTT TTAGTAGTAG | |
| GGCAGCAAGT AATATTGAGC CTGCGGTTGG TGCTTCGACA | |
| TGGGCTTTGG G | |
| GGGCAGGTAC CAGAGCAAAA TTAAGATGCA GACAGAAGCA | 1152 |
| CAGCACGAAA GAGAACTCCA GAAGCTGGAG CAGAGGGTGT | |
| CACTGCGCAG GGCACATCTG GAACAAAAGA TTGAAGAGGA | |
| GCTCGCCGCC CTCCAGAAGG AACGCAGCGA GAGGATCAAG | |
| TTTCTGTTGG AAAGGCAGGA GCGAGAGATT GAAACGTTTG | |
| ACATGGAGAG CTTACGAATG GGCTTTGGGA ATTTGGTCAC | |
| ATTAGATTAT CCCAAGGAGG ACTATAGATG AGACGAAATT | |
| TCTTTGCCAT TTAACAAAAA CCAGACAAAA TCAAACAAAA | |
| TAGTTACAAA ACTTGCAAAA CCAACATTCC CCATGTTAAC | |
| GGGCGTGCTC TCTCTCTTTC TGTCTCTCTT ACTGACATCG | |
| TGTCGGACTA GTGCCTGTAT ATTCTTACTC CATCAGGGGT | |
| CCCCTTTCCC CCTGTGTCAA GTCCCGGTGC AGGACAGCTC | |
| CTGGCGGTCT TTTCCATAGT ATGTCACAGT ATTGATGTCT | |
| CTGTGCAATG ATTAAAAATG TTTCAGTGAA AAACTTTGGA | |
| GACGATTTTA ATGGAGAAAA AAGA | |
| ACCGCCGCCT GCGATAGGGA CGGCGCTGCT GGGCTTGGCC | 1153 |
| TTCCGGGATG TTCTCTGCTC CTTCGTTCTT CTCCCCACTC | |
| TCACTGTTCT GGTAGTTTTG CTGGTAGTTG CGTGGAGGAC | |
| CCCTGCGACG CGGATATCGT CTGTAATGGT TACGGTCTGC | |
| TGCGTATTTG CTGCCTTGCA CTGGAACGCC ACCAGGCCCT | |
| GTCACGTTCG CTGCCTCCGC ACCCTTCTCT CCTTCAACCA | |
| CATCAAACTC CACGGTCTCT CCGTCTCCTA CGCTGCGGAG | |
| GTACCTCAAA AACCCACTCA TTTCAGTCTT AGATATTCAC | |
| ACATCTCGGT AAACAAAACT AAAACTACAT TATTTTTTAA | |
| TGGCCAGCAT GCTGTATTCT CTGAGGGCTC TAGGCTTGTG | |
| CAGTAGATAC ACATACCACA AATGTAATGC TTCCTCACCG | |
| CTGTTAATTG AAAATCTTCT AAGTTATTTG CTATTGAGGC | |
| TGTGGAAATT GTTACCACCT GACAAGACTC CAACAGTGGT | |
| TAAATGACTA GTGTCGGTAG TGTAGGAAGC TGTATAAAGA | |
| TACCTTCTGA GCTTTCCTTA AATTGTCACG T | |
| ACTGTATTTT CTGCTTCTCT GCCTTTTGAA GCCAGGGACT | 1154 |
| GTCGGGATTT CTTTATTCTG TGGGATACTT TACTTCTCAG | |
| TCTGAAAAGC TACTTCCTTC TACAAAGGCA AGACCAAAGA | |
| CTTTATGCTG GTCCAATTTG TAGAGCATAG AGGCCCCCCC | |
| CGACTATTTA AGTTTGACAA TCTTAATGAA TTTGTCATCT | |
| TTAGAGGGAA GCAAAAGCAT AAACCATACC AAAGCAAAGG | |
| AAATGCTATA TTTTTAAATA AGAAATAATA ATAATCACAG | |
| GTCATTAGGA TATCGTCAGT TCCATGGTTC TTTAGT | |
| ACAGCTGGAT GAGGAGCTGG GAGGCAGCCC TGTGCAGAAA | 1155 |
| CGAGTAGTGC AAGGAAAAGA GCCACCTCAT CTGATGAGCA | |
| TGTTTGGTGG AAAGCCTTTG ATTGTTTACA AGGGTGGAAC | |
| ATCTCGGGAA GGAGGTCAGA CCACACCGGC ACAAACACGG | |
| CTCTTCCAGG TCCGGTCCAG CACCTCGGGA GCTACCAGAG | |
| CTGTAGAGCT GGATCCTGCT GCCAGTCAGC TGAACTCCAA | |
| CGATGCTTTT GTCCTGAAAA CTCCCTCTGC TGCTTACCTC | |
| TGGGTTGGCC GTGGCTCCAA CAGTGCAGAG CTGTCAGGAG | |
| CACAAGRGCT GCTGAAGGTT CTGGGAGCTC GTCCAGTACA | |
| AATAGGTCCT ATATACGTTA AGATTCTTTG GAAACTGCTA | |
| AGTATAAAGG AGTTTGTAAT CCAGACTACT ATCTTTTTGG | |
| CTACTCCAAA AAATCTGCGG GAGTTTCCAG CTTATCATCA | |
| GTTGAAATCT GTTTTGCAAT TGCCAAATAA ATGCAAGTAA | |
| AAT | |
| TGAAGGTTCT TTTACAGCTT CTGGGGTGCT CGGGAAAGAC | 1156 |
| AGATCTTCAG CACTGCCTTC ACTGTCTCTT TCATAATCCT | |
| TTAGCACTGT CAGCTTATCT ACACCACATT TCTCATCTAA | |
| ACCAGTTTCC AGTTTGCCAT CTATTTTACT TCCAACATCC | |
| TCTCTCAAGC TGCTCAGTCC ATGACCAGCA CCTTTTACTT | |
| CCCATATTGG CTCATACGGT TTGAAGTCCA CATACTCTTC | |
| CCTTCGAATA GCATCTTCTT TCAATGCCTT TTTGTCATCA | |
| TGATCACCAT CTCTGCCTTT GTCTTTGCTA GAAAAGGTAT | |
| CTTCCTCATT TTTCTCAAAC AAATCTTTAG GAGTTTCTGG | |
| AGACATACTC AGAGGCTGTG CAGGCATTTT CTCCAGATCA | |
| CATAATTCTT TAGGGCCCTC TAGCTGCATT TCTTCTTCAG | |
| CTTGAAATAA AATGGCTGCT TCTGCTTTAG TAAGTGAGGG | |
| TTCCATTTCT GAGTATTTCA TCTCGGAAGC GTCTCTCTTA | |
| TTTCCCTCTG TAAGGAATGG ATTTCTTACA TTTTCCATTG | |
| TTTCTTTACA AACCTCACTT GCACTTTCTT TGTAAATGCC | |
| TCTTGCAGGG AATCCATCTG AGAGTGAATC AAAGGCTGTG | |
| TG | |
| ACTCTGTCTG GATTGGAGGC TCTATCCTGG CCTCTCTGTC | 1157 |
| CACCTTCCAG CAGATGTGGA TCAGCAAGCA GGAGTATGAT | |
| GAATCCGGAC CCTCCATTGT CCACCGCAAA TGCTTCTAAA | |
| CCGGACTGTT ACCAACACCC ACACCCTTGT GATGAAACAA | |
| AACCCATAAA TGCGCATAAA ACAAGACGAG ATTGGCATGG | |
| CTTTATTTGT TTTTTCTTTT GGCGCTTGAC TCAGGATTAA | |
| AAAACTGGAA TGGTGAAGGT GTCAGCAGCA GTCTTAAAAT | |
| GAAACATGTT GGAGCGAACG CCCCCAAAGT TCTACAATGC | |
| ATCTGAGGAC TTTGATTGT | |
| ACTCTGTCTG GATTGGAGGC TCTATCCTGG CCTCTCTGTC | 1158 |
| CACCTTCCAG CAGATGTGGA TCAGCAAGCA GGAGTATGAT | |
| GAATCCGGAC CCTCCATTGT CCACCGCAAA TGCTTCTAAA | |
| CCGGACTGTT ACCAACACCC ACACCCTTGT GATGAAACAA | |
| AACCCATAAA TGCGCATAAA ACAAGACGAG ATTGGCATGG | |
| CTTTATTTGT TTTTTCTTTT GGCGCTTGAC TCAGGATTAA | |
| AAAACTGGAA TGGTGAAGGT GTCAGCAGCA GTCTTAAAAT | |
| GAAACATGTT GGAGCGAACG CCCCCAAAGT TCTACAATGC | |
| ATCTGAGGAC TTTGATTGT | |
| ACTCTGTCTG GATTGGAGGC TCTATCCTGG CCTCTCTGTC | 1159 |
| CACCTTCCAG CAGATGTGGA TCAGCAAGCA GGAGTATGAT | |
| GAATCCGGAC CCTCCATTGT CCACCGCAAA TGCTTCTAAA | |
| CCGGACTGTT ACCAACACCC ACACCCTTGT GATGAAACAA | |
| AACCCATAAA TGCGCATAAA ACAAGACGAG ATTGGCATGG | |
| CTTTATTTGT TTTTTCTTTT GGCGCTTGAC TCAGGATTAA | |
| AAAACTGGAA TGGTGAAGGT GTCAGCAGCA GTCTTAAAAT | |
| GAAACATGTT GGAGCGAACG CCCCCAAAGT TCTACAATGC | |
| ATCTGAGGAC TTTGATTGT | |
| ACGCATGTGT TATCTACCTC AAGGTAACAG CAGTATGTGG | 1160 |
| CAAAACATTA ACCACCCATA GTGCTTCTCA TTATGCACTT | |
| CTATTTAGCC AGCATTATTG TAGTAGCTAT TCTTATTGAA | |
| AACCATTCAA TATTTATAAA TGTTCTGGTA TGCATTCTTT | |
| ATAGTGAAGT GTTAATATGC AGCACTTTTA TTTATTTTAG | |
| CAAATAAATA AGTATATTTC TGTAATTATA GAAAGTCAAC | |
| TTAATTTTTG AGTTACGTTT CAGATAAAAG TTTTTGTTTA | |
| GCACTATGGT TTTATTGCCT ACATAGCTGG ATATATATTA | |
| ACATCGGCTT ATTCTGAGGC TATCCAATAC ATTTTTTTTC | |
| TAGTTTTCAT TTCAAGTAAA GCACTCACTG TGTATAGGAA | |
| TTTGTAATTG GAGGTGCTTG ATCTCTACAA AAGAAATTAG | |
| GAATCGCTTT ATTATAAAAT GCTCCTAGAA GTCTTAATTG | |
| TGTTCATTTC TAAAAAATTT TGTAATGTTA GTTGTGTGCA | |
| TGGAAATAAT TAAGGT | |
| ACCATTTCGC TCAGCAAGGT CCCCGACTCC GCGCATCCAA | 1161 |
| TGCCATGATG AATAACAATG ACCTAGTGAG GAAGAGAAGA | |
| CTTGCGGAGC TGAACGGGCC TATTTTTCCC AAGTGCAGGA | |
| CTGGAGTGTA GCTCCCAGGC AGAGGTCGTT CCCAGCGGGT | |
| CTGTGCTACT GTGACAACCT AAGGCAAAGA AGTGCCTTGA | |
| GAGAGTTATT TGTGGTGCCT CGGTTCTGTT TCATGCACTA | |
| ACAGTTTAAA GTAACTAGTG GCTGTAGTTG AAGATTTTTA | |
| TCCAGTAGCA CTGTTGTTTT CTGTAGAGCT GGAAGCTATC | |
| CAAGCCAGTA ACCTGCCAGT GTTGTGCAGC CTCAGCTGAG | |
| CGT | |
| ACCTGCTACT TTAAAACAAA TTTTAACTGC AGCTACTTTT | 1162 |
| CACTAAGCAA GATGGATAAA GCATGCCATT TATATTTTGC | |
| CTTCTCAAGA GATTATTTTC AGAAACATAT ATTATTCCAC | |
| CGCAATCTGA CACTTCCTGT CATGCTTTCA TCTTGTAAAA | |
| CCTGAATTCC AATTTTAGGC TATTCCAGGC TTATGCTTAA | |
| ATGACAGTGC CTTGGTAAGA GAAAAAATAA TTGTGCTGCC | |
| TTTTTCTCCC ATAGTGCCTG AAAACATATT GGGCATACAT | |
| ATATTATATA TATTCTTACA AATGTCCAGG TCATGTATAC | |
| CAGCTGAAAT TCTTTTAATG TGGGGGTGTT TGCATTGTGA | |
| GATTTAATCA AGACATTAAC ATGAGTAGAA GGTTGTTGTT | |
| TTNAGACAGA AGTTTGAGAA TCANCTCA 428 | |
| ACACCTCTAC CCCGACAAGC ATTACATTTC TGAACAGCTC | 1163 |
| CAGCCTTCCC TCCTTGACCA TTACATGCAC TACAAAGGAC | |
| ATTCTTGCTA AGTTGTAGTT TAGTTGTCTT TCCATTATAT | |
| AAATCTTCTA AAGAGACTTT GAGAGGATGC ATCATATCTT | |
| CTCCTCTTCT TCTACCATTA CGACTTCTAC TCTGACCACC | |
| CATGAAGTTG AACAATCCAC CACCAAAGAT GTGGGAGAAA | |
| ATATCGTCCA TTCCACTGCT TCCACCACTG CCTTCTCGAA | |
| GGCCCTGTTC TCCATATCTA TCATATAACT CACGTTTCTC | |
| TGGATTTGAC AATACTTCAT AGGCAAAGCT TATTTCTTTA | |
| AATTTGTCAC CTGCATTTGG ATTCTTATCA GGATGGTATT | |
| CCTTGGCCAG TTTTCTATAA GCCTTCTTGA GCTCGTTGTC | |
| GGAGGCTCCG GGCGGCACGC CCAGGATATC GT | |
| ACAGATCATC CAGCTTGCGG AAGGCGCTTT CAGTCCAAAT | 1164 |
| GCAGAAACGC CCAACGTGGC CACCAGGAGC AAGTCTCAGC | |
| AGGTTCAGCT TGTTCACATC AAGAAGAGTA ATCCCCGGGA | |
| TATTCCGGAA AGCTCTAATG ATGCCGTTGT CCTCGTTGTA | |
| GATGATGCAA GGTCCCCTGC GCTGGATGCG ACGGCGATTC | |
| CTCATCTTAC CCTTCCCGGC CCTCATGCGC TGAGAGGCAT | |
| AAACCTTTTT TATGTCATTC CAAGCTTTAA GCTTCTTAAG | |
| AAGGAGAACA GCTTCCTTTG TTTTCTTGTA ACTCTCAACT | |
| TTGTCCTCAA CAACCAGAGG AAGTTCTGGG ATCTCCTCAA | |
| TGCGGTGACC TTTAGACATG ACCAGTGCTG GAAGAGCTGA | |
| TGCTGCCAAG GCAGAACAGA TGGCGTAACG TTTCTGAGTT | |
| ACGTTCACTC TGCGGTGCCA GCGTCGCCAA GTCTTGGTTG | |
| GGGCAAACAT GCGGCCTCCA CGGCACATAT TTCCAAAGGC | |
| ACCCTGGCCA GAGCGGTGAG TTCCACCACC TCGT | |
| ACAGATCATC CAGCTTGCGG AAGGCGCTTT CAGTCCAAAT | 1165 |
| GCAGAAACGC CCAACGTGGC CACCAGGAGC AAGTCTCAGC | |
| AGGTTCAGCT TGTTCACATC AAGAAGAGTA ATCCCCGGGA | |
| TATTCCGGAA AGCTCTAATG ATGCCGTTGT CCTCGTTGTA | |
| GATGATGCAA GGTCCCCTGC GCTGGATGCG ACGGCGATTC | |
| CTCATCTTAC CCTTCCCGGC CCTCATGCGC TGAGAGGCAT | |
| AAACCTTTTT TATGTCATTC CAAGCTTTAA GCTTCTTAAG | |
| AAGGAGAACA GCTTCCTTTG TTTTCTTGTA ACTCTCAACT | |
| TTGTCCTCAA CAACCAGAGG AAGTTCTGGG ATCTCCTCAA | |
| TGCGGTGACC TTTAGACATG ACCAGTGCTG GAAGAGCTGA | |
| TGCTGCCAAG GCAGAACAGA TGGCGTAACG TTTCTGAGTT | |
| ACGTTCACTC TGCGGTGCCA GCGTCGCCAA GTCTTGGTTG | |
| GGGCAAACAT GCGGCCTCCA CGGCACATAT TTCCAAAGGC | |
| ACCCTGGCCA GAGCGGTGAG TTCCACCACC TCGT | |
| CAGGCTCACG CTCTGCTGAT CCAGAAGCTC TTGGCTTAGG | 1166 |
| CTCCTGATTT AGCACTGGCA AGTTTTGTTT GCATTTCTGT | |
| CACAATTAAA AAGTGTTCCT GAACCGCAAT CGCCAAAGCA | |
| GGGGTGAATT ACAGGATATA GCACGACAAA TGCATTTTTC | |
| TGAGAGCAAC ACAACCTATG CATGTGCTGA CTAGATACAG | |
| CTTCCTAGAA AAAGAATAGC TTTTTCAAAA TAAGAGATAC | |
| GATTCTTCAC TTCTGATAGA GTAACTTCTT CTT | |
| CTGCTTGTTA TGTTGGTGTC TGCGATACGG ATATAGAAAG | 1167 |
| CCTCTTCATC CCTTGGAAWG CYTCCNTTTK CAATGCCCTG | |
| AGCTCTTGAG TGGATCGTTG CCYAGTTCT | |
| ACCACCTGAG AGGACRTTRT TRGCATAMAG ATCCTTWCKR | 1168 |
| ATRTCWATRT CRCACTTCAT GATGCTGTTG TARGTRGTTT | |
| CATGAATRCC AGCAGATTCC ATRCCRATRA ARGATGGCTG | |
| GAAKARRGTY TCTGGGCAGC GGAAGCGTTC ATTTCCGATG | |
| GTGATGACCT GGCCATCAGG AAGCTCATAG CTTTTTTCCA | |
| GAGAGGAAGA AGAGGCAGCA GTGGCCATCT CATTTTCAAA | |
| GTCCAGGGCC ACATAACACA GTTTCTCCTT GATATCACGG | |
| ACAATTCCAC GCTCCGCAGT GGTAACAAAG GAGTAGCCAC | |
| GTTCTGTCAG GATCTTCATG AGGTAGTCTG TCAGGTCACG | |
| GCCAGCCAGG TCCAGACGCA TGATGGCGTG TGGCAAAGCA | |
| TAGCCTTCAT AAATGGGCAC GTTGTGGGTG ACACCATCCC | |
| CAGAGTCAAG CACAATCCCT GTGGT | |
| ACCACCTGAG AGGACRTTRT TRGCATAMAG ATCCTTWCKR | 1169 |
| ATRTCWATRT CRCACTTCAT GATGCTGTTG TARGTRGTTT | |
| CATGAATRCC AGCAGATTCC ATRCCRATRA ARGATGGCTG | |
| GAAKARRGTY TCTGGGCAGC GGAAGCGTTC ATTTCCGATG | |
| GTGATGACCT GGCCATCAGG AAGCTCATAG CTTTTTTCCA | |
| GAGAGGAAGA AGAGGCAGCA GTGGCCATCT CATTTTCAAA | |
| GTCCAGGGCC ACATAACACA GTTTCTCCTT GATATCACGG | |
| ACAATTCCAC GCTCCGCAGT GGTAACAAAG GAGTAGCCAC | |
| GTTCTGTCAG GATCTTCATG AGGTAGTCTG TCAGGTCACG | |
| GCCAGCCAGG TCCAGACGCA TGATGGCGTG TGGCAAAGCA | |
| TAGCCTTCAT AAATGGGCAC GTTGTGGGTG ACACCATCCC | |
| CAGAGTCAAG CACAATCCCT GTGGT | |
| ACCACCTGAG AGGACRTTRT TRGCATAMAG ATCCTTWCKR | 1170 |
| ATRTCWATRT CRCACTTCAT GATGCTGTTG TARGTRGTTT | |
| CATGAATRCC AGCAGATTCC ATRCCRATRA ARGATGGCTG | |
| GAAKARRGTY TCTGGGCAGC GGAAGCGTTC ATTTCCGATG | |
| GTGATGACCT GGCCATCAGG AAGCTCATAG CTTTTTTCCA | |
| GAGAGGAAGA AGAGGCAGCA GTGGCCATCT CATTTTCAAA | |
| GTCCAGGGCC ACATAACACA GTTTCTCCTT GATATCACGG | |
| ACAATTCCAC GCTCCGCAGT GGTAACAAAG GAGTAGCCAC | |
| GTTCTGTCAG GATCTTCATG AGGTAGTCTG TCAGGTCACG | |
| GCCAGCCAGG TCCAGACGCA TGATGGCGTG TGGCAAAGCA | |
| TAGCCTTCAT AAATGGGCAC GTTGTGGGTG ACACCATCCC | |
| CAGAGTCAAG CACAATCCCT GTGGT | |
| CGCACAAACT GTGTAGTGTC AGACCTGATT ATGGGCAATG | 1171 |
| AGTATTTCTT TCGAGTCTTC AGTGAAAATT TGTGTGGATT | |
| GAGTGAAACT GCTGCAACTA CCAAAAATCC TGCCTATATC | |
| CAAAAAACAG GCACCACTTA CAAGCCACCT AGTTACAAAG | |
| AACACGACTT CTCTGAACCT CCTAAATTCA CTCACCCTTT | |
| AGTAAACCGG TCTGTGATTG CAGGATACAA CGCTACACTC | |
| AGCTGTGCAG TGAGAGGAAT CCCTAAGCCA AAGAT | |
| ACTTCTGGGA ATCAAAAGTG AAATGGAACT AATCCTAGTT | 1172 |
| TAATTGCAAT TGCTATTGTT AGTAGCAGAC ATGATGTGGG | |
| GTTGTTTAGT TGTGTAATGT CTCACTGTCC TGTAGATCAG | |
| GCATTGGTAA GGCTTGAGAA TAGGATTAAG GCTGATGCAG | |
| TTGATTGGGT GAGGAAGTAT TTAATGGCAG CTTCAATTGC | |
| TCGAGGGTGG TGGGATTTTG AGATGAGTGG GATAATAGCT | |
| AAGGTGTTGA CTTCTAAGCC TGTCCAGGCC AAGATTCAAT | |
| GGTTGCTGGA CATAGTAATG CTGGTTCCTA TAATTAAGCT | |
| TATGGTGGAG ATTAGTTTTG CG | |
| AGGTACTCCT TATGATTTTG GGACATGCTC ATTGCACAAG | 1173 |
| CTCTCCAGTT TCTATTAAAG CTGAGGATAG TTGCCATCTC | |
| TTCTTTAGTC AGCTCAAAGT CAAACACCTT GAAGTTCTCC | |
| ACAATGCGCT GTGGTGTGAC AGACTTGGGG ATCACAATCA | |
| CATTTCTCTG GATGTGGAAC CGAATGAGAA CCTGTGCTGC | |
| TGTTTTGTTG TGCTTGGCTG CAATCTCTTT AATTTTAGGG | |
| TCATCCAGAA GTGAGGGATC CTCTGGCTTA GCCCATGGTC | |
| TGTCAGGAGA GCCAAGGGGG CTATATGCTG TCACAGAGAT | |
| CCCTTTGGAT TGACAGTAGT TGATCAGCTT CTCCTGGGTC | |
| AGGTATGGAT GGCATTCAAC CTGGTTGTTT GCAGGTTTGT | |
| ATTTCAGTCC TGGCTTGTTC AAGATTCTTT CTATCTGCTC | |
| ATGGTTGAAG TTGGAAATCC CAACAGCTTT TGCCAGACCA | |
| GCATCCACCA GTTCTTCCAT GGCCTCCCAT GTTTGTAGAA | |
| GATCCGTGTT GCCAGGTATT GACATGCCTT TGTCATCTGC | |
| AGGAAATAGG TCCTCTCCTG CCTTAAATCC AACAGGCCAG | |
| TGGATGAGGT AGAGATCCAG ATAATCCAGT TTCGGGCTGG | |
| CAAGAGTCTT CTGGCAGGCT CCTTTCACCA ATGATTTTTC | |
| AT | |
| AGGTCGGAAT AATTCTTCAG CTTCAGGGTT GGGCTCTTTC | 1174 |
| AGCCATTTTG CCCTGTTATA GTCCACAAAT CTAATCAACA | |
| AAGGGTGGAG GGGATAGAAG TCCCGTGCCA AAGTGGTGAA | |
| CTCATCTAGG ATGAGCAGTT CAGCCTCCGA CATGTCACCT | |
| CTGCCTTCTG CTCTTAGATG CTCTGCATCC GATACCANCC | |
| ACTGCTGCTT TTTTCTT | |
| ACAGATTTTG GTTTCACAGA AATTTAACTG CAGAAACAGG | 1175 |
| AAAAGCACTC CAAGATATCA GTTGTAGGTC ATGTAGCGGG | |
| GAGTAGCACT GAATTTTGCA TAGGATTTAA TGACTTTGTT | |
| CACTGCTTCT AAGAAGTCTT TCTCAGTTGC AATTTTTCGG | |
| CGTGCTCGGA TTGCAAACAT GCCTGCTTCT GTGCAGACAC | |
| TGCGAATCTC AGCTCCTGTG CTATTAGGAC ACAGTCGAGC | |
| CAACAGCTCA AATCTTATGT CCCTTTCCAC ACTCATGGAG | |
| CGAGCGTGTA TCTTGAATAT GTGAGTCCGC CCCTCAAGAT | |
| CAGGCAAGCT AAACTCTATC TTCCTATCCA ACCTCCCAGG | |
| CCTCATCAGA GCTGGATCCA GAGTATCAGG CCTGTTTGTA | |
| GCCATCAACA CTTTGATATT GCCTCGTGGG TCAAAACCAT | |
| CCAACTGGTT GATCAGCTCC AGCATCGTGC GCTGCACTTC | |
| ATTGTCACCC CCAGCACCAT CATCAAAGCG AGCACCTCCA | |
| ATGGCATCAA TTTCATCAAA GAATATAAGA CAAGCTTTTT | |
| TAGTTCTGGC CATTTCAAAG AGTTCGCGAA CCATTCGAGC | |
| TCCCTCTCCC ACATACTTCT GCACCAGCTC AGATCCAATG | |
| ACTCTGATGA AGCAGG | |
| CGCCGGCGGT GCGGCTGCAG ACATGGCGAT CCGCTACCCT | 1176 |
| ATGGCCGTCG GCCTCAACAA GGGCTACAAG GTGACGAAGA | |
| ACGTATCCAA GCCCAGGCAC TGCCGCCGCC GAGGGCGCCT | |
| GACCAAACAC ACCAAGTTTG TGCGAGACAT GATCAGGGAG | |
| GTCTGTGGCT TCGCGCCCTA CGAACGACGT GCTATGGAAC | |
| TGCTGAAAGT TTCCAAAGAT AAACGTGCTC TGAAGTTCAT | |
| CAAGAAACGG GTTGGCACTC ACATTCGGGC CAAGCGAAAG | |
| CGGGAAGAAC TCAGCAATGT CCTGGCAGCC ATGAGGAAAG | |
| CTGCTGCAAA GAAGGATTGA GTTGT | |
| CCATGAAAAC CTGCACCTAT TGACACCAAG GGGAGAAAGA | 1177 |
| AAAACACRGG GCACTTCAGA ATGGATTCAG GGAATTTCCA | |
| CTGACCTTTT AAGAAATGGC TTGTGGCCAC CTTGATCCTG | |
| AGAGATTGTG GTTTTAATTT GAAAGAATTC ATAGATTGAA | |
| CACTTGTAAA AATTAATAAG CACCTACGAC AACGAAGAGT | |
| ACACACGAGG ATTTAAAGGG TAGGGATTTT TTTTTACGGG | |
| TCTGACTTAT CTTCCCGGGG NAAAATGGTT TTATAAAACT | |
| TNCANAGAAC TTTTTTAAGA GCCGT | |
| AGGTACCACA GCCAGGAGCT GATTCACATT TCGGATTTGC | 1178 |
| AATCTGAGGT GCCTCCCATT CGCCATCCAT ATCCTCATCC | |
| CAATCTTCAG GCTTCTCAGC ATCAGGGTCT GCTACGTATT | |
| CTGGCTCATC ATCCAGCCAG CCCTCTGGTT TCACAGCATT | |
| TTCATCTGCT ATCTTTGCAG GAGCATCTTC ATCCCAGTCA | |
| TCTGGTTTAA CAGCATCTGG ATCTGGGATT TTGGGTCTCT | |
| CATCCCAATC CTCAGGCTTC TGGTCATTTG GGTCCTCAAT | |
| CTCTCGAGGT GGATTCACAG GAGGAGACAT ATCATTTAGC | |
| AAATTCCCAC TGTTGACAAC CATTTGATCA ACCAGAATTT | |
| CAAAACTATT ATCAGGATTC AAAACCAGAG TATAAAGGTG | |
| TGTCTTCTTA TCAGAGAAGT AGGTCTNCAA GTCTGCATCT | |
| GGACGCTTCR CGTGCTTCTC CTCATAT | |
| GTCCTGCTGA AGGCTGGGAT TCCTCTTGGG ACTTTGTTGA | 1179 |
| CTTGTGGATA GCAGGCGTGC TCTGCTGATT CATTTTCATC | |
| ACTGTCAAAA TGGTCATCAA AGTCTTTGTA GCCACATCTT | |
| CGGGGTCTAC AGCGATCAAA AAGAAACAGC AAGAGGTAGT | |
| GGGCTTTTTA GAGGCCAACA AGATTGACTT TCAGCAAATG | |
| GACATAGCAG GTGATGAGGA CAACAGGAAA TGGATGAGAG | |
| AGAATGTTCC TGGAGAAAAA AAGCCTCAAA ACGGAATTCC | |
| TCTTCCTCCA CAGATCTTCA ATGAGGAGCG GT | |
| ACTTAGCAGC AATCCCAAAG CGCGTGTTGT TACTGCCAGC | 1180 |
| AGTCCATGCA AGATTCACTG ATGTCTCAAC CTTATTATTA | |
| ACCTTCTGAT AAATAGACCC ACCAAACTCT GTGCCATCAT | |
| TCACGTTTGT GTGCAGCTGA AAGTCTCCTG CCTTATATCC | |
| CAAGGCAAAG TTGTTTTGGG AAAGCTTAGA TTTGGCTGTA | |
| TCAAAAGCCA TCTGATAGCC AGCAAGCCAG CCTTCATAAC | |
| CCAGCACTGC CCAGCCATAG ATGGTTGGTC CAGAGAGATC | |
| AATGTCTATA TTGCAGCCTA GGTTTACGTA TTCTCTTTTG | |
| TAGGAAGTCT TCAATTTTCC ACTCTTCTTC CCTGTATTTG | |
| GT | |
| ACCAAGTGGA ATAAAATACC TTTATCTTCG AAACAATATG | 1181 |
| ATTGAGGCCA TTGAAGAGAA CGCATTTGAC AATGTAACAG | |
| ATCTGCAGTG GCTGATCCTA GATCACAATC ATCTGGAAAA | |
| TTCAAAAATT AAGGGAAGAG TCTTCTCTAA ACTAAAGAAT | |
| CTGAAGAAAC TTCACATTAA CTACAACAAT TTGACTGAAG | |
| CTGTTGGACC GCTCCCCAAA ACTCTGGATG ACCTGCAATT | |
| AAGTCACAAC AAGATCACAA AAGTCAATCC TGGTGCACTT | |
| GAGGGGCTGG TAAATCTGAC TGTCATTCAT CTCCAGAACA | |
| ACCAGCTGAA AGCAGATTCT ATTTCTGGGG CATTTAAAGG | |
| TCTGAATTCA CTTTTGTATC TAGACTTAAG CTTCAATCAA | |
| CTTACAAAGC TACCAACAGG GCTGCCTCAC TCCTTACTCA | |
| TGCTGTATTT TGACAATAAC CAGATCTCCA ATATTCCTGA | |
| TGAGT | |
| CTGATTCCAG CSGCCCCCCN GGCAGGTACT TTCTGATCTG | 1182 |
| ATGGTTATTA CATCACCSTT GATGCTGATA GTTAAATTAG | |
| GTTTGGSCAC ACCAGACCAT CTTCCTGGGA GGAAACCCCA | |
| CTCCCAGCTC TTTCATATAG TCCTCAAAGT TTTCACTTGA | |
| AAGGAGCTTC CAGGTGCCCA CAAACTGGGT CGCACATTTT | |
| GTCA | |
| ACCTTGGCTT TAGTTTTATT AGCATGAAAC ACCTTTGGCA | 1183 |
| TGCTTAGCTT CCAGGTAACT GGTAACTCCC TACCTGTATC | |
| AGAATTCATT TTACGTAGTT CCACAGAACA TGAAGATCCT | |
| TATTTGCTAA GCCTTTGAAA GCTGACATTC TTTTTCATAA | |
| GAGGGTGTAT TTAAATGGAT GTTCACCAAT AGACAATAGG | |
| CCAGCTTACT AGTGGTGAGC TAAAACTGCT AAATGAATGT | |
| CCAACATGAT TGTAAGGCTT ATGTCACTCA AGATTTTATC | |
| CTTTGGATTT TCATGATCAA ATTTCATATA CTATTGTATA | |
| GACTTCTGCT TTGTAGTGTA CCTGCA | |
| ACAGGTTGAA GATTTTTGCA GCATTAGTGC TCGTCACTGC | 1184 |
| CACAAACTGG TTTTCATCCA TCTTTCCTGT AGCCACTGCT | |
| TTGTCCCAGA TGACAGACAT CCGTTCCTCG ATGCCATTGG | |
| TCCCCTCTGG GATCGCTGTG AAGTTGTCTT TTCCAATTGC | |
| TTTCTGCGCA GTGCTGAACG TGCAGTGGGC ACTTCCTGAC | |
| ACCTGCAGGC CACCACTGGC CAGGAGGGAG TTAATGTAGT | |
| CAGGGGTTGT GGGATCTGGG CTTAGGGGGG GCGAGACCAC | |
| AAATGCTGCA GCCTTGGCCC AGTTCTTGCT CCAGTAGTGT | |
| GTTCCATCAG T | |
| AGGTACTTAT TGCACAGCTA ATTGGCTATT AACATCACTG | 1185 |
| CCATGCTARC ATCCATCCAG CAGGAGGAAG CAGCTGTTGA | |
| AGGCACTGAA GGAACTAAAC TTGCTCTATT AAAAAGAGGA | |
| AAAACCTGTT ACTTAGACAT CCACATCTGC CATTGCTCTC | |
| TGCAGAGGAT CAACAGACCA GTAGTCGTCA TCCCAGCCCA | |
| GGAACCAGCC AGAGAAGGTT GGAGGCTCAA GCCCTTGCTT | |
| AACGAGTGTG ACTGGAGTTC TCTTATCACG GCTGGCTGGA | |
| TCAGTTTCAA TGTATCGYTT AGCAGATTTC AAAGCCTCGG | |
| TCTTTTCTTC CTCCTGGGCA TCTTTTCCAA TCCATACAAA | |
| CACCTGATCC CATGTGTCAA GGATCATAAC ATCATCTGTA | |
| GCAAGGTCAT CCTGGGTCAG GTCTCCAGGG ACTTCTTCAA | |
| TAGTGAAGCG TCCACTCTTG TTGGAGCATG CAAAAAGACG | |
| AGGGGGGTGA GCATCCATCT TCTTGTCCTT CAGCCGANGA | |
| GAAGT | |
| ACTTGTTACA ATACAGCATG GAGAAGTTAC CAAGCGATTG | 1186 |
| GACACAACAA CCTTTTCTAC TTTCTTCTCA AGGATATCTT | |
| TCATTATTTT GCAAAGGTTT TCAAACTTGG CTTTTTTCTC | |
| TTCCTGTTTC TTTTTCTCCT CTTCATCTTC TGGAAGCTCT | |
| AAGCCCTCTT TTGTTATAGA AACCAGAGTC TTGCCTTCAA | |
| ATTCCTTCAG CTGTTGCACA CAGT | |
| ACTTGTTACA ATACAGCATG GAGAAGTTAC CAAGCGATTG | 1187 |
| GACACAACAA CCTTTTCTAC TTTCTTCTCA AGGATATCTT | |
| TCATTATTTT GCAAAGGTTT TCAAACTTGG CTTTTTTCTC | |
| TTCCTGTTTC TTTTTCTCCT CTTCATCTTC TGGAAGCTCT | |
| AAGCCCTCTT TTGTTATAGA AACCAGAGTC TTGCCTTCAA | |
| ATTCCTTCAG CTGTTGCACA CAGT | |
| ACAGACATAG GTGTAACTGC AGTTCACTAA CAGCAGCTTA | 1188 |
| ACTCCTTGGT GTTGACAGTG GACATTGTGC TGGGGGCACT | |
| CGAATCCCAG TGCTGAAATT AACACTAGTG GAATCTGTCC | |
| TTCATCTTTG CACTGTGGTA TATCTATGCC ATGTTATTAA | |
| TCCCGTTCTG TGCAATCAGC AGTGTGCTAA CCTGCTTTTT | |
| TTCTTCTGTA AGCATTTCGC ATTATTGGGC TTCATTACCT | |
| GCCTTGCTTT GTATACCAAG GCTGGTTCTC TTGCACATCT | |
| TACGCTTTTA TACCTTTAAC TTTTTGAATG GTCAGATACT | |
| GAACTGGACA GTCAAACAAC TTGTGTTCTT TAGGGAGTCG | |
| TAGCTACTGT TGTATTTTAA CACTACAGCT GAGGGCTTCT | |
| TTGAGGGCGG GTTTCTTCTT GGAGAGT | |
| ACTGGTTCTG AGAGAGCTTT TAAGGTCCCA GAGAAGCAAG | 1189 |
| CTGCTCCAAC CTAAGTCATT ACAACAAACT ACTATGTCAT | |
| ATACTTGTTT GTAAAACCCA GTAGAGTTTT TTGTTGTTGT | |
| TGTGTTATTT TTAAATATTT GTTTCTTGGT TTAAGCAAAA | |
| TGACAAGCGG TTATGGTGAT TAGATATAGA GTGGGGCAAA | |
| TTAAGTGAGT TGATTTAGTT GTGTGTATAA ATAAGTAGTG | |
| TGTGAAAGTG CTCAACTGCC TAATGGAATT TAGGACTTTT | |
| CTAAATGTTT ATGCAGACTT AGCTATTGCA TAACTATTGG | |
| CCTGATAACC AGAGCGGCTG AGGATGTGGA ACAAACTACA | |
| TATCAGAGTT CACTGGGATG AATATATGGT ATCTTTGGAT | |
| GGAAGAAGTT CGGTAAGGAT TAGTTATTTC AGCTCCACAT | |
| AAATTACTTT GAAGGAGTTA GGCTGTCAGA AAGTGCCAAA | |
| TACTCACTTT TGGGCTCCAG T | |
| ACATCAGGAG AATGAGATGC TTATTTGTCA CTTCCACATA | 1190 |
| AAGCCACCAG GATGGTTTCA TAATCCCCTT TGAGTTCATC | |
| CATAATTGCT TGGCGAAGAG ATATACCATA CAGACTCTTA | |
| TAATAGGCTT TGATCTCATT CAAGTCTACT TCATGGCGTG | |
| AAACCATGAT TCTGATAAGC TGTTTGTGTC GTGTCCCACT | |
| TCCCTTCATG GCCAAGTGGA GTTTTTCAGC AAAGAAAGCT | |
| GGCTTGCTTG TGGCACACTT CACAAGGGCA GTCAAGCAGT | |
| TTTCAATATC ACCTTTCAGC TCCAAATCAA GT | |
| ACAAGTTCTT CAAGGGAAAG AACCGCCATG CTTCCTGCAG | 1191 |
| TGCTTCCAAG GAGGGATGAT TGTGCATGCT GGAAGAAGGG | |
| AAGAGGAAGA AGAAAACGCA CAAAGTGACT GGAGACTATA | |
| TTGTGTGCGA GGAGAAGTTC CCAATGAAGG AAACTTACTT | |
| GAAGTAGCAT GTCACTGCAG CAGCCTGCGT TCCCGGACAT | |
| CCATGATTGT CCTCAATATA AATAAAGCTC TTATCTATCT | |
| GTGGCATGGC TGCAAAGCAC AATCTCACAC CAAGGATGTA | |
| GGAAGAACAG CAGCCAATAA AATAAAAGAA CAATGTCCGC | |
| TGGAAGCAGG GCTGCACAGC AGCAGCAAGG TGACAATACA | |
| TGAATGTGAT GAAGGGTCAG AGCCTTTGGG ATTCTGGGAT | |
| GCATTAGGAA GGCGAGATCG AAAGGCCTAT GATTGCATGT | |
| TGCAAGATCC AGGAAAGTTT AATTTCACCC CCCGCCTGTT | |
| CAGCCTCAGT AGTTCTTCAG GAGAATTCTC AGCCACTGAG | |
| TTCGTTTACC CTTCAAGAGA CCCTGCTGTC ATCAATTCTA | |
| TGCCCTTCTT GCAAGAGGAT CTTTACACTG CC | |
| ACCATCGAAA GTTGATAGGG CAGACATTCG AATGGGTCGT | 1192 |
| CGCCGCCACG GGGGCGTGCG ATCGGCTCGA GGTTATCTAG | |
| AGTCACCAAA GCCGCCGGGC GAGCCCGGGT TGGTTTTGGT | |
| CTGATAAATG CACGCGTCCC CGGAGGTCGG CGCTCGTCGG | |
| CATGTATTAG CTCTAGAATT ACCACAGTTA TCCAAGGAAC | |
| GGGAGGGGAG CGACCAAAGG AACCATAACT GATTTAATGA | |
| GCCATTCGCA GTTTCACTGT | |
| GACTCTGTCC GCTGTGGGTT CGGTGCCGCC ATGGCCAAGT | 1193 |
| CCAAGAACCA CACCACGCAC AATCAGTCCC GTAAGTGGCA | |
| CAGAAATGGC ATCAAGAAGC CCAGATCCCA TAGATATGAG | |
| TCCCTCAAGG GGGTTGATCC CAAGTTTCTG AGAAACATGA | |
| GATTTGCCAA GAAACACAAC AAGAAGGGGC TGAAGAAGAT | |
| GCAGGCCAAC AATGCCAAGC AGGCAGCTCT ACAGAAAAAG | |
| GACTGACCTG GTTTAAGACA AAGAACCAGT TTGCCTTTTG | |
| GCATGTGTGT TTAAAGCATT TTTGT | |
| CAGGTACTGA AAAACTTCTA GGCTTCCAGC TTCACCGACT | 1194 |
| CTAGAATGGA ACGTGCTTCT TTATACTCTT CAGCTTCTTC | |
| CAACTCTTTT TCATTTTCTA GTAGTTGAGT GAGATCTGCA | |
| TGTGCAATTT CTAATCTCCG CTGACAGTCT GGAATCATCA | |
| TTCGAGACTC TTGTAAGATC TCAACCTGCT TTTTTATTCC | |
| ATAGTCATCA CATGCTTCAG CTTTCATTTT TTCAATCCTC | |
| TCTTCTTGTT GTTTTGCTTC WTTTTCRTAC ATAACTTTTT | |
| CTTTTGCCAA TCGCTTCACG ACGCCGGTTT TGATCTTGAT | |
| CTGCCTCAGA CGGGGATCGG MCATGGCGGG GCHGCAGCGC | |
| G | |
| CCGGGCAGGT ACGTTCTTGA AGGGTTAATG GTATGTGATT | 1195 |
| TATACTGTGC CTTAATTGTT ATGCTATTTA AAAACAAATA | |
| TTTATTTTGA AAGTTTTACT ATGCTGTGCT CTAAAGAAAG | |
| CAACTTTAGA TGTGACACTG TATAATTATG TATTCATCTC | |
| ATGGCATAAA TTATTTAGTA GACTTAGATG TMGCATATTA | |
| AATATKAACC TAATTAACTA AGGATGTTGA CTTGGATTTA | |
| TTTAAATTCW GTATGTGCAC TGTATGAGGG T | |
| CTGCAGCTCC AGCAGCGCCC GGTCCATCTT GTTCATCATC | 1196 |
| AGGACAGGTT TGATCCTCTC AGCAATGGCC TGACGCAGCA | |
| CGGTTTCTGT CTGCACACAC ACACCAGAGA CGCAGTCGAC | |
| AACAACCAGG GCACCGTCAG TGACCCGCAG AGCAGCAGTG | |
| ACCTCTGAAG AGAAATCCAC GTGCCCAGGA GAGTCGATCA | |
| GGTTGATCAA GAAACCAGAA CCATCTTTGC TCTGCTTGAT | |
| GAACGCCAGA TCGTTTTCAG AGAGCTCGTA | |
| AGGTACAGGC TAGCATCTTG CAGAGGAAGA GCTTACTTCC | 1197 |
| TCTGGTCTAG TTTCCTTACA CTTAAAATGA AAGGCAATAC | |
| AGAATCTTAT TCTACTTCTG CCTTGAGAAA AACAAAATAA | |
| TTTACTTTCC TTATATAGCT TAGTGCTCTG AAAACTTAGT | |
| TCTTAAGTTA AACCAGAATT ATTTTACACG AACCTTTTCA | |
| TCAGATGCAA TCTTACCACT TGTCAGACTC TTCCCCAGTA | |
| TACATTACAA AGCTGCTTAG TAAGAAAAGT TGTGTGAAAG | |
| CAGCTTCTAA TTAATGGATC ACATGAGATC CTGCATCATC | |
| CCCAGTAGCA GCAGTCTGCT AGCAACCRCA GAAATACATT | |
| AGCAAAGGTT ACACCGAAGC AGTCATGTCT GACAGCTAAT | |
| ACAGCACTAT AACATACAGA CCTTTCRNAN GCAGGTCAGT | |
| ATGTAGAAAT AATTCTTTAN CATGTAAACA GGAAAACTGA | |
| TCTGTCAGTT ACRTAGATCA ACAGCTGAAG CTATT | |
| AGGTACCGCC TGCAGAGGGA GAAGGAATTC AAAGCCAAGG | 1198 |
| AAGCAGCGGC GCTTGGATCT CATGGCAGCT GTGTACAAGC | |
| TTTTTTKTTT KTTTTTTTTT TTTTTTTKTT GGNTTTTTTT | |
| TTTTTTTTTT TTTCCACAAA AAAAAAACTT TCTTATGKKT | |
| CTTTCTGTTG ACGAGCTTTC ATCTTGGAGG AACGGGTTCT | 1199 |
| GTGAAGCATT CTTCAGAGTG AAGTGGTCCT AATTCTTCCT | |
| GGAACCATTG CAACCCATTC CACTCAGGGA GCCAATCCTA | |
| TCAATTCTTC TGCCGAAGCA GCCAGAATCT CTCATCATCC | |
| GGGGCATCTG CACCCCCCTC AGTCTCTTGA GGAAGGGGTT | |
| CCTGTAGGAC AGAGGAGTGT TGGATGCTAG CTTGGGTTCA | |
| GCCTTCTGCT CATCGCTGTC ATCTATGAGT TCTGGTGGAA | |
| TCTCCTCTCG GGTTTGGGGC TCTTGTAGGT CAGGATTGGA | |
| CTCCAGGGCT TCAATCAGTG CGAATTTGTC CTCCAGTCTC | |
| TCCAGAAGAG CCTCCATGCT GGCCAGTTCT TTGGCAGGGC | |
| TGAGGTTGTA GATGGGGTTG GCCCTGCTGG GCTGCAGCTG | |
| GACAAGAAGC AGCAAGAGGA AACCACAGGA AAATGAGCCT | |
| CTAGTGTCCA TGGCGCTGGG TTCGTTGGGA ATATGGGAAG | |
| TTCAAGCTGT TTCTTCTGAG ATGGCTCTTC AGGTCTCTCT | |
| CTTACTTGGA CGAAGGCCGG TTCTTCGAAA GTGTGGTATG | |
| GGGGTGGACA TCCGTGGATT CATTCAATGT TGGTAGAGGT | |
| TAGTWCAGGR YGTMGTCCAC TCTAACAAAC CTATTGACCA | |
| TAACTCTATC CTACATAATC CCAATCCTAA TCGCCGTGGC | |
| CTTCTTAACA CTTGTAGAAC GAAAAATCCT CAGCTACATA | |
| CAGGCCCGAA AGGGCCCAAA CATTGTGGGC CCTTTTGGTC | |
| TACTTCAACC CATTGCAGAC GGAGTAAAAC TCTTTATCAA | |
| AGAGCCCATC CGCCCATCTA CCTCCTCCCC TTTCCTCTTC | |
| ATCATAACAC CCATCCTAGC CCTACTTTTA GCCCTCACAA | |
| TTTGAACACC CCTCCCACTC CCTTTCCCCC TTGCAGACTT | |
| AAATCTAGGA CTACTATTTT TATTAGCAAT ATCAAGCCTA | |
| ACTGTCTACT CCTTACTTTG ATCTGGGTGA GCCTCTAACT | |
| CCAAATATGC TCTAATTGGG GCCCTCCGAG CCGTTGCCCA | |
| AACAATCTCA TATGAAGTCA CCTTAGCCAT CATCTTACTA | |
| GCCACAATTA TACTGAGCGG GAATTACACR CTAAGTACAA | |
| CACAAAAAGC AAGCAAGCTG GAGGGCCTGT TGATGTAGGT | |
| CCCGAGTTTC AGAAAGACAT GAATGAATCA CTTGCCAGGC | |
| TTCAGCGGAT GT | |
| ACTACATTCA CAAAGTCTTC CGATACGTCC TTCATTACAT | 1200 |
| CTGCATGCTC CACATTCAAA TGTCCCATTT CCGTCGTGGC | |
| AGGCGGGGCT GTTTGGTTCT CCTTCGCTTT GACACAGACA | |
| ATCACGGATG AACTGGAGAT TGATTTCCAC TTCTTCAGTG | |
| AACCCCAGTG GTTTAATTTT AATGGTTTCA TTTTGTCCTT | |
| TCTTTGGACA TTCATTTGCT GTTACATTAA TCTCAAACCT | |
| AACCTCATCT CCTATTGAAA TGTTGGAACA TTTTCTTCCG | |
| TCTTCCTGCG TGTCGTTGAC TCCATTCTTG CAGTATGATT | |
| TGTAACTGAT TGTCACTCCT TTTGGTAGCT TACTGTTTTC | |
| CAGGATCACC TCTGAAGAAA GGGAATTGTA TGCATCAATG | |
| ATCAACTGAA TGACATTGCT GGAATTGGAA GACAACGTTC | |
| CTACTGCTGA TTTTGGTATG AGGTTTTTCA GTTCCTTATA | |
| AACTGCCTGA AACTCTTCAG TAACAGCAAA AATTGTCTGA | |
| ATATTGTTCT CACTAAGCTT CTGT | |
| ACTGTATAAA AACTTGTGTT GAGTTGGAGG TATAAAAGCC | 1201 |
| CAGTTGTCTG TATCAATAAT CAATGATGTT TTTGGGAATT | |
| TTAGAATAGC TGCTGAGAAA TTCACCCACT TACTGATAAG | |
| AGGCAACAGC TGCTGCTCAT CGCTTTGATC ACAGATTTTG | |
| TAAGGCTTTT TTTTTCCAGC AACTGTTTGG GCCTACAGCT | |
| TCTCTATCAA TATTGCAGAA GCACCTCCTC CTCCATTGCA | |
| AATTCCTGCA AGACCATACT GCCCTTGTTT CAGTGCATGG | |
| ACCATGTGAA CGACAATTCT CGCTCCAGAC ATTCCTATCG | |
| GATGCCCGAG AGAGACAGCG CCGCCATTGA TATTTACTTT | |
| TTGTGGATCG ATACCCAGCA TTTTAATATT GGCCAGCACC | |
| ACAACACTGA AGGCTTCATT GATTTCCCAC ATTGCGATGT | |
| CTTCTTTTTT CAGACCTGTC TCACTTAGAA TCTTGGGAAC | |
| AGCGTGTGCA GGTGCAATGG GAAAGTCAAT AGGATCAACA | |
| GCGGCATCTG CAAAAGCAAC TACCCGTGCC AGTGGTTTAA | |
| CTTTCAGTCT CTTGGCTGCC TCTGTAGTCA TCAGAACCAA | |
| AGCAGCTGCT CCATCATTCA NAGT | |
| AGGTGAACGC ATTCAAGGTG TTTGATCCAG AGGGCAAAGG | 1202 |
| GCTGAAATCT GCCTACATCA AAGAAATGCT GATGACACAG | |
| GGCGAGAGGT TTTCCCAGGA AGAGATCGAT CAGATGTTTG | |
| CTGCCTTCCC TCCAGATGTC TCTGGCAACC TCGACTACAA | |
| AAACCTCGTC CACGTCATCA CACATGGAGA GGAGAAGGAC | |
| TAATCCATGG ATTCAGCACT GGGGTTAGCA CTGTGGGATC | |
| ACCTCCATGT GGGTCACACT GCAGGTTCCC TTTGTCCCTC | |
| TCCCTGGAGC TGCAGAGCTG TTCTTCATGG GGATAACAAC | |
| CCAGAACAGC AGCCACATAC AATAAAGTGC ATTTTGGTGA | |
| GAGTAAAAAA AA | |
| AGGTACTAGA AACACATGCT ATGTATGTCA TTTAGAAATG | 1203 |
| TAGTGCTGCT TCTAGATGAG ACAACTCTTG AAGGTGAAGT | |
| ATAGTTTCAC GTAGCTCTAC GTCCCTTCCC AGAGAGTAAA | |
| ACAATTCCCT TCACCCTTAA CTTCCCATTT ACTTTATCCA | |
| AAATCAGGAG GAACCAACAA CGCACCATAG ATTCTCTACA | |
| GTCCACCCTT GATTCTGAAG CCCGGAGCAG AAATGAGGCT | |
| ATCCGTCTGA AGAAGAAGAT GGAAGGAGAC CTCAACGAGA | |
| TGGAAATCCA GCTCAGCCAT GCTAACAGAC ATGCTGCAGA | |
| AGCAACCAAG TCAGCACGTG GCCTGCAGAC ACAAATTAAG | |
| GAGCTCCAGG TGCAGCTGGA TGACTTGGGA CACCTGAATG | |
| AAGACTTGAA GGAGCAGCTG GCAGTCTCTG ACAGGAGGAA | |
| CAACCTTCTC CAGTCAGAGC TGGATGAGCT GAGGGCTTTG | |
| CTGGACCAGA CTGAACGGGC GAGGAAGCTG GCAGAGCATG | |
| AGCTGCTGGA AGCCACTGAA CGTGTGAACC TGCTGCACAC | |
| TCAGGTTGGC TTTTCCTGGG TTAAACTGAG CTTCACCTGT | |
| TAAGCACTGA CACTGGGA | |
| TGCAATGGAA GGAGTTTTCA CAAGACGTGC TTCCTCTGCA | 1204 |
| TGGCTTGCAG GAAGGCTCTG GACAGCACCA CAGTGGCAGC | |
| TCACGAATCT GAAATCTACT GCAAAACTTG CTACGGGAGA | |
| AAATACGGCC CCAAAGGTGT TGGCTTTGGA CAAGGGGCCG | |
| GATGTCTCAG CACCGACACT GGGGACCATC TGGGCCTAAA | |
| CCTGCRACAG GGATCACCAA AGTCTGCTCG CCCTTCTACA | |
| CCAACTAATC CTTCAAAGTT TGCCAAAAAG ATCGTTGATG | |
| TGGATAAATG TCCCCGGTGT GGCAAATCGG TGTATGCTGC | |
| AGAGAAGATA ATGGGAGGAG GAAAACCTTG GCATAAAACA | |
| TGCTTCCGCT GTGCTATCTG TGGAAAGAGT TTAGAGTCTA | |
| CAAATGTTAC AGACAAAGAT GGAGAGCTCT ACTGTAAAGT | |
| TTGCTACGCA AAGAATTTTG GTCCCAAAGG AATTGGTTTT | |
| GGTGGCCTCA CTCAAGTGGA AAAGAAAGAA TGAAGCCTTC | |
| TGAAGCCTTC TGAAGAAAAA GCAAGTTTTC TTAGAATATA | |
| GTGTTTCAGT TTTGTTATTG T | |
| CGCTGGGGCC GTTGACGTGC AGCAGGAACA CTATAAAGGC | 1205 |
| GAGATGGTGA AAGTCGGAGT CAACGGATTT GGCCGTATTG | |
| GCCGCCTGGT CACCAGGGCT GCCGTCCTCT CTGGCAAAGT | |
| CCAAGTGGTG GCCATCAATG ATCCCTTCAT CGACCTGAAC | |
| TACATGGTTT ACATGTTCAA ATATGATTCC ACACATGGAC | |
| ACYTCAAGGG CACTGTCAAG GCTGAGAATG GGAAACTTGT | |
| GATTAATGGG CATGCCATCA CTATCTTCCA GGAGCGTGAC | |
| CCCAGCAACA TCAAGTGGGC AGATGCAGGT GCTGAGTATG | |
| TTGTGGAGTC CACTGGTGTC TTTACTACCA TGGAGAAGGC | |
| TGGGGCTCAT CTGAAGGGTG GTGCTAAGCG TGTTATCATC | |
| TCAGCTCCCT CAGCTGATGC TCCCATGTTT GTGATGGGTG | |
| TCAACCATGA GAAATATGAC AAATCCCTGA AAATTGTCAG | |
| CAATGCCTCG TGCACCACCA ACTGCCTGGC ACCCTTGGCC | |
| AAGGTCATCC ATGACAACTT TGGCATTGTG GAGGGTCTTA | |
| TGACCACTGT CCATGCCATC ACAGCCACGC AGAAGACAGT | |
| GGATGGCCCC TCTGGGAAGC TGTGGAGGGA TGGCAGAGGT | |
| GCTGCCCAGA ACATCATCCC AGCATCCACT GGGGCTGCTA | |
| AGGCTGTAGG GAAAGTCATC CCTGAGCTCA ATGGGAAGCT | |
| TACTGGAATG GCTTTCCGTG TGCCAACCCC CAATGTCTCT | |
| GTTGTTGACC TGACCTGCCG TCTGGAGAAA CCAGCCAAAT | |
| ATGATGACAT CAAGAGGGTA GTGAAGGCTG CTGCTGATGG | |
| GCCCCTGAAG GGCATCCTAG GATACACAGA GGACCAGGTT | |
| GTCTCCTGTG ACTTCAATGG TGACAGCCAT TCCTCCACCT | |
| TTGATGCGGG TGCTGGCATT GCACTGAATG ACCATTTTGT | |
| CAAGCTTGTT TCCTGGTATG ACAATGAGTT TGGATACAGC | |
| AACCGTGTTG TGGACTTGAT GGTCCACATG GCATCCAAGG | |
| AGTGAGCCAG GCACACAGCC CCCCTGCTGC CTAGGGAAGC | |
| AGGACCCTTT GTTGGAGCCC CTTGCTCTTC ACCACCGCTC | |
| AGTTCTGCAT CCTGCAGTGA GAGGCCAGTT CTGTTCCCTT | |
| CTGTCTCCCC CACTCCTCCA ATTTCTTCCT CAGCCTGGGG | |
| GAGGTGGGAG AGGCTGATAG AAACTGATCT GTTTGTGTAC | |
| CT | |
| ACAACATTAC TACCAGCTTT TTGATGCAGA CAGGACTCAG | 1206 |
| TTAGGAGCAA TATATATTGA TGCATCATGC CTTACGTGGG | |
| AAGGACAGCA GTTCCAGGGC AAAGCAGCTA TCGTTGAAAA | |
| ACTCTCTAGC CTTCCTTTCC AAAAAATACA ACACAGCATC | |
| ACAGCACAAG ACCACCAACC TACACCTGAC AGCTGTATAC | |
| TCAGTATGGT AGTGGGACAG CTTAAGGCTG ATGAAGATCC | |
| TATCAYGGGA TTCCACCAGA TATTTCTATT AAAGAACATC | |
| AACRATGCCT GGGTTTGCAC CAATGACATG TTCAGGCTAG | |
| CATTGCACAA CTTTGGCTGA GCTGGCGACC CCGAGGCACC | |
| TGTTCTTTTT TTCTTCTTCT CTCCTCTTAC TGATATTATT | |
| CACACTCACA GAACATTCCA AATATCATAC ACAAACCTGC | |
| AGCACTGCAG AGCGTGAGCA AGCAAGAGCT GTGACCTGCC | |
| CTTCTGCTGA GTTTACATTG TCACTAGATG AGTTCCTTGT | |
| GCATGATGTT TGGAAGTTAG TTAGCTGCAT TTGACAAGAG | |
| AAATTTGTGT TGT | |
| AGGTATGATC CTCCAATGGA AGCTGCTGGC TTCACTGCAC | 1207 |
| AGGTTATTAT CCTGAATCAC CCTGGCCAAA TCAGTGCTGG | |
| TTATGCCCCC GTGCTGGATT GCCACACTGC TCACATTGCC | |
| TGCAAGTTTG CTGAGCTCAA AGAGAAGATT GATCGTCGTT | |
| CTGGCAAGAA GCTGGAGGAT GGCCCTAAGT TCCTGAAATC | |
| TGGAGATGCT GCCATTGTTG ATATGATTCC TGGCAAACCC | |
| ATGTGTGTTG AGAGCTTCTC TGATTATCCT CCTCTGGGTC | |
| GTTTTGCTGT GCGTGACATG AGGCAGACGG TTGCTGTTGG | |
| TGTCATCAAG GCCGTCGACA AGAAGGCTGG TGGAGCTGGC | |
| AAGGTCACAA AGTCTGCTCA GAAGGCCCAG AAGGCTAAAT | |
| GAAAATTCTG T | |
| AGGTATGATC CTCCAATGGA AGCTGCTGGC TTCACTGCAC | 1208 |
| AGGTTATTAT CCTGAATCAC CCTGGCCAAA TCAGTGCTGG | |
| TTATGCCCCC GTGCTGGATT GCCACACTGC TCACATTGCC | |
| TGCAAGTTTG CTGAGCTCAA AGAGAAGATT GATCGTCGTT | |
| CTGGCAAGAA GCTGGAGGAT GGCCCTAAGT TCCTGAAATC | |
| TGGAGATGCT GCCATTGTTG ATATGATTCC TGGCAAACCC | |
| ATGTGTGTTG AGAGCTTCTC TGATTATCCT CCTCTGGGTC | |
| GTTTTGCTGT GCGTGACATG AGGCAGACGG TTGCTGTTGG | |
| TGTCATCAAG GCCGTCGACA AGAAGGCTGG TGGAGCTGGC | |
| AAGGTCACAA AGTCTGCTCA GAAGGCCCAG AAGGCTAAAT | |
| GAAAATTCTG T | |
| ACTGGGAGAA GCTCTCCACA CACATCGGCT TGCCAGGAAT | 1209 |
| CATCTCCACG ATGGCCGCAT CGCCTGATTT CAGGGATTTG | |
| GGGTTGTCCT CCAGCTTCTT GCCGGAGCGC CGGTCGATCT | |
| TCTCCTTCAG CTCAGCGAAC TTGCAGACGA TGTGTGCGGT | |
| GTGGCAGTCG ATGACAGGTG AGTATCCAGC ACTGATCTGC | |
| CCGGGGTGGT TCAGGATGAT CACCTGAGAT GTGAACTGTG | |
| CTGCCTCCTG CGGCGGATC | |
| GAGATGAAGA TCACATATGC ACAATGTGGA GATGTCTTGA | 1210 |
| GGGCTTTGGG GCAGAATCCA ACCCAGGCTG AGGTCATGAA | |
| GGTCCTTGGC AGACCCAAAC AAGAAGACAT GAACTCCAAG | |
| ATGATTGACT TTGAGACCTT CCTGCCCATG CTCCAGCATA | |
| TCGCCAAGAC AAAAGACACR GGCACCTATG AAGACTTTGT | |
| GGAGGGTCTR CGTGTGTTTG ACAAGGAAGG AAATGGAACA | |
| GTGATGGGGG CTGAACTCCG CCACGTTTTG GCTACACTGG | |
| GTGAAAGGTT GACTGAAGAG GAAGTTGATA ARCTAATGGC | |
| TGGCCAGGAA GATGCCAATG GTTGTATCAA CTATGAAGCT | |
| TTTGTGAAAC RTATCATGGC TAACTGAACA CCAGGACAAG | |
| ACAGGCGTGG AGAAGCCCGG ATTCTGGCCT TGGATTTTGA | |
| TTTATTGGAA TGTCCTCTCA TTTTTCAGTC CAGATTCCTA | |
| CTTCAAAGCT ATAAAATGTA TTGTCCCTGA AGTTATTTGG | |
| ATAAATGCTT GTTTGTTTTG TCTTGTTTCC TCATGGGAAG | |
| AAAAAAGGAA ATTGAACAAA CAGAACCAGA ACCATGAATA | |
| CCTTATTGCA TTGTATGCAA TAAGG | |
| GAGGATGGCA GCGGCACTGT GGACTTTGAT GAGTTCCTTG | 1211 |
| TTATGATGGT CCGGTGTATG AAAGATGATA GCAAAGGGAA | |
| AACTGAAGAG GAACTATCAG ATCTTTTCAG GATGTTTGAT | |
| AAGAATGCTG ATGGCTACAT TGATCTTGAG GAACTGAAGA | |
| TCATGCTGCA GGCAACTGGA GAGACGATCA CTGAGGATGA | |
| CATAGAAGAA CTGATGAAAG ATGGGGACAA AAACAATGAT | |
| GGCAGGATTG ACTATGACGA GTTCCTGGAG TTCATGAAGG | |
| GAGTTGAATA AATCTGAGGC CAGATGGACA GCCCGAATCT | |
| CTGAAACTCC TTCTGCTCTC TGACTCAGCT CCTTGGTTCC | |
| ATCCCCTGGC TGCCAGCATG AAGACTGAGC ACTGAGAAGG | |
| GTGGCCGTAG GGAAAATAAA GCACATTGCT GTCAAAAAAA | |
| AAAAAAAAAA AAA | |
| ACCTGATTCT TCTTAACAAA TGGAGGAAAT GATGCCCCAT | 1212 |
| CAGTGCCGTT AACCAAATCG CAGTAACCTT CCCAGTAAGA | |
| CAGATTCCTT TTGTTTTTAT AACTTTCAAT TATTGCTGTT | |
| TTGCTTATGT CTTCTTTCCC AGTATACACT CTGTAAAGTC | |
| CATCAGATGT CCCATTATAC GGGTAGAAGA CTCCCAGAAC | |
| TGGGTCCAAG GGGAAGGGAA CCTTGCTTAA GAAGGGGTCT | |
| TTGTATCCCC ATAGTATTTC TTTCACTGTT CTGTTCTGCA | |
| GCATGTTTGA TTTAGAAGAT TTAATCCAAG TATTTAAAAG | |
| TAGGAGGATG AAATTGTTTG TATACAGGGC AGGTGCAGCA | |
| ACAACAGCGA GGTTGAGGCA CGTGATGGTG TCATTTTCTG | |
| TCCCAACAGA CATATCAGGT TCAAAACGAG CAGCGTTAGG | |
| CAACATGTAA GATATTGTGC CATTAGAGTT TTCTGTAATA | |
| TTTTCTTTAG GTAAATATCG CACCCTATAT GTGTAAGGTC | |
| CTCTTTGTTC AAGTTTTGGA CGTGCTCCAT AGTTCAAAAC | |
| CTCTGATGGA TTTTCCACAT TAAAGATCCA AAATTGCCTG | |
| TAAACAGAGC TTCCTGGCAC AAGCCAATTA TCATATGCAA | |
| TGGT | |
| ACTGCCACCA AACCCAGAAC CAAGGCCAGC ATCTTCCATG | 1213 |
| ACTCAAAGCT TGTTTTCAAG AAAAGGCAGT GAGCTTTGGA | |
| GCGGAGAGAA GGTAAAAAGC AGCAGCTATC TTGTAGAACT | |
| TCATCATGAG ATTGGCAATG GACATCCTCT TGTTACTGCA | |
| CAACCTTCTA TCACCAGCAC ATTTTATTCT GAACCAACCT | |
| CTCACCCTAC AATTGCTGAA TGAGAGTAGA AACACAGGTT | |
| TGCAGATTAT TCTGTCAACT GCAGAAGT | |
| ACTTATCTTC AAAAACAGCC ATAGCTGCCA GTGAGCCAGA | 1214 |
| ACCCATGGTA ACATAAGGCA ACTTGTCAGT TGATCCATGG | |
| GGATATATGC TGTAAAGGTG AGGTCCAGTG ACATCTACAC | |
| CTCCTAAAAC CAAGGCAGCA CCAATGTAGC CTTGATACCT | |
| GAAAAGCATT TGCTTTAGCA TTCGATTAGC TGTGACCACA | |
| CGTGGAAG | |
| ACCCTTCCTA TTAAAGATCC TCACGTAGAC AGTGCATCTC | 1215 |
| CAGTGTATCA GGCTGTTCTC AAAACTCAAA ACAAGCCTGA | |
| AGATGAAACT GAAGATTGGA GCCGCCGTTC TGCCAACCTG | |
| CAGTCTAAGT CTTTTCGCAT CCTTGCCCAG ATGACTGGAA | |
| CGGAGTTCAT GCAAGATCCA GATGAAGAAG CCCTGAGGAG | |
| ATCAAGGGAA AGGTTTGAAA CGGAACGTAA CAGCCCACGC | |
| TTTGCCAAAT TGCGCAACTG GCATCACGGC CTGTCGGCGC | |
| AAATCCTTAA TGTTAAGAGT TAAAAGCCCA CGTTCAGTGG | |
| GCAAAGATGT GAGAGAGAAT TACAGGAAAG AAATAACTGC | |
| TATCCTGAGT TAGAGCCTAA CAACGTAACA CACGT | |
| ACATTGAAGG TCTCAAACAT TATCTGGGTC ATCTTTTCAC | 1216 |
| GATTGGCTTT AGGGTTCAAG GGTGCTTCTG TGAGCAAGGT | |
| GGGGTGCTCC TCAGGGGCCA CACGGAGTTC ATTGTAGAAA | |
| GTGTGGTGCC AGATCTTTTC CATATCATCC CAGTTGGTGA | |
| TGATGCCATG TTCAATGGGA TATTTCAAAG TAAGGATACC | |
| TCTTTTGCTC TGTGCTTCAT CACCCACATA GGAATCTTTT | |
| TGACCCATAC CAACCATAAC ACCCTGGTGC CTGGGGCGGC | |
| CAACGAT | |
| ATATTCATTC CAAGAACTTC ATCCACCGGG ATGTGAAGCC | 1217 |
| AGACAACTTC CTTATGGGGC TTGGTAAAAA AGGCAACCTA | |
| GTATACATCA TTGATTTTGG TTTGGCCAAG AAGT | |
| ACACAATGCA GGGTGCACGA GCCTGTGCTT CTCTGAAGAG | 1218 |
| GCTCCGGACT CGTGCGGCTC CAAGACCTCC TATCACCTCC | |
| ACAAATTCAG AGCCTGCCAT GGCCAAGAAA GGCACCTGTG | |
| CTTCTGTGGC CACTGCCTTT GCCAACAACG TCTTCCCGCA | |
| GCCTGGTGGT CCAAGCAACA AGGCACCCTT GGGCACTTTA | |
| GCACCGAGCT GAAGGTAGCG ATCAGGATTC TTTAGGTAGT | |
| CCACAAATTC TTTGACTTCC ATTTTTGCCT CGTGCATTCC | |
| TGCTACGTCC TTGAAGCCAA TTCCTTTCCC GGATTTTCCG | |
| TCCACAATGG TGAAACGAGC CATTTTCAGC TGATTAAAAG | |
| CATTGAATCC TCCTGCCCGG CCCGCAACCC TGATAAGGCG | |
| GAAGATGCTC CACAACATGG ACAGAGCCAC CAGTGTCACT | |
| ATCAGGGAAA TGACATCATT TCCGTAAAAC CCGGGGTGTT | |
| TGTAGGAAAC AGGGATTCTC TCTCTCTCAT CAATATTCAG | |
| CTCGTCCTCC GCAGCTCTCA GCTTCTCTTC GAACTTGTCG | |
| ATGTTTGCCA CTCGCATGGT GT | |
| CAGCTTTGGA AAACACTATC TTTAACATTA AGGTGTAAAG | 1219 |
| GATGAACAAC ACAAAATTAA AGTGTGTGCT GTATTGCTAG | |
| AATGCATCCC TTCTCTCTGT TCTCCACAAG GATATGTTCC | |
| CATTAACAGT CTAGTCTATG AAACAAATGT TTTTCCCAAT | |
| GAAAACTTGA AATTGTTCCA TTGTGGACCA ATTCTTAAGA | |
| GAGCAGTAGC AGGAGATGCC TCTGAATCTG CACTTCTGAA | |
| ATGCATTGAA TTGTGCTGTG GTTCTGTCAA AGAAATGAGA | |
| GAAAGATATC CCAAAGTGGT GGAAATACCG TTTAACTCTA | |
| CCAATAAGT | |
| ACGGGTCAAG CAAAGAAGTC ACAGTTAGGG GCCATAACTG | 1220 |
| TCCAAAACCA ATAATAAACT TCTATGAAGC TAACTTTCCT | |
| GCAAATGTTA TGGAAGTGAT TCAGAGGCAG AACTTCACCG | |
| AGCCTACTGC AATTCAGGCA CAAGGATGGC CTGTTGCCTT | |
| GAGTGGATTG GACATGGTTG GAGTTGCACA GACTGGATCA | |
| GGGAAAACAC TGTCTTACTT GTTGCCTGCT ATTGTGCATA | |
| TAAATCATCA GCCATTCCTG GAAAGAGGAG ATGGACCTAT | |
| TTGTCTTGTG CTGGCACCAA CTCGTGAACT GGCTCAGCAA | |
| GTGCAGCAGG TAGCTGCTGA ATATAGCAGA GCATGTCGCT | |
| TGAAGTCTAC ATGTATTTAT GGAGGTGCTC CAAAGGGACC | |
| ACAAATTCGT GATTTAGAAA GAGGTGTGGA AATTTGCATT | |
| GCAACACCTG GAAGACTTAT AGACTTCTTA GAAGCTGGAA | |
| AGACCAATCT CAGGAGGTGC ACTTACCTTG TCCTTGATGA | |
| AGCTGACAGG ATGCTTGACA TGGGATTTGA ACCTCAAATC | |
| AGAAAAATTG TGGATCAGAT AAGACCTGAC AGGCAGACTC | |
| TGATGTGGAG TACCACATGG CCGAAGGAAG TTAGGCAGCT | |
| GGCTGAAGAC TTTTTAAAAG A | |
| ACTTGAGCAC GACAAGTTTA ACCTTCTTCC TCTTATGCTT | 1221 |
| GTTCTTCTTG GGGGTGGTGT AAGACTTCTT CTTTCTTTTC | |
| TTAGCACCAC CACGCAGTCT CAGCACAAGG TGAAGAGTTG | |
| ATTCTTTCTG GATGTTGTAG TCAGACAGCG TGCGGCCATC | |
| TTCCAGCTGC TTCCCAGCAA AAATCAGTCG CTGCTGATCA | |
| GGAGGAATTC CTTCCTTATC CTGGATCTTA GCTTTCACAT | |
| TTTCTATAGT ATCAGAGGGC TCGACCTCGA GGGTGATGGT | |
| CTTCCCCGTG AGGGTCTTCA CGAAGATCTG CATGTCGAGG | |
| CCCGCACCCG CGGGGAAGAG GCG | |
| ACTTGAGCAC GACAAGTTTA ACCTTCTTCC TCTTATGCTT | 1222 |
| GTTCTTCTTG GGGGTGGTGT AAGACTTCTT CTTTCTTTTC | |
| TTAGCACCAC CACGCAGTCT CAGCACAAGG TGAAGAGTTG | |
| ATTCTTTCTG GATGTTGTAG TCAGACAGCG TGCGGCCATC | |
| TTCCAGCTGC TTCCCAGCAA AAATCAGTCG CTGCTGATCA | |
| GGAGGAATTC CTTCCTTATC CTGGATCTTA GCTTTCACAT | |
| TTTCTATAGT ATCAGAGGGC TCGACCTCGA GGGTGATGGT | |
| CTTCCCCGTG AGGGTCTTCA CGAAGATCTG CATGTCGAGG | |
| CCCGCACCCG CGGGGAAGAG GCG | |
| CCGGGCAGGT ACCTTTTAAC CCCATGGAAA AAATATCTAA | 1223 |
| CGTTCATTAC TACCAATAAC AGGAAGAAGA TTTTGCTTCG | |
| AGAATGACAA ACCCATCATG GTGAAGTTTA GGCACGCTCC | |
| CCACGAATGC GGCGTGCTAG CTGGATATCT TTTGGCATGA | |
| TTGTGACACG TTTGGCATGG ATAGCACACA GGTTGGTATC | |
| TTCAAACAGG CCAACCAAGT AGGCTTCACT TGCCTCCTGC | |
| AAAGCACCGA TAGCAGCGCT CTGGAAGCGC AGATCTGTTT | |
| TGAAGTCCTG AGCAATTTCA CGCACCAGAC GTTGGAAGGG | |
| AAGTTTGCGG ATCAAAAGTT CGGTAGACTT CTGATAGCGC | |
| CTGATTTCAC GGAGGGCCAC AGT | |
| ACATGAGGAC TCCAACTGCT CCTGCCTCTT TGGCATTTGC | 1224 |
| AACCTTCTCA GCAAGTGTTA TTTTTCCAGC TCTGACAATG | |
| ACTATGGTTC CATTCAATGG AGTCACTGAC TTCTGTATTG | |
| TCTCAATATC TTTTTTCAGT CCATAGTTCA CATAGACAGG | |
| TTTGCCAGAA AAAGAGCCAC TCTCACTGTA GGCCACGTAT | |
| GCATCAGGCA TCTCCAAGAT CTCCTCGCTA TCATTGATCA | |
| AAACGGACAC TTTGTTCTTG GTGCTGCCTC TGATTTGCAA | |
| CTTAATATAG TGTTCATCGT TCCACACTTT ATCCAAGAAG | |
| AAACTGTTGA ATTGCTCATG AATGTAGGTG GCCATGTTTG | |
| TATCTTCAGC CTCACCAGCC TCAAAGGAGT CCAAACCTGC | |
| CCTTTGCCTC AAGCGGTCTC CAAGGTTCTT GGCTAACAGC | |
| TTATCTGACA ACATGGCTTT TAATTGAGGC CAG | |
| GTTTGTTGCT GGAACACATC AATTGTATCT TCATCCTCCA | 1225 |
| TTTCCAACTG KGCGGGGGTG TCTGTTTCAT TAATTGGCTG | |
| CCCATCGAAC CGGAATCTGA TTTGCCTCAT CGACAACCCC | |
| TGTCGTTCAC AATAGGCTTT CATTAGTTTA CTAAGKGGGG | |
| TATGCCTCTT AATCTTAAAC TGCAC | |
| ACCCTCGGGC AGCTTAGGCA GTCTCACCGT TTCTGCATCG | 1226 |
| AGCAAAAGCA CACCATCACT GCTGTGCAGT TTGATGTCCA | |
| TCTGGGAAAG AGCTTCAATA TTCCCTGCTT GCGCTTTGAT | |
| GTTAATGCCT CTTGGAGCAT CCATGCTCAG AGACCGAGTT | |
| GGTGACTCCA GCCTTAGCTG TTTAAAAGCT TCTGCCTTCA | |
| CGAGAGGTGT TTCTACAGAG TGTTCAAAAA GTGCTCCTTC | |
| AGGCCCTGTG ACTCGAAGTT TATCTGTTCC AATGACAACC | |
| TCATTTTCAT CCACTGTAAA AAGTGGCTTG CCATCCTTTG | |
| AGTTGATCTG AAATTGC | |
| ACTCCTTCAT GACCTGAATA AAGTATGGCA TGGCAAAGTC | 1227 |
| CATGATGTTG TGCCTCCACG CTGTTTCCAA GACAACGTCA | |
| GGCCTTAAAA GATCATAGCA GGTGAAGAGA CAAGCACCGA | |
| AGCACTCTTT CTTGTTCTCC TGCAAGAACC ACTGCAACAA | |
| TTCTTCTGCC AACTCAGTAT CTTTGGATTC TGAAGCATAT | |
| TGCATTGCAT CCTTATACAG TCTGTCCTTC TTACAGAGTT | |
| CCACACTTTG CTTCCAGCGG TTGTTTCCTT TGAACAGATA | |
| TGCAGCAATC CTTCGGAACT CTATCAGCTC GTGTTTCTCC | |
| AAACGTTGAG CAAGAGAAAT GTTGTCAAAG TTGTCATAAG | |
| CATCTATAGA AGTCCTAAGG GCCTGGTAGT CTTCTTCAAT | |
| AATGAAGAGG TTGTTCAGGG ACTCATTCAC TGATTTGTTG | |
| TTGTGATTTT GAACAGAGCG CAAGTAAGGT TTAACCAGGG | |
| GTAACTGTTT AACCTTGGTG AAGAAGGTAA CAGCACGAGT | |
| ATGGTCAAGT CGAGGGGACA ATACCATCAG CAGATCATTG | |
| AGCAACAGAG GTTTGAATTC CAAATAGAAT TGAACTGCTT | |
| TGTAGT | |
| ACTGAGCCTG CTCAGGAGGC AGCTCTCGAC GTAACTCATC | 1228 |
| TGCCAGGATA TACGGCTTAT CTGAAGCCAG GATTCTGAAG | |
| GAAGCTATCA CTTGCTCAGC CGTGTCCGTA TCTGCTGTTT | |
| CTCTGGTCAT GAAGTCAATG AAGGACTGGA AAGTGACGGT | |
| TCCTTGTCCA TTGGGGTCAA CCAGAGACAT GATTCTAGCA | |
| AACTCAGCTT CGCCCAAATC ATAACCCATT GAAATCAAGC | |
| AAGCTCTGAA ATCATCATGG TCCATCAGTC CATTCTTCCT | |
| CCTGTCAAAG TGATTAAATG ATGCTCTGAA GTCATTCATT | |
| TGCTCCTGGG TAATACCCTT GGCATCTCTT GTGAGGATCT | |
| GAG | |
| ACTGTGATGA GACTGCCTTA CTCAACACCT TCTTTTGAAA | 1229 |
| GAGGAGGTGT TAAATAAAGA TTAAGGTTTC TGAGTCATTA | |
| CAGTTCTTGT AAATGCACTC ACTTATTCTT ATGAATGGCT | |
| GAGAGACTTA TACTTTATCC ACTGGTTGGG GCCACGCACT | |
| TCAAAGGGCG GCTCATTGAA GTAAATGTGG TTACCGAGTT | |
| CTTCCAGTGG GGGCTCTGGG TCAGTGGTAG CAAACTGAGC | |
| AGCTTCCTCT ATCTCTTTTC TCACTGCCAC ATCGATTTCC | |
| TTTAATTCTT CAACGCTAGC AAGGTTATTG TTGATCATTC | |
| TGTCCTTCAG CAAAGTAATG GGATCACTTT TGCTTCTCAC | |
| TTCTTGAATT TCCTCTCTAG TACTTTTTTT CTCATCCTGT | |
| GCTTATGCCC AGGAAGGAAT TTCTGTTATC TATTATTTTG | |
| T | |
| ACATTCATGA CAGGTTTCTT TTTTCTTTCT AAAGAAAAAG | 1230 |
| GCCTTTCTGT TTTGTTAGCA CTTTGGTGTT TCAATGTGAT | |
| AATATTTAAA AACTGGATTT AAATAAGAGG TTAGTCAGGA | |
| AAAACACACA ACAATGCTTG CAAAGTGCTC CACACCGCTG | |
| TAGCCACAGG AGTGTGAACA CACTCTAGAA CACGGGGGCA | |
| TCCAGCCACG GTGCTCTGT | |
| GTATCATTGT ATTTTTCTTC TGCAATTTTC TTGATCATAT | 1231 |
| CAAGAGTTTT ACGAACAAGC TTCTTTCTGA TCACCTTTAA | |
| TAACTTATGC TGCTGAAGTG TTTCACGAGA TACATTCAAA | |
| GGAAGATCAT CAGAATCCAC AACACCCTTA ACAAAGTTAA | |
| GATATTTGGG CATCATGTCA TGGAAGTCAT CAGTGATGAA | |
| CACTCTTCTA ACATACAGCT TAATGAAATC ACTTTTTTTG | |
| GATCCATACT CATCAAACAA GCCACGTGGA GCAGAATTAG | |
| GAACAAACAA GATTGATTTG AAAGTTACTT CCCCTTCAGC | |
| AGTAAAATGG ATGTAAGCCA TTGGATCATC ATGTTCCTTG | |
| GAAAATGTTT TGTAAAAAGC TTTATATTCA TCCTCTTCAA | |
| CTTCTTTAGA TGGTCTCTGC CAGATTGGTT TTATGTCATT | |
| CATGAGCTCC CAATCCCAGA CAGTCTTCTC AACCTTCTTA | |
| GTTTTTGGTT TCTTCTCTTC CTCCTCTTCT TCAACTGCAG | |
| CTTCATCATC ATCTGTTTCT TCTTTTTCCT CCTTTGCTCC | |
| CTCCTCTTCA ATGGG | |
| ACCAGTAAGC ATATGAACTT CAAAATGCAC AATTGCCACA | 1232 |
| GACAGTTGAC TTGAATACAG TAATGGTGGT TGGTTGCACA | |
| CTTAGAGACG ACTTTTAGAT TCTTCCACTC TCAAATGGCT | |
| TTGCATTTCT GGATCATCTA GTCATGCACT GGAGAGGAAT | |
| TCCACAGCTG TCTCCTTCTC TTCAGTTAAC TCCTTAGCAG | |
| TCAGATCCAT CTTCTCACGA GAAAAGTCAT TAATAGGAAG | |
| ACCCTCAACA AACTTCCAGG TCTTGTCCTT GATCACAACA | |
| GGGAATGAAT ATAGCAAGTC TTCAGGAACA CCATAAGAAT | |
| TGCCATCAGA AATGACTCCC ATGGAAACAA ATTCTCCCGC | |
| TGGAGTGCCA AACCAGATGT CTCTCACATG ATCACAGATT | |
| GCTTTGGCAG CTGACATTGC ACTGGACAGC TTCCTAGCCT | |
| TAATAACAGC TGCTCCACGT TGCTGAACAG TCAGGATAAA | |
| GTCTCCCTTC AGCCAGCTGT CATCTTTTAT AGCTTCATAA | |
| ACTCCAACTT CCTTTCCTTT CACATTCACC TTTGCATGGT | |
| TAACATCTGG ATATTGAGTG GAGGAGTGGT TGCCCCANAT | |
| GATGACATTC TTCACATCG | |
| ACGTTGACAA CCATATTGGT ATCTCAATTG CCGGACTTAC | 1233 |
| AGCTGATGCA AGACTCTTGT GCAATTTTAT GCGTCAGGAG | |
| TGTCTGGATT CTAGATTTGT GTTTGATAGA CCTCTTCCAG | |
| TGTCTCGTTT GGTGTCACTA ATCGGAAGCA AAACGCAGAT | |
| ACCAACACAG CGCTATGGCA GAAGACCATA TGGTGTAGGA | |
| CTGCTCATTG CAGGTTATGA TGATATGGGC CCTCACATCT | |
| TCCAAACTTG TCCCTCAGCA AACTATTTTG ACTGTAAAGC | |
| AATGTCCATT GGTGCTCGTT CGCAGTCAGC ACGAACTTAC | |
| TTGGAAAGGC ACATGACTGA ATTTACTGAC TGTAATCTAA | |
| ATGAGCTAGT TAAACATGGA CTGCGTGCCC TGAGAGAGAC | |
| TCTTCCTGCT GAACAGGATC TGACCA | |

1. An isolated polynucleotide selected from the group consisting of SEQ ID NOS.: 1-1143 or SEQ. ID NOS.: 1144-1233.

2. The isolated polynucleotide of claim 1 further comprising the complement of the isolated polynucleotide to provide a double stranded polynucleotide.

3. An array comprising:
a substrate;
at least one polynucleotide disposed on the substrate that is selected from the group consisting of SEQ. ID NOS.: 1-1143 or SEQ. ID NOS.:1144-1233.

4. The array of claim 3, in which the array is configured as a cDNA chip.

5. The array of claim 4, in which the cDNA chip comprises at least one contiguous nucleotide that is complementary to the polynucleotide selected from the group consisting of SEQ. ID NOS.: 1-1143 or SEQ. ID NOS.: 1144-1233.

6. A kit comprising:
at least one polynucleotide of claim 1; and
at least one enzyme.

7. The kit of claim 6, further comprising at least one polynucleotide that is complementary to a polynucleotide that is selected from the group consisting of SEQ. ID NOS.: 1-1143 or SEQ. ID NOS.: 1144-1233

8. The kit of claim 6, further comprising a cDNA chip configured with one or more contiguous nucleotides from the isolated polynucleotide selected from the group consisting of SEQ. ID NOS.: 1-1143 or SEQ. ID NOS.: 1144-1233.

9. The kit of claim 8, further comprising a cDNA chip configured with one or more contiguous nucleotides that are complementary to the isolated polynucleotide selected from the group consisting of SEQ. ID NOS.: 1-1143 or SEQ. ID NOS.: 1144-1233.

10. A primer comprising an effective amount of contiguous nucleotides from a polynucleotide selected from the group consisting of SEQ. ID NOS.: 1-1143 or SEQ. ID NOS.: 1144-1233.

11. The primer of claim 8, in which at least 50 contiguous nucleotides of the polynucleotide comprise the primer.

12. A vector comprising at least one polynucleotide selected from the group consisting of SEQ. ID NOS.: 1-1143 or SEQ. ID NOS.: 1144-1233.

13. A host cell comprising the vector of claim 12.

14. A method of diagnosing heart failure, the method comprising:
exposing a patient sample to a polynucleotide selected from the group consisting of SEQ. ID NOS.: 1-1143 or SEQ. ID NOS.: 1144-1233; and
determining if a gene or gene product in the patient sample binds to the polynucleotide.

15. The method of claim 14, further comprising determining if a gene or gene product in the patient sample is up-regulated or down-regulated.

16. A method of diagnosing idiopathic dilated cardiomyopathy, the method comprising:
exposing a patient sample to at least one polynucleotide selected from the group consisting of SEQ. ID NOS.: 1-143 or SEQ. ID NOS.: 1144-1233; and
determining if a gene or gene product in the patient sample binds to the polynucleotide.

17. The method of claim 16, further comprising determining if a gene or gene product in the patient sample is up-regulated or down-regulated.

18. A method of diagnosing heart failure in a female subject, the method comprising determining if at least one female heart failure gene is up-regulated using at least one polynucleotide selected from the group consisting of SEQ. ID NOS.: 1-1143 or SEQ. ID NOS.: 1144-1233

19. A method of diagnosing heart failure in a female, the method comprising determining if at least one female heart failure gene is down-regulated using at least one polynucleotide selected from the group consisting of SEQ. ID NOS.: 1-1143 or SEQ. ID NOS.: 1144-1233

20. An antibody effective to bind to a polynucleotide selected from the group consisting of SEQ. ID NOS.: 1-1143 or SEQ. ID NOS.: 1144-1233.

21. A first ribonucleic acid molecule effective to bind to and inhibit translation of a second ribonucleic acid molecule transcribed from a polynucleotide selected from the group consisting of SEQ ID NOS.: 1-1143 or SEQ. ID NOS.: 1144-1233.


SEQUENCE LISTING (1233 sequences)

SEQ ID NO: 1 - DNA, 70 bp, Homo sapiens
ggaaaacagccggtgatcttctaccaataaagccagtggaaattgccatagaggcatggtgggtggtgca

SEQ ID NO: 2 - DNA, 70 bp, Homo sapiens
tttgcaaatatgtgtataaccacattggtggggagcattccgctgtgatcccagagctggcagccacagt

SEQ ID NO: 3 - DNA, 70 bp, Homo sapiens
cctggcttggagacccctctctgccatctgttgactggctctgtaattctggaaaacaccctttctaaac

SEQ ID NO: 4 - DNA, 70 bp, Homo sapiens
actggtaagaccatcactctcgaggtggagccgagtgacaccattgagaatgtcaaggcaaagatccaag

SEQ ID NO: 5 - DNA, 70 bp, Homo sapiens
tactaaaaatacaaaaattagcccggtgtggtggcaggtgactgtagtcccagctactcggcaggctgag

SEQ ID NO: 6 - DNA, 70 bp, Homo sapiens
aaattgtcctaataatatgtggtgctcatgagtgcgggacctgactgggctcagctaggcggttctcact

SEQ ID NO: 7 - DNA, 70 bp, Homo sapiens
atacaaaaaattagcccagtgtggtggcacatgcctgtagtccctcagacctgtaagctactcaggaggc

SEQ ID NO: 8 - DNA, 70 bp, Homo sapiens
gtatgtagaagacttcaaagccctagaggatggcagagccaccagctggacaaaaactgggcccagaatt

SEQ ID NO: 9 - DNA, 70 bp, Homo sapiens
catcccactccatcccttctgggatgtgaatcatccgtttgtccagcgtattcacgctatatatgctccc

SEQ ID NO: 10 - DNA, 70 bp, Homo sapiens
agatatcgagctcaggactattaagcacgcctgtctacccacagcacagtactgatcattacagggcgca

SEQ ID NO: 11 - DNA, 70 bp, Homo sapiens
actcatacctcccatcttccagctgaagggctctcaagcccgctaagcaagcttctttatttactcggct

SEQ ID NO: 12 - DNA, 70 bp, Homo sapiens
gtaccgccccatgtataaggctttccggagtgacagttcattcaatttcttcgttttcttcttcattttc

SEQ ID NO: 13 - DNA, 70 bp, Homo sapiens
tttagccacagacgtaggctacaagacagcggaacatcactttacggctttgcccacagacatgaaggtg

SEQ ID NO: 14 - DNA, 70 bp, Homo sapiens
cagggcgtagggcctgggccggggtcggcggcgcccccggggctggaggcggcccggcagaagctggcgc

SEQ ID NO: 15 - DNA, 70 bp, Homo sapiens
gttggggtccatccctctctgatgtgctttttccacaacacatatctggtcctctggcaggattgtggat

SEQ ID NO: 16 - DNA, 70 bp, Homo sapiens
ctgtggttggagtccgtgcggctggagtaccgtgcggggctgaagaacatcgcaaatacactcatggcca

SEQ ID NO: 17 - DNA, 70 bp, Homo sapiens
gtccctgggcagccctccatttgagaaacctaatattgagcagggtgtgctgaactttgtgcagtacaag

SEQ ID NO: 18 - DNA, 70 bp, Homo sapiens
acctcgcccatcttcacttagccttcgtatttgtgaaggattcagccaccttccttcttcaccccatgct

SEQ ID NO: 19 - DNA, 70 bp, Homo sapiens
caatgaagatattttagagtacaaaagaagaaatgggctggaataaacttttgaaacactaatgtagtat

SEQ ID NO: 20 - DNA, 70 bp, Homo sapiens
cgcgtcgctagctagtcgttctgaagcggcggccagagaagagtcaagggcacgagcatcgggtagccat

SEQ ID NO: 21 - DNA, 70 bp, Homo sapiens
cgcaagcttggcagcctttggtagagggtagcgagaacaagggaatgttgagagaatatggagagacaga

SEQ ID NO: 22 - DNA, 70 bp, Homo sapiens
atttaccaacctgggggattgatacgaccggggaaaatgttcctaaaccaggaagctgcgttagccgatc

SEQ ID NO: 23 - DNA, 70 bp, Homo sapiens
gcagtgtgggacaaagtccttagacaagaagcagcccagggtatccaataattgaaaaaggaggctgggg

SEQ ID NO: 24 - DNA, 70 bp, Homo sapiens
tgtgggagtatacatcggtgcaggcttcctggatgacagttgggtgatatgtgtcatgtggcctaaaagc

SEQ ID NO: 25 - DNA, 70 bp, Homo sapiens
cccctctcccaggtgtccccttgtagcatatgcattatgtcatctgaattgaggcctttctgtgaacagc

SEQ ID NO: 26 - DNA, 70 bp, Homo sapiens
attttacactttgttactaatttgcagaactctattaattgggtaggatttcacccattcctagctaagt

SEQ ID NO: 27 - DNA, 70 bp, Homo sapiens
tgccacgtatagctggaattaagtgttgtcttggagctgttgtacatttaagaataaacttttgtaaaaa

SEQ ID NO: 28 - DNA, 70 bp, Homo sapiens
tgggtcggtagtagcgatggcgggtctgactgacttgcagcggctacaggcccgagtggaagagctggag

SEQ ID NO: 29 - DNA, 70 bp, Homo sapiens
gtcatccagccctgctgtaaaatatgaagctgctgggacattagtgacactctctagtgcaccaactgca

SEQ ID NO: 30 - DNA, 70 bp, Homo sapiens
cctctgaggagccctcctggatgaatggagggaggcactcggctaacaaattagggcttctcgacgtcct

SEQ ID NO: 31 - DNA, 70 bp, Homo sapiens
caggaagcagcgtctcatcaggacagaaggtaggatgaagacatggggtaatgtgagagagtagaacacc

SEQ ID NO: 32 - DNA, 70 bp, Homo sapiens
tgaatcccactcccaccagagaattagcgcgggcggacgagcaaagtgaaacttagtagcccggaacttc

SEQ ID NO: 33 - DNA, 70 bp, Homo sapiens
ccctggagctgagcacaaagagtcatgtgacsgaagaggaggaggaggaagaggaagaagaatcagattc

SEQ ID NO: 34 - DNA, 70 bp, Homo sapiens
gcgtgagacacatcacatttgtggacaatgccaagatctcctactccaatcctgtgaggcagcctctcta

SEQ ID NO: 35 - DNA, 70 bp, Homo sapiens
agggcctgctccatcccaccttcctttctgctgcctgatgtctcaatggcttctgaatgactgttctaat

SEQ ID NO: 36 - DNA, 70 bp, Homo sapiens
gcggacgctatctacgaccacatcaacgaggggaagctgtggaaacacatcaagcacaagtatgagaaca

SEQ ID NO: 37 - DNA, 70 bp, Homo sapiens
tatgggaccacactgtgctgagaagcttcctgaggcccctcaacctgaaggccctgctacaagcagttca

SEQ ID NO: 38 - DNA, 70 bp, Homo sapiens
gcactcccttggtgtagacaaataccagttcccattggtgttgttgcctataataaacactttttctttt

SEQ ID NO: 39 - DNA, 70 bp, Homo sapiens
gcaccattgaattctgcagttcctagtgctggtgcttccgtgatacagcccagctcatcaccattagaag

SEQ ID NO: 40 - DNA, 70 bp, Homo sapiens
atctgtgcggaagtagcttgcctcacttctgcttaggaaagcggctgttgctccataactctaaccagca

SEQ ID NO: 41 - DNA, 70 bp, Homo sapiens
gcgcgcacgcacgccttgagcagtcagcattgcacctgctatggagaagggtattcctttattaaaatct

SEQ ID NO: 42 - DNA, 70 bp, Homo sapiens
gacctactgtattagacagtaacctctaacctcacctccaagcccaagtatatggccctgctgggttacc

SEQ ID NO: 43 - DNA, 70 bp, Homo sapiens
catatctgtttcctccatcggagcaaaaccactgagatcatccattcaaccctgaatcccacgtgggacc

SEQ ID NO: 44 - DNA, 70 bp, Homo sapiens
ttacatccatctatgagtggaaagggaagatcgaggaagacagtgaggtgctgatgatgattaaaaccca

SEQ ID NO: 45 - DNA, 70 bp, Homo sapiens
gtattcagcctttaggatgatcagaaaagcagaaagagagagtggccggatggggctgaggggagaaaga

SEQ ID NO: 46 - DNA, 70 bp, Homo sapiens
aggccccgcagtccctctcccaggaggaccctagaggcaattaaatgatgtcctgttccattggcaaaaa

SEQ ID NO: 47 - DNA, 70 bp, Homo sapiens
tactaataattattagctacaggcgggcgcagtggctcacaaccgtaatcccagcagtttgggaggctga

SEQ ID NO: 48 - DNA, 70 bp, Homo sapiens
gccgccactccagcctaatcccaaccccagggcgaacgttttcttatttatttccgttttctcgccacta

SEQ ID NO: 49 - DNA, 70 bp, Homo sapiens
ggatctgggcagtcagcactctttttagatctttgtgtggctcctatttttatagaagtggagggatgca

SEQ ID NO: 50 - DNA, 70 bp, Homo sapiens
tcgcccactaagccaatcactttattgactcctagccgcagacctcctcattctaacctgaatcggagga

SEQ ID NO: 51 - DNA, 70 bp, Homo sapiens
gccaatggtggcagcagaagtaggcgtatgggataactattgtgtaaagaaacagcttcttcactcctgc

SEQ ID NO: 52 - DNA, 70 bp, Homo sapiens
cttatgacattatctctaggctgccacttaaagtatggtttgaagacagggagaacggggcggcggagtg

SEQ ID NO: 53 - DNA, 70 bp, Homo sapiens
actagccgtgttttctcagactccacctttgtttgcactctgttgcctgtgaggagctttctggcatgtg

SEQ ID NO: 54 - DNA, 70 bp, Homo sapiens
gaggagctctcgacttagaggtaatatgaacagatgaacagacactgtggctggagccccaaagtgtgga

SEQ ID NO: 55 - DNA, 70 bp, Homo sapiens
tgccccactgagaagggtctagcggagcacaggtcaccagctgggcaacattcagaaagttagtcttcct

SEQ ID NO: 56 - DNA, 70 bp, Homo sapiens
cgggaggacaaccagaccaaccgcctgcaggaggctctgaacctcttcaagagcatctggaacaacagat

SEQ ID NO: 57 - DNA, 70 bp, Homo sapiens
tgaggcatgtactccccatgaggccacacaagagctgtgctttcttagatctggatcccactaccacata

SEQ ID NO: 58 - DNA, 70 bp, Homo sapiens
tgagccaggcctactcgtccagccagcgcgtgtcctcctaccgccgcaccttcggcggggccccgggctt

SEQ ID NO: 59 - DNA, 70 bp, Homo sapiens
cggcttcgaccctatatcccccgcccgcgtccctttctccataaaattcttcttagtagctattaccttc

SEQ ID NO: 60 - DNA, 70 bp, Homo sapiens
gaacagccaagctttgtgctactatgggatttcgttttctgcggttccaagtcttgatccacgtcctgcc

SEQ ID NO: 61 - DNA, 70 bp, Homo sapiens
catgtcatgcagctcagctgggagctgcttaggtggaaaactccaaataaagtgcgcctgtcgcagaaaa

SEQ ID NO: 62 - DNA, 70 bp, Homo sapiens
cccagaagcagttaagtctccaaaacgagtgaaatctccagaaccttctcacccgaaagccgtatcaccc

SEQ ID NO: 63 - DNA, 70 bp, Homo sapiens
ccaggaaagatttgccctcaagaacctcaaatgtagagagaaaagcatctcagcaacaatggggtcgggg

SEQ ID NO: 64 - DNA, 70 bp, Homo sapiens
ctgtggccaggggtccaaacagaaaataaccggagaagacaaggaggtcaaaggatcagggaactaagca

SEQ ID NO: 65 - DNA, 70 bp, Homo sapiens
aaccctggggattgggtgccatctctctaggggtaacacaaagggcaagaggttgctatggtatttggaa

SEQ ID NO: 66 - DNA, 70 bp, Homo sapiens
aagagcgtcaagcagacctgtgacaagtgtaacaccatcatctgggggctcattcagacctggtacacct

SEQ ID NO: 67 - DNA, 70 bp, Homo sapiens
cctctgaccgtttcagcaccctgggttgttaccacgtcctacaactctgacatttcttgttctcaagcgt

SEQ ID NO: 68 - DNA, 70 bp, Homo sapiens
attggtgagctgaagtctgtccttgcaccatgttatcatctgtttctcgtgtccgcctggttgaggagga

SEQ ID NO: 69 - DNA, 70 bp, Homo sapiens
aagctcacctgggcaggtctctgccacctccttgctctgtgagctgtcagtctaggttattctctttttt

SEQ ID NO: 70 - DNA, 70 bp, Homo sapiens
gagaatgatttacaacccctgctagcctggcttaaggtcatggagaaagcccacatcaacctggtgaggt

SEQ ID NO: 71 - DNA, 70 bp, Homo sapiens
cattttctgttgcaggaagccactccaccacagaatgctaatatgccagtggtacccagtacctcttgta

SEQ ID NO: 72 - DNA, 70 bp, Homo sapiens
tcccttgatcattatctctgaagtccctacctgcacttccctgattgccctgtagcaacaccagcatggt

SEQ ID NO: 73 - DNA, 70 bp, Homo sapiens
ttctgacaggaaaggggctccggaaaatcataaaacaagcaggtgaacaagaccaggtgtgtcggcacct

SEQ ID NO: 74 - DNA, 70 bp, Homo sapiens
ttttctcacaagaacccagttagctgatgttttattgtaattgtcttaatttgctaagaacaagtaataa

SEQ ID NO: 75 - DNA, 70 bp, Homo sapiens
gggtttgtgaaaagtgtatgtatttaaatttgctgtaaaacataatcactaataatatgcaataaatatt

SEQ ID NO: 76 - DNA, 70 bp, Homo sapiens
tttgggagagacttgttttggatgccccctaatccccttctcccctgcactgtaaaatgtgggattatgg

SEQ ID NO: 77 - DNA, 70 bp, Homo sapiens
ctccgtgagagcaaggatcctcctgtttaccctgtacctccaatgtctggcacttgtaggtgctcaaata

SEQ ID NO: 78 - DNA, 70 bp, Homo sapiens
ctcggagcagaacccaacctccgagcagtacatgctaagacttcaccagtcaaagcgaactactatactc

SEQ ID NO: 79 - DNA, 70 bp, Homo sapiens
gggactgccagcccctaactgaaatctgaagctttttatcgcttatttttcctcgccctgtctcctccct

SEQ ID NO: 80 - DNA, 70 bp, Homo sapiens
accacggctgtgctactcacggtcatgctggagggatgcagaaactaaatgaatccacagctacttactc

SEQ ID NO: 81 - DNA, 70 bp, Homo sapiens
ctccccagccacaaggagtagaaccagtagctcaaggaattgtttcacagcagttgcctgcagttagttc

SEQ ID NO: 82 - DNA, 70 bp, Homo sapiens
acgaggagacagggaaagtgaaggcccactcacagactgaccgagagaacctgcggatcgcgctccgcta

SEQ ID NO: 83 - DNA, 70 bp, Homo sapiens
cgaaatgaagtttatcataggaaaatcatctcttggtttggtgattcccccttggctctttttggcttac

SEQ ID NO: 84 - DNA, 70 bp, Homo sapiens
gctgaaagatgtactgcagtcagcttcagggcagcttcctgccacagcagcattaaatgaagttggaatt

SEQ ID NO: 85 - DNA, 70 bp, Homo sapiens
gtacttggagttgggacctcacctggctctcccttatctttccggctgccattttttcccctttctaact

SEQ ID NO: 86 - DNA, 70 bp, Homo sapiens
tagatctctaagcccctcctggaaccctcattttccccactctcaatgtcccagtgtccagcgtgactaa

SEQ ID NO: 87 - DNA, 70 bp, Homo sapiens
ccagggactgccccagctgtcctgggcacaagtctctccagcatctttgttcattgattcaacaaagtat

SEQ ID NO: 88 - DNA, 70 bp, Homo sapiens
aataatgcctggtcattgggtgacctgcgattgtcagaaagaggggaaggaagccaggttgatacagctg

SEQ ID NO: 89 - DNA, 70 bp, Homo sapiens
tacggccgcgcctttgtgttcctgtcttctctccaccaccaaaagcaaaagatgatttcccattcactgc

SEQ ID NO: 90 - DNA, 70 bp, Homo sapiens
atcatcatgtcctagcacagatggccccaagcaggggaagtacaatactgcaggctgcaaatccatgtca

SEQ ID NO: 91 - DNA, 70 bp, Homo sapiens
cccagactcaccggacaggataactgtggcctcttcattaaactgcaccgtgttcaccttctgagaaagt

SEQ ID NO: 92 - DNA, 70 bp, Homo sapiens
atttgcatctgaaaggtcccaaggtgaagggcgatgtggatgtttctctgcccaaagtggaaggtgacct

SEQ ID NO: 93 - DNA, 70 bp, Homo sapiens
gatggaaagagtctcacttgcagttgcttcagtcacaacccaggcgtctgccttaatagcatcacctgtg

SEQ ID NO: 94 - DNA, 70 bp, Homo sapiens
aatacagcaattttggcaataactcttatcactcctcagggcttagggtggtcccaggtacccaggggtc

SEQ ID NO: 95 - DNA, 70 bp, Homo sapiens
gaaatggtgcgttggtggtcatacttagtgttctaggctgtgaaatcatggagttcttccacttccaaga

SEQ ID NO: 96 - DNA, 70 bp, Homo sapiens
tgttgggccctgaaaaattagtccgattttgtggtggtaatgggagaaggacatcccaggagcagggtct

SEQ ID NO: 97 - DNA, 70 bp, Homo sapiens
ccatgaccctgaaactagaacaacacgtctcctccctaagtctgcagcttccagatcctcgaattgcaac

SEQ ID NO: 98 - DNA, 70 bp, Homo sapiens
ccctctgccaggcgctagacatgtacagaggtttttctgtggttgtaaatggtcctatttcacccccttc

SEQ ID NO: 99 - DNA, 70 bp, Homo sapiens
gggggagttgagcaggcgccagggctgtcatcaacatggatatgacatttcacaacagtgactagttgaa

SEQ ID NO: 100 - DNA, 70 bp, Homo sapiens
cccatccctaataggctgggctttgcaggaaatggcatgaaatcagctcttctgagtgcacagaagaacc

SEQ ID NO: 101 - DNA, 70 bp, Homo sapiens
gggggttccttcctgttgctaaggtttggaggtgttctgttatttacctgaagtgctgcagctgggaatc

SEQ ID NO: 102 - DNA, 70 bp, Homo sapiens
atcattgaaaggtcctctctgccagcagtggtgccaccctttggtttgctgtggtactttgctgtgtact

SEQ ID NO: 103 - DNA, 70 bp, Homo sapiens
gccggtttttccatgtcatacaaaaaagtcctggctgtttctccgaactggctgcctgcattcccgtctt

SEQ ID NO: 104 - DNA, 70 bp, Homo sapiens
ctgagaggaacctggacatggtcccgggcatctgaatgatctgtaggggagggagttcaaataaagcttt

SEQ ID NO: 105 - DNA, 70 bp, Homo sapiens
tcaaaaccctgagccctgtgcatgctttctcagtcttgtggtgggactggatacaatgactaacttcccc

SEQ ID NO: 106 - DNA, 70 bp, Homo sapiens
ggttgcactggggaggtctgggaagatagctgtttctgaagacttgccgctgtggacacagttaactaaa

SEQ ID NO: 107 - DNA, 70 bp, Homo sapiens
ggagaaagaagagctcgctgtgaaaaacgctccacaatgctgcagagccttgtgaaggtggaagagtact

SEQ ID NO: 108 - DNA, 70 bp, Homo sapiens
ataaagttgttacaaagtgaccttgagtgtcttccttggtgcacccgaaaccccgccttcttcatccggg

SEQ ID NO: 109 - DNA, 70 bp, Homo sapiens
agccagtcctgttggtggaggggatcaccgagagtgtctgtatcattttgtagcccttttctctgacgtt

SEQ ID NO: 110 - DNA, 70 bp, Homo sapiens
gggcatctgagggcagtaaggaacaggtgtccaaaggaggaatgttggtgcctatgagtatgttttccag

SEQ ID NO: 111 - DNA, 70 bp, Homo sapiens
aaatggccaccaccattctccttccccaccccaccacaaaaagagaagctgtgtctttagacaaccctga

SEQ ID NO: 112 - DNA, 70 bp, Homo sapiens
gcccgcagttggagttggactgtcttaacagtagcgtggcacacagaaggcactcagtaaatacttgttg

SEQ ID NO: 113 - DNA, 70 bp, Homo sapiens
ctctgaagcgagctggtttagttgtagaagatgctctgtttgaaactctgccttctgacgtccgggagca

SEQ ID NO: 114 - DNA, 70 bp, Homo sapiens
cccacctgtagatccatagcaacagtggatcagggcaggaagcaagcacataaagtggagtttcccttct

SEQ ID NO: 115 - DNA, 70 bp, Homo sapiens
ctgagcctagagcagggagtcccgaacttctgcattcacagaccacctccacaattgttataaccaaagg

SEQ ID NO: 116 - DNA, 70 bp, Homo sapiens
gcctggggaacgtggttggctcagggtttgacagagaaaagacaaataaatactgtattaataagatgtt

SEQ ID NO: 117 - DNA, 70 bp, Homo sapiens
gcaccgttaggtttcagatctcccgtgtggtgtttgatgtcggcttttgttcctaccttgggagtttgga

SEQ ID NO: 118 - DNA, 70 bp, Homo sapiens
gtaagtaacttgtgctagtcactgggggacctgggtttcagactgggcaatctggctgatcattttccag

SEQ ID NO: 119 - DNA, 70 bp, Homo sapiens
caccttggcctctgaaagtgctagaattacgggcatgagccaccgcatccagccagaaagatacatatct

SEQ ID NO: 120 - DNA, 70 bp, Homo sapiens
atccattctcacatttaaactactgtccagggccgggcgcagtgggtcacgtctgtaattccagcacttt

SEQ ID NO: 121 - DNA, 70 bp, Homo sapiens
gtttggactatagaaatgcggctgttcgctgcaaccaatcaaaaccctctgtggtttaggctagcgggct

SEQ ID NO: 122 - DNA, 70 bp, Homo sapiens
ggccaaagagaacaccagaagacccttaattttacaggcagagttgcctcaggccaatgactggctccaa

SEQ ID NO: 123 - DNA, 70 bp, Homo sapiens
cattctcctaaaggtgactccagtcctgtgctgagtcctgtgcattctcctaaaggtgactctagtcctg

SEQ ID NO: 124 - DNA, 70 bp, Homo sapiens
gcgtcaggagccggctgtgtccttcctgccacactcggggattcattccttagaaactgaaataaattct

SEQ ID NO: 125 - DNA, 70 bp, Homo sapiens
gggcactaatggagatactcatctggggtggagaagactttgaccagcgtgtcattggacacttcatcaa

SEQ ID NO: 126 - DNA, 70 bp, Homo sapiens
ccaggtctctgtagtacttggcaaacctgaaattgtagccaggagatacgttgtgctcaacgtcccgtgt

SEQ ID NO: 127 - DNA, 70 bp, Homo sapiens
ccataaaatgtttctcttctgaacaagccccatcatttggtgaacctccaccctaacaaagtaggatggg

SEQ ID NO: 128 - DNA, 70 bp, Homo sapiens
tcaagtggagcttcatgaataagccctcagatggcaggcccaagtatctggtggtgaacgcagacgaggg

SEQ ID NO: 129 - DNA, 70 bp, Homo sapiens
gctataggttgcagcttggctctatctgctgtctcaataacagcctttgaactgtccacgtatctyaaaa

SEQ ID NO: 130 - DNA, 70 bp, Homo sapiens
tcctccaggtttttcaattaaacggattattttttcagaccgaaaagagatggtctgagtttgtcttaga

SEQ ID NO: 131 - DNA, 70 bp, Homo sapiens
ctgctgccatgtgagtatgtgggcccagtgttgccagatcacctgcttttatacgaagaccctaaactct

SEQ ID NO: 132 - DNA, 70 bp, Homo sapiens
tataaaaattagccagtactaggaaggctgaggcaggataatcgctggaacccgggaggtggaggttgca

SEQ ID NO: 133 - DNA, 70 bp, Homo sapiens
aaaagaaattagctgcacattgtggtgagcgcctgtaatcccagctactcaggaggctgaggcaggagaa

SEQ ID NO: 134 - DNA, 70 bp, Homo sapiens
gcactctcaaatttctacgctcaaacaatccttccacctcaggctcctgagtagctgggactacaggcat

SEQ ID NO: 135 - DNA, 70 bp, Homo sapiens
aaaaaagagcccgcatcgccaagtcaatcttaagccaaaagaacaaagccagaggcatcacactacctga

SEQ ID NO: 136 - DNA, 70 bp, Homo sapiens
aattcccggttctcagaattgttatcactctggtgcatgctgtcacaggggccgttgcgtttggctttgt

SEQ ID NO: 137 - DNA, 70 bp, Homo sapiens
gtggatggatcacaaggtcaggagatcgagaccatcctggctaatacggtgaaaccccgtctctactaaa

SEQ ID NO: 138 - DNA, 70 bp, Homo sapiens
ctatcaaagggtggggtggtgccacctccgtgctgtgcaggagtcaaaaagttgaacggtatggctcaaa

SEQ ID NO: 139 - DNA, 70 bp, Homo sapiens
ttagtgcctgcacctcaccacgatattgaggaagcacaggacatccaagggtactctccagtttggctgt

SEQ ID NO: 140 - DNA, 70 bp, Homo sapiens
tcatcttttagagcagctgccatcacatcggacatattggaggcccttggaagagacggtcacttcacac

SEQ ID NO: 141 - DNA, 70 bp, Homo sapiens
caggccacctactcatgcacctaattggaagcgccaccctagcaatatcaaccattaaccttgcctctac

SEQ ID NO: 142 - DNA, 70 bp, Homo sapiens
  misc_feature (50)..(50): n is a, c, g, or t
  misc_feature (56)..(56): n is a, c, g, or t
aagcccctattttttccaagcacgaagccaccagtcttccccagggagcnatcagnagggacatggatgt

SEQ ID NO: 143 - DNA, 70 bp, Homo sapiens
ctgcgcctgaggggtggctgttaattcttcagtcatggcattcgcagtgcccagtgatggcattactctg

SEQ ID NO: 144 - DNA, 70 bp, Homo sapiens
taggcttaaaaacagatgcaattcccggacgtctaaaccaaaccactttcaccgctacacgaccgggggt

SEQ ID NO: 145 - DNA, 70 bp, Homo sapiens
ctggaaaagggacagactatcagagagttgcactgttgcggtatgggccaaatccaacataatacccgct

SEQ ID NO: 146 - DNA, 70 bp, Homo sapiens
tcgcaccactgcactccagcctgggcaacagagcaagactgtgtcttgacagcaacaaaaaaagaagata

SEQ ID NO: 147 - DNA, 70 bp, Homo sapiens
cgctgatttcctgaaatagagatacccctttgagtgataaatttgcaaaatgctgtcttcattttctgta

SEQ ID NO: 148 - DNA, 70 bp, Homo sapiens
ccgctggggaggtcctccatgcgcagtcatgagtcgcttcaagtttatcgtttatgattacaggtggaaa

SEQ ID NO: 149 - DNA, 70 bp, Homo sapiens
caaatacttttcctgcctccaccaaacccctacagaacatcacctggaattgccactcacactgggttgg

SEQ ID NO: 150 - DNA, 70 bp, Homo sapiens; identical to SEQ ID NO: 13

SEQ ID NO: 151 - DNA, 70 bp, Homo sapiens
ggatattctgttggtgataccaaacaccaaggggcctccagctgggtttcagtagtacgatgagtcactg

SEQ ID NO: 152 - DNA, 70 bp, Homo sapiens
catccaaacccagtcgtctgccctggatcgttttaatgccatgaactcagccttggcgtcagattccatt

SEQ ID NO: 153 - DNA, 70 bp, Homo sapiens
tttctttagacccatacttactgttcctcaaatgcctgcagtttgcccgggagtcgtctctgcaactggc

SEQ ID NO: 154 - DNA, 70 bp, Homo sapiens; identical to SEQ ID NO: 50

SEQ ID NO: 155 - DNA, 70 bp, Homo sapiens
gggtaccacccaagtattgactcacccatcaacaaccgccatgtatttcgtacattactgccagccacca

SEQ ID NO: 156 - DNA, 70 bp, Homo sapiens; identical to SEQ ID NO: 50

SEQ ID NO: 157 - DNA, 70 bp, Homo sapiens
taagcagtgatctttgctgctgctttccccctttgtctgcccttaggtcactaaggattgtagggccttc

SEQ ID NO: 158 - DNA, 70 bp, Homo sapiens
aagcacaagactgacctcaaccatgaaaacctcaagggtggagacgacctggaccccaactacgtgctca

SEQ ID NO: 159 - DNA, 70 bp, Homo sapiens
tgtgaggttttacagtattctgcaagggaagctcaagattcaaaaaaggtggtagaggacattgaatacc

SEQ ID NO: 160 - DNA, 70 bp, Homo sapiens
agaaatggatgtgggaacagatgaagaagaaacagcaaaggaatctacagctgaaaaagatgaattgttg

SEQ ID NO: 161 - DNA, 70 bp, Homo sapiens
cctacctgccaacctctcctctgctggcagattgtatcatccccattactgatatcagggctttcacaac

SEQ ID NO: 162 - DNA, 70 bp, Homo sapiens
aagtgtgacaacttgatctactagcgaggctgcatggggaaacaggcactttcataggtagctggtggga

SEQ ID NO: 163 - DNA, 70 bp, Homo sapiens
ttgtaatccaggacatctgatctcctacatcaaaaactccaatggggccaggtgtggtggcacttgcctg

SEQ ID NO: 164 - DNA, 70 bp, Homo sapiens
gccatgaaggcactgagtctgtctggtttcctgagggttaaaagattagggctgggatcaccacagcatt

SEQ ID NO: 165 - DNA, 70 bp, Homo sapiens
gacctccccagcatccctgaggtgtggctgcttagttttcgatacttaccttgttaccagatgtcagact

SEQ ID NO: 166 - DNA, 70 bp, Homo sapiens
gaattgcccagtgctgccagagtgagtgagtgtaattctcctttcaggtaaagataggctatctcaacac

SEQ ID NO: 167 - DNA, 70 bp, Homo sapiens
aaatagggctggatcttatcactgccctgtctccccttgtttctctgtgccagatcttcagtgccccttt

SEQ ID NO: 168 - DNA, 70 bp, Homo sapiens
atgtcataacttctgttactcctttggcccatagctaaggtcatccttccccacaggggtggctttggga

SEQ ID NO: 169 - DNA, 70 bp, Homo sapiens
cttgccagaagatgatcttagagttgttttctaaggtgccatccttggtaggaagctttattagaagcca

SEQ ID NO: 170 - DNA, 70 bp, Homo sapiens
ctggtcaccgtttcaccatcatgctttgatgttcccctgtctttccctcttctgctctcaagagcaaagg

SEQ ID NO: 171 - DNA, 70 bp, Homo sapiens
agagtgttgtccagatgtttctgtactggcatagaaaaaccaaataaaaggcctttatttttaaacaaaa

SEQ ID NO: 172 - DNA, 70 bp, Homo sapiens
aatggaaacatctgccccacgtgccggaagccaagtggtggcgacaactgcgcgccactccgcggcctac

SEQ ID NO: 173 - DNA, 70 bp, Homo sapiens
tagtgccactaacggttgagttttgactgcttggaactggaatcctttcagcaagacttctctttgcctc

SEQ ID NO: 174 - DNA, 70 bp, Homo sapiens
taatcctgccagtctttctcttcaagccagggtgcatcctcagaaacctactcaacacagcactctaggc

SEQ ID NO: 175 - DNA, 70 bp, Homo sapiens
tttcacatatgttgtgaattttccttggttctttttaaaggaatgataataaagttacttgctttaggaa

SEQ ID NO: 176 - DNA, 70 bp, Homo sapiens
gggtgtccgctgctgctttccttcggaatccagtgcttccacagagattagcctgtagcttatatttgac

SEQ ID NO: 177 - DNA, 70 bp, Homo sapiens
ctgttgcaactcggctgttctggactctgatgtgtgtggagggatggggaatagaacattgactgtgttg

SEQ ID NO: 178 - DNA, 70 bp, Homo sapiens
gattgctgtgtaccctgcctttgaagcacctcctcagtacgttttgccaacctatgaaatggccgtgaaa

SEQ ID NO: 179 - DNA, 70 bp, Homo sapiens
cacacgtagtggcttaaagcaacgaacattcactctctcacagtctgtgtcagtcggggatttgggagtg

SEQ ID NO: 180 - DNA, 70 bp, Homo sapiens
tcctctaactaggactccctcattcctagaaatttaaccttaatgaaatccctaataaaactcagtgctg

SEQ ID NO: 181 - DNA, 70 bp, Homo sapiens
gaaatgggtccctgggtgacatgtcagatctttgtacgtaattaaaaatattgtggcaggattaatagca

SEQ ID NO: 182 - DNA, 70 bp, Homo sapiens
attattgcaaatactatgggtaccgcaatccttcctgtgaggatgggcgccttcgggtgttgaagcctga

SEQ ID NO: 183 - DNA, 70 bp, Homo sapiens
aagctacactcaaagacactcccaccaggctctttctcccttttcctcttgctcactgccctggaatcaa

SEQ ID NO: 184 - DNA, 70 bp, Homo sapiens
acttgaaaaattacacctggcagctgcgtttaagccttcccccatcgtgtactgcagagttgagctggca

SEQ ID NO: 185 - DNA, 70 bp, Homo sapiens
ccaatcttttacaaagcatgggagtgcagctgcctgacaacaccgatcacagaccaacaagtaagccaac

SEQ ID NO: 186 - DNA, 70 bp, Homo sapiens
cccagctcatccagggagggcggcttatcaaacacgagatgactaaaacggcatctgcataacaatggaa

SEQ ID NO: 187 - DNA, 70 bp, Homo sapiens
atttggatctcacgctgcctctgtggttccctccctcatttttcctggacgtgatagctctgcctattac

SEQ ID NO: 188 - DNA, 70 bp, Homo sapiens
tggaggcctgtggtttccgcacccgctgccacccccgcccctagcgtggacatttatcctctagcgctca

SEQ ID NO: 189 - DNA, 70 bp, Homo sapiens
ggacaagaaagaaatggccatcaatgactgcagcaaagcaattcaattaaaccccagctatatcagggca

SEQ ID NO: 190 - DNA, 70 bp, Homo sapiens
gtccccaacctagcttggtgagggctgtaactgtttccaagtacttgtacattggaagtctgaatgtgta

SEQ ID NO: 191 - DNA, 70 bp, Homo sapiens
ggctggctaactcgtaggaagagagcactgtatggtatccttttgctttattcaccagcattttggggga

SEQ ID NO: 192 - DNA, 70 bp, Homo sapiens
gcggccggcatcatgaccctgtttcacttcgggaactgcttcgctcttgcctacttcccctacttcatca

SEQ ID NO: 193 - DNA, 70 bp, Homo sapiens
atcatgcatgaagcgccaaagatgcaccatgtagaattttcactttgtactggcaggctcgttttacctc

SEQ ID NO: 194 - DNA, 70 bp, Homo sapiens
tctcctctagaccaaggcaggcagccccgacatctgcttcctctatcgcccaatgcaaaatcgatgaaat

SEQ ID NO: 195 - DNA, 70 bp, Homo sapiens
ggtccggtgaccccctggccccagatggcactgagtttttcattcattgaagatttgatttccttgaaaa

SEQ ID NO: 196 - DNA, 70 bp, Homo sapiens
caaggtactctggtgagtcaccacttcagggctttactccgtaacagattttgttggcatagctctgggg

SEQ ID NO: 197 - DNA, 70 bp, Homo sapiens
gaaattagggcctcctctgatctctcgctatctgcgggtcctgtccttttctcaagaccttcaccattac

SEQ ID NO: 198 - DNA, 70 bp, Homo sapiens
ccttccttgccaggacctagagtttgttcagttccaccccacaggcatatatggtgctggttgtctcatt

SEQ ID NO: 199 - DNA, 70 bp, Homo sapiens
gaagatggagacaccctctgggggtcctctctgagtcaaatccagtggtgggtaattgtacaataaattt

SEQ ID NO: 200 - DNA, 70 bp, Homo sapiens
gtgtagggaaaaggatccactgggtgaatcctccctctcagaaccaataaaatagaattgaccttttaaa

SEQ ID NO: 201 - DNA, 70 bp, Homo sapiens
tcaggctttctgtgcatgtactaaaaaaggagaaattataataaattagccgtcttgcggcccctaggcc

SEQ ID NO: 202 - DNA, 70 bp, Homo sapiens
ggtgctaggagaggatggtctccacccatctttctatttccagtacacgtcacattattttaccggtgag

SEQ ID NO: 203 - DNA, 70 bp, Homo sapiens
ggccaaaaacatacagaggtgcatggctggcagtcttgaaattgtcactcgcttactggatccaagcgtc

SEQ ID NO: 204 - DNA, 70 bp, Homo sapiens
tttcccctgctcggaagggttggcctgcctggctggggaggtcagtaaactttgaatagtaagccaaaaa

SEQ ID NO: 205 - DNA, 70 bp, Homo sapiens
aacatggtattaaactctataaacctctcattctccctgtgactcaggccccaatcttcatctccttctt

SEQ ID NO: 206 - DNA, 70 bp, Homo sapiens
ggcactgtgcatattttcaaccagatcaccaggagctgagatcttcttcagtccctagccaggaataccc

SEQ ID NO: 207 - DNA, 70 bp, Homo sapiens
aattcggcacgaggcccgacgctgtggttgctgtaaggggtcctccctgcgccacacggccgtcgccatg

SEQ ID NO: 208 - DNA, 70 bp, Homo sapiens
agatggacgtgcacattactccggggacccatgcctcagagcatgcagtgaacaagcaacttgcagataa

SEQ ID NO: 209 - DNA, 70 bp, Homo sapiens
actgaggggcaagattagcgagcaggacaaaaacaagatcctcgacaagtgtcaggaggtgatcaactgg

SEQ ID NO: 210 - DNA, 70 bp, Homo sapiens
gccaccagagactgagtggaaatcgccccttttgaaggtgccattcttatgagccaaaagtttgtcattt

SEQ ID NO: 211 - DNA, 70 bp, Homo sapiens
cagtatgagaaaaatattcaagtaacactttaaaaccagttacccaaaatctgattagaagtataaggtg

SEQ ID NO: 212 - DNA, 70 bp, Homo sapiens
cggccatgcctttcttggacatccagaaaaggttcggccttaacatagatcgatggttgacaatccagag

SEQ ID NO: 213 - DNA, 70 bp, Homo sapiens
gaggttgctcagctcaagaaaagtgcagataccctgtgggacatccagaaggacctaaaagacctgtgac

SEQ ID NO: 214 - DNA, 70 bp, Homo sapiens
caaaggaaatcagcagtgatagatgaagggttcgcagcgagagtcccggacttgtctagaaatgagcagg

SEQ ID NO: 215 - DNA, 70 bp, Homo sapiens
tatcagaggtgtggaagaagaggaagaagatggggaaatgagagaatagcatcttttgtgggggattttt

SEQ ID NO: 216 - DNA, 70 bp, Homo sapiens
gaacaagtggttcttccagaaactgcggttttagatgctttgttttgatcattaaaaattataaagaaaa

SEQ ID NO: 217 - DNA, 70 bp, Homo sapiens
agggatccactgtgcggtgccaaaaaagaggcggaggctcgcggcacagctctcccggcgcagctctcgg

SEQ ID NO: 218 - DNA, 70 bp, Homo sapiens
aatgttctccgaaacaggatcaacgataaccagaaagtctccaagacccgcgggaaggctaaagtcaccg

SEQ ID NO: 219 - DNA, 70 bp, Homo sapiens
agttccttcttgaaccctggtgcctcctaccctatggccctgaatggtgcactggtttaattgtgttggt

SEQ ID NO: 220 - DNA, 70 bp, Homo sapiens
aggttttcattcgcacggaacaccttttggcatgcttaacttcctggtaacaccttcacctgcattggtt

SEQ ID NO: 221 - DNA, 70 bp, Homo sapiens
ccagcccttaaaatgaaattaacttcctactcaggcaccctgcttaggtgcacagctgttcaatatacac

SEQ ID NO: 222 - DNA, 70 bp, Homo sapiens
aggacagtcatcagaggctctcaggctgagctcaagtgccccgtgtgtcttttggaatttgaggaggagg

SEQ ID NO: 223 - DNA, 70 bp, Homo sapiens
tgacgacttcgccgcgcgttggtcagccatggccaccgctctcgcgctacgtagcttgtaccgagcgcga

SEQ ID NO: 224 - DNA, 70 bp, Homo sapiens
gcctagagccttcagtcactggggaaagcagggaagcagtgtgaactctttattcactcccagcctgtcc

SEQ ID NO: 225 - DNA, 70 bp, Homo sapiens
attatatccccattaaggcaactgctacaccctgctttgtattctgggctaagattcattaaaaactagc

SEQ ID NO: 226 - DNA, 70 bp, Homo sapiens
ccttctgtgacatgtgtttataaaaaatggttaagtatataataaattgaacatctttgagattggagaa

SEQ ID NO: 227 - DNA, 70 bp, Homo sapiens
gccgccatgggagtggagggctgcaccaagtgcatcaagtacctgctcttcgtcttcaatttcgtcttct

SEQ ID NO: 228 - DNA, 70 bp, Homo sapiens
cccttggggaggggccacctgtagtatttgccttgatttggtggggtacagtggatgtgaatactgtaaa

SEQ ID NO: 229 - DNA, 70 bp, Homo sapiens
ctatggttggatctcagctggaagttctgtttggagcccatttctgtgagaccctgtatttcaaatttgc

SEQ ID NO: 230 - DNA, 70 bp, Homo sapiens
gggaccctgttacagacataccctatgccactgctcgagccttcaagatcattcgtgaggcttacaagaa

SEQ ID NO: 231 - DNA, 70 bp, Homo sapiens
ttaaggaacgctagcagggcatggcacgtgagctccggaatagatgtcttcatcacttcttccactgtgt

SEQ ID NO: 232 - DNA, 70 bp, Homo sapiens
aatgtctgtcagtaacgaggcttttgatgtgttgagctggaggtgagtggaccgggggctgtgttttaag

SEQ ID NO: 233 - DNA, 70 bp, Homo sapiens
gcgccgctgagttgtctggccccgccgacccacggcccacgacccaccgacccacgaatcggcccggccg

SEQ ID NO: 234 - DNA, 70 bp, Homo sapiens
ccgatactcccagatctgtgcaaaagcagtgagagatgcactgaagacagaattcaaagcaaatgctgag

SEQ ID NO: 235 - DNA, 70 bp, Homo sapiens
gcaccctcctgaaaactgcagcttccttctcaccttgaagaataatcctagaaaactcacaaaatgtgtg

SEQ ID NO: 236 - DNA, 70 bp, Homo sapiens
ggagtttctgactaatcaaagctggtatttccccgcatgtcttattcttgcccttcccccaaccagtttg

SEQ ID NO: 237 - DNA, 70 bp, Homo sapiens
atttacaagacaggttttaactcagccgaggtgggaaatggtgtccctgtccctcccaaagcacagagca

SEQ ID NO: 238 - DNA, 70 bp, Homo sapiens
ccttgcttctgactttcgcctctgggacaagtaagtcaatgtgggcagttcagtcgtctgggttttttcc

SEQ ID NO: 239 - DNA, 70 bp, Homo sapiens
tgaagcagatgatgaaaactctcaacaacgacctgggccccaactggcgggacaagttggaatacttcga

SEQ ID NO: 240 - DNA, 70 bp, Homo sapiens
tcatttcctcaatgggacggagcgagtgtggaacctgatcagatacatctataaccaagaggagtacgcg

SEQ ID NO: 241 - DNA, 70 bp, Homo sapiens
ctgcagagaagaaacctactacagaggagaagaagcctgctgcataaactcttaaatttgattattccat

SEQ ID NO: 242 - DNA, 70 bp, Homo sapiens
gaccatttggaagaaaagatgcctttagaagatgaggtcgtgcccccacaagtgctcagtgagccgaatg

SEQ ID NO: 243 - DNA, 70 bp, Homo sapiens
gaatgagacatccagcagatttccagccttctactgctctcctccacctcaactccgtgcttaaccaaag

SEQ ID NO: 244 - DNA, 70 bp, Homo sapiens
tagttcttcaccttttaaattatgtcactaaactttgtatgagttcaaataaatatttgactaaatgaaa

SEQ ID NO: 245 - DNA, 70 bp, Homo sapiens
ccgaggaagatactgagggagcacaggagcagtcaccgctgccactgctactgccgctactgctgccggc

SEQ ID NO: 246 - DNA, 70 bp, Homo sapiens
ggggcagcactgggcctggccccccgggtatttattgctgtacatagtgtatgtttgtgatatataaggt

SEQ ID NO: 247 - DNA, 70 bp, Homo sapiens
caaacattagatcctaacaatatgaccatactcaataggacttttcaagatgagccactaattatggatt

SEQ ID NO: 248 - DNA, 70 bp, Homo sapiens
gaggggaagccacttaataaggagtcagacctaaaagggggtgggggacattttcttacctcacccaaga

SEQ ID NO: 249 - DNA, 70 bp, Homo sapiens
aatccactcacgttcataaagagaatgttgatggcgccgtgtagaagccgctctgtatccatccacgcgt

SEQ ID NO: 250 - DNA, 70 bp, Homo sapiens
gccatcctaagattaggacttcttcttgactgcccgagactcgccatttctgcccgtgaatttgtgtctg

SEQ ID NO: 251 - DNA, 70 bp, Homo sapiens
taaagcaaggggaccttggcactctcagctttccctgccacatccagcttgttgtcccaatgaaatactg

SEQ ID NO: 252 - DNA, 70 bp, Homo sapiens
gagggctcactgagaaccatcccagtaacccgaccgccgctggtcttcgctggacaccatgaatcacact

SEQ ID NO: 253 - DNA, 70 bp, Homo sapiens
ctatgaatctttgtgagcaattatgctcccaaatctaagcaagtaaaatacacattttgtctttcttaaa

SEQ ID NO: 254 - DNA, 70 bp, Homo sapiens
acaacaggcatttaagcaatgaagatatgtttagagaagtggatgaaatagatgagataaggagagtcag

SEQ ID NO: 255 - DNA, 70 bp, Homo sapiens
taaccaggccagtgacagaaatggattcgaaataccagtgtgtgaagctgaatgatggtcacttcatgcc

SEQ ID NO: 256 - DNA, 70 bp, Homo sapiens
aatctggcagccagttccgtcctgacagagttcacagcatatattggtggattcttgtccatagtgcatc

SEQ ID NO: 257 - DNA, 70 bp, Homo sapiens
ctgccccctgaaacttatttttttctgattgtaacgttgctgtgggaacgagaggggaagagtgtactgg

SEQ ID NO: 258 - DNA, 70 bp, Homo sapiens
aacaaatggtacagtcataagagccatctgtcacggacccacgcccagaggaacgtgcagaaaaaagcag

SEQ ID NO: 259 - DNA, 70 bp, Homo sapiens
aggatagttggcttcctgcctctctcctctaaaatagcaagtctgggaaatcctggggtgagtggagtca

SEQ ID NO: 260 - DNA, 70 bp, Homo sapiens
tgcgattggttcttctgccatggcttcaacaagtggcctagtaatcacctctccttccaacctcagtgac

SEQ ID NO: 261 - DNA, 70 bp, Homo sapiens
gctcccagcacactcggagcttgtgctttgtctccacgcaaagcgataaataaaagcattggtggcctta

SEQ ID NO: 262 - DNA, 70 bp, Homo sapiens
ccacatatatgcgaatctataagaaaggtgatattgtagacatcaagggaatgggtactgttcaaaaagg

SEQ ID NO: 263 - DNA, 70 bp, Homo sapiens
ttcagtcagcctcagaggttgacttctacattgataaggacatgatccacatcgcggacaccaaggtcgc

SEQ ID NO: 264 - DNA, 70 bp, Homo sapiens
ccttccattttcccccactactgcagcacctccaggcctgttgctatagagcctacctgtatgtcaataa

SEQ ID NO: 265 - DNA, 70 bp, Homo sapiens
gcacctctagtgctactgctagatatcacttactcagttagaattttcctaaaaataagctttatttatt

SEQ ID NO: 266 - DNA, 70 bp, Homo sapiens
acgctcactgcctggcttggaaaagttaagaagcccctcaggaagagaatcgaggccaagttcctctgcg

SEQ ID NO: 267 - DNA, 70 bp, Homo sapiens
ggcttttgaatcgtaatagcaatgtgagggtgaggtacacctacagacattaaataatttgctgtgaaaa

SEQ ID NO: 268 - DNA, 70 bp, Homo sapiens
tcgcctacacaattctccgatccgtccctaacaaactaggaggcgtccttgccctattactatccatcct

SEQ ID NO: 269 - DNA, 70 bp, Homo sapiens
ttcatctctggatgacaagccccagttcccaggggcctcggcggagtttatagataagttggaattcatc

SEQ ID NO: 270 - DNA, 70 bp, Homo sapiens
gcactgctctcagactatgttctccacaacagcaacaccatgagacttggttccatctttgggctaggct

SEQ ID NO: 271 - DNA, 70 bp, Homo sapiens
aagtggtggaatcggctatccataccctcgtgcccctgtttttcctggccgtggtagttactcaaacaga

SEQ ID NO: 272 - DNA, 70 bp, Homo sapiens
gtttaacactaaaccaaggtcatgagcattcgtgctaagataacagactccagctcctggtccacccggc

SEQ ID NO: 273 - DNA, 70 bp, Homo sapiens
tagtgtcagtcaccaaagaaggcctggaacttccagaggatgaagaagagaaaaagaagcaggaagagaa

SEQ ID NO: 274 - DNA, 70 bp, Homo sapiens
cccactgtctggggcagggggagaaggtattttcgagataaagcacaggcaccacaaataaaagtcgtga

SEQ ID NO: 275 - DNA, 70 bp, Homo sapiens
gaggtaatctgggtgcacagaatttatctgagtctgctgctgtgaaggagatactgaaggagcaggaaaa

SEQ ID NO: 276 - DNA, 70 bp, Homo sapiens
acagtcatgcgcagggacgatccttgttctctgctgtaaactgtaaaaagtttatggagacttaaagtct

SEQ ID NO: 277 - DNA, 70 bp, Homo sapiens
tactggaacagccagaaggacatcctggaagacaagcgggccgcggtggacacctactgcagacacaact

SEQ ID NO: 278 - DNA, 70 bp, Homo sapiens
cacctgaggtcgggagttcgcgaccagcctgaccaacatggaaaaaccccgtctctactaaaaatacaaa

SEQ ID NO: 279 - DNA, 70 bp, Homo sapiens
agtcgggctacccactgattttccttcccttacttcccctgagcccttgggcccacttcccagcctaccg

SEQ ID NO: 280 - DNA, 70 bp, Homo sapiens
cagagaaacggcaggaagacccttactactgtccaagggatcgctgatgattacgataaaaagaaactag

SEQ ID NO: 281 - DNA, 70 bp, Homo sapiens
ccccatcttaactgatttaacccctgaaacaacccgacgctggaagttgggttctcatccccactctaca

SEQ ID NO: 282 - DNA, 70 bp, Homo sapiens
tgggctaccatctgcatggggctggggtcctcctgtgctatttgtacaaataaacctgaggcaggaaaaa

SEQ ID NO: 283 - DNA, 70 bp, Homo sapiens
gggcccaattcttctccacgacaatgcccgaccgcatgttgcacaacccacacttcaaaagttgaatgaa

SEQ ID NO: 284 - DNA, 70 bp, Homo sapiens
cacgtctgacagccatgtccacctgtgcccacagcttccgcccacagacctccagggacaggagcaaatt

SEQ ID NO: 285 - DNA, 70 bp, Homo sapiens
ttaaaaaagttgggttttctccattcaggattctgttccttaggattttttccttctgaagtgtttcacg

SEQ ID NO: 286 - DNA, 70 bp, Homo sapiens
gaggggaggggcctagggagccgcaccttgtcatgtaccatcaataaagtaccctgtgctcaaccaaaaa

SEQ ID NO: 287 - DNA, 70 bp, Homo sapiens
ggatactgcgagtatggcggcgtcaaaggtgaagcaggacatgcctccgccggggggctatgggcccatc

SEQ ID NO: 288 - DNA, 70 bp, Homo sapiens
ttaggttaggagttcatagttggaaaacttgtgcccttgtatagtgtccccatgggctcccactgcagcc

SEQ ID NO: 289 - DNA, 70 bp, Homo sapiens
ggcctcaagaggtttggagcaggtatgttaagaagttaggggattttgctaagccggagaatattgactt

SEQ ID NO: 290 - DNA, 70 bp, Homo sapiens
gatcttccctgtctcacacttcttttctcccatcccggttgcaatctcactcagacatcacagtaccacc

SEQ ID NO: 291 - DNA, 70 bp, Homo sapiens
acagattgttcctcccattccccttgccgctttttgcctatcgatgggtagcaagagtctttgaaataag

SEQ ID NO: 292 - DNA, 70 bp, Homo sapiens
gggcccccagcctcatctccggctccagcccctaagttttctccagtgactcctaagtttactcctgtgg

SEQ ID NO: 293 - DNA, 70 bp, Homo sapiens
ccttcagctaatttctgctcccctgagattcgtccttcagccccatcatgtgctttgggatgagtgtaaa

SEQ ID NO: 294 - DNA, 70 bp, Homo sapiens
agtggcccatctttgttggcctacgaactttggtttgatgccagtcaggtgccacatgagaacctttgct

SEQ ID NO: 295 - DNA, 70 bp, Homo sapiens
ccccctgccctcccctctctgcaccgtactgtggaaaagaaacacgcacttagtctctaaagagtttatt

SEQ ID NO: 296 - DNA, 70 bp, Homo sapiens
acaaatgcgacgaacctctgaacatcctggtgaggaataacaagggccgcagcagcacctacgaggtgcg

SEQ ID NO: 297 - DNA, 70 bp, Homo sapiens
tagcccaggctgtggaggggcccagtgagaatgtcaggaagctgtctcgtgcagacttgaccgagtacct

SEQ ID NO: 298 - DNA, 70 bp, Homo sapiens
ctcaattttgtgaggctgtgttggaaataacccgcctctagtgctgttggtatgcaaggcagcggtgctt

SEQ ID NO: 299 - DNA, 70 bp, Homo sapiens
tgctcaaattaccctccaaaagcaagtagccaaagccgttgccaaaccccacccataaatcaatgggccc

SEQ ID NO: 300 - DNA, 70 bp, Homo sapiens
gactccgctgggagagtgcaggagcacgtgctgttttttatttggacttaacttcagagaaaccgctgac

SEQ ID NO: 301 - DNA, 70 bp, Homo sapiens
cgcagcttagagagactcaccagcgagcgtcattgttgtctttctgggaactcattcccatgagatcaga

SEQ ID NO: 302 - DNA, 70 bp, Homo sapiens
cagtggaactgtcccacaagaattcacaggtctcaaagcaggaacagtgggtttgtgtctcacctgagta

SEQ ID NO: 303 - DNA, 70 bp, Homo sapiens
agctagtgccgactcccgcctagctcttttgactctgttcgcgggaagaatggggaaacagtaaggttgc

SEQ ID NO: 304 - DNA, 70 bp, Homo sapiens
acactgtttggaagaaagctaaaccctgaagatcagtagcccctaatcacatgtgctgcaaatagccttc

SEQ ID NO: 305 - DNA, 70 bp, Homo sapiens
ttgtggtcggggagctggggtacaggtttggggagggggaagagaaatttttatttttgaacccctgtgt

SEQ ID NO: 306 - DNA, 70 bp, Homo sapiens
gatctggttacctgtgcagttgtgaatacccagaggttgggcagatcagtgtctctagtcctacccagtt

SEQ ID NO: 307 - DNA, 70 bp, Homo sapiens
tgctccaactgaccctgtccatcagcgttctataaagcggccctcctggagccagccacccagagcccgc

SEQ ID NO: 308 - DNA, 70 bp, Homo sapiens
ccccgcttccccagtctttaaacattggacgctatttactcagctacccagtagagcttgaagctgacct

SEQ ID NO: 309 - DNA, 70 bp, Homo sapiens
ctttcagtctttatgtcacctcaggagacttatttgagaggaagccttctgtacttgaagttgatttgaa

SEQ ID NO: 310 - DNA, 70 bp, Homo sapiens
aggcccctgctggattggcaggccctgtccgaggagttgggggaccatcccagcaggtaatgactccaca

SEQ ID NO: 311 - DNA, 70 bp, Homo sapiens
aagacagcgccgcccgcgcaccgccagcgacccccgccgcagagtcccaccgccacaggcctcgggccag

SEQ ID NO: 312 - DNA, 70 bp, Homo sapiens
tctgttctgtttgtacatggctgacggaaatctctttggtacaaccgaataaagcctggtggcagtgctg

SEQ ID NO: 313 - DNA, 70 bp, Homo sapiens
ccaagtaccataggacagtcacataggagcgtgtagtcgtgactgaataaagaaagcaaaagcctgaaaa

SEQ ID NO: 314 - DNA, 70 bp, Homo sapiens
agtggctaaattgcagtagcagcatatctttttttctttgcacaaataaacagtgaattctcgtttaaaa

SEQ ID NO: 315 - DNA, 70 bp, Homo sapiens
aagcatcttgcttggttgctacattctggtgtgatgggtgcagtggtggctcctctgacaatattagggg

SEQ ID NO: 316 - DNA, 70 bp, Homo sapiens
ttttaattggagaagggtatagaggtagtccaggtgggaacgccagaagtgctgattgcccagccattgg

SEQ ID NO: 317 - DNA, 70 bp, Homo sapiens
aagcctgtgagatcttgtgttgcagcgtggtttggccctagcgttcttgcatgctaacctaaggtagaag

SEQ ID NO: 318 - DNA, 70 bp, Homo sapiens
aaaagcgtacaaaagatacttaaaagggctcctggggtacacaagcccagcaggtcctgagtgaagccgt

SEQ ID NO: 319 - DNA, 70 bp, Homo sapiens
aagacctggaccagtctcctctggtctcgtcctcggacagcccaccccggccgcagcccgcgttcaagta

SEQ ID NO: 320 - DNA, 70 bp, Homo sapiens
ggggcatgcaccctcctttctgtaccgtgtgtgctggctccatagttctctcttctgtacatataagcat

SEQ ID NO: 321 - DNA, 70 bp, Homo sapiens
gaaggctcagcctcaagattcacagcatctcagacacagcctaggccgcaccaggatgtcggacaccgag

SEQ ID NO: 322 - DNA, 70 bp, Homo sapiens; identical to SEQ ID NO: 99

SEQ ID NO: 323 - DNA, 70 bp, Homo sapiens
tcagccagcaccaagccttgttgggcactatcagggctgagggaaagatctcagaacaatcagatgcaaa

SEQ ID NO: 324 - DNA, 70 bp, Homo sapiens
ggccacgggaacaggaccatggttaagcaaccatatagaaagctttgttgaaagaaagtatggcatcttg

SEQ ID NO: 325 - DNA, 70 bp, Homo sapiens
acaacttggagaaatttggaaaactcagtgcgttccccgaacctcctgaggatgggacgctgctatcgga

SEQ ID NO: 326 - DNA, 70 bp, Homo sapiens
cccatggggggtggatgatttgcactttggttccctgtgttttgatttctcattaaagttcctttccttc

SEQ ID NO: 327 - DNA, 70 bp, Homo sapiens
tgggtcctgggaatgctgctgcttcaaccccagagcctaagaatggcagccgtttcttaacatgttgaga

SEQ ID NO: 328 - DNA, 70 bp, Homo sapiens
tggcaaaaacggccaggtacaacacctttttcatacaaggcccaggaggcttagtccagtctgtgctcct

SEQ ID NO: 329 - DNA, 70 bp, Homo sapiens
gcccactgtagtatccacagtgcccgagttctcgctggttttggcaattaaacctccttcctactggttt

SEQ ID NO: 330 - DNA, 70 bp, Homo sapiens
gagcaaaagaccgtgagtcccctagaagttactcatccactttgactgacatggggagaagtgcaccaag

SEQ ID NO: 331 - DNA, 70 bp, Homo sapiens
ggggtgagtgtagttctggcctagcagcaccctcttgtggcttgttctagcgtgtattaaaacttgacac

SEQ ID NO: 332 - DNA, 70 bp, Homo sapiens
gtgtgagagtgtgaatgcacaggtgggtatttaatctgtattattccccgttcttggaattttcttcccc

SEQ ID NO: 333 - DNA, 70 bp, Homo sapiens
gccttccctcagtgatgggttcagttccggaaggtgtcttagaggacattaaagcgcgtacttgctttgt

SEQ ID NO: 334 - DNA, 70 bp, Homo sapiens
ccatatgtcactgggggaaaggctgcctgtacctctcaagctttgcattttactggaaactgaggcgtca

SEQ ID NO: 335 - DNA, 70 bp, Homo sapiens
agaatacagttgtctagccaagccatcaagtgtctgaaattcaatattggtttatgcaaatacagcaaac

SEQ ID NO: 336 - DNA, 70 bp, Homo sapiens
cggctctggttgttggcagctttggggctgtttttgagcttctcattgtgtagaatttctagatcccccg

SEQ ID NO: 337 - DNA, 70 bp, Homo sapiens
tggtggcataattggagccttgctgggcactcctgtaggaggcctgctgatggcatttcagaagtactct

SEQ ID NO: 338 - DNA, 70 bp, Homo sapiens
atacggtgttttctgtccctcctactttccttcacaccagacagcccctcatgtctccaggacaggacag

SEQ ID NO: 339 - DNA, 70 bp, Homo sapiens
cccttatctgctaccctgaatcacctgtcctggtcttgctgtgtgatgggaacatgcttgtaaactgcgt

SEQ ID NO: 340 - DNA, 70 bp, Homo sapiens
cctagcgcgcggggggcgccccccagcccggaggctggctttgctacagctgaccactccggtcaggaga

SEQ ID NO: 341 - DNA, 70 bp, Homo sapiens
gtgaagtgttgcaggttgtgaactctgtagacatctttattgcttggctaagagtagatttaataaatgt

SEQ ID NO: 342 - DNA, 70 bp, Homo sapiens
ccccatagtcaggtgtaccagccagccaaaccaacaccacttcctagaaaaagatcagaagctagtcctc

SEQ ID NO: 343 - DNA, 70 bp, Homo sapiens
gttgctgccatcgtaaactgacacagtgtttataacgtgtacatacattaacttattacctcattttgtt

SEQ ID NO: 344 - DNA, 70 bp, Homo sapiens
ggggaccagcagataaatcccacccttccttgagctgtcgctgtactctgaagttcagccagctcagatt

SEQ ID NO: 345 - DNA, 70 bp, Homo sapiens
gagaaggacaaaatcaccaccaggacactgaaggcccgaatggactaaccctgttcccagagcccacttt

SEQ ID NO: 346 - DNA, 70 bp, Homo sapiens
atctatgatgacgattttttccaaaacctagatggcgtggccaatgccctggacaacgtggatgcccgca

SEQ ID NO: 347 - DNA, 70 bp, Homo sapiens
gcttggagtgaaagtgactctcaggtggtggggtggggaatgtgaataaacatgatttcttgccgggcaa

SEQ ID NO: 348 - DNA, 70 bp, Homo sapiens
gcgtttaaaataaaatatgcaacaaaatggatgacttagtggagatggaagcccattaattgggttcccc

SEQ ID NO: 349 - DNA, 70 bp, Homo sapiens
cactccctaatcccctacccctgtctccccttcaaggacttctcccttgtggttttgtaaagtgcaaact

SEQ ID NO: 350 - DNA, 70 bp, Homo sapiens
gtgaatttttgcacattctacacacagtgcctgtaaatctcatttgtattttcagtttgcccttaatttt

SEQ ID NO: 351 - DNA, 70 bp, Homo sapiens
tttttactccccttcagccccccggctgatgccatctctggttctggacaattatcaaatatatcagtgg

SEQ ID NO: 352 - DNA, 70 bp, Homo sapiens
ggatatagaccacgattccgcaggggccctcctcgccaaagacagcctagagaggacggcaatgaagaag

SEQ ID NO: 353 - DNA, 70 bp, Homo sapiens
caggagggcagtggtggagctggacctgcctgctgcagtcacgtgtaaacaggattattattagtgtttt

SEQ ID NO: 354 - DNA, 70 bp, Homo sapiens
tatttgacagtgtaggaaattgtctattcctgatataattactgtagtactcttgcttaaggcaagagtt

SEQ ID NO: 355 - DNA, 70 bp, Homo sapiens
cgaaggagttgcggttgctccatgttctgacttagggcaatttgattctgcacttggggtctgtctgtac

SEQ ID NO: 356 - DNA, 70 bp, Homo sapiens
cattatgacctgctagagaagaacattaacattgttcgcaaacgactgaaccggccgctgaccctctcgg

SEQ ID NO: 357 - DNA, 70 bp, Homo sapiens
taatttgtaagttatgttagcgggatcctcaaggccttgctttgccccgtggagacgcttgctcggatga

SEQ ID NO: 358 - DNA, 70 bp, Homo sapiens
gcacagatgaaactgagctgggactggaaaggacagcccttgacctgggttctgggtataatttgcactt

SEQ ID NO: 359 - DNA, 70 bp, Homo sapiens
gagacagagtaatttgcagtttgtttgatttatacttttgtttatctacaacccaataacagacatgagg

SEQ ID NO: 360 - DNA, 70 bp, Homo sapiens
ctggggaagcatttgactatctggaacttgtgtgtgcctcctcaggtatggcagtgactcacctggtttt

SEQ ID NO: 361 - DNA, 70 bp, Homo sapiens
gccaaggggccagctgcccctcatttatcactctgaccttcacagggacagatctgatttatttattttg

SEQ ID NO: 362 - DNA, 70 bp, Homo sapiens
gtgggagcagcagagatgtccagggtacagatgcaagtcttgatgaggaacttgatcgagtcaagatgag

SEQ ID NO: 363 - DNA, 70 bp, Homo sapiens
taaaggcccgggagcggctagagctctgtgatgagcgtgtatcctctcgatcacatacagaagaggattg

SEQ ID NO: 364 - DNA, 70 bp, Homo sapiens; identical to SEQ ID NO: 128

SEQ ID NO: 365 - DNA, 70 bp, Homo sapiens
taaaaacaccttgggggcaggcaggggcatttaaaaatgtaggacctatcgtccagactcacagagtggg

SEQ ID NO: 366 - DNA, 70 bp, Homo sapiens
gtggctttccttactgcgaagaatgctaagacccctcagcaggaggagacaacttactaccaaacagcac

SEQ ID NO: 367 - DNA, 70 bp, Homo sapiens; identical to SEQ ID NO: 36

SEQ ID NO: 368 - DNA, 70 bp, Homo sapiens
tcccttctgggttccgaggcccaagcccttggcagtgtttgtgagtggaagggaggtcacgctatcgtcc

SEQ ID NO: 369 - DNA, 70 bp, Homo sapiens
ttatttcccttccacagtgtggtttcttcctctgcggtaaaggacttggtctgttctaccccctgctcca

SEQ ID NO: 370 - DNA, 70 bp, Homo sapiens
gagcattcatcgtgaggggtctttgtcctctgtactgtctctctccttgcccctaacccaaaaagcttca

SEQ ID NO: 371 - DNA, 70 bp, Homo sapiens
ttcatcaagaaccacgcctttcgcctgctgaagccggggggcgtcctcacctactgcaacctcacctcct

SEQ ID NO: 372 - DNA, 70 bp, Homo sapiens
ctgtgaaaataccccctttctccattagtggcatgctcattcagctcttatctttatattccagtaagtt

SEQ ID NO: 373 - DNA, 70 bp, Homo sapiens
cgtccacggactctccgttattttaggaggtccctggccaaagatttatttctcttgacaaccaagggcc

SEQ ID NO: 374 - DNA, 70 bp, Homo sapiens
cgatgagaaggtttactacactgcaggctacaacagtcctgtcaaattgcttaatagaaataatgaagtg

SEQ ID NO: 375 - DNA, 70 bp, Homo sapiens
tgggtgatctctttgctgaattaatgagttcttaacatgtggacccaactgcctgtgtgagatctgtgtc

SEQ ID NO: 376 - DNA, 70 bp, Homo sapiens
ctcacagcggcccgcgggccgggcgtcatgggcggcctcttctggcgctccgcgctgcgggggctgcgct

SEQ ID NO: 377 - DNA, 70 bp, Homo sapiens
tgatcccgcacggcacatcactggggagaagctcggagagctgtataagagctttatcaagaactatcct

SEQ ID NO: 378 - DNA, 70 bp, Homo sapiens
aatacacatttgaaaatttccagtatcaatctagagcgcaaataaatcacagtattgctatgcagaatgg

SEQ ID NO: 379 - DNA, 70 bp, Homo sapiens
ctcatccacagaaagggaggatgggcgatgacagttgtttctatgccttctgacccagtttcccagttta

SEQ ID NO: 380 - DNA, 70 bp, Homo sapiens
acgtctggtaggaagattgttagtgcctcaagttacacctgtgcagcttgggtctgagttttgatagaac

SEQ ID NO: 381 - DNA, 70 bp, Homo sapiens
gaatgtttaggggcctgtgtgaacgcaccaatggttcaaataaatgacaattactatgaggatttgacag

SEQ ID NO: 382 - DNA, 70 bp, Homo sapiens
ggggctgttaagtctgaccatacatcactgtgatagaatgtgggctttttcaagggtgaagatacaagtc

SEQ ID NO: 383 - DNA, 70 bp, Homo sapiens
cgcgctgctccgccgcccgggacttggccgcctcgtccgccacgcccgtgcctatgccgaggccgccgcc

SEQ ID NO: 384 - DNA, 70 bp, Homo sapiens
ctttgttgggaggcggtttgggagaacacatttctaatttgaatgaaatgaaatctattttcagtgaaaa

SEQ ID NO: 385 - DNA, 70 bp, Homo sapiens
ggtgacctctgccccagataggtggtgccagtggcttattaattccgatactagtttgctttgctgacca

SEQ ID NO: 386 - DNA, 70 bp, Homo sapiens
gtttttaaaatcagtactttttaatggaaacaacttgaccaaaaatttgtcacagaattttgagacccat

SEQ ID NO: 387 - DNA, 70 bp, Homo sapiens
gcccctggcttcaccctgtcaggccagctccactccaggactgaataaaggtctttgacagctctaaaaa

SEQ ID NO: 388 - DNA, 70 bp, Homo sapiens
attggcagatcaagcgccagaatggagatgatcccttgctgacttaccggttcccaccaaagttcaccct

SEQ ID NO: 389 - DNA, 70 bp, Homo sapiens
gccaggaggccctgggttccattcctaactctgcctcaaactgtacatttggataagccctagtagttcc

SEQ ID NO: 390 - DNA, 70 bp, Homo sapiens
ttgtggacttcctcattggctccggcctcaagaccatgtccatcgtgagttacaaccacctgggcaacaa

SEQ ID NO: 391 - DNA, 70 bp, Homo sapiens
tgttagagatgctatttgatacaactgtggccatgactgaggaaaggagctcacgcccagagactgggct

SEQ ID NO: 392 - DNA, 70 bp, Homo sapiens
acaaagtgaaaaacagccttttgagtctttctgatacctgagtttttatgcttataatttttgttctttg

SEQ ID NO: 393 - DNA, 70 bp, Homo sapiens
ccgcaatgttggtttcactgagagctgcctcctggtctcttcaccactgtagttctctcatttccaaacc

SEQ ID NO: 394 - DNA, 70 bp, Homo sapiens
gggaggaagcatgtgttctgtgaggttgttcggctatgtccaagtgtcgtttactaatgtacccctgctg

SEQ ID NO: 395 - DNA, 70 bp, Homo sapiens
caaggaaggggtagtaattggcccactctcttcttactggaggctatttaaataaaatgtaagacttcaa

SEQ ID NO: 396 - DNA, 70 bp, Homo sapiens
gttggtgaggtaacatacgtggagctcttaatggacgctgaaggaaagtcaaggggatgtgctgttgttg

SEQ ID NO: 397 - DNA, 70 bp, Homo sapiens
gaaagcacctgctccaaaggcatctggcaagaaagcataagtggcaatcataaaaagtaataaaggttct

SEQ ID NO: 398 - DNA, 70 bp, Homo sapiens
tgcttgtgaacgtgctaagcgtaccctctcttccagcacccaggccagtattgagatcgattctctctat

SEQ ID NO: 399 - DNA, 70 bp, Homo sapiens
ctgccttgttttgcgacattgtcccattcacacagatattttgggataataaaggaaaataagctacaaa

SEQ ID NO: 400 - DNA, 70 bp, Homo sapiens
gatatttaaagttttggcagtaaaatactctgtttttaagtatgaatgtatttcattcatatttcctctc

SEQ ID NO: 401 - DNA, 70 bp, Homo sapiens
tggttgattttgtactttggaactgtaccttggatggttttgtttattaaaagagaaacctgaaccaaaa

SEQ ID NO: 402 - DNA, 70 bp, Homo sapiens
ggaggcagaaccagcaacaactctgggcgtgcctgtgtctgcacatgtggatgtacatatgtctgtatat

SEQ ID NO: 403 - DNA, 70 bp, Homo sapiens
aggcggcgagcggggcccggcgccgaccctgagtgcagcctgacccgccctcgcgcgcgcgccctccccg

SEQ ID NO: 404 - DNA, 70 bp, Homo sapiens
gtgaaaagcctaaatgacatcacagcaaaagagaggttctctcccctcactaccaacctgatcaatttgc

SEQ ID NO: 405 - DNA, 70 bp, Homo sapiens
cgccacccttgacgcttgcagcttcggagtcacgggtttgaaacttcaaggggccacgtgcaacaacaac

SEQ ID NO: 406 - DNA, 70 bp, Homo sapiens
gcccagggaagacacatgattaatgatttagctccctccatacctcgaacatcagttgggatccctcctc

SEQ ID NO: 407 - DNA, 70 bp, Homo sapiens
cgattccactggtggtagtttgctagtgcttctaaaagttgctccctagcactgagaggtgtgggtaggt

SEQ ID NO: 408 - DNA, 70 bp, Homo sapiens
atgggccgacctggctgggactcgtgaatctggagaagagctggagaatggatagtattgtctgtatttg

SEQ ID NO: 409 - DNA, 70 bp, Homo sapiens
gaggacccctacacatcttttgtgaagttgctacctctgaatgattgccgatatgctttgtacgatgcca

SEQ ID NO: 410 - DNA, 70 bp, Homo sapiens
gcgagcgcgcctgcgcgctgggtgattttttcacgtgtcgccagggccggactgcgagtctctttgcggc

SEQ ID NO: 411 - DNA, 70 bp, Homo sapiens
gactacaaatggacgagagaggcggccgtccattagttagcggctccggagcaacgcagccgttgtcctt

SEQ ID NO: 412 - DNA, 70 bp, Homo sapiens; identical to SEQ ID NO: 286

SEQ ID NO: 413 - DNA, 70 bp, Homo sapiens
tttaggctggaagcgccttagaggagccatttttccaggtggggccccaggcagaggctccgacagggag

SEQ ID NO: 414 - DNA, 70 bp, Homo sapiens
cactaccgtggagatcccaactggtttatgaagaaagcgcaggagcataagagggaattcacagagagcc

SEQ ID NO: 415 - DNA, 70 bp, Homo sapiens
cgcttaaatcatgtgaaagggttgctgctgtcagccttgcccactgtgacttcaaacccaaggaggaact

SEQ ID NO: 416 - DNA, 70 bp, Homo sapiens
gagttcgagaccagcctgagcaacatggcgaaaccccgtctctactaaaaatacaaaaatcacccgggtg

SEQ ID NO: 417 - DNA, 70 bp, Homo sapiens
tgaggatggcttgacccgagtcggcttccgcacagtgttgctgagaatacgagaacagtggaaacagaac

SEQ ID NO: 418 - DNA, 70 bp, Homo sapiens
atgttgggcgagtcactgcgtctcgggcattggtgtcctgtcagtaaagagataataatggctgtacctc

SEQ ID NO: 419 - DNA, 70 bp, Homo sapiens
gtgcacgtgtgaagccccctcactcttccgctagggataaagcagatgtggatgccctttaagagatatt

SEQ ID NO: 420 - DNA, 70 bp, Homo sapiens
caggaacctgcttcactgtattaactagtccatgggctgagaccggggcatctcttttcttcatactgca

SEQ ID NO: 421 - DNA, 70 bp, Homo sapiens
cagcatacccccgattccgctacgaccaactcatacacctcctatgaaaaaacttcctaccactcaccct

SEQ ID NO: 422 - DNA, 70 bp, Homo sapiens
gctgcctgccctcctcctctcacccgatgtccaggtgggattttaaagtctgcattggttataataacag

SEQ ID NO: 423 - DNA, 70 bp, Homo sapiens
ataaggtttccagtaagcgggagggcagatccaactcagaaccatgcagataaggagcctctggcaaatg

SEQ ID NO: 424 - DNA, 70 bp, Homo sapiens
ctagttattaagcccagcatgcattagctctttttcctgatgctctccctcccttcatcatccgccctcc

SEQ ID NO: 425 - DNA, 70 bp, Homo sapiens
ctacttctaagtctgaatccagtcagaaataagattttttgagtaacaaataaataagatcagactccaa

SEQ ID NO: 426 - DNA, 70 bp, Homo sapiens
cccacgcgcacttacacgagaagacattcatggctttgggcagaaggattgtgcagattgtcaactccaa

SEQ ID NO: 427 - DNA, 70 bp, Homo sapiens
gaacccctgtggcgcaggactggcctgtgtctgttattttggttgtaaatcattctcctgtggaattggc

SEQ ID NO: 428 - DNA, 70 bp, Homo sapiens
cctgaattcactcgggtatattgattggctggatgatcttggtgccgcccacttgacgtttccagaagag

SEQ ID NO: 429 - DNA, 70 bp, Homo sapiens
gcacaaaggaggctttttctgtgctttgacattctagcacttcagggatgagagggagggagaatcctgg

SEQ ID NO: 430 - DNA, 70 bp, Homo sapiens
gcatccacaccaagagggtgttgtgatgaggtgccggtgtgcaaagggaactttagtttttccactggtt

SEQ ID NO: 431 - DNA, 70 bp, Homo sapiens
gtgtgaaacttgctctactctctgaaatgattcaaatacactaattttccatactttatacttttgttag

SEQ ID NO: 432 - DNA, 70 bp, Homo sapiens
taagcgctgacgcatgcgcatagctaaccgcacccggttcagctcgcctttcttggccagaggcgccggt

SEQ ID NO: 433 - DNA, 70 bp, Homo sapiens
atactttggacttcctctcgccaaagaccttccagcagattctggagtatgcatatacagccacgctgca

SEQ ID NO: 434 - DNA, 70 bp, Homo sapiens
tgtacacttgacaagtgcttactcagcaagtcccagacccacggccttttatctcccaagactggctttg

SEQ ID NO: 435 - DNA, 70 bp, Homo sapiens
gcgccgcccattggtcccgagcgcgatgacttggcgggcggagcaggaaggaaaccgctcccgagcacgg

SEQ ID NO: 436 - DNA, 70 bp, Homo sapiens
cgtggccgcacatcctacagttggaaatccatccagaggccatgttccaataaacaggaggtcgtgtaaa

SEQ ID NO: 437 - DNA, 70 bp, Homo sapiens
tctacgccccagggctgtcgccagacactatcatggagtgtgcaatgggggaccgcggcatgcagctcat

SEQ ID NO: 438 - DNA, 70 bp, Homo sapiens
ccttaagtctaataaggtcatggctgagtctctcagagtgtggacctgcccccttctactctgggcggtt

SEQ ID NO: 439 - DNA, 70 bp, Homo sapiens; identical to SEQ ID NO: 104

SEQ ID NO: 440 - DNA, 70 bp, Homo sapiens
ggcgggggccttggggcagtccgagggtgcggtgaagaggtgacggagggctggctatgggcggccggcc

SEQ ID NO: 441 - DNA, 70 bp, Homo sapiens
gtggtggcaggtgtttaatgacgaccttaccaagccaatcattgataatattgtgtctgatctcattcag

SEQ ID NO: 442 - DNA, 70 bp, Homo sapiens
caaccctgacccgtttgctacatctttttttctatgaaatatgtgaatggcaataaattcatctagacta

SEQ ID NO: 443 - DNA, 70 bp, Homo sapiens
actttgcagtggatcctgaccagccgctgagcgccaagaggaaccccattgacgtggaccccttcaccta

SEQ ID NO: 444 - DNA, 70 bp, Homo sapiens
cttcttcttctctcccagctgaacccgaggctaaagaagatgaggcaagagaaaatgtaccccaaggtga

SEQ ID NO: 445 - DNA, 70 bp, Homo sapiens
cacatggctgggctgacagcatcccctacacccccttcttcaagcataattacttactgactttcctcca

SEQ ID NO: 446 - DNA, 70 bp, Homo sapiens
ccaccctggagccaagggtctttcacatcacctatccctacatacataccaaatggaaaagtggccatcc

SEQ ID NO: 447 - DNA, 70 bp, Homo sapiens
ttaagactttccaaagatgaggtccctggtttttcatggcaacttgatcagtaaggatttcacctctgtt

SEQ ID NO: 448 - DNA, 70 bp, Homo sapiens
aggagcagtaaacatagccaaggcctaagggatcaaggaaaccaagagcaggatccaaatatttccaatg

SEQ ID NO: 449 - DNA, 70 bp, Homo sapiens
agaagggccccaatgccaactcttaagtcttttgtaattctggctttctctaataaaaaagccacttagt

SEQ ID NO: 450 - DNA, 70 bp, Homo sapiens
acattccagatggctatcctgcttcagtacaacacggaagatgcctacactgtgcagcagctgaccgaca

SEQ ID NO: 451 - DNA, 70 bp, Homo sapiens
ggggcatcagagtcttggctgggctgaatctgctgcttgttggttcagtgtttcttatgaacaagagcca

SEQ ID NO: 452 - DNA, 70 bp, Homo sapiens
gagagttcgatatgattcttgggaaactagagaatgacggaagtagaaagcctggagtcatagataagtt

SEQ ID NO: 453 - DNA, 70 bp, Homo sapiens
gagagttgctgcctttgatagacccatgctgaccacagcctgatattccagaacctggaacagggacttt

SEQ ID NO: 454 - DNA, 70 bp, Homo sapiens
gtctgagcaaggggtgtacacctgcacagcacagggcatttggaagaatgaacagaagggagagaagatt

SEQ ID NO: 455 - DNA, 70 bp, Homo sapiens
agagaccgctggcagcaccagtattcccaagaggaagaagtctacacccaaggaggaaacagttaatgac

SEQ ID NO: 456 - DNA, 70 bp, Homo sapiens
ctaagactcgcggcaggttctctttgagtcaatagcttgtcttcgtccatctgttgacaaatgacagatc

SEQ ID NO: 457 - DNA, 70 bp, Homo sapiens
gccagatagctaggtttctggttcccccacagtaggtgttttcacataagattagggtccttttggaaag

SEQ ID NO: 458 - DNA, 70 bp, Homo sapiens
aagcacgttgcccaaggttgcacagcaagaaaagggagaagttgagattcaaacccaggctgtctagctc

SEQ ID NO: 459 - DNA, 70 bp, Homo sapiens
ctgcaaagaggccaacacactagaaatcagaaatcttgactcctagcccaccgtcccctaaaacatgggc

SEQ ID NO: 460 - DNA, 70 bp, Homo sapiens
cgcggtttggtttgcagcgactggcatactatgtggatgtgacagtggcgtttgtaatgagagcactttc

SEQ ID NO: 461 - DNA, 70 bp, Homo sapiens
tcggactcctgcctcactcatttacaccaaccacccaactatctataaacctagccatggccatcccctt

SEQ ID NO: 462 - DNA, 70 bp, Homo sapiens
aggagcagcccatggagacgacgggcgccaccgagaacggacatgaggccgtccccgaaggcgagtcgcc

SEQ ID NO: 463 - DNA, 70 bp, Homo sapiens
gcttcttgctgccgccatatgaagaaggacgtgttcgcttccccttcctccatgattgtaagtttcctga

SEQ ID NO: 464 - DNA, 70 bp, Homo sapiens
catgttaaaatggggaaggatgatagctacatgtatgccggtcctactcacgcgacacccgtgtgctcaa

SEQ ID NO: 465 - DNA, 70 bp, Homo sapiens
acatgaccccagcaactgtggtggtatctagaggtgaaacaggcaagtgaaatggacacctctgctgtga

SEQ ID NO: 466 - DNA, 70 bp, Homo sapiens
gcccctggcaaatgcacacacctcatgctagcctcacgaaactggaataagccttcgaaaagaaattgtc

SEQ ID NO: 467 - DNA, 70 bp, Homo sapiens
gtggttgatggcgccttcaaagaggtgaagctgtcggactacaaagggaagtacgtggtcctctttttct

SEQ ID NO: 468 - DNA, 70 bp, Homo sapiens
tccttcctagtaatactttgcctttttcactgtgtatggaatgaaacatgtaaagctgtcacaatcaatg

SEQ ID NO: 469 - DNA, 70 bp, Homo sapiens
gcaagactcttacgccccacactgcaatttggtcttgttgccgtatccatttatgtgggcctttctcgag

SEQ ID NO: 470 - DNA, 70 bp, Homo sapiens
ccacagaagacacgtgtttttgtatctttaaagacttgatgaataaacactttttctggtcaatgtcaaa

SEQ ID NO: 471 - DNA, 70 bp, Homo sapiens
gcagctttgaactagggctggggttgtgggtgcctcttctgaaaggtctaaccattattggataactggc

SEQ ID NO: 472 - DNA, 70 bp, Homo sapiens
cccaggctttgtcccaggctttctggtgtgtgccctcctggtaacagtgaaattgaagctacttactcat

SEQ ID NO: 473 - DNA, 70 bp, Homo sapiens
caggtgcctagtcttgagtgaattgttagatgtgcactgaactcgggatgttggggattggagagagaga

SEQ ID NO: 474 - DNA, 70 bp, Homo sapiens
aaaagtattttgtggtgaccataagaatgtccctccccaaacaagtaaacttgtgaaagtttaatttgga

SEQ ID NO: 475 - DNA, 70 bp, Homo sapiens
atgatcctgttagctcttccagctctccaggcgccaacaaccatatggtctcggtaacgactgctcccca

SEQ ID NO: 476 - DNA, 70 bp, Homo sapiens
gagaatacaagatattatgtataaaatgtaacactgatgataggttaataaagatgattgaatccaaaaa

SEQ ID NO: 477 - DNA, 70 bp, Homo sapiens
taccccttccactgctcactttgtggatggtagcatgagctgtctaccaagaagaaacctgctgctctct

SEQ ID NO: 478 - DNA, 70 bp, Homo sapiens
caactggatgaaaaggaaaaggatttggtgggcctggctcagatcgcagaggtcctcgagatgttcgatt

SEQ ID NO: 479 - DNA, 70 bp, Homo sapiens
ctaatccccttgatgagctttcacgaagtctcacggcttctctagggactccatggtcttcagagtcgtt

SEQ ID NO: 480 - DNA, 70 bp, Homo sapiens
agatgggatagtttactgactagttggagcatttgtaagcacatggtgaaatcagcccctgcccaccaaa

SEQ ID NO: 481 - DNA, 70 bp, Homo sapiens
cctgggattctttttctagggatgtaatacatatatttacaaataaaatgcctcatggactctggtgaaa

SEQ ID NO: 482 - DNA, 70 bp, Homo sapiens
tttaatcgctttgaataaatactcccttaagtagttaaatataggaggagaaagaatacatcggttgtta

SEQ ID NO: 483 - DNA, 70 bp, Homo sapiens
ggcaatgcctacccccagcgttatttttggggagggagggctgtgcatagggacatattctttagaatct

SEQ ID NO: 484 - DNA, 70 bp, Homo sapiens
tggaataaaaggagagaagggtttccccggattccctggactggacatgccgggccctaaaggagataaa

SEQ ID NO: 485 - DNA, 70 bp, Homo sapiens
ggccaaccgagcgccatgaaccagatagagcccggcgtgcagtacaactacgtgtacgacgaggatgagt

SEQ ID NO: 486 - DNA, 70 bp, Homo sapiens
gccaaagtgctcagagaccttctatgacacattagtgtcacatggttgcgtgtccagccgaagcagtgta

SEQ ID NO: 487 - DNA, 70 bp, Homo sapiens
atacaaaagtggcacatgcctgtaatgccagctactggggaggctgaggtaggagaattgcttgaacctg

SEQ ID NO: 488 - DNA, 70 bp, Homo sapiens
ccggcagttcttgggtcaaatgacacaattaaaccaactcctgggagaggtgaaggaccttctgagacag

SEQ ID NO: 489 - DNA, 70 bp, Homo sapiens
gtggctggcccggcctccacagcaccccaccccatatcttctttccatttatttcgtaccaaaaacaatt

SEQ ID NO: 490 - DNA, 70 bp, Homo sapiens
cattttttgtaatttttgtaaaacaaaagtaccaatctgttttgtaaataaaaatcatcctaaaattcga

SEQ ID NO: 491 - DNA, 70 bp, Homo sapiens
tgatctttctggctccactcagtgtctaaggcaccctgcttcctttgcttgcatcccacagactatttcc

SEQ ID NO: 492 - DNA, 70 bp, Homo sapiens
tcataactggcttctgcttgtcatccacacaacaccaggacttaagacaaatgggactgatgtcatcttg

SEQ ID NO: 493 - DNA, 70 bp, Homo sapiens
agccaggatttccctcagtgcaacaccattgagaatacaggaactaaacagtccacctgtagtccagggg

SEQ ID NO: 494 - DNA, 70 bp, Homo sapiens
cgtagactcgctcatctcgcctgggtttgtccgcatgttgtaatcgtgcaaataaacgctcactccgaat

SEQ ID NO: 495 - DNA, 70 bp, Homo sapiens
gacactggcccctctcaggtcagaagacatgcctggagggatgtctggctgcaaagactatttttatcct

SEQ ID NO: 496 - DNA, 70 bp, Homo sapiens
tttgcccagcacgccaacgccttccaccagtggatccaagagaccaggacatacctcctcgatgggtcct

SEQ ID NO: 497 - DNA, 70 bp, Homo sapiens
cacaacatgaaagaaatggtgctacccagctcaagcctgggcctttgaatccggacacaaaaccctctag

SEQ ID NO: 498 - DNA, 70 bp, Homo sapiens
atccccatgcccttgacctcttctggcattctcctgtgctctgacaaactgagccagccttttagatcta

SEQ ID NO: 499 - DNA, 70 bp, Homo sapiens
aagtttccgaccctggcttataggcaccacacctcatgtactcctcatggcttggatctctgtattcagc

SEQ ID NO: 500 - DNA, 70 bp, Homo sapiens
aaggtctgacgccacctcaaggtgacagctcatctccagcacagcacaggcgtgtgcacacagaggtgtt

SEQ ID NO: 501 - DNA, 70 bp, Homo sapiens
cggagcagagacaggccctcggggtggaggtctttggtttcataagagcctgagagagatttttctaaga

SEQ ID NO: 502 - DNA, 70 bp, Homo sapiens
ataagtcacattggttccatggccacaaaccattcagatcagccacttgctgaccctggttcttaaggac

SEQ ID NO: 503 - DNA, 70 bp, Homo sapiens
ctacttcggagtctatgatactgccaaggggatgctgcctgaccccaagaacgtgcacatttttgtgagc

SEQ ID NO: 504 - DNA, 70 bp, Homo sapiens
actgttgcttgctggtcgcagactccctgacccctccctcacccctccctaacctcggtgccaccggatt

SEQ ID NO: 505 - DNA, 70 bp, Homo sapiens
gacagggccagtgcagtttggtgtgtcctccgcctttccaggagaagaacctgaagaactatttttcgtt

SEQ ID NO: 506 - DNA, 70 bp, Homo sapiens
ggtcagggactgaatcttgcccgtttatgtatgctccatgtctagcccatcatcctgcttggagcaagta

SEQ ID NO: 507 - DNA, 70 bp, Homo sapiens
gggagagtgccgggcggtcggcgggtcagggcagcccggggcctgacgccatgtcccggaacctgcgcac

SEQ ID NO: 508 - DNA, 70 bp, Homo sapiens
tgctctaagggaccttggagacaggcctttcaggtggatgttcatgtttctgaccttgcactaccccaat

SEQ ID NO: 509 - DNA, 70 bp, Homo sapiens
attcgccgttcgaaagcagggactaaaagccccacttcgtcttacgttccgaaaggaaggcgtctgttga

SEQ ID NO: 510 - DNA, 70 bp, Homo sapiens
ggtggagttgttagtgtcctatggcaacaccttctttgtggttctcattgtcatccttgtgctgttggtc

SEQ ID NO: 511 - DNA, 70 bp, Homo sapiens
ttgcctcatcaccttgtccaaatgagctagacctccctgtcccggagggaaaaacatctgaaaagcagac

SEQ ID NO: 512 - DNA, 70 bp, Homo sapiens
tttttaagctcaagcaaatgtttggtaatgcagacatgaatacatttcacaccttcaaatttgaagatcc

SEQ ID NO: 513 - DNA, 70 bp, Homo sapiens; identical to SEQ ID NO: 76

SEQ ID NO: 514 - DNA, 70 bp, Homo sapiens
gaactgtggccacctagaaaggggcccattcagcctcgtctctttacagaagtagttttgttcatgaaat

SEQ ID NO: 515 - DNA, 70 bp, Homo sapiens
actccaagaagtacattgccttctgcatcagcatcttcacggccatcctggtgaccatcgtgatcctcta

SEQ ID NO: 516 - DNA, 70 bp, Homo sapiens
aagacctgaaccagagatccatcatggagagcccagccaacagtattgagatgcttctgtccaacttcgg

SEQ ID NO: 517 - DNA, 70 bp, Homo sapiens
tatttttcttaacatgttagtacttctacgactttggagccactgatgggtccactcatggcctcagctg

SEQ ID NO: 518 - DNA, 70 bp, Homo sapiens
ggtcagcaaaggaaagtggaagttggattctgaaagatcgaggtgcccacaggaattttatggtcgtcgg

SEQ ID NO: 519 - DNA, 70 bp, Homo sapiens
agcacacccgtctatgtagcaaaatagtgggaagatttataggtagaggcgacaaacctaccgagcctgg

SEQ ID NO: 520 - DNA, 70 bp, Homo sapiens
aaggaaaaccggccccagaaacaggggtgtgctttcccaccaataaaaggccgtggaacccgagggcttt

SEQ ID NO: 521 - DNA, 70 bp, Homo sapiens
gggatatagggtcgaagccgcactcgtaaggggtggatttttctatgtagccgttgagttgtggtagtca

SEQ ID NO: 522 - DNA, 70 bp, Homo sapiens
gctcctttgttttacagagcagggtcacttgatttgctagctggtggcagaattggcaccattacccagg

SEQ ID NO: 523 - DNA, 70 bp, Homo sapiens
tgaacaaaagaagccacgaggtggaacaaggtctctgtcagtcacaggcacccctgagaaccgggaacat

SEQ ID NO: 524 - DNA, 70 bp, Homo sapiens
gctactgagggtctaagtccgggcagccgaagagtgtggtaggtaacggtcctcagcgcaagggtcattt

SEQ ID NO: 525 - DNA, 70 bp, Homo sapiens
tctacaaagggttcatgccctcctttctccgcttgggttcctggaacgtggtgatgttcgtcacctatga

SEQ ID NO: 526 - DNA, 70 bp, Homo sapiens
tttatccccagaccaggcatcacctatgagccacccaactataaggccctggacttctccgaggccccaa

SEQ ID NO: 527 - DNA, 70 bp, Homo sapiens
atgccgtcggaaatggtgaagggagactcgaagtactctgaggcttgtaggagggtaaaatagagaccca

SEQ ID NO: 528 - DNA, 70 bp, Homo sapiens
tttttaagtagcctcctttccactatttagtaattggctgtgagctgggctgggggagaaatggggcggg

SEQ ID NO: 529 - DNA, 70 bp, Homo sapiens
tttttgagacagagttttgctctcgttgcccaagcttgagtgtaatggcatggtcttggctcactgcact

SEQ ID NO: 530 - DNA, 70 bp, Homo sapiens
  misc_feature (8)..(9): n is a, c, g, or t
accctggnnagatagacttccctgtttccaaggggcgtgggactttctaccacgtccatcaactcgtggc

SEQ ID NO: 531 - DNA, 70 bp, Homo sapiens
atagtgtttggcttattttccatcccagttctgggaggtcttttaagtctccttcctttggttgccccac

SEQ ID NO: 532 - DNA, 70 bp, Homo sapiens
aactatttgcgcaatctgtgggtctgtggattcacggggctttctgtgtgggtgctgcagttgcttttgt

SEQ ID NO: 533 - DNA, 70 bp, Homo sapiens
tgggccttgtgacattgtctacctgtggtcattccttaactgctttggcctcaactttgagctctggatg

SEQ ID NO: 534 - DNA, 70 bp, Homo sapiens
ttttaaaaatccacttatggctgggcacagaagctcacgcctgtaatcccagcactttgggaggctgagg

SEQ ID NO: 535 - DNA, 70 bp, Homo sapiens
aaaccccatctctactaaaaatacaaaaaattagccgggcgtggtagcgggcgcctgtagtcccagctac

SEQ ID NO: 536 - DNA, 70 bp, Homo sapiens
ctggatcttggcctttacattttctatcgtatccgagggttcaacctcgagggtgatggtcttccccgta

SEQ ID NO: 537 - DNA, 70 bp, Homo sapiens
ggacaagaacacagtcaactttggctttgcttggaaagctgcttcagatacataactcccggcccctcct

SEQ ID NO: 538 - DNA, 70 bp, Homo sapiens; identical to SEQ ID NO: 444

SEQ ID NO: 539 - DNA, 70 bp, Homo sapiens
atccaaccctttaagatgagtgccactggttgcccattttacagatgagaaactgggctcacagacacac

SEQ ID NO: 540 - DNA, 70 bp, Homo sapiens
ggacatttgggttggttccaagtctttgctattgtgaatagtgccgcaataaacatacgtgtgcatgtgt

SEQ ID NO: 541 - DNA, 70 bp, Homo sapiens
atcagccgtaagcctagaagcagagcgggatcgaggcgtttttaataattcgagttgggaagacccggat

SEQ ID NO: 542 - DNA, 70 bp, Homo sapiens
gctctagctacttggactattcagggagctgcaaatgccctctctggtgacgtttgggacattgacaatg

SEQ ID NO: 543 - DNA, 70 bp, Homo sapiens
gcggagattcaaggacctaagcttccaggaggagtacagcacactgttccctgcctcggcacagccgtag

SEQ ID NO: 544 - DNA, 70 bp, Homo sapiens
cagtgttcgaatcatcgacaaaaatggcatccatgacctggataacatttccttccccaaacagggctcc

SEQ ID NO: 545 - DNA, 70 bp, Homo sapiens
tatcttgctggtcaaaatatacaagatgtgagcctggaaagccttcggagggcagtgggagtggtacctc

SEQ ID NO: 546 - DNA, 70 bp, Homo sapiens
  misc_feature (10)..(10): n is a, c, g, or t
  misc_feature (20)..(20): n is a, c, g, or t
  misc_feature (36)..(36): n is a, c, g, or t
  misc_feature (57)..(57): n is a, c, g, or t
ggcttctggnaagctgttgnagcccaattgaaccanaaagtttggtggcctatcagntggaccttgtatg

SEQ ID NO: 547 - DNA, 70 bp, Homo sapiens
accttcactgtcagcgcctggaaaacttggctcacgaaaccagggaacgaagaaaaacctccaggggaac

SEQ ID NO: 548 - DNA, 70 bp, Homo sapiens
ccctctggtccagcccctcacgcctcctctcagtctactcaattgtgactgtccctcctgatgtattttt

SEQ ID NO: 549 - DNA, 70 bp, Homo sapiens; identical to SEQ ID NO: 136

SEQ ID NO: 550 - DNA, 70 bp, Homo sapiens
ctctgtgtttcatgtgtcccaggtcccccaaaaaacaggtggtggtggattatacatggctttcagtagt

SEQ ID NO: 551 - DNA, 70 bp, Homo sapiens
agtacctgcacaaccagcacatcctgcacctggacctgaggtccgagaacatgatcatcaccgaatacaa

SEQ ID NO: 552 - DNA, 70 bp, Homo sapiens
  misc_feature (18)..(18): n is a, c, g, or t
caccactctgaacagctncttgatggtgtcattcaagttattgggctttctctcccgctggagcctcagc

SEQ ID NO: 553 - DNA, 70 bp, Homo sapiens
ggacgtgtaaacagacggtaccctactcttgtggcaatcactaagtttcagccaaccaaagacagcgaac

SEQ ID NO: 554 - DNA, 70 bp, Homo sapiens
ratcataagtgagahtcykcccagtyttmtttgtgcttytcttttgggragawttagtaaytgtgccact

SEQ ID NO: 555 - DNA, 70 bp, Homo sapiens
ctacaataagggcaactgcagtctcatatgtccaacatcgagcaacattacggattgtgtagccacctcc

SEQ ID NO: 556 - DNA, 70 bp, Homo sapiens
kgyaccacaggrttgagccgtcgaggggkgagtgctgttattatwtcttaaaaaatctgatgacccgggv

SEQ ID NO: 557 - DNA, 70 bp, Homo sapiens
cactgacagggatcaagtttgtggttctagcagatcctaggcaagctggaatagattctcttctccgaaa

SEQ ID NO: 558 - DNA, 70 bp, Homo sapiens
aaatggacaaggccaggtatagcgaatggctttgctcctgtagagaaccgtcactcggtcagamaarcct

SEQ ID NO: 559 - DNA, 70 bp, Homo sapiens; identical to SEQ ID NO: 527

SEQ ID NO: 560 - DNA, 70 bp, Homo sapiens
atcacctgctgtatgccgatcatctcagaaagggctgtgtagagtagggccctgttctccttaggatgtt

SEQ ID NO: 561 - DNA, 70 bp, Homo sapiens
cgacatcattgttggcgatggtgatgaccacatctgggacattgtagggagtgtctaggtgactctccat

SEQ ID NO: 562 - DNA, 70 bp, Homo sapiens
ctctgtatgagaactccccagagttcacaccttacctggagacaaacctcggacagccaacaattcagag

SEQ ID NO: 563 - DNA, 70 bp, Homo sapiens
agcctggggtgcttcgtgggctcccgctttgtccacggcgagggtctccgctggtacgccggcctgcaga

SEQ ID NO: 564 - DNA, 70 bp, Homo sapiens
tcatcgacatgctcatggagaacatctccaccaagggcctggactgtgacattgacatccagaagacatc

SEQ ID NO: 565 - DNA, 70 bp, Homo sapiens
tgagtcccgggtagttggagcctgtcagtcgccgggtcagtaggtcgcggagtctgcgagaagccactat

SEQ ID NO: 566 - DNA, 70 bp, Homo sapiens
aagttacgcagatcccataaagctacggtcttatccgcagagccggtggctagaataaaatcgcctgtag

SEQ ID NO: 567 - DNA, 70 bp, Homo sapiens
aagattattttttaaatcctgaggactagcattaattgacagctgacccaggtgctacacagaagtggat

SEQ ID NO: 568 - DNA, 70 bp, Homo sapiens
gaagccagactacactgcttacgttgccatgatccctcagtgcataaaggaggaagacaccccttcagat

SEQ ID NO: 569 - DNA, 70 bp, Homo sapiens
acccacaggtcctaaactaccaaacctgcattaaaaatttcggttggggcgacctcggagcagaacccaa

SEQ ID NO: 570 - DNA, 70 bp, Homo sapiens
gcatgaaaaactccaaataagagatcyctcaggattataaaagttgtaaatgcactgtwtkctggsaaaa

SEQ ID NO: 571 - DNA, 70 bp, Homo sapiens
ccttctgcacatctaaacttagatggagttggtcaaatgagggaacatctgggttatgccttttttaaag

SEQ ID NO: 572 - DNA, 70 bp, Homo sapiens
agggtcttctcgtcttgctgtgtcatgcccgcctcttcacgggcaggtcaatttcactggttaaaagtaa

SEQ ID NO: 573 - DNA, 70 bp, Homo sapiens
cctcttccggagatgtagcaaaacgcatggagtgtgtattgttcccagtgacacttcagagagctggtag

SEQ ID NO: 574 - DNA, 70 bp, Homo sapiens
atgtgtaccttggagtcatcctcttggtcttgtattcatattgtgggacagtgggaatagcagcttgtag

SEQ ID NO: 575 - DNA, 70 bp, Homo sapiens
accttttctggcaagactgctctgcatttctgctgccctcatacctcacccagccaacctaccaaacatt

SEQ ID NO: 576 - DNA, 70 bp, Homo sapiens
ccataaagactccgtgtaactgtgtgaacacttgggatttttctcctctgtcccgaggtcgtcgtctgct

SEQ ID NO: 577 - DNA, 70 bp, Homo sapiens
acctgttgttacagggcaggatcggatgatggacactgaagtcctcagcttgctaagttcagttgctctc

SEQ ID NO: 578 - DNA, 70 bp, Homo sapiens
tgtttctaccaacactgcaccttatcccaggaacctgccctagacctccagagaccatattttctctccc

SEQ ID NO: 579 - DNA, 70 bp, Homo sapiens
aacttgaacctaaaaattagcccctcatagtgtagccgccggactttgctcatagctggcaggctggact

SEQ ID NO: 580 - DNA, 70 bp, homo sapiens
gtaggagctcgtcactcttttgacaaaaagggggtgattgtggttgaagtggaggacagagagaagaagg

SEQ ID NO: 581 - DNA, 70 bp, Homo sapiens
gacagtgtgggtatcaagagccaatgtgatccagcgccggggccgggcgggccgctgccagtccggcttt

SEQ ID NO: 582 - DNA, 70 bp, Homo sapiens; identical to SEQ ID NO: 423

SEQ ID NO: 583 - DNA, 70 bp, Homo sapiens
  misc_feature (15)..(15): n is a, c, g, or t
ctgcagtcctcactngagaaaatcactccctctgggagattggaagttgctggaaagaaaacaggtccaa

SEQ ID NO: 584 - DNA, 70 bp, Homo sapiens
aatctggccaaaagagttcgcgctttcccccatggatgttttctaccacaagaatataagtgctgaaaat

SEQ ID NO: 585 - DNA, 70 bp, Homo sapiens
cagagtacttcgagtctcccttcaccatttccgacggcatctacggctcaacattttttgtagccacagg

SEQ ID NO: 586 - DNA, 70 bp, Homo sapiens
cgcccacggacttacatcctcattactattctgcctagcaaactcaaactacgaacgcactcacagtcgc

SEQ ID NO: 587 - DNA, 70 bp, Homo sapiens
tgcgacaggcacgcagcctactaggtgtggcggcgaccctggccccgggttcccgtggctaccgggcgcg

SEQ ID NO: 588 - DNA, 70 bp, Homo sapiens
tgaatggtcagcttgtccacagggtgaatcttgttgtagtcagccgggtcagcgaaggtcagaggcagca

SEQ ID NO: 589 - DNA, 70 bp, Homo sapiens
cccatcatactctttcacccacagcaccaatcctacctccatcgctaaccccactaaaacactcaccaag

SEQ ID NO: 590 - DNA, 70 bp, Homo sapiens
gcacccaatacaggagcagccagattcataaagcaagtcctgagtgacctacaaagagacttagactccc

SEQ ID NO: 591 - DNA, 70 bp, Homo sapiens
agctctctgctctcccagcgcagcgccgccgcccggcccctccagcttcccggaccatggccaacctgga

SEQ ID NO: 592 - DNA, 70 bp, Homo sapiens
ttcagcgtggggcgcccacaatttgcgcgctctctttctgctgctccccagctctcggatacagccgaca

SEQ ID NO: 593 - DNA, 70 bp, Homo sapiens
cccaacccgtcatctactctaccatctttgcaggcacactcatcacagcgctaagctcgcactgattttt

SEQ ID NO: 594 - DNA, 70 bp, Homo sapiens
gagtacaccgactacggcggactaatcttcaactcctacatacttcccccattattcctagaaccaggcg

SEQ ID NO: 595 - DNA, 70 bp, Homo sapiens
ctggagccggagcaccctatgtcgcagtatctgtctttgattcctgcctcatcctattatttatcgcacc

SEQ ID NO: 596 - DNA, 70 bp, Homo sapiens
cttcgaatgtgtggtaggggtggggggcatccatatagtcactccaggtttatggagggttcttctacta

SEQ ID NO: 597 - DNA, 70 bp, Homo sapiens
tctcaacttagtattatgcccacacccacccaagaacagggtttgttaagatggcagagcccggtaatcg

SEQ ID NO: 598 - DNA, 70 bp, Homo sapiens
agcattcctgcacatctgtacccacgccttcttcaagccatactatttatgtgctccggggtcatcatcc

SEQ ID NO: 599 - DNA, 70 bp, Homo sapiens; identical to SEQ ID NO: 593

SEQ ID NO: 600 - DNA, 70 bp, Homo sapiens
ccgccatcttcagcaaaccctgatgaaggctacaaagtaagcgcaagtacccacgtaaagacgttaggtc

SEQ ID NO: 601 - DNA, 70 bp, Homo sapiens
ccgggatcgtcatctactctaccatctttgcaggcacactcatcacagcgctaagctcgcactgattttt

SEQ ID NO: 602 - DNA, 70 bp, Homo sapiens
acttttgaaattcacacattgtgaagcctgccagtccccgccaggtgaagagctcatggtatccaccttc

SEQ ID NO: 603 - DNA, 70 bp, Homo sapiens
ctggtgaagccccagctatcatggcagtgaagggctctggctagatttggatgtcaactgctgagttcta

SEQ ID NO: 604 - DNA, 70 bp, Homo sapiens
cgctggaccggtccggattcccgggatgtccacacaggcagacttgaccttgacagatagtcttcaagat

SEQ ID NO: 605 - DNA, 70 bp, Homo sapiens; identical to SEQ ID NO: 569

SEQ ID NO: 606 - DNA, 70 bp, Homo sapiens
caccctagtaggctcccttcccctactcatcgcactaatttacactcacaacaccctaggctcactaaac

SEQ ID NO: 607 - DNA, 70 bp, Homo sapiens
acagagctccttcaaacttcagaacggcctatgaaggagtcccgtggaaacatctgggaggactttcaag

SEQ ID NO: 608 - DNA, 70 bp, Homo sapiens
tagttagggccctcggccacactcaagttctgctcctccaacagggcctgaaagttttttcggaagcgaa

SEQ ID NO: 609 - DNA, 70 bp, Homo sapiens
tggtcgtgggagggctgaacacacattaccgctacattggcaagaccatggattaccggggaaccatgat

SEQ ID NO: 610 - DNA, 70 bp, Homo sapiens
acgcgtccgctctgacttcttggactacatggggatcaaaggccccaggatgcctctgggcttcacgttc

SEQ ID NO: 611 - DNA, 70 bp, Homo sapiens
acagaatatcctgtagaaaaactaatgagggatgccaaaatctatcagatttatgaaggtacttcacaaa

SEQ ID NO: 612 - DNA, 70 bp, Homo sapiens
cccacgcgtccgagcaagttgaaaatggattgagactgcatggtggcataaatgagaaattgcctgtagc

SEQ ID NO: 613 - DNA, 70 bp, Homo sapiens
caaagtagtgatggattcagtactcctcaaccactctcctaatgattggaacaaaagcaaacaaaaaaga

SEQ ID NO: 614 - DNA, 70 bp, Homo Sapiens
tacccagcacatcccactataccagatgagtggcttctatggcaagggtccctccattaagcagttcatg

SEQ ID NO: 615 - DNA, 70 bp, Homo sapiens
aagaacagtacaaagaacatccgtgtacccagtaccctgactaccgactacctacaacccgtccctgccc

SEQ ID NO: 616 - DNA, 70 bp, Homo sapiens
ccttaccaccaaacataccaaaatgcacctctttcataagtgagttactaagatttctatacctggaata

SEQ ID NO: 617 - DNA, 70 bp, Homo sapiens
cctatttggaccagaaaccctgatgacatcacccaagaggagtatggagaattctacaagagcctcacta

SEQ ID NO: 618 - DNA, 70 bp, Homo sapiens
acgggagaggtactgaggacaaatcagttctctgtgaccagacatgaaaaggttgccaatgggctgttgg

SEQ ID NO: 619 - DNA, 70 bp, Homo sapiens
taccctagccaaccccttaaacacccctccccacatcaagcccgaatgatatttcctattcgcctacaca

SEQ ID NO: 620 - DNA, 70 bp, Homo sapiens
tacagagtcacactcaatcctccgggcaccttccttgaaggagtggctaaggttggacaatacacgttca

SEQ ID NO: 621 - DNA, 70 bp, Homo sapiens
acccttggccataatatgatttatctccacactagcagagaccaaccgaacccccttcgaccttgccgaa

SEQ ID NO: 622 - DNA, 70 bp, Homo sapiens
gcccacttcttaccacaaggcacacctacaccccttatccccatactagttattatcgaaaccatcagcc

SEQ ID NO: 623 - DNA, 70 bp, Homo sapiens
cacttctggttgccaggagacagcaagcaaagccagcaggacatgaagttgctattaaatggacttcgtg

SEQ ID NO: 624 - DNA, 70 bp, Homo sapiens; identical to SEQ ID NO: 214

SEQ ID NO: 625 - DNA, 70 bp, Homo sapiens
tgacctggcctctcccccacaggaacaaaacactgcctccagagtctttaaattctcagttatcaacgcc

SEQ ID NO: 626 - DNA, 70 bp, Homo sapiens
gagaaggtaagcacatttgaggccacctagcctttgcttctctgttcaaatcaattatatttcaaaagct

SEQ ID NO: 627 - DNA, 70 bp, Homo sapiens
ggtgtacactcaaaacctgtccccggcagccagtgctctctgtatagggccataatggaattctgaagaa

SEQ ID NO: 628 - DNA, 70 bp, Homo sapiens
gggacatgcttccccttgtccacctttgcagcctgtttctgtcatgtagtttcaacaagtgctacctttg

SEQ ID NO: 629 - DNA, 70 bp, Homo sapiens
acgctcttcgctgtcgtttgtggtctcgcgcagggcggccccggttctggtgtttggcgtcggaattaaa

SEQ ID NO: 630 - DNA, 70 bp, Homo sapiens
taagagacgacagggaccgaagaggacctccactcagatcagaacgtgaagaagtaagttcttggagacg

SEQ ID NO: 631 - DNA, 70 bp, Homo sapiens
gcacaggctgtggcttgcactccagccgctctagtctctcaggaatttgcttgttacttgtactgtgtaa

SEQ ID NO: 632 - DNA, 70 bp, Homo sapiens
ccaaaccaactctttgccagcagccacaacatgcattgacagcggcacagtgagatataactgatgggct

SEQ ID NO: 633 - DNA, 70 bp, Homo sapiens
tatatattgtgcatcaactctgttggatacgagaacactgtagaagtggacgatttgttctagcaccttt

SEQ ID NO: 634 - DNA, 70 bp, Homo sapiens
tcagaaatgaggtgtaattccccaacccctgcccgcaagagctaagtaggatcttactgtaagttgaagg

SEQ ID NO: 635 - DNA, 70 bp, Homo sapiens; identical to SEQ ID NO: 111

SEQ ID NO: 636 - DNA, 70 bp, Homo sapiens
  misc_feature (36)..(36): n is a, c, g, or t
gcatttcttctatgcacttatcagaaagatcaaagnctttaatactttcactaattttgctactgctatc

SEQ ID NO: 637 - DNA, 70 bp, Homo sapiens
accggcgtcaaagtatttagctgactcgccacactccacggaagcaatatgaaatgatctgctgcagtgc

SEQ ID NO: 638 - DNA, 70 bp, Homo sapiens
ttgagaagtatcctgaggcatggggggttcatagtagaagagcgatggtgagagctaaggtcggggcggt

SEQ ID NO: 639 - DNA, 70 bp, Homo sapiens
tccccctacacctgtgtcagctgcggtgcccgggcaggtacatctaccttcgaggcaggagccctctcat

SEQ ID NO: 640 - DNA, 70 bp, Homo sapiens
tttccatcaattagctcccgcacaagtgtggtctcttgcccgtcccatttctgcaggtgaacaagtttcc

SEQ ID NO: 641 - DNA, 70 bp, Homo sapiens
ggaaatgtgagccctgcattctgaatgagttttaggattatattctgattcactaattctcctttcaacc

SEQ ID NO: 642 - DNA, 70 bp, Homo sapiens
gggagtgtaaaatgcttcagccactttagaaaatagttttgcagtttcttacaacattaaaaatatattt

SEQ ID NO: 643 - DNA, 70 bp, Homo sapiens
ttctgctccacgggaggtttctgtcctccctgagctcgccttaggacacctgcgttaccgtttgacaggt

SEQ ID NO: 644 - DNA, 70 bp, Homo sapiens
acattctgtgggaataaacacaacttgcttgccctgatgctcaatagcagtgtaatcaccatgtttaaac

SEQ ID NO: 645 - DNA, 70 bp, Homo sapiens
ctttgatgccttgacctatgatttcaatacagcgcattactttggctgctaatttttctgggagggcaca

SEQ ID NO: 646 - DNA, 70 bp, Homo sapiens
gtttctaaaaatagtgttataggctggactgtgtccccttcctgtgccccgccgccatgcatatatggac

SEQ ID NO: 647 - DNA, 70 bp, Homo sapiens
gtcttattattttttgtcgatcaatgcttatcctcgtgttcgttttgatatattaatgtatatgtgttgg

SEQ ID NO: 648 - DNA, 70 bp, Homo sapiens
catctttagtgaaagagtaaatggtggccgagggctccttttgtgagggatgtgccttggtgaagaaggc

SEQ ID NO: 649 - DNA, 70 bp, Homo sapiens
cggatcacttgaggtcaggagttcgagaccagcctccaacatggcaaagccctgtctctactaaaaatac

SEQ ID NO: 650 - DNA, 70 bp, Homo sapiens
catctctccaatctacccaagaggaacccagttacccgaaggcaggttccacagcccactcccagcagca

SEQ ID NO: 651 - DNA, 70 bp, Homo sapiens
ttccctgacccccataccctcacccttaaaattctcctgtaactcaactaacaaaatcaagcctgattca

SEQ ID NO: 652 - DNA, 70 bp, Homo sapiens
ggtcttctaagccaggcaggtgaggcaatttcatgtctgtgatgtgcatccgctccactttatcccttgt

SEQ ID NO: 653 - DNA, 70 bp, Homo sapiens
cacgacggtctaaacccagctcacgttccctattagtgggtgaacaatccaacgcttggtgaattctgct

SEQ ID NO: 654 - DNA, 70 bp, Homo sapiens
gcatcagcaggcagttggttgaagtcagcggaggggtgttccattctttgtttttccagggcttgttttc

SEQ ID NO: 655 - DNA, 70 bp, Homo sapiens
tctatccaactttgccatcttagactagccttctttaccctactgacccatacattggtctctgtatcct

SEQ ID NO: 656 - DNA, 70 bp, Homo sapiens
ctccaccccggtggtgctggtccggaaggacgacctgcacagaaagagactgcacaacacgatagcactg

SEQ ID NO: 657 - DNA, 70 bp, Homo sapiens
ctaaccattcgtgattattaagatagggttgggtcagggcttagggagggggcagaaatattggggatag

SEQ ID NO: 658 - DNA, 70 bp, Homo sapiens
gactacttcccaattaactccaactcacagtgatcctttcaactcatgcggcatctatttttgccaccac

SEQ ID NO: 659 - DNA, 70 bp, Homo sapiens
agccctcagtagacacgtctagggcaggcttgagagatcagatggcgtgaaaggcttgtgatctgttcgt

SEQ ID NO: 660 - DNA, 70 bp, Homo sapiens
gggcctggaatttcctttccacttgatagaagtatatattaggaagtccagttaatagtatttttattta

SEQ ID NO: 661 - DNA, 70 bp, Homo sapiens
cgtccatgccctgagtccaccccggggaaggtgacagcattgcttctgtgtaaattatgtactgcaaaaa

SEQ ID NO: 662 - DNA, 70 bp, Homo sapiens
ttgggatctgaggggtcctctctgtgcccatcacagtttgagcttcagggaaaagaagaagaggtctttg

SEQ ID NO: 663 - DNA, 70 bp, Homo sapiens
cgcaggcaaccaaaactaaagcacccgacgacttagttgctccggtcgtgaagaaaccacacatctatta

SEQ ID NO: 664 - DNA, 70 bp, Homo sapiens
aaacagatagccacaagaggttgggacagaggagggtaaaggctcagaaggaggttcaacctctgactca

SEQ ID NO: 665 - DNA, 70 bp, Homo sapiens
ccacgtggtctcacgttttcatgttgacagccagtcagagtcaagagctcagctgtatcgacagatcgtc

SEQ ID NO: 666 - DNA, 70 bp, Homo sapiens
tgtgaagccaggtgtgggttctactcagtgccatagatagactgagtcttctctcgtaggttaccattac

SEQ ID NO: 667 - DNA, 70 bp, Homo sapiens
gtccaacagaggagggatgtggagagcgtttcaggtgcttttcaggtcagtgcatcagcaaatcattggt

SEQ ID NO: 668 - DNA, 70 bp, Homo sapiens
gaaaacataaccagccattggctatttaaacttgtatttttttatttacaaaatataaatatgaagacat

SEQ ID NO: 669 - DNA, 70 bp, Homo sapiens
agggtgtgggtggctcccctccagggatggctgctccacggtttgcattaaaggttctgtataaggccaa

SEQ ID NO: 670 - DNA, 70 bp, Homo sapiens
agcatggaaacaagatgaaattccatttgtaggtagtgagacaaaattgatgatccattaagtaaacaat

SEQ ID NO: 671 - DNA, 70 bp, Homo sapiens
ccttggttccctaaccctaattgatgagaggctcgctgcttgatggtgtgtacaaactcacctgaatggg

SEQ ID NO: 672 - DNA, 70 bp, Homo sapiens
caatctgaaataaaagtgggatgggagagcgtgtccttcagatcaagggtactaaagtccctttcgctgc

SEQ ID NO: 673 - DNA, 70 bp, Homo sapiens
tgatggcgccttcaaagaggtgaagctgtcggactacaaagggaagtacgtggtcctctttttctaccct

SEQ ID NO: 674 - DNA, 70 bp, Homo sapiens
ggagtagctgagatcttagaagccgtcacctacactcaagcctcgcccaaagaagcaaaagttgaaccca

SEQ ID NO: 675 - DNA, 70 bp, Homo sapiens
agggaatagaaatgaaacaaattatctctcatcttttgactatttcaagtctaataaattcttaattaac

SEQ ID NO: 676 - DNA, 70 bp, Homo sapiens
cccagaaaacagaagtttctactgtctcgtctacccaagttggccccaactgaggacccaatattggcct

SEQ ID NO: 677 - DNA, 70 bp, Homo sapiens
cgtgtgattggtgcaggagaattcggtgaagtctgcagtggccgtttgaaacttccagggaaaagagatg

SEQ ID NO: 678 - DNA, 70 bp, Homo sapiens
caaaactggatggcatccgaattgtctggaagttttgtcttgggcatgatgggctgggccaaatgaaatg

SEQ ID NO: 679 - DNA, 70 bp, Homo sapiens
cacccttcaggggatgagaagttttcaaggggtattactcaggcactaaccccaggttagatgacagcac

SEQ ID NO: 680 - DNA, 70 bp, Homo sapiens
tggaggaccgaaccgtagtacgctaaaaagtgcccggatggacttgtggatagtggtgaaattccaatcg

SEQ ID NO: 681 - DNA, 70 bp, Homo sapiens
taagcttgcgttgattaagtccctgccctttgtacacaccgcccgtcgctactaccgattggatggttta

SEQ ID NO: 682 - DNA, 70 bp, Homo sapiens
catcattcagatggctttccagatgaccaggacgagtgggatattttgcccccaacttggctcggcatgt

SEQ ID NO: 683 - DNA, 70 bp, Homo sapiens
ctgactattactgtaactcccgggacagcagtggtaaccgtctggtattcggcggagggaccaagctgac

SEQ ID NO: 684 - DNA, 70 bp, Homo sapiens
gcgcgctcgccccgccgctcctgctgcagccccaggcccctcgccgccgccaccatggacgccatcaaga

SEQ ID NO: 685 - DNA, 70 bp, Homo sapiens
agcgagttctacatcctaacggcagcccactgtctctaccaagccaagagattcgaaggggaccggaaca

SEQ ID NO: 686 - DNA, 70 bp, Homo sapiens
gatctcggatgaccaaaccagccttcggagcgttctctgtcctacttctgactttacttgtggtgtgaca

SEQ ID NO: 687 - DNA, 70 bp, Homo sapiens
tggtcctgcgcttgagggggggtgtctaagtttccccttttaaggtttcaacaaatttcattgcactttc

SEQ ID NO: 688 - DNA, 70 bp, Homo sapiens
aagacgaatagtcaaaagggagcctcttctacctggatgaaggcaattgtgtcatcggggacactaggtg

SEQ ID NO: 689 - DNA, 70 bp, Homo sapiens
cctacattccctctcctgcccagatgccctttggaaagccattgaccacccaccatattgtttgatctac

SEQ ID NO: 690 - DNA, 70 bp, Homo sapiens
aacatgacaaggaattcttccacccacgctaccaccatcgagagttccggtttgatctttccaagatccc

SEQ ID NO: 691 - DNA, 70 bp, Homo sapiens
accgtgacaattggcctccgggggccactttcggcggagggaccaaggtggagatcaaacataccaccgg

SEQ ID NO: 692 - DNA, 70 bp, Homo sapiens
ccaactaccgcgcttatgccacggagccgcacgccaagaaaaaatctaagatctccgcctcgagaaaatt

SEQ ID NO: 693 - DNA, 70 bp, Homo sapiens
agattccaggcggtgcaaccctggtgttcgaggtggagctgctcaaaatagagcgacgaactgagctgta

SEQ ID NO: 694 - DNA, 70 bp, Homo sapiens
gcttcgagggtgtgaagggaaagaagaagatgtcagcagcagaggcagtgaaagaaaaatggctcccgta

SEQ ID NO: 695 - DNA, 70 bp, Homo sapiens
ataggaagtagacctctttttcttaccagtctcctcccctactctgccccctaagctggctgtacctgtt

SEQ ID NO: 696 - DNA, 70 bp, Homo sapiens
agcgccgctgtgactcggggtgacctccgcatcctgcctgaggcccatcagcgcacatggcatgcctgga

SEQ ID NO: 697 - DNA, 70 bp, Homo sapiens
gcaagtggtcaacagcaggtggctgtcgagacgtctaatgaccattctccatatacctttcaacctaata

SEQ ID NO: 698 - DNA, 70 bp, Homo sapiens
ccgctaggggtgcggggttggggaggaggccgctagtctacgcctgtggagccgatactcagccctctgc

SEQ ID NO: 699 - DNA, 70 bp, Homo sapiens
gcgcagatagcacttcagctcggccatctcagatcccaactccagtgaataacaacacaaagaagcgaga

SEQ ID NO: 700 - DNA, 70 bp, Homo sapiens
tagctggacaggccctgcccctcaccagcaagaggcatgattggatggagcttctaatgtcattcaaaaa

SEQ ID NO: 701 - DNA, 70 bp, Homo sapiens
gtatatctttaattctgggagaaatgagataaaagatgtacttgtgaccattgtaacaatagcacaaata

SEQ ID NO: 702 - DNA, 70 bp, Homo sapiens
tcaagactttaccactggttgactcaaaagattcaatgatcctgctgggctcggtggagcggtcggaact

SEQ ID NO: 703 - DNA, 70 bp, Homo sapiens
atggggacttgtgaatttttctaaaggtgctatttaacatgggaggagagcgtgtgcgctccagcccagc

SEQ ID NO: 704 - DNA, 70 bp, Homo sapiens
catgtctcccatcagaaagattcattggcatgccacagggattctcctccttcatcctgtaaaggtcaac

SEQ ID NO: 705 - DNA, 70 bp, Homo sapiens
agccgccgcgtcccctcgccgagtcccctcgccagattccctccgtcgccgccaagatgatgtgcggggc

SEQ ID NO: 706 - DNA, 70 bp, Homo sapiens
cataaatcaactgtccatcaggtgaggtgtgctccatacccagcggttcttcatgagtagtgggctatgc

SEQ ID NO: 707 - DNA, 70 bp, Homo sapiens
tatctatattttacataaatttagtattttgtttcagtgcactaatatgtaagacaaaaaggactactta

SEQ ID NO: 708 - DNA, 70 bp, Homo sapiens
ctcgcgtctcactcagtgtaccttctagtcccgccatggccgctctcacccgggacccccagttccagaa

SEQ ID NO: 709 - DNA, 70 bp, Homo sapiens
cgaaatatcaatgcaaactaggatatgtaacagcagatggtgaaacatcaggatcaattagatgtgggaa

SEQ ID NO: 710 - DNA, 70 bp, Homo sapiens
tttactaagtaaaagggtggagaggttcctggggtggattcctaagcagtgcttgtaaaccatcgcgtgc

SEQ ID NO: 711 - DNA, 70 bp, Homo sapiens
atagatcttggccctgttaaggcatccacttcacagttctgaaggctgagtcagccccactccacagtta

SEQ ID NO: 712 - DNA, 70 bp, Homo sapiens
gcggatcagtgatagccatgaggacactgggattctggacttcagctcactgctgaaaaagagagacagt

SEQ ID NO: 713 - DNA, 70 bp, Homo sapiens
aaaaaagactttgagctgaatgctctcaacgcaaggattgaggatgaacaggccctcggcagccagctgc

SEQ ID NO: 714 - DNA, 70 bp, Homo sapiens
catcaactatgaagcatttgtgaagcacatcatgtccagctaaacctcgtgcccagaagccaggaaggct

SEQ ID NO: 715 - DNA, 70 bp, Homo sapiens
gaaattcttggaaacttccattaagtgtgtagattgagcaggtagtaattgcatgcagtttgtacattag

SEQ ID NO: 716 - DNA, 70 bp, Homo sapiens
agcccttgcaaaaacacggcttgtggcattggcatacttgcccttacaggtggagtatcttcgtcacaca

SEQ ID NO: 717 - DNA, 70 bp, Homo sapiens
tttacttggtataatatacatggttaaaatgcttatgtgacttcgagtaggtgaatcttaaagaaataaa

SEQ ID NO: 718 - DNA, 70 bp, Homo sapiens
ctgctatagcggtgtcatgttggatcgctttgtgactgttcatctgtccttgacagtggctgtcatcttg

SEQ ID NO: 719 - DNA, 70 bp, Homo sapiens
acaaggagtcagacattttaagatggtggcagtagaggctatggacagggcatgccacgtgggctcatat

SEQ ID NO: 720 - DNA, 70 bp, Homo sapiens
caaagccagacaagccaacgacacagctaaagatgtactggcacagattacagagctccaccagaacctc

SEQ ID NO: 721 - DNA, 70 bp, Homo sapiens
aggcgcggaggtctggcctataaagtagtcgcggagacggggtgctggtttgcgtcgtagtctcctgcag

SEQ ID NO: 722 - DNA, 70 bp, Homo sapiens
ttggaggcattcctacttacggggttggagctgggggctttcccggctttggtgtcggagtcggaggtat

SEQ ID NO: 723 - DNA, 70 bp, Homo sapiens
ccgtctgtcctttgtccacaaggaatttccctgggcgctaattatgagggaggcgtgtagcttcttatca

SEQ ID NO: 724 - DNA, 70 bp, Homo sapiens
ctggtattatctctctatcagataagattttgttaatgtactattttactcttcaataaataaaacagtt

SEQ ID NO: 725 - DNA, 70 bp, Homo sapiens
agatgggtgctggtcctgttgatcccagtctctgccagaccaaggcgagtttccccactaataaagtgcc

SEQ ID NO: 726 - DNA, 70 bp, Homo sapiens
gtgctacacccttttccagctggatgagaatttgagtgctctgatccctctacagagcttccctgactca

SEQ ID NO: 727 - DNA, 70 bp, Homo sapiens
atcccagtggaggggacccttttacttgccctgaacatacacatgctgggccattgtgattgaagtcttc

SEQ ID NO: 728 - DNA, 70 bp, Homo sapiens
aaaagccacggaccgttgcacaaaaaggaaagtttgggaagggatgggagagtggcttgctgatgttcct

SEQ ID NO: 729 - DNA, 70 bp, Homo sapiens
atgccggcctccctgttgtccactgccccagccacatcatccctgtgcgggttgcagatgctgctaaaaa

SEQ ID NO: 730 - DNA, 70 bp, Homo sapiens
ccccaaaccataaaaccctatacaagttgttctagtaacaatacatgagaaagatgtctatgtagctgaa

SEQ ID NO: 731 - DNA, 70 bp, Homo sapiens
tgcactccagccggggtgacagaagagaccttgtctcgaaaacgaatctgaaaacaatggaaccatgcct

SEQ ID NO: 732 - DNA, 70 bp, Homo sapiens
gaggacctccgctgcaaatacatctccctcatctacaccaactatgaggcgggcaaggatgactatgtga

SEQ ID NO: 733 - DNA, 70 bp, Homo sapiens
gaggccttgtgtcctttaatcactgcatttcattttgattttggataataaacctggctcagcctgagcc

SEQ ID NO: 734 - DNA, 70 bp, Homo sapiens
tccaaggcaggtcatcctgacactgcaacccactttggtggctgtgggcaagtccttcaccattgagtgc

SEQ ID NO: 735 - DNA, 70 bp, Homo sapiens
taatgctctgggaggatggggagaactacagaattcggtaaagacatttggggagacacatcctttcacc

SEQ ID NO: 736 - DNA, 70 bp, Homo sapiens
ttccccaattatcctccttcactccctgtcatagttaccgatggtgtcccgttgtgtgggtttactctgt

SEQ ID NO: 737 - DNA, 70 bp, Homo sapiens
cgtaagggctacagtcgaaaagggtttgaccggcttagcactgagggcagtgaccaagagaaagaggatg

SEQ ID NO: 738 - DNA, 70 bp, Homo sapiens; identical to SEQ ID NO: 241

SEQ ID NO: 739 - DNA, 70 bp, Homo sapiens
tgagagctaaacccagcaattttctatgattttttcagatatagataataaacttatgaacagcaactaa

SEQ ID NO: 740 - DNA, 70 bp, Homo sapiens
ggctggaaccatggagggtgtagaagagaagaagaaggaggttcctgctgtgccagaaacccttaagaaa

SEQ ID NO: 741 - DNA, 70 bp, Homo sapiens
aagaacttgccactaaactgggttaaatgtacactgttgagttttctgtacataaaaataattgaaataa

SEQ ID NO: 742 - DNA, 70 bp, Homo sapiens
ggtgctgtggaatgcccagccagttaagcacaaaggaaaacatttcaataaaggatcatttgacaactgg

SEQ ID NO: 743 - DNA, 70 bp, Homo sapiens
cgggccagccgaggctacaaaaactaaccctggatcctactctcttattaaaaagatttttgctgacaaa

SEQ ID NO: 744 - DNA, 70 bp, Homo sapiens
taatcatgtcgtcgccaagtcccgcttctggtactttgtatctcagttaaagaagatgaagaagtcttca

SEQ ID NO: 745 - DNA, 70 bp, Homo sapiens
gaggagatcatcaagactttatccaaggaggaagagaccaagaaataaaacctcccactttgtctgtaca

SEQ ID NO: 746 - DNA, 70 bp, Homo sapiens
ttctcgtggtaataccagagtagaaggagagggtgactttaccgaactgacagccattggggaggcagat

SEQ ID NO: 747 - DNA, 70 bp, Homo sapiens
tcttgctgatataatggccaagaggaatcagaaacctgaagttagaaaggctcaacgagaacaagctatc

SEQ ID NO: 748 - DNA, 70 bp, Homo sapiens
tgaggaaatctgaaatagagtactatgctatgttggctaaaactggtgtccatcactacagtggcaataa

SEQ ID NO: 749 - DNA, 70 bp, Homo sapiens
cccaggctgtttggcgctgcccaggaatggtatcaattcccctgtttctcttgtagccagttactagaat

SEQ ID NO: 750 - DNA, 70 bp, Homo sapiens
ctgtccaatagaaaaagttggtgtgctggagctacctcacctcagcttgagagagccagttgtgtgcatc

SEQ ID NO: 751 - DNA, 70 bp, Homo sapiens
gccaaggaagagtcggaggagtcggacgaggatatgggatttggtctctttgactaatcaccaaaaagca

SEQ ID NO: 752 - DNA, 70 bp, Homo sapiens
gagcgcggcggcaagatggcagtgcaaatatccaagaagaggaagtttgtcgctgatggcatcttcaaag

SEQ ID NO: 753 - DNA, 70 bp, Homo sapiens
tcggacgccggattttgacgtgctctcgcgagatttgggtctcttcctaagccggcgctcggcaagttct

SEQ ID NO: 754 - DNA, 70 bp, Homo sapiens
gccaagctgactcctgaggaagaagagattttaaacaaaaaacgatctaaaaaaattcagaagaaatatg

SEQ ID NO: 755 - DNA, 70 bp, Homo sapiens
ttcctctccagcccctgcgtaatcgataaggaaacccggacgctgctgcccctttctttttttcaggcgg

SEQ ID NO: 756 - DNA, 70 bp, Homo sapiens
tttcgttgcctgatcgccgccatcatgggtcgcatgcatgctcccgggaagggcctgtcccagtcggctt

SEQ ID NO: 757 - DNA, 70 bp, Homo sapiens
tcttttaccaaggacccgccaacatgggccgcgttcgcaccaaaaccgtgaagaaggcggcccgggtcat

SEQ ID NO: 758 - DNA, 70 bp, Homo sapiens
aacgacgcaaacgaagccaagttcccccagctccgaacaggagctctctatcctctctctattacactcc

SEQ ID NO: 759 - DNA, 70 bp, Homo sapiens
gttgaggtggaagtcaccattgcagatgcttaagtcaactattttaataaattgatgaccagttgttaaa

SEQ ID NO: 760 - DNA, 70 bp, Homo sapiens
gctggtgaagatgcatgaataggtccaaccagctgtacatttggaaaaataaaactttattaaatcaaaa

SEQ ID NO: 761 - DNA, 70 bp, Homo sapiens
gcctcgtcgaaggtgctaaaaagatcaaagttgcagaactgttagccaacatgccagaccccactcagga

SEQ ID NO: 762 - DNA, 70 bp, Homo sapiens
ctattccctcaaatctgagggagctgagtaacaccatcgatcatgatgtagagtgtggttatgaacttta

SEQ ID NO: 763 - DNA, 70 bp, Homo sapiens
tatttgtatgtggggagtaggtgtttgaggttcccgttctttcccttcccaagtctctgggggtggaaag

SEQ ID NO: 764 - DNA, 70 bp, Homo sapiens
cggagaagaatcggatcaataaggccgtatctgaggaacagcagcctgcactcaagggcaaaaagggaaa

SEQ ID NO: 765 - DNA, 70 bp, Homo sapiens
agtgggtggaggcagccagggcttacctgtacactgacttgagaccagttgaataaaagtgcacacctta

SEQ ID NO: 766 - DNA, 70 bp, Homo sapiens
gaccggttaaggagaagccagagttagagtaggagaggactaattctcagcagcagtggaggtgagttct

SEQ ID NO: 767 - DNA, 70 bp, Homo sapiens
tattgatgggcccaagcgtaaccaggctcttctgattggccggtgtacttcagtttccgtccaaggtccg

SEQ ID NO: 768 - DNA, 70 bp, Homo sapiens
tcttttgtggttgttgctggcccaatgagtccctagtcacatcccctgccagagggagttcttcttttgt

SEQ ID NO: 769 - DNA, 70 bp, Homo sapiens
gagggcagggaccgtatcttatttactgttagtatccgttgcatctagtgtggtgcacctggcacacagt

SEQ ID NO: 770 - DNA, 70 bp, Homo sapiens
tggcaagagagcctcacacctcactaggtgcagagagcccaggccttatgttaaaatcatgcacttgaaa

SEQ ID NO: 771 - DNA, 70 bp, Homo sapiens
ggagcctctttgtagggactgtgcctaggtagcatgtcctaacatttgttctggtcttgcataacttcag

SEQ ID NO: 772 - DNA, 70 bp, Homo sapiens
gtatcccgcgggtggaggccggggtggcgccggccggggcgggggagcccaaaagaccggctgccgcctg

SEQ ID NO: 773 - DNA, 70 bp, Homo sapiens
gtagggatggggctgtggggatagtgaggcatcgcaatgtaagactcgggattagtacacacttgttgat

SEQ ID NO: 774 - DNA, 70 bp, Homo sapiens; identical to SEQ ID NO: 460

SEQ ID NO: 775 - DNA, 70 bp, Homo sapiens
gcctgcaccagtgccgtcctgctgatgtggtaggctagcaatattttggttaaaatcatgtttgtggccg

SEQ ID NO: 776 - DNA, 70 bp, Homo sapiens
tcactccttaaattcacactttgccacttaactccagtgtggatgacagagcgagaccctgcctcaaaaa

SEQ ID NO: 777 - DNA, 70 bp, Homo sapiens
gccctgggcagccagcattcattgtaagttccctctttgaaaactggtgtgtgggtgttcagttctgtgt

SEQ ID NO: 778 - DNA, 70 bp, Homo sapiens
agaaaaaagtcacgttaaatggtttcttggacacgcttatgtcagatcctcccccgcagtgtctggtctg

SEQ ID NO: 779 - DNA, 70 bp, Homo sapiens
catgtgggcaaagccttcaatcagggcaagatcttcaagtgaacatctcttgccatcacctagctgcctg

SEQ ID NO: 780 - DNA, 70 bp, Homo sapiens
atgaagccaggattcagtccccgtgggggtggctttggcggccgagggggctttggtgaccgtggtggtc

SEQ ID NO: 781 - DNA, 70 bp, Homo sapiens
gcagctatttcaaagtgtgttggattaattaggatcatccctttggttaataaataaatgtgtttgtgct

SEQ ID NO: 782 - DNA, 70 bp, Homo sapiens
gaccagttgttatttacagctctgtaacctcccgttgcgtcaagtctaaaccaagattatgtgacttgca

SEQ ID NO: 783 - DNA, 70 bp, Homo sapiens
cacttcacagtaaatgccaaagctgctggcaaaggcaagctggacgtccagttctcaggactcaccaagg

SEQ ID NO: 784 - DNA, 70 bp, Homo sapiens
aggaagttatgggaatacctgtggtggttgtgatccctaggtcttgggagctcttggaggtgtctgtatc

SEQ ID NO: 785 - DNA, 70 bp, Homo sapiens
ctcactgggtggctttgcctatgtggagatcagctccaaagagatgactgtcacttacatcgaggcctcg

SEQ ID NO: 786 - DNA, 70 bp, Homo sapiens
agcgtgagattgtccgggacatcaaggagaaactgtgttatgtagctctggactttgaaaatgagatggc

SEQ ID NO: 787 - DNA, 70 bp, Homo sapiens
tagaatcctcaaccgtgcggaccatcaaccttcgagaaattccagttgtctttttcccagccgcatcctg

SEQ ID NO: 788 - DNA, 70 bp, Homo sapiens
caaccacgacaaaggaagttgacctaaacatgtaaccatgccctaccctgttaccttgctagctgcaaaa

SEQ ID NO: 789 - DNA, 70 bp, Homo sapiens
gaggctctgtaaccttatctaagaacttggaagccgtcagccaagtcgccacatttctctgcaaaatgtc

SEQ ID NO: 790 - DNA, 70 bp, Homo sapiens
taggcggagcctcggccgcgggccgccttggtatatctgcgtgcgcgcgtctgctgggccagtcgggaca

SEQ ID NO: 791 - DNA, 70 bp, Homo sapiens
ctagcggttacgccaacgcgcgcgtgcgcccttgcgcgtttctctcttcccactcgggtttgacctacag

SEQ ID NO: 792 - DNA, 70 bp, Homo sapiens
cgcaaggaggggctgcttctgaggtcggtggctgtctttccattaaagaaacaccgtgcaacgtgaaaaa

SEQ ID NO: 793 - DNA, 70 bp, Homo sapiens
ggagttggtcaaatgagggaacatctgggttatgccttttttaaagtagttttctttaggaactgtcagc

SEQ ID NO: 794 - DNA, 70 bp, Homo sapiens
aggcatctggagagtccaggagaggagactcacctctgtcgcttgggttaaacaagagacaggttttgta

SEQ ID NO: 795 - DNA, 70 bp, Homo sapiens
aactaatccatcaccggggtggtttagtggctcaacattgtgttcccatttcagctgatcagtgggcctc

SEQ ID NO: 796 - DNA, 70 bp, Homo sapiens
acaatttgtttcagagaagagagttgaacagtggtgagctgggctcacagctccatccatgggccccatt

SEQ ID NO: 797 - DNA, 70 bp, Homo sapiens
tgttgacacaggtctttcctaaggctgcaaggtttaggctggtggcccaggaccatcatcctactgtaat

SEQ ID NO: 798 - DNA, 70 bp, Homo sapiens
ctgggggagtggaatagtatcctccaggtttttcaattaaacggattattttttcagaccgaaaagaaaa

SEQ ID NO: 799 - DNA, 70 bp, Homo sapiens; identical to SEQ ID NO: 261

SEQ ID NO: 800 - DNA, 70 bp, Homo sapiens
ggccacttttcactaacagaagtcacaagccaagtgagacactcatccaagaggaaggatggccagtatc

SEQ ID NO: 801 - DNA, 70 bp, Homo sapiens
agaccagagatagtggggagacttcttggcttggtgaggaaaagcggacatcagctggtcaaacaaactc

SEQ ID NO: 802 - DNA, 70 bp, Homo sapiens
ccatggatgagaaagtcgagttgtatcgcattaagttcaaggagagctttgctgagatgaacaggggctc

SEQ ID NO: 803 - DNA, 70 bp, Homo sapiens
gtttttcagctcacttcaagggtacctgaagcgaattggcaccaaagcagcagctgtattgccgcagttc

SEQ ID NO: 804 - DNA, 70 bp, Homo sapiens
ggatagataattttatttgaaattttacacactgaaagctctaaataaacagatacattcacattcaaaa

SEQ ID NO: 805 - DNA, 70 bp, Homo sapiens
agtttccccaccagtgaatgaaagtcttgtgactagtgctgaagcttattaatgctaagggcaggcccaa

SEQ ID NO: 806 - DNA, 70 bp, Homo sapiens
ggggaggcatcagtgtccttggcaggctgatttctaggtaggaaatgtggtagctcacgctcacttttaa

SEQ ID NO: 807 - DNA, 70 bp, Homo sapiens
gacgcggctcaaaaggaaaccaagtggtcaggagttgtttctgacccactgatctctactaccacaagga

SEQ ID NO: 808 - DNA, 70 bp, Homo sapiens
cggccgaacccagacccgaggttttagaagcagagtcaggcgaagctgggccagaaccgcgacctccgca

SEQ ID NO: 809 - DNA, 70 bp, Homo sapiens
cttgaaattgtccccgtggtctcttactttcctttccccagcccagggtggacttagaaagcaggggcta

SEQ ID NO: 810 - DNA, 70 bp, Homo sapiens
caggggccaggggaacccgtgaggatcactctcaaatgagattaaaaacaaggaagcagagaatggtcag

SEQ ID NO: 811 - DNA, 70 bp, Homo sapiens
ccaagtccctgaagtctggagacgcggccatcgtggagatggtgccgggaaagcccatgtgtgtggagag

SEQ ID NO: 812 - DNA, 70 bp, Homo sapiens
acaaggtggggacagacttgctggaggaggagatcaccaagtttgaggagcacgtgcagagtgtcgatat

SEQ ID NO: 813 - DNA, 70 bp, Homo sapiens
aacgcattaagaggtttatttgggtacatggcccgcagtggcttttgccccagaaaggggaaaggaacac

SEQ ID NO: 814 - DNA, 70 bp, Homo sapiens
cctgccctgcacccttgtacagtgtctgtgccatggatttcgtttttcttggggtactcttgatgtgaag

SEQ ID NO: 815 - DNA, 70 bp, Homo sapiens
gaagaagggccccaatgccaactcttaagtcttttgtaattctggctttctctaataaaaaagccactta

SEQ ID NO: 816 - DNA, 70 bp, Homo sapiens
aaagtgtgaatgtgggtgtcggctgcggcattaaattcatcatctcaacccagagtgtctggtctccctg

SEQ ID NO: 817 - DNA, 70 bp, Homo sapiens
cttttccctatccacaggggtgtttgtgtgtgtgcgcgtgtgcgtttcaataaagtttgtacactttcaa

SEQ ID NO: 818 - DNA, 70 bp, Homo sapiens
atgcgcagcagcggcgccgacgcggggcggtgcctggtgaccgcgcgcgctcccggaagtgtgccggcgt

SEQ ID NO: 819 - DNA, 70 bp, Homo sapiens
ggggtcaaaaggtacctaagtatatgattgcgagtggaaaaataggggacagaaatcaggtattggcagt

SEQ ID NO: 820 - DNA, 70 bp, Homo sapiens
atcagttcttaatttaatttttaagtattgttttactcctttttattcatacgtaaaattttggattaat

SEQ ID NO: 821 - DNA, 70 bp, Homo sapiens
aaagagggtccatcaaagagatgagccatcaccccccaggacacacagtggtcaaggatagaagccattt

SEQ ID NO: 822 - DNA, 70 bp, Homo sapiens
gcacggcatggattaacacggcagaggaacaaaggtgtgctctgagcttcttcatatttcaccttcaccc

SEQ ID NO: 823 - DNA, 70 bp, Homo sapiens
ggctatgcaacagctctcacctacgcgagtcttactttgagttagtgccataacagaccactgtatgttt

SEQ ID NO: 824 - DNA, 70 bp, Homo sapiens
gtacagtcgccgcgtgcggagcttgttactggttacttggcctcatggcggtccgagcttcgttcgagaa

SEQ ID NO: 825 - DNA, 70 bp, Homo sapiens
agcatattgtctggggattgttgggacaggttttggtgactctgtgcccttgctctctaacttctgagcc

SEQ ID NO: 826 - DNA, 70 bp, Homo sapiens
agacacatggaacaaagaagctgtgaccccagcaggatgtctaatatgtgaggaaatgagatgtccacct

SEQ ID NO: 827 - DNA, 70 bp, Homo sapiens
agttcgttgtgctgtttctgactcctaatgagagttccttccagaccgttagctgtctccttgccaagcc

SEQ ID NO: 828 - DNA, 70 bp, Homo sapiens
gctccaggttgggtgctcacagaacccttttcctgactctcatggaagatggtggaaggaaaatagactg

SEQ ID NO: 829 - DNA, 70 bp, Homo sapiens
aaaaaatcttacacatctgccaccggaaataccatgcacagagtccttaaaaaatagagtgcagtattta

SEQ ID NO: 830 - DNA, 70 bp, Homo sapiens
gttgaaggggctggtgccactgggacccgaatcaagtcgacacactacgttgagtttattaacaaaagcc

SEQ ID NO: 831 - DNA, 70 bp, Homo sapiens
agaagacaaagagcaaggggccctacatctgcgctctgtgcgccaaggagttcaagaacggctacaatct

SEQ ID NO: 832 - DNA, 70 bp, Homo sapiens
cctaccccgaactccaaaaattacacctggagtcaggtgcagaagggaaccttgtatttcacaggcctca

SEQ ID NO: 833 - DNA, 70 bp, Homo sapiens
accacagtggtgtccgagaagtcaggcacgtagctcagcggcggccgcggcgcgtgcgtctgtgcctctg

SEQ ID NO: 834 - DNA, 70 bp, Homo sapiens
acagtaagattgaggatgagcaggcgctggcccttcaactacagaagaaactgaaggaaaaccaggcacg

SEQ ID NO: 835 - DNA, 70 bp, Homo sapiens
tgaggctcccaaggaacctgcctttgaccccaagagtgtaaagatagacttcactgccgaccagattgaa

SEQ ID NO: 836 - DNA, 70 bp, Homo sapiens
ccaaaatacttgcatccaaggttctagtctctgttgctgtgctggtctttagccccactgctggcactga

SEQ ID NO: 837 - DNA, 70 bp, Homo sapiens
gagtgtgtctcatgctttcagatgtgcatatgagcagaattaattaaacatttgcctatgactccaacaa

SEQ ID NO: 838 - DNA, 70 bp, Homo sapiens
atattgcaaaaggatgtgtgtctttctccccgagctcccctgttccccttcattgaaaaccaccacggtg

SEQ ID NO: 839 - DNA, 70 bp, Homo sapiens; identical to SEQ ID NO: 623

SEQ ID NO: 840 - DNA, 70 bp, Homo sapiens
taatcattttctagaaagtatgggtatctatactaatgtttttatatgaagaacataggtgtctttgtgg

SEQ ID NO: 841 - DNA, 70 bp, Homo sapiens
aatgtaactatttagccctggattatacatactgtccaattttcattaaatttttgtcttataactataa

SEQ ID NO: 842 - DNA, 70 bp, Homo sapiens
ttggctgccggtgagttgggtgccggtggagtcgtgttggtcctcagaatccccgcgtagccgctgcctc

SEQ ID NO: 843 - DNA, 70 bp, Homo sapiens
ttactactgtgggtttaaagccactgcagcgggagttaaacaaactgagtcaaccagcttccttgaaaaa

SEQ ID NO: 844 - DNA, 70 bp, Homo sapiens
gcagccatctcgccgtgagacagcaagtgtcgcgcagccgtgcgatgttgtcctctacagccatgtattc

SEQ ID NO: 845 - DNA, 70 bp, Homo sapiens
ggaagtgagtggacagcctttgtgtgtatctctccaataaagctctgtgggccaagtcctctaggaaaaa

SEQ ID NO: 846 - DNA, 70 bp, Homo sapiens
aaatctgggttcaaccagcccctgccatttcttaagactttctgctgcactcacaggatcctgagctgca

SEQ ID NO: 847 - DNA, 70 bp, Homo sapiens
taaggtagcaggcagtccagccctgatgtggagacacatgggattttggaaatcagcttctggaggaatg

SEQ ID NO: 848 - DNA, 70 bp, Homo sapiens; identical to SEQ ID NO: 303

SEQ ID NO: 849 - DNA, 70 bp, Homo sapiens
catcttgggttacccactctgtccactcccataggctacagaaaaagtcacaagcgcatggtttccaacc

SEQ ID NO: 850 - DNA, 70 bp, Homo sapiens
tttttccaccctggctccttcagacacgtgcttgatgctgagcaagttcaataaagattcttggaagttt

SEQ ID NO: 851 - DNA, 70 bp, Homo sapiens
tgccatgtactattttacctatgacccgtggattggcaagttattgtatcttgaggacttcttcgtgatg

SEQ ID NO: 852 - DNA, 70 bp, Homo sapiens
gccctgccaccgtggggagtctggtttttctcttcatcctgtctctctcctccttactcttggataaata

SEQ ID NO: 853 - DNA, 70 bp, Homo sapiens
aggccgagctctgcagagcttacaattgagactgctaacccctacctttgaagggatcaacggattgttg

SEQ ID NO: 854 - DNA, 70 bp, Homo sapiens
ccatctctaggatgtcgtctttggtgagatctctattatatcttgtatggtttgcaaaagggcttcctaa

SEQ ID NO: 855 - DNA, 70 bp, Homo sapiens
tgttggtttattgctggcaacgtgaattctctcaggggtctaggaggggcattttggagactgcctgaca

SEQ ID NO: 856 - DNA, 70 bp, Homo sapiens; identical to SEQ ID NO: 414

SEQ ID NO: 857 - DNA, 70 bp, Homo sapiens
atggttccaggactacaatgtctttatttttaactgtttgccactgctgccctcacccctgcccggctct

SEQ ID NO: 858 - DNA, 70 bp, Homo sapiens
gaccatcacatcccttcaagagtcctgaagatcaagccagttctccttccctgcagagctttggccatta

SEQ ID NO: 859 - DNA, 70 bp, Homo sapiens
aggagggtcttcgaggggcctgggggcgggggactaagatggacgcctgggaagggaactgggaggcagc

SEQ ID NO: 860 - DNA, 70 bp, Homo sapiens
tgtcctcaaccccaaatcccccgactccctccccagatctgtcctgggggatgcaaataaagcctgctct

SEQ ID NO: 861 - DNA, 70 bp, Homo sapiens
gccgtgcttctgcccctacaaggtttgggccgaggtgggggagggtcctggttgccggccccgcccggtc

SEQ ID NO: 862 - DNA, 70 bp, Homo sapiens; identical to SEQ ID NO: 62

SEQ ID NO: 863 - DNA, 70 bp, Homo sapiens
gtgcttgtggacatcaggcctcctgccagcagttcttgaagcttctttttcattcctgctactctacctg

SEQ ID NO: 864 - DNA, 70 bp, Homo sapiens
ggcgggaggatcacttgaggccaggactttgagaccagccagggcaacataataagacttttctctactt

SEQ ID NO: 865 - DNA, 70 bp, Homo sapiens
cccaagtgcactcatccaggtcagtgctcagatgtgtttaaggagaccctatattcagggaagttgcgtg

SEQ ID NO: 866 - DNA, 70 bp, Homo sapiens
cctaggttcagagcatgggtgctctgagggacaaagttggattagtataagggagctggagcagctgata

SEQ ID NO: 867 - DNA, 70 bp, Homo sapiens
ggcaggacctgtggccaagttcttagttgctgtatgtctcgtggtaggactgtagaaaagggaactgaac

SEQ ID NO: 868 - DNA, 70 bp, Homo sapiens
ggtgcctgatacctctcagcatttgagggccttttctcttcctgcttcatctctaaaggtccttctagga

SEQ ID NO: 869 - DNA, 70 bp, Homo sapiens
tcaccacgtctggtcgaaagatggcagagctgccggtggaccccatgctgtccaaaatgatcttagcctc

SEQ ID NO: 870 - DNA, 70 bp, Homo sapiens
aaaggataaaccccgatattgggacctcacagtgggtgtctgaaaggacagatcactccggagtatcagg

SEQ ID NO: 871 - DNA, 70 bp, Homo sapiens
aagagaaatacacacttctgagaaactgaaacgacaggggaaaggaggtctcactgagcaccgtcccagc

SEQ ID NO: 872 - DNA, 70 bp, Homo sapiens
aatgaggagtgatcatggctacctcagagctgagctgcgaggtgtcggaggagaactgtgagcgccggga

SEQ ID NO: 873 - DNA, 70 bp, Homo sapiens
gaattctcagctcttgggaacccccttgctcccaggggaggggaaacctttttcattcaacattgtaggg

SEQ ID NO: 874 - DNA, 70 bp, Homo sapiens
aaattcctaaaactgtggaatggatcacgtagacatgtaacccagcagcagtttgcttctgttgtccact

SEQ ID NO: 875 - DNA, 70 bp, Homo sapiens
gtaccattcagaatggactgtttgtacgaagcatgtataatgcagttatcttctttctttcgtcgcagcc

SEQ ID NO: 876 - DNA, 70 bp, Homo sapiens
cttctcctcgaccagccatcatgacatttaccatgaatttacttcctcccaagagtttggactgcccgtc

SEQ ID NO: 877 - DNA, 70 bp, Homo sapiens
cctggcttcattctgctctctcttggcacccgacccttggcagcatgtaccacacagccaagctgagact

SEQ ID NO: 878 - DNA, 70 bp, Homo sapiens
agttatcattaccatgttggtgacctgttcagtttgctgctatctcttttggctgattgcaattctggcc

SEQ ID NO: 879 - DNA, 70 bp, Homo sapiens
ccgcgagatctagcatctctgaaatcctggctgtcgaggctttgaagcatgtgttacctggttaagcttg

SEQ ID NO: 880 - DNA, 70 bp, Homo sapiens
acgaggaaaatggcgctagctcggaagctaccgaggtgctaggagttgccgaagcaagtccggaagctac

SEQ ID NO: 881 - DNA, 70 bp, Homo sapiens
ttgaaaattaaacgtgcttggggttcagctggtgaggctgtccctgtaggaagaaagctctgggactgag

SEQ ID NO: 882 - DNA, 70 bp, Homo sapiens
tgaagctggtggtgtctcggggcggcctgttgggagatcttgcatccagcgacgtggccgtggaactgcc

SEQ ID NO: 883 - DNA, 70 bp, Homo sapiens
tccatgtttgatgtatctgagcaggttgctccacaggtagctctaggagggctggcaacttagaggtggg

SEQ ID NO: 884 - DNA, 70 bp, Homo sapiens
gccattccattcccagcagctttggagacctccaggattatttctctgtcagccctgccacatatcacta

SEQ ID NO: 885 - DNA, 70 bp, Homo sapiens
gataaaagggggagacaaaagatgtacagaaatgatttcctggctggccaactggtggccagtgggaggt

SEQ ID NO: 886 - DNA, 70 bp, Homo sapiens
caataatcagtggtgcttttgtacctaggttttatgtgattttaatgaaacatggatagttgtggccacc

SEQ ID NO: 887 - DNA, 70 bp, Homo sapiens
tacactgctgtacccagatgcctacaaccatccctgccacatacaggtgctcaataaacacttgtagagc

SEQ ID NO: 888 - DNA, 70 bp, Homo sapiens
tcgggaactggcccaacaggtgcagcaagtagctgctgaatattgtagagcatgtcgcttgaagtctact

SEQ ID NO: 889 - DNA, 70 bp, Homo sapiens
taactctgggaggggctcgagagggctggtccttatttatttaacttcacccgagttcctctgggtttct

SEQ ID NO: 890 - DNA, 70 bp, Homo sapiens
gattaagctgaagatgtttattacaatcactctctgtggggggtggccctgctgctcctcagaatcctgg

SEQ ID NO: 891 - DNA, 70 bp, Homo sapiens
catctacccctgctagaaggttacagtgtattatgtagcatgcaaatgtgtttatgtagtggcttaataa

SEQ ID NO: 892 - DNA, 70 bp, Homo sapiens
ccgctgtcgccgccgcggagacaaagatggctgcgagagtcggcgccttcctcaagaatgcctgggacaa

SEQ ID NO: 893 - DNA, 70 bp, Homo sapiens
ttccatgggagatgactcttaagccataggggctggttttccgtactccaaaccatcaggtggacacagt

SEQ ID NO: 894 - DNA, 70 bp, Homo sapiens
attgtttttatctggttacatatatatttctttgtctaatttaatatgtcaaataaatgagttcatctaa

SEQ ID NO: 895 - DNA, 70 bp, Homo sapiens
tctgcgtgggtggtgatgggggttcacctgaacacagagtgtattttcttattgaggccctgtaccttct

SEQ ID NO: 896 - DNA, 70 bp, Homo sapiens
gaatacatttctgcctgataatcatgctgggttctaataagccctacttccacctaatctgtttacagtc

SEQ ID NO: 897 - DNA, 70 bp, Homo sapiens
ggcccagaagaaatttaagcgtcttatgctgcatcggataaagtgggatgaacagacatctaacacaaag

SEQ ID NO: 898 - DNA, 70 bp, Homo sapiens
gccgagtgtattataaaatcgtgggggagatgcccggcctgggatgctgtttggagacggaataaatgtt

SEQ ID NO: 899 - DNA, 70 bp, Homo sapiens
gcagcgcctcccttgtctcagatggtgtgtccagcactcgattgttgtaaactgttgttttgtatgagcg

SEQ ID NO: 900 - DNA, 70 bp, Homo sapiens
gcatacaggttattggagaaattttccttttgttgcatttgtggaagttagttttctggcccgtggcctt

SEQ ID NO: 901 - DNA, 70 bp, Homo sapiens
ttggcgtagccatggcgtctcgtgtcctttcagcctatgtcagccgcctgcccgcggcctttgcgccgct

SEQ ID NO: 902 - DNA, 70 bp, Homo sapiens
cttcaaatatggccgccaagctccgttctcttttaccgcctgatctacggctacaattctggcttcatgc

SEQ ID NO: 903 - DNA, 70 bp, Homo sapiens
agtgtgtcaaacagatctgcgtggtcatgttggagactctctcccagtcccccccgaagggcgtgaccat

SEQ ID NO: 904 - DNA, 70 bp, Homo sapiens
gggccagggctggatggacagacacctccccctacccatatccctcccgtgtgtggttggaaaacttttg

SEQ ID NO: 905 - DNA, 70 bp, Homo sapiens
cctacttcttcagctgacaccccgtgagccttgtcagtgtgtaaataaagctcttttgccaccccccaaa

SEQ ID NO: 906 - DNA, 70 bp, Homo sapiens
ggcccaacacaattcttcttccaacgtggcccagagaagccaaaagattggatacgcatcagacagatgg

SEQ ID NO: 907 - DNA, 70 bp, Homo sapiens
atcccaacgatgacaaggacagtggcttctttccccgaaacccatcgagctccagcatgaactcggttct

SEQ ID NO: 908 - DNA, 70 bp, Homo sapiens
ttcctcgggcatcgacgtgctcatttccaaagatgatggtgcaggtgaccttttccatcgtgagctaaga

SEQ ID NO: 909 - DNA, 70 bp, Homo sapiens
aaaggttttcacaccagacactgcagcagacacccatgataagtaccatgactccaatgagtgcccaggg

SEQ ID NO: 910 - DNA, 70 bp, Homo sapiens; identical to SEQ ID NO: 307

SEQ ID NO: 911 - DNA, 70 bp, Homo sapiens
gaccataggatgggaggatagggagcccctcatgactgagggcagaagaaattgctagaagtcagaacag

SEQ ID NO: 912 - DNA, 70 bp, Homo sapiens
actactctctgaaggagtccaccactagtgagcagagtgccaggatgacagccatggacaatgccagcaa

SEQ ID NO: 913 - DNA, 70 bp, Homo sapiens
agccgggcgagcgctgtgggccaagcaggggttgcagggtagtaggagtgcagactgaaaaaatgcagac

SEQ ID NO: 914 - DNA, 70 bp, Homo sapiens
gccccagcggtaaccaccaatcttcttttgccaatagacctcgaaaatcatcagtaaatgggtcatcagc

SEQ ID NO: 915 - DNA, 70 bp, Homo sapiens
ctagttatgatcagagcagttactctcagcagaacacctatgggcaaccgagcagctatggacagcagag

SEQ ID NO: 916 - DNA, 70 bp, Homo sapiens
aaaaatgtataatataaaattgtaatacactcaaatgattataaaagtaaaagttggtaatttaggcaaa

SEQ ID NO: 917 - DNA, 70 bp, Homo sapiens
actacctttttcgagagtgactcccgttgtcccaaggcttcccagagcgaacctgtgcggctgcaggcac

SEQ ID NO: 918 - DNA, 70 bp, Homo sapiens
ggtgaacctatgggtcgtggaacaaaagttatcctacacctgaaagaagaccaaactgagtacttggagg

SEQ ID NO: 919 - DNA, 70 bp, Homo sapiens
ggggaagcatttgactatctggaacttgtgtgtgcctcctcaggtatggcagtgactcacctggttttaa

SEQ ID NO: 920 - DNA, 70 bp, Homo sapiens
agcaggctgtgcagagcgcgttgaccaagactcataccagagggccacacttttcaagtgtatatggtaa

SEQ ID NO: 921 - DNA, 70 bp, Homo sapiens
ctcggacgggactttcttggtgcggcagagggtgaaggatgcagcagaatttgccatcagcattaaatat

SEQ ID NO: 922 - DNA, 70 bp, Homo sapiens
cttcaggttcctcttactatgataatgtccggcctctggcctatcctgattctgatgctgtgctcatctg

SEQ ID NO: 923 - DNA, 70 bp, Homo sapiens
cactgtgtaccccgagcaacattctaagggtgtgctttcgccttggctaactcctttgacctcattcttc

SEQ ID NO: 924 - DNA, 70 bp, Homo sapiens
gaatctaagttaccatcccttggaaattctggagaaggagtctcatgcaccacctatcacactccctcac

SEQ ID NO: 925 - DNA, 70 bp, Homo sapiens
gccaggattgctacagttgtgattggaggagttgtggccatggcggctgtgcccatggtgctcagtgcca

SEQ ID NO: 926 - DNA, 70 bp, Homo sapiens
gtcttcaactggttagtgtgaaatagttctgccacctctgacgcaccactgccaatgctgtacgtactgc

SEQ ID NO: 927 - DNA, 70 bp, Homo sapiens
caagaggagagtgaagaggaagaggtcgatgaaacaggtgtagaagttaaggacatagaattggtcatgt

SEQ ID NO: 928 - DNA, 70 bp, Homo sapiens
caaggtgcagaatggtttggaaagtagctgtattcctcagtgtggccctgggcattggtgccattcctat

SEQ ID NO: 929 - DNA, 70 bp, Homo sapiens
cctcgtcagcagcgaggaaggaaacagcggcgacagccctgtactgtgtctgaaattttccatttttgtt

SEQ ID NO: 930 - DNA, 70 bp, Homo sapiens
atgtacacacgtgcacgtacacacatgcatgctcgctaagcggaaggaagttgtagattgcttccttcat

SEQ ID NO: 931 - DNA, 70 bp, Homo sapiens
aacaaaccctcatctcatgaaggacggggtgtgtgtgtggcgttgatctttagcctgtctcacaccagtt

SEQ ID NO: 932 - DNA, 70 bp, Homo sapiens
aattttctgcagcattaaagctggcgcttaataagaataagtaataataaagaaatttctaacattccaa

SEQ ID NO: 933 - DNA, 70 bp, Homo sapiens
gcctggaacaaggaccgcacccagattgccatctgccccaacaaccatgaggtgcatatctatgaaaaga

SEQ ID NO: 934 - DNA, 70 bp, Homo sapiens
ggagtgcttccatccctctccaccccttccccccaaaaggttttctttgcaagtgcttttggaactaaga

SEQ ID NO: 935 - DNA, 70 bp, Homo sapiens
agcagctgcctcaccgcccagacattgatttgttcagatgtttcaatgcctcatgatacaataaaaccac

SEQ ID NO: 936 - DNA, 70 bp, Homo sapiens
agaacaggttttcaaagtggcctcctcagacctggtcaacatgggcatcagtgtggttagctacactctg

SEQ ID NO: 937 - DNA, 70 bp, Homo sapiens
ttctctgctggtaattcctgaagaggcatgactgcttttctcagccccaagcctctagtctgggtgtgta

SEQ ID NO: 938 - DNA, 70 bp, Homo sapiens
gagaccagcctggagcctagatctggtgcttcttctgtgctgtggtttaccccaaacctttaggttgttt

SEQ ID NO: 939 - DNA, 70 bp, Homo sapiens
ggatgggaatagcaatgtgtgttcagagagaatgacaatgtgtgttcagagagaatgaattgcttaaact

SEQ ID NO: 940 - DNA, 70 bp, Homo sapiens
acgcatttgagcgattgctctgtgaagagttgtacactgaacactttcaggggaggctgtttacccaggc

SEQ ID NO: 941 - DNA, 70 bp, Homo sapiens
tgactctctgaggctcattttgcagttgttgaaattgtccccgcagttttcaatcatgtctgaaccaatc

SEQ ID NO: 942 - DNA, 70 bp, Homo sapiens
cggaggtggtcaaggctaaagccggagcaggctctgccaccctctccatggcgtatgccggcgcccgctt

SEQ ID NO: 943 - DNA, 70 bp, Homo sapiens
ctgggtcctggggcagggcgagtccaagtgtgaggctgttgatttgttttcaatatttcttttcgtgctg

SEQ ID NO: 944 - DNA, 70 bp, Homo sapiens
cttaagccttccaggacactaaggtcgtgggagcgggactgcaacaagcaatgccagataactgagaaat

SEQ ID NO: 945 - DNA, 70 bp, Homo sapiens
tatttatcccttcttgcctgtgaggactgcggcttttcgctgtggctcgtccttaacgtttctgaaccac

SEQ ID NO: 946 - DNA, 70 bp, Homo sapiens; identical to SEQ ID NO: 230

SEQ ID NO: 947 - DNA, 70 bp, Homo sapiens
gcagcccctttccgggacacctgggttcacacagctttttagcttacataactggtgcagattttctgtg

SEQ ID NO: 948 - DNA, 70 bp, Homo sapiens
gcaaaatgaattcctggcttcagttagctattatttttttaatgacaacatagactgtgctctaagttta

SEQ ID NO: 949 - DNA, 70 bp, Homo sapiens
aatgcaagctcaccaaggtcccctctcagtccccttccctacaccctgaccggccactgccgcacaccca

SEQ ID NO: 950 - DNA, 70 bp, Homo sapiens
tatgatgtatttctgagctaaaactcaactatagaagacattaaaagaaatcgtattcttgccaagtaac

SEQ ID NO: 951 - DNA, 70 bp, Homo sapiens
attttacctctttaccctgtcgctcataatgaggcatcatatatcctctcactctctgggacaccatagc

SEQ ID NO: 952 - DNA, 70 bp, Homo sapiens
gacacctatctaagccattttaaccctcgggattacctagaaaaatattacaagtttggttctaggcact

SEQ ID NO: 953 - DNA, 70 bp, Homo sapiens
attgaaagctaagtgagagagccagagggcctccttggtggtaaaagagggttgcatttcttgcagccag

SEQ ID NO: 954 - DNA, 70 bp, Homo sapiens
acattcacatctagtcaagggcataggaacggtgtcatggagtccaaataaagtggatattcctgctcgg

SEQ ID NO: 955 - DNA, 70 bp, Homo sapiens
caagggcgcaagagtagcggtccaagcctgcaactcatctttcattaaaggcttctctctcaccagcaaa

SEQ ID NO: 956 - DNA, 70 bp, Homo sapiens
agcaccgccgcggagaacaaggccagccccgcggggacagcggggggacctggggctggagcagctgctg

SEQ ID NO: 957 - DNA, 70 bp, Homo sapiens
ctagaagactgcaggctggatcatgctttatatgcactgcctgggccaaccatcgtggacctgaggaaaa

SEQ ID NO: 958 - DNA, 70 bp, Homo sapiens
gagaaatcgaatattctggagcactgattgcagcagggtggctcctttgtgtgcagcaggtgtagtagtc

SEQ ID NO: 959 - DNA, 70 bp, Homo sapiens
cactgctgttgtcattgctccgtttgtgtttgtactaatcagtaataaaggtttagaagtttgaccctaa

SEQ ID NO: 960 - DNA, 70 bp, Homo sapiens
ctcggacaatttctgggtggtgactgagtacccctttagtgagtacccctttagtgctatatttgtgcca

SEQ ID NO: 961 - DNA, 70 bp, Homo sapiens; identical to SEQ ID NO: 415

SEQ ID NO: 962 - DNA, 70 bp, Homo sapiens
gtatgttcaccaggggaatggctgggatttctcggcactctgcatcatccatcttttcttataggtggga

SEQ ID NO: 963 - DNA, 70 bp, Homo sapiens
cctcattcccttttttctttacccaggattggtttcttcaataaatagataagatcgaatccatttaaaa

SEQ ID NO: 964 - DNA, 70 bp, Homo sapiens
cagtggccatcatcctcccgccaggagcttcttcgttcctgcgcatatagactgtacgttatgaagaata

SEQ ID NO: 965 - DNA, 70 bp, Homo sapiens
agcacaagcagttggagcttccacccctacgaccagtagcccagcacctgcagtatccacttcaacatca

SEQ ID NO: 966 - DNA, 70 bp, Homo sapiens
gcctatcacctccagcacaatcccagcgaaaaaggtgtgaagcacccaccatgttcttgaacaatcaggt

SEQ ID NO: 967 - DNA, 70 bp, Homo sapiens
gggaacagtggtactaacccacgattctgagccctgagtatgcctggacattgatgctaacatgacatgc

SEQ ID NO: 968 - DNA, 70 bp, Homo sapiens
aacagaagccgcagtcccgtggggtctggagacgcagtttccttgttaatgacaataaatccctgctccc

SEQ ID NO: 969 - DNA, 70 bp, Homo sapiens
ctgccacagggcccttcctacctttggatctgtgagaaggtgaatacaaagcagcaggcagagtaaaatc

SEQ ID NO: 970 - DNA, 70 bp, Homo sapiens
ttcccacatgccgtgactctggactatatcagtttttggaaagcagggttcctctgcctgctaacaagcc

SEQ ID NO: 971 - DNA, 70 bp, Homo sapiens
cttcctctttccctcggagcgggcggcggcgttggcggcttgtgcagcaatggccaagatcaaggctcga

SEQ ID NO: 972 - DNA, 70 bp, Homo sapiens
tcctccactataagtctaatgttctgactctctcctggtgctcaataaatatctaatcataacagcaaaa

SEQ ID NO: 973 - DNA, 70 bp, Homo sapiens
gaatcgacgtctcaagaggttctccatggtggtacaggatggcatagtgaaggccctgaatgtggaacca

SEQ ID NO: 974 - DNA, 70 bp, Homo sapiens
cccctgtccccactcgcgttccgcatggaggatactgaggccttacccctaaccccgatcctctacccaa

SEQ ID NO: 975 - DNA, 70 bp, Homo sapiens
atcactgtaaatggtaatcagttggaattctcctaaatgtcttccagacactagtaaaaaacgacctgaa

SEQ ID NO: 976 - DNA, 70 bp, Homo sapiens
gcaggaaaactagcatgaaatattgtttcaggccctgggttctatgtgacactacattaggaattggatt

SEQ ID NO: 977 - DNA, 70 bp, Homo sapiens
gaaaatcgggttcacaggctccacagaggtgggcaagcacatcatgaaaagctgtgccataagtaacgtg

SEQ ID NO: 978 - DNA, 70 bp, Homo sapiens
gagtgattctgatatatgtacttgtcacattggtgttggacacatttgcgccaaaagtatggtaattcta

SEQ ID NO: 979 - DNA, 70 bp, Homo sapiens
ggcatggcagtacccatgttgatttgacatctctctagcccatccattgcttacagtagaagagtggggc

SEQ ID NO: 980 - DNA, 70 bp, Homo sapiens
gcctctcagtcttaggggacatggcagagatgaaagaaagaaagagtgggtttcagaagtgtcagggtgg

SEQ ID NO: 981 - DNA, 70 bp, Homo sapiens
gatgcggggcctggcggtcttcatctcggatatccgcaactgtaaaagtaaagaagcagaaataaaaagg

SEQ ID NO: 982 - DNA, 70 bp, Homo sapiens
ccacctggtcatatactctgcagctgttagaatgtgcaagcacttggggacagcatgagcttgctgttgt

SEQ ID NO: 983 - DNA, 70 bp, Homo sapiens
attgaacatggtcttgtggatgagcagcagaaagttcggaccatcagtgctttggccattgctgccttgg

SEQ ID NO: 984 - DNA, 70 bp, Homo sapiens
gagggcagtaggccatcccccaggagaatgacagaagcaaaggacttgttactaagcagatttaagggtc

SEQ ID NO: 985 - DNA, 70 bp, Homo sapiens
cccccctctgaattttactgatgaagaaactgaggccacagagctaaagtgacttttcccaaggtcgccc

SEQ ID NO: 986 - DNA, 70 bp, Homo sapiens
aatgttgctgatagggataaatcttgaggctgagggcgggtggtacagatgtgtatgggaaaccccaacc

SEQ ID NO: 987 - DNA, 70 bp, Homo sapiens
cgtgctgcctctcttctgtgtcgttttgttgccaaggcagaatgaaaagtccttaaccgtggactcttcc

SEQ ID NO: 988 - DNA, 70 bp, Homo sapiens
ggccaggcgcgctctgcccagcccagcctacagtgcggataaaggtgcggatgctgctggccctgaaaaa

SEQ ID NO: 989 - DNA, 70 bp, Homo sapiens
agatctgctgcctcgcctctagatatggtgccctggtcttcatggatgaatgccatgccactggcttcct

SEQ ID NO: 990 - DNA, 70 bp, Homo sapiens
cccaagtgaagagaacgtcatgagtgtaagtgcaaatcagtggaaggagcggcaaactgggacatgcaga

SEQ ID NO: 991 - DNA, 70 bp, Homo sapiens
tgtggagggcgagctgagccctggccgccgccacaatgggccgcgagtttgggaatctgacgcggatgcg

SEQ ID NO: 992 - DNA, 70 bp, Homo sapiens
ccccctgaagtcaggaccagtgcctgtgatctccattactttattttcctggaggtattagccaacacag

SEQ ID NO: 993 - DNA, 70 bp, Homo sapiens
tgggaggcgggcgcagggtagctgttggcgccgccgcgtttctgggcctggccaactcacgtgaccgacg

SEQ ID NO: 994 - DNA, 70 bp, Homo sapiens
ggaaagacctgcccccgtgattaaattatttcccaccaggcccctcccacaacatggaataatgggagat

SEQ ID NO: 995 - DNA, 70 bp, Homo sapiens
ggtgtggattattgggccaaaagaggaagaggtcgtggtacttttcaacgtggcagagggcgctttaact

SEQ ID NO: 996 - DNA, 70 bp, Homo sapiens
ctgcccttggtgcattagcaagggtcctgagagaagactggaagcaaagtgtcgagttagctacaaacat

SEQ ID NO: 997 - DNA, 70 bp, Homo sapiens
atcgagatgccaagaagggctatggaactatgcaggtggctagtggtcagactgaagtcaccagctgaat

SEQ ID NO: 998 - DNA, 70 bp, Homo sapiens
catcatacaaaccacattacttctgtcacttcagggcatcgggactggctggcgcccttgttatgtgcta

SEQ ID NO: 999 - DNA, 70 bp, Homo sapiens
tcactcgcccagtcttcagtctcctgacttagagatacaatcacgtcacaggtctcttggcctcaatctg

SEQ ID NO: 1000 - DNA, 70 bp, Homo sapiens
aggggctgctgtccacagcttggggctgaagactcccaggccattaaccccttagcttttaggaagatta

SEQ ID NO: 1001 - DNA, 70 bp, Homo sapiens
ctcacgctgatggcttggcagagcaccttcggttaacttgcatctccagattgattactcaagcagacag

SEQ ID NO: 1002 - DNA, 70 bp, Homo sapiens
cgagtggtctgtgttcctattgctggtggggtgatagggtgggctaaaaaccatgcactctggaatttgt

SEQ ID NO: 1003 - DNA, 70 bp, Homo sapiens
gaggtgctcaataagcaaaagtggtcggtggctgctgtattggacagcacagaaaaagatttccatcacc

SEQ ID NO: 1004 - DNA, 70 bp, Homo sapiens
attactgtggagcagctttcattcctacccacttgcaaaccttggcgctgttgtctgagattgctgcagc

SEQ ID NO: 1005 - DNA, 70 bp, Homo sapiens
tggacagtgcaatgaaggaagaagtgcagaggctgcagtccagggtggacctgctggaggagaagctgca

SEQ ID NO: 1006 - DNA, 70 bp, Homo sapiens
tcgctcaagctttcgaagacacatgatggcacacactggagatggccctcataaatgcacagtatgtggg

SEQ ID NO: 1007 - DNA, 70 bp, Homo sapiens
acccaatgtggacttcttttaaacctttctaatgcccataacccagcctcagacccatggagcccacgag

SEQ ID NO: 1008 - DNA, 70 bp, Homo sapiens
aggtcctctgaggatcagatcatgcatgcgccattttttacttaatgcagctgttaaattggcaaagctc

SEQ ID NO: 1009 - DNA, 70 bp, Homo sapiens
cagcccataagagacattctcagatgaaactctgttttcttgccccagtcaggctcaagccctgtggttg

SEQ ID NO: 1010 - DNA, 70 bp, Homo sapiens
gctctgtatgtcctcaggggactgacaacatcctccagattccagccataaaccaataactaggctggac

SEQ ID NO: 1011 - DNA, 70 bp, Homo sapiens
aattccagtggcaaaaattcgaacagaacaggaaagcaaaggccctatgacccgccgactgctgctgcat

SEQ ID NO: 1012 - DNA, 70 bp, Homo sapiens
ccaatactttagaagtttggtcgtgtcgtttgtatgaaaatctgaggctttggtttaaatctttccttgt

SEQ ID NO: 1013 - DNA, 70 bp, Homo sapiens
ttttctagagcaaagcaaagtagcttcgggtcttgatgcttgagtagagtgaagaggggagcacgtgccc

SEQ ID NO: 1014 - DNA, 70 bp, Homo sapiens
gctctaggccctcacctcaaaccttgccattggttgccgtatttcaaggtcaatatagtttccctcactt

SEQ ID NO: 1015 - DNA, 70 bp, Homo sapiens
gctccattaaatagccgtagacggaacttcgcctttctctcggccttagcgccatttttttggaaacctc

SEQ ID NO: 1016 - DNA, 70 bp, Homo sapiens
tgacaacgaaggccgcgcctgcctttcccatctgtctatctatctggctggcagggaaggaaagaacttg

SEQ ID NO: 1017 - DNA, 70 bp, Homo sapiens
ggggagcacatattggatgtatatgttaccatatgttaggaaataaaattattttgctgaaacttggaaa

SEQ ID NO: 1018 - DNA, 70 bp, Homo sapiens
ctttggatccatttcatgcaggattgtgttgttttaactgttgttgaggaagctaataaataattaaatt

SEQ ID NO: 1019 - DNA, 70 bp, Homo sapiens
gaacccaatggtagtcttaaagagttttgtgccctggctctatggcggggaaagccctagtctatggagt

SEQ ID NO: 1020 - DNA, 70 bp, Homo sapiens
ccttctccaacatacatcctgcattacatgaatggattattcctaataattaataaaaaggtattttttc

SEQ ID NO: 1021 - DNA, 70 bp, Homo sapiens
gacaacacaaaactagagccaggggcctccgtgaactcccagagcatgcctgatagaaactcatttctac

SEQ ID NO: 1022 - DNA, 70 bp, Homo sapiens
gcacagagtcaggatctcacatttcaccccaggctcaactgaggatgtggcttattaaacacggaagtgc

SEQ ID NO: 1023 - DNA, 70 bp, Homo sapiens
tcccgtgcaacagcagaatcaaattggatatccccaaccttatggccagtggggccagtggtatggaaat

SEQ ID NO: 1024 - DNA, 70 bp, Homo sapiens
gctcccacggaggggagcaggaatgctgcactgtttacaccctgactgtgcttaaaaacactttcactaa

SEQ ID NO: 1025 - DNA, 70 bp, Homo sapiens
taaaaatacaaaaattagccgggcgtggtggcttacgcctgtaatcccagcactttgggaggccaaggtg

SEQ ID NO: 1026 - DNA, 70 bp, Homo sapiens
tggctgtgcttattgccctcaccatttatgacgaagatgtgttggctgtggaacatgtgctgaccaccgt

SEQ ID NO: 1027 - DNA, 70 bp, Homo sapiens
ggaaaagcattggcacgcaacgcagcatgtggcttcattgaggcagttgatggagttaaaccatctgctc

SEQ ID NO: 1028 - DNA, 70 bp, Homo sapiens
cgtgccttcttgctgtcatgcaatgaccccgccttatgttgccgaaataagcaactcttaggtttgcctg

SEQ ID NO: 1029 - DNA, 70 bp, Homo sapiens
gagttttcctcggaaacactcttgaatgtctgagtgagggtcctgcttagctctttggcctgtgagatgc

SEQ ID NO: 1030 - DNA, 70 bp, Homo sapiens
gaggcagaatggctctgctgagcctcctacccatgacaacaccccaataaacagaacattcagagccaaa

SEQ ID NO: 1031 - DNA, 70 bp, Homo sapiens
gggttttcctgggagcgaatatcaagtgcctgagagcaactacaggactaactgtgtttgggttgggtgt

SEQ ID NO: 1032 - DNA, 70 bp, Homo sapiens
ggaaccccaggttcgcggcccgtgtttccgaccggcggagggggctcagcggcccgatcccacggaagcg

SEQ ID NO: 1033 - DNA, 70 bp, Homo sapiens
tctccagatgaggttgcaaggaccaaccagtgcctacccgcccatgctcccccgaaactgggaactgaca

SEQ ID NO: 1034 - DNA, 70 bp, Homo sapiens
ccctgctattagaccaccccctcatggcacaactgcccctcacaagaattcagcttcagtgcaaaattca

SEQ ID NO: 1035 - DNA, 70 bp, Homo sapiens
acattcttcctttgcatttgctggtctggcctttgcgtccttctacctggcagggaagttacactgcttc

SEQ ID NO: 1036 - DNA, 70 bp, Homo sapiens
acaggggaagatcccgagtgcaagaaagagacaaagagcccctacaggaacgctttttccgaccacattt

SEQ ID NO: 1037 - DNA, 70 bp, Homo sapiens
gggcagtcgctgcagggagcaccacggccagaagtaacttattttgtactagtgtccgcataagaaaaag

SEQ ID NO: 1038 - DNA, 70 bp, Homo sapiens
gctgcaatgatgttagctgtggccactgtggatttttcgcaagaacattaataaactaaaaacttcatgt

SEQ ID NO: 1039 - DNA, 70 bp, Homo sapiens
aattcatgacccacaaacttaaacatactgagaatactttcagccgccctggagggagggccagcgtgga

SEQ ID NO: 1040 - DNA, 70 bp, Homo sapiens
agttggtcgggatcctgctcagcgccctgctaggggttgccctgggacaccgcacgcggtgctatgactg

SEQ ID NO: 1041 - DNA, 70 bp, Homo sapiens
gataatatctctcacccggatccctcctcacttgccctgccactttgcatggtttgattttgacctggtc

SEQ ID NO: 1042 - DNA, 70 bp, Homo sapiens
tggccgccatgaggaaagctgctgccaagaaagactgagcccctcccctgccctctccctgaaataaaga

SEQ ID NO: 1043 - DNA, 70 bp, Homo sapiens
caaggccagtagaaagctatggctgcaaaaccctggggtggacgatgtttgatgattagacggtcatctc

SEQ ID NO: 1044 - DNA, 70 bp, Homo sapiens
cccaggagtttgaggccagcctgggcaacatggtgaaacccggtgtctaccaaaaatacaaaatgtatcc

SEQ ID NO: 1045 - DNA, 70 bp, Homo sapiens
ctgtttttctgtatgctctgtgctagtagggtggattcagtaataaatatgtgaaagcttttgtttccaa

SEQ ID NO: 1046 - DNA, 70 bp, Homo sapiens
tgaattctacaaccggttcaagggccgcaatgacctgatggagtacgcaaagcaacacgggattcccatc

SEQ ID NO: 1047 - DNA, 70 bp, Homo sapiens
cgctgtaaaactccgaaatctggcacaaacccaacacggagctacgcaatactgctggagagcatttgct

SEQ ID NO: 1048 - DNA, 70 bp, Homo sapiens
acttcaccgaagaccagaccgcagatctgatcccaagcactgagttcaaggaggccttccagctgtttga

SEQ ID NO: 1049 - DNA, 70 bp, Homo sapiens
cgaggcctggggagatgttgttttcatgctgcttccaccatcacactggggtttctggatgggaaataaa

SEQ ID NO: 1050 - DNA, 70 bp, Homo sapiens
cacgcagccatggttgtgcctgccgttcatggtggtctttcaggttatcttggcaacatgtacattgctt

SEQ ID NO: 1051 - DNA, 70 bp, Homo sapiens
gtgtggctgcggttgggtatggatcaagcaagggttcagattacatcattgtgaagaattcttggggacc

SEQ ID NO: 1052 - DNA, 70 bp, Homo sapiens
tgccttctaaatgtggtgtcgatctcccttacaagttcagcccttccactgactgcgacagtatccagtg

SEQ ID NO: 1053 - DNA, 70 bp, Homo sapiens
acgggcttatgatccctcgagcactatttatccgtgatttgatgtggctcactggttcgctatgggcaac

SEQ ID NO: 1054 - DNA, 70 bp, Homo sapiens
cgccctgaaggagtacatcgtctagtgagggacagaccaagcacgcaaaacaaattgcaatataatgtga

SEQ ID NO: 1055 - DNA, 70 bp, Homo sapiens
gctcatttgagataaagtcaaatgccaaacactagctctgtattaatccccatcattactggtaaagcct

SEQ ID NO: 1056 - DNA, 70 bp, Homo sapiens
tctttccttctgatctgagaagacatgaacgttttctcttcaccgccgtggggtgtattgactggtcccc

SEQ ID NO: 1057 - DNA, 70 bp, Homo sapiens
ctttcccagaagatggaggagagtatatgtgtaaagcagtcaacaataaaggatctgcagctagtacctg

SEQ ID NO: 1058 - DNA, 70 bp, Homo sapiens
tcacattttcccaaaaaaagttgatctctcccagtgggctgtaggcagggtcctccatgggtttccaacc

SEQ ID NO: 1059 - DNA, 70 bp, Homo sapiens
aaaattccagagtgaccgtggcacttgggtgtacaggtaattcctccagagctgtttgctggcttcagga

SEQ ID NO: 1060 - DNA, 70 bp, Homo sapiens
gtggggaagagctattgtaggctccccctcctctgacttatgtaatcaaagccacttttgtgtgtgtcta

SEQ ID NO: 1061 - DNA, 70 bp, Homo sapiens
tcatcttgcttgggcttaccaaatgcattagtctttgtgtttgggtcgacagcgagtgtgcctgtgctgg

SEQ ID NO: 1062 - DNA, 70 bp, Homo sapiens
ctgttcttgtttcaaagcaccacttggaggctgcggaagatacccgtgtaaaggaaccactgtcttcagc

SEQ ID NO: 1063 - DNA, 70 bp, Homo sapiens
cctctgcagtccgtgggctggcagtttgttgatcttttaagtttccttccctacccagtccccattttct

SEQ ID NO: 1064 - DNA, 70 bp, Homo sapiens
gtgccacttcatggtgcgaagtgaacactgtagtcttgttgttttcccaaagagaactccgtatgttctc

SEQ ID NO: 1065 - DNA, 70 bp, Homo sapiens
aaccctccataaacctggagtgactatatggatgccccccaccctaccacacattcgaagaacccgtata

SEQ ID NO: 1066 - DNA, 70 bp, Homo sapiens; identical to SEQ ID NO: 594

SEQ ID NO: 1067 - DNA, 70 bp, Homo sapiens; identical to SEQ ID NO: 621

SEQ ID NO: 1068 - DNA, 70 bp, Homo sapiens
tcgcccacgggcttacatcctcattactattctgcctagcaaactcaaactacgaacgcactcacagtcg

SEQ ID NO: 1069 - DNA, 70 bp, Homo sapiens
cttcccacctcagcctcctgaatagctgggactaccagcacgctccaccatgccttgctaattatttttt

SEQ ID NO: 1070 - DNA, 70 bp, Homo sapiens
ttgctactttggcaaaaactagcgaggggtagcagaaacctgcaccaaggattgtccctatgtcttggcc

SEQ ID NO: 1071 - DNA, 70 bp, Homo sapiens
tatcaataaagttgctcacttgttgccggcccgctagcccgaaaggttgcgcgcgcagaccgagaagtct

SEQ ID NO: 1072 - DNA, 70 bp, Homo sapiens
aagattgatggaagcctcgggcctaaagaatcacagagttatggagaaggtagctcggagagcctcctga

SEQ ID NO: 1073 - DNA, 70 bp, Homo sapiens
actaaccctatgttgcacacgctgggttcctgatcttggtgcgatgttttggttacatggcatctggcag

SEQ ID NO: 1074 - DNA, 70 bp, Homo sapiens
taaagataaagccagaagctaagctgcagtgaggctgtgattgggcgtagaagtgggagcattgggacct

SEQ ID NO: 1075 - DNA, 70 bp, Homo sapiens
acaaccagaaggcccttaactatcaccagtgcatcacatctgcacactctcttctccattccctagcagg

SEQ ID NO: 1076 - DNA, 70 bp, Homo sapiens
cagtatccaaaaatagccctgcaaaaattcagagtccttgcaaaattgtctaaaatgtcagtgtttggga

SEQ ID NO: 1077 - DNA, 70 bp, Homo sapiens; identical to SEQ ID NO: 822

SEQ ID NO: 1078 - DNA, 70 bp, Homo sapiens
agagtgtcatggacctgataaagcgaaactccggatgggtgtttgagaatccctcaataggcgtgctgga

SEQ ID NO: 1079 - DNA, 70 bp, Homo sapiens
cagagtgttgggtctgtagccagcaaattacttcatcatctagattatccattcagttgatcctaattag

SEQ ID NO: 1080 - DNA, 70 bp, Homo sapiens
ttgctcaccctcggtaaagagagagagggctgggaggaaaagtagttcatctaggaaactgtcctgggaa

SEQ ID NO: 1081 - DNA, 70 bp, Homo sapiens
ccgctcccacctccctgctgggaaaccacagcattatcacagcattattgtgacagccacgaacccattg

SEQ ID NO: 1082 - DNA, 70 bp, Homo sapiens
ggagaggtaggtgacatagtgctttggagcccagggagggaaaggttctgctgaagttgaattcaagact

SEQ ID NO: 1083 - DNA, 70 bp, Homo sapiens
gccggggcccgaatccaggcactgctgggctgcctgctcaaggtgctgctctgggtggcctctgccttgc

SEQ ID NO: 1084 - DNA, 70 bp, Homo sapiens; identical to SEQ ID NO: 31

SEQ ID NO: 1085 - DNA, 70 bp, Homo sapiens
gaggcgtccagcgagccgccgctggatgctaagtccgatgtcaccaaccagcttgtagattttcagtgga

SEQ ID NO: 1086 - DNA, 70 bp, Homo sapiens
aaacaggagccttacccaggaactcttttttatgccagaacgcttcctctcccctgctgtctctggggct

SEQ ID NO: 1087 - DNA, 70 bp, Homo sapiens
gagcgcggctgcgccggcgcgtcgaggggagaggcagcagccgcgatggacgtgttcctcatgatccggc

SEQ ID NO: 1088 - DNA, 70 bp, Homo sapiens
cggaaaaaattgtattgaaaacacttagtatgcagttgataagaggaatttggtataattatggtgggtg

SEQ ID NO: 1089 - DNA, 70 bp, Homo sapiens
cacggaccaggttcccgcaaaacattgccagctagtgaggcataatttgctcaaagtatagaaacagccc

SEQ ID NO: 1090 - DNA, 70 bp, Homo sapiens
tcagcttccaggaccttggctggctggtaattgctgactctccttgtttctgtgccgcaccacaggcagg

SEQ ID NO: 1091 - DNA, 70 bp, Homo sapiens
ggtagcggccgaggtacactcggcttggctgttggagttgcttgtggcatgtgcctgggctggagccttc

SEQ ID NO: 1092 - DNA, 70 bp, Homo sapiens
tcaaagtatatgtagagatgactattttatattacatgacccaatcctgtatttatttctaccccctttt

SEQ ID NO: 1093 - DNA, 70 bp, Homo sapiens
attaaagttctttttattgcagtttggaaagcatttgtgaaactttctgtttggcacagaaacagtcaaa

SEQ ID NO: 1094 - DNA, 70 bp, Homo sapiens
tcattaagaacttttcaaaagtgaattagtgaggattcagcttaatacctgtatcaaatgaggaagtggt

SEQ ID NO: 1095 - DNA, 70 bp, Homo sapiens
ccccgatcatcgtgcttatctaatacctcacgaccttctctcggcgggccctggtttcctgctgaacgat

SEQ ID NO: 1096 - DNA, 70 bp, Homo sapiens
acatgatgagttggcattagcttctccaggcatgggaacttaacagatgaggttaagaaccgtagacagt

SEQ ID NO: 1097 - DNA, 70 bp, Homo sapiens
catcagaagtgtttcttattattattttatattgagttgaatattgaactctaacagttttctacataca

SEQ ID NO: 1098 - DNA, 70 bp, Homo sapiens
agacataatgtagacatagaggaggaacagctgagagtctctgcatcacagaaagagaaacctgagcaaa

SEQ ID NO: 1099 - DNA, 70 bp, Homo sapiens
aatgtcctagaaacaaatatagaaaaatatattcatgagcttaggagaatgtaggcaaagttttcctggc

SEQ ID NO: 1100 - DNA, 70 bp, Homo sapiens
cggccctgtgtgcctcagggcagatatagcaagctctttcgaccatagttgatggtaggacattttagac

SEQ ID NO: 1101 - DNA, 70 bp, Homo sapiens
ggacattgtatttgatggcatcgctcagatccgtggtgagatcttcttcttcaaggaccggttcatttgg

SEQ ID NO: 1102 - DNA, 70 bp, Homo sapiens
gggatgagggatcatgcatgatcagttaagtcactctgccactttttaaaataatacgattcacatttgc

SEQ ID NO: 1103 - DNA, 70 bp, Homo sapiens
aacatcattctcaccaccagtctcttctctgtgcctttcttcctgacgtggagtgtggtgaactcagtgc

SEQ ID NO: 1104 - DNA, 70 bp, Homo sapiens
ccgcacctggccttccctgcttcctctctagaatccaattagggatgtttgttactactcatattgatta

SEQ ID NO: 1105 - DNA, 70 bp, Homo sapiens
aaggagattgagtacgaggtggtgagagacgcctatggcaactgtgtcacggtgtgtaacatggagaact

SEQ ID NO: 1106 - DNA, 70 bp, Homo sapiens
atgttcaagttccacattggtcttcaactctctggcggggtcagaggaccatctgtgctcgctcagatat

SEQ ID NO: 1107 - DNA, 70 bp, Homo sapiens
acagcggcagtcgcgcccacacgtccatgactggtcgtcctagattttaggtgtcgatgaatacggccca

SEQ ID NO: 1108 - DNA, 70 bp, Homo sapiens
ccttctgtgactccctgcagccactgcttcttgaagcctttgtctctaagcttctgtccagctcaaaccc

SEQ ID NO: 1109 - DNA, 70 bp, Homo sapiens
cggaaacgggaaggcctgctgcattccagccacatctcggaggagctgaccacaactacagagatgatga

SEQ ID NO: 1110 - DNA, 70 bp, Homo sapiens
catgaaaaccatgaaggggccttttggctgaaattccccacctgcctttggatgaaagactccgttggga

SEQ ID NO: 1111 - DNA, 70 bp, Homo sapiens
agaggagcccacgtcgcctgtcacccaatatctccagccgcgcagtcccgaagagtgtaagatgttcgcc

SEQ ID NO: 1112 - DNA, 70 bp, Homo sapiens
gagtatgaaggagagaagagggtactgaccatgcgtttcaacataccaactgggaccaatttaccccctg

SEQ ID NO: 1113 - DNA, 70 bp, Homo sapiens
taaatacatccaaacatgatcatcgttggagccggaggtggcaggagtcgaggcgctgatccctaaaatg

SEQ ID NO: 1114 - DNA, 70 bp, Homo sapiens
gattcctcctttcccccccaaatattaactccagaaactaggcctgactggggacaccctgagagtagta

SEQ ID NO: 1115 - DNA, 70 bp, Homo sapiens
tactaaacataaaaaaattagcctggcatggtggtgtacgcctgtaatcccagtgacttgggaggctgag

SEQ ID NO: 1116 - DNA, 70 bp, Homo sapiens
ggtggagaggaattgccggagctctgaaaatcctaatgaagtgttccgcttcttggtggaggaaaggatc

SEQ ID NO: 1117 - DNA, 70 bp, Homo sapiens
agctgctctatagcaatgtttctaactttgcccgcctggcttccaccttggttcacctcggtgagtatca

SEQ ID NO: 1118 - DNA, 70 bp, Homo sapiens
tcccaacagattgggctgggtgggggttgacaatggggtcagatactaaagggtcagaatttctaagcag

SEQ ID NO: 1119 - DNA, 70 bp, Homo sapiens
agccacatctgcctctgagctgcctgcgtcctctcggtgagctgtgcagtgccggccccagatcctcaca

SEQ ID NO: 1120 - DNA, 70 bp, Homo sapiens
tggcctgcttggcaaggcaagtagcggcggcgcttcaagatgcgctgcctgaccacgcctatgctgctgc

SEQ ID NO: 1121 - DNA, 70 bp, Homo sapiens
gagagcattccgcaaagctgcttgttttccaatttcttcattcttccccttagcactggtgcagctgaat

SEQ ID NO: 1122 - DNA, 70 bp, Homo sapiens
ctggtggccattagtcactcttcatttggctggaactaccgcacggaccctttgaagatatgtgtggatg

SEQ ID NO: 1123 - DNA, 70 bp, Homo sapiens
gccctgggccttaagagccagctcttcctatcctgtagcgtgtagaaaacgtggactcatttcactatgt

SEQ ID NO: 1124 - DNA, 70 bp, Homo sapiens
agattcatatgggctggtgttcctgtgcgctgtgggtgtggtgattcagcctggcatttctaccataagt

SEQ ID NO: 1125 - DNA, 70 bp, Homo sapiens; identical to SEQ ID NO: 1088

SEQ ID NO: 1126 - DNA, 70 bp, Homo sapiens
cgagcccggccccgccagcccagcccagcccagccctactccctccccacgccagggcagcagccgttgc

SEQ ID NO: 1127 - DNA, 70 bp, Homo sapiens
gcttgggtaagtacgcaacttacttttccaccaaagaactgtcaccacctgcctgcttttctgtgatgta

SEQ ID NO: 1128 - DNA, 70 bp, Homo sapiens
ttccctgaggaggcgaatccggcgggtatcagagccatcagaaccgccaccatgacggtgggcaagagca

SEQ ID NO: 1129 - DNA, 70 bp, Homo sapiens
cgacaaggaagatttgcatgatatgcttgcttcattggggaagaatccaactgatgagtatctagatgcc

SEQ ID NO: 1130 - DNA, 70 bp, Homo sapiens
tgattattactgtgcagcatgggatgacagcctgaatggtgtggtattcggcggagggaccaagctgacc

SEQ ID NO: 1131 - DNA, 70 bp, Homo sapiens
ctacagttggaaatccatccagaggccatgttccaataaacaggaggtcgtgtatttggtcacgacattt

SEQ ID NO: 1132 - DNA, 70 bp, Homo sapiens
ctaccgcccagtcactcaaatccgtggactacgaggtgttcggaagagtgcagggtgtttgcttcagaat

SEQ ID NO: 1133 - DNA, 70 bp, Homo sapiens
ctctgaaaaaaatgatttcaaggcatggaagttctctgtgatacaacaatacgtattcttcaaatgcgcc

SEQ ID NO: 1134 - DNA, 70 bp, Homo sapiens
gcccatctcagcaagttccatgtcagccttggcagaagcctctttctttcctcttccccataagagacat

SEQ ID NO: 1135 - DNA, 70 bp, Homo sapiens
ggggacagcgcttgccttggtcagaccttcccacatctacatactctcaaatacatgaccaggtgatcaa

SEQ ID NO: 1136 - DNA, 70 bp, Homo sapiens
gcatctgtaggagacagagtcaccatcacttgccgggcaagtcagagcattagcagctatttaaattggt

SEQ ID NO: 1137 - DNA, 70 bp, Homo sapiens
ggcagcgaactgagtgaaggggaattggaaaagcgcagaagaacccttttggagcaactggatgatgatc

SEQ ID NO: 1138 - DNA, 70 bp, Homo sapiens
atggcctattcacacagatccatcagcgcactgccagcaagcttctcggtcactagaatgagattaaaaa

SEQ ID NO: 1139 - DNA, 70 bp, Homo sapiens
aagctcgcaactgtgtaggatgaattctgtacacttttatttccctctgttctcctttcctatttgaaag

SEQ ID NO: 1140 - DNA, 70 bp, Homo sapiens
aggagcgtcggtagttcttgcagtaggcactttatcaggacctgacctgttgctgggtgattttagtctc

SEQ ID NO: 1141 - DNA, 70 bp, Homo sapiens
tcgcacgaggatgcttggcacgtaccccgtctacatacttcccaggcacccagcatggaaataaagcacc

SEQ ID NO: 1142 - DNA, 70 bp, Homo sapiens
taggagattttcattttgtgtgactcccatggggaggaacagactggcaggaagcacaccggggttaaca

SEQ ID NO: 1143 - DNA, 70 bp, Homo sapiens
cactccaacccaaactagctgggagttcagaaccatggtggaataaagaaatgtgcatctgctcgtgccg

SEQ ID NO: 1144 - DNA, 798 bp, Gallus gallus
aggtaccaataaaacgtaggcttttgactttaggaaaatacaagaaattttaatgcaacagacaggagagaggtccagcatagatatctaacgtgttagttttatttaaagatggtctcacggtgcyagagtaagaaatgttattaagaaaacgagagagagagggagagagatcaaataaataaataaataaataaataaaaataggaataactttctgttgacgagctttcatcttgggaggaacgggttctgtgaagcattcttcagagtgaagtggtcctaattcttcctggaaccattgcaacccattccactcagggagccaatcctatcaattcttctgccgaagcagccagaatctctcatcatccggggcatctgcacccccctcagtctcttgaggaaggggttcctgtaggacagaggagtgttggatgctagcttgggttcagccttctgctcatcgctgtcatctatgagttctggtggaatctcctctcgggtttggggctcttgtaggtcaggattggactccagggcttcaatcagtgcgaatttgtcctccagtctctccagaagagcctccatgctggccagttctttggcagggctgaggttgtagatggggttggccctgctgggctgcagctggacaagaagcagcaagaggaaaccacaggaaaatgagcctctagtgtccatggcgctgggttcgttgggaatatgggaagttcaagctgtttcttctgagatggctcttcaggtctctctcttsgckgggaccasgctaatcas

SEQ ID NO: 1145 - DNA, 367 bp, Gallus gallus
ctgattagcctggtccccgcccaggtactccaagggacagaagaggagctacagataaagagaaactgccagctaaagctgtatatgactttaaagctcaaacttctaaagagctgtcctttaagaaaggagatactgtctatatcctcaggaaaattgatcaaaactggtatgaaggtgagcaccatggaagagttggaatattcccaatttcttatgttgagaaactttctcctccagaaaaagcacagyctgccaggccgcctccccctgcacagattggagagattggagaagctattgccaaatataatttcagtgcagacacaaatgtggagttatcacttagaaagggagacagagtcgt

SEQ ID NO: 1146 - DNA, 146 bp, Gallus gallus
acagtaaagtctttattaaagttattgttgggtgatcacataactttctttttaaaaaaaaaaaacaactgctgtctgaattggaaaccagatcatacctttttgtattgggattttggaccatatatcttatgtttctgtacctc

SEQ ID NO: 1147 - DNA, 587 bp, Gallus gallus
acatgctgtttttctactgctgcaatctaacatgtgagttacagatccttaagatctttctggatgctccacaatgtgtctgcactcttcttcagctgagccacttcatcatccttcaacttttggttgatgacacttgtcaatccagaggcactcaggacacaaggcaggctcaggaagacatcgttctcaatgccatacatgccctttaccagtgttgacacagaatgaactctgcacaagttcttcagcatcgtctcacagagctcagcaacgctaaggccaatggcccagtttgtataacccttgagtctgattacctcataggcactttcaacaacctgcttgtggacttccttccagttctcactgtctttgtcagttcccatggcaggattcagctcctggagagaaacacctgccacattaactccgctccaaacagccacactagaatcaccatgttctcctaaaatccagccatcgcagctggttgggtggatatcaagtctctcagccatcaggtagcggaatctagctgtgtctagattgcagccacttccaatcacacggtgctttggcaggcc

SEQ ID NO: 1148 - DNA, 738 bp, Gallus gallus
aggtaccatgctgaggaaattcagtgcaatggaaggagttttcacaagacgtgcttcctctgcatggcttgcaggaaggctctggacagcaccacagtggcagctcacgaatctgaaatctactgcaaaacttgctacgggagaaaatacggccccaaaggtgttggctttggacaaggggccggatgtctcagcaccgacactggggaccatctgggcctaaacctgcaacagggatcaccaaagtctgctcgcccttctacaccaactaatccttcaaagtttgccaaaaagatcgttgatgtggataaatgtccccggtgtggcaaatcggtgtatgctgcagagaagataatgggaggaggaaaaccttggcataaaacatgcttccgctgtgctatctgtggaaagagtttagagtctacaaatgttacagacaaagatggagagctctactgtaaagtttgctacgcaaagaattttggtcccaaaggaattggttttggtggcctcactcaagtggaaaagaaagagtgtgagtgaaaaggagtggatgcaacagagtcaacctgctgctgatgtcagcagataatagttgtcaagtaaaaccaaatcaccacctactgctcataacctagggcattcattaaatattttccatcttgcaggaagccttctgaagccttctgaagaaaaagcaagttttcttagaatatagtgtttcagttttgttattgt

SEQ ID NO: 1149 - DNA, 489 bp, Gallus gallus
acctaaatggctttgttaattatggggatggccaggtgatgttattttttttaaagccgttcaggcagtcagtgtgtcagaagcagcacccaaacagtgagcgaacaaagtgctggccgcttgcactttgttcaaaaattgtcagtcaggtatggaaattacgacataatcagatccaatgtaaaacattaaagtaatacttacaggagggtaattgtaaatatcaccagtgccttacatttctgattcaacatagttatttgtgcatgtatgaaatactatgcacagtatcctctcttggtggtaggtaatcttcaacagggagcctctctacctgttgaggcattcttaaacatcaacaatgagttgaggcacaaaaattagttaaatgttgagcaggatagtcgtttgccaggaaactttctcctaccaactgttaattccaaaagttacatttcaaaatgtcataaaacaaatggtacctcggcc

SEQ ID NO: 1150 - DNA, 635 bp, Gallus gallus
caggtacaaaacctgttcaacactttgacctttggtcacatactcattgccaactttcactctagggtgtaataaacccttaactaaatcagaggagttgattcccattaggtaggcagctttgtcagcactttcagtgccatcagcctctgcctgctcttctctgggtcgctgtttgaatttcatgttcccaaagtgcataatggcacctgtgagcttgtaggcaccagacttctcatcttgcacaaatcccaaaatgtccatggcttgatctgttgccatcaattcttctccgtcatccaagttgtccactgtaactactccttgggagcaaaagtggtagtcatatgggttggtggagaccaatagcatatccagtaactctggcttctttcctgataagatctggtagaagatgtgatagtctctctcacccggttgctgaaaaatcactcgggatttctccagtaaatagatctcaatatcagcagatgacagcttgcccgtggttccaaaatggattcgaataaatttaccaaaacgtgaggagttgtcatttctcagggktttggcgtttccaaaagcttctagggctgggtttgcttgaatgatttgatcttccaaggttcccccag

SEQ ID NO: 1151 - DNA, 611 bp, Coturnix japonica
gggtaggtacctcgttgagtggataggagtatataaagggtgtaggaagcagttaagagcgttgcagttccggttaagataattgtgggaggggatcagttaaataaggcaattataattgttaattctgccattagattggttgttggagggagggctatgttggttaggttagctaggagtcatcatgtggctattaggggtaggaggggttgtaggcctcgtgtaagaatgagaatgcggctatgcgttcgttcgtagttcgtgttagctaagcagaataggagtgatgatgtaagtccgtgggagattattaggattattgcccctgaaaatgatcattgagtttggatcatgcttgcggcgattacgagtcctatgtggcttacggatgagtaggcaatgagagattttaagtctgtttggcgtaagcagatggagctggtcattagggcccctcatagggctagggtgaggaaagggaagtgtagatagttgcatacgggctgtatgagtagtgttactcgtataataccgtagccgcctagttttagtagtagggcagcaagtaatattgagcctgcggttggtgcttcgacatgggctttggg

SEQ ID NO: 1152 - DNA, 584 bp, Gallus gallus
gggcaggtaccagagcaaaattaagatgcagacagaagcacagcacgaaagagaactccagaagctggagcagagggtgtcactgcgcagggcacatctggaacaaaagattgaagaggagctcgccgccctccagaaggaacgcagcgagaggatcaagtttctgttggaaaggcaggagcgagagattgaaacgtttgacatggagagcttacgaatgggctttgggaatttggtcacattagattatcccaaggaggactatagatgagacgaaatttctttgccatttaacaaaaaccagacaaaatcaaacaaaatagttacaaaacttgcaaaaccaacattccccatgttaacgggcgtgctctctctctttctgtctctcttactgacatcgtgtcggactagtgcctgtatattcttactccatcaggggtcccctttccccctgtgtcaagtcccggtgcaggacagctcctggcggtcttttccatagtatgtcacagtattgatgtctctgtgcaatgattaaaaatgtttcagtgaaaaactttggagacgattttaatggagaaaaaaga

SEQ ID NO: 1153 - DNA, 591 bp, Gallus gallus
accgccgcctgcgatagggacggcgctgctgggcttggccttccgggatgttctctgctccttcgttcttctccccactctcactgttctggtagttttgctggtagttgcgtggaggacccctgcgacgcggatatcgtctgtaatggttacggtctgctgcgtatttgctgccttgcactggaacgccaccaggccctgtcacgttcgctgcctccgcacccttctctccttcaaccacatcaaactccacggtctctccgtctcctacgctgcggaggtacctcaaaaacccactcatttcagtcttagatattcacacatctcggtaaacaaaactaaaactacattattttttaatggccagcatgctgtattctctgagggctctaggcttgtgcagtagatacacataccacaaatgtaatgcttcctcaccgctgttaattgaaaatcttctaagttatttgctattgaggctgtggaaattgttaccacctgacaagactccaacagtggttaaatgactagtgtcggtagtgtaggaagctgtataaagataccttctgagctttccttaaattgtcacgt

SEQ ID NO: 1154 - DNA, 316 bp, Gallus gallus
actgtattttctgcttctctgccttttgaagccagggactgtcgggatttctttattctgtgggatactttacttctcagtctgaaaagctacttccttctacaaaggcaagaccaaagactttatgctggtccaatttgtagagcatagaggccccccccgactatttaagtttgacaatcttaatgaatttgtcatctttagagggaagcaaaagcataaaccataccaaagcaaaggaaatgctatatttttaaataagaaataataataatcacaggtcattaggatatcgtcagttccatggttctttagt

SEQ ID NO: 1155 - DNA, 523 bp, Gallus gallus
acagctggatgaggagctgggaggcagccctgtgcagaaacgagtagtgcaaggaaaagagccacctcatctgatgagcatgtttggtggaaagcctttgattgtttacaagggtggaacatctcgggaaggaggtcagaccacaccggcacaaacacggctcttccaggtccggtccagcacctcgggagctaccagagctgtagagctggatcctgctgccagtcagctgaactccaacgatgcttttgtcctgaaaactccctctgctgcttacctctgggttggccgtggctccaacagtgcagagctgtcaggagcacaagrgctgctgaaggttctgggagctcgtccagtacaaataggtcctatatacgttaagattctttggaaactgctaagtataaaggagtttgtaatccagactactatctttttggctactccaaaaaatctgcgggagtttccagcttatcatcagttgaaatctgttttgcaattgccaaataaatgcaagtaaaat

SEQ ID NO: 1156 - DNA, 642 bp, Gallus gallus
tgaaggttcttttacagcttctggggtgctcgggaaagacagatcttcagcactgccttcactgtctctttcataatcctttagcactgtcagcttatctacaccacatttctcatctaaaccagtttccagtttgccatctattttacttccaacatcctctctcaagctgctcagtccatgaccagcaccttttacttcccatattggctcatacggtttgaagtccacatactcttcccttcgaatagcatcttctttcaatgcctttttgtcatcatgatcaccatctctgcctttgtctttgctagaaaaggtatcttcctcatttttctcaaacaaatctttaggagtttctggagacatactcagaggctgtgcaggcattttctccagatcacataattctttagggccctctagctgcatttcttcttcagcttgaaataaaatggctgcttctgctttagtaagtgagggttccatttctgagtatttcatctcggaagcgtctctcttatttccctctgtaaggaatggatttcttacattttccattgtttctttacaaacctcacttgcactttctttgtaaatgcctcttgcagggaatccatctgagagtgaatcaaaggctgtgtg

SEQ ID NO: 1157 - DNA, 339 bp, Gallus gallus
actctgtctggattggaggctctatcctggcctctctgtccaccttccagcagatgtggatcagcaagcaggagtatgatgaatccggaccctccattgtccaccgcaaatgcttctaaaccggactgttaccaacacccacacccttgtgatgaaacaaaacccataaatgcgcataaaacaagacgagattggcatggctttatttgttttttcttttggcgcttgactcaggattaaaaaactggaatggtgaaggtgtcagcagcagtcttaaaatgaaacatgttggagcgaacgcccccaaagttctacaatgcatctgaggactttgattgt

SEQ ID NO: 1158 - DNA, 339 bp, Gallus gallus; identical to SEQ ID NO: 1157

SEQ ID NO: 1159 - DNA, 339 bp, Gallus gallus; identical to SEQ ID NO: 1157

SEQ ID NO: 1160 - DNA, 536 bp, Gallus gallus
acgcatgtgttatctacctcaaggtaacagcagtatgtggcaaaacattaaccacccatagtgcttctcattatgcacttctatttagccagcattattgtagtagctattcttattgaaaaccattcaatatttataaatgttctggtatgcattctttatagtgaagtgttaatatgcagcacttttatttattttagcaaataaataagtatatttctgtaattatagaaagtcaacttaatttttgagttacgtttcagataaaagtttttgtttagcactatggttttattgcctacatagctggatatatattaacatcggcttattctgaggctatccaatacattttttttctagttttcatttcaagtaaagcactcactgtgtataggaatttgtaattggaggtgcttgatctctacaaaagaaattaggaatcgctttattataaaatgctcctagaagtcttaattgtgttcatttctaaaaaattttgtaatgttagttgtgtgcatggaaataattaaggt

SEQ ID NO: 1161 - DNA, 363 bp, Gallus gallus
accatttcgctcagcaaggtccccgactccgcgcatccaatgccatgatgaataacaatgacctagtgaggaagagaagacttgcggagctgaacgggcctatttttcccaagtgcaggactggagtgtagctcccaggcagaggtcgttcccagcgggtctgtgctactgtgacaacctaaggcaaagaagtgccttgagagagttatttgtggtgcctcggttctgtttcatgcactaacagtttaaagtaactagtggctgtagttgaagatttttatccagtagcactgttgttttctgtagagctggaagctatccaagccagtaacctgccagtgttgtgcagcctcagctgagcgt

SEQ ID NO: 1162 - DNA, 428 bp, Gallus gallus
  misc_feature (403)..(403): n is a, c, g, or t
  misc_feature (424)..(424): n is a, c, g, or t
acctgctactttaaaacaaattttaactgcagctacttttcactaagcaagatggataaagcatgccatttatattttgccttctcaagagattattttcagaaacatatattattccaccgcaatctgacacttcctgtcatgctttcatcttgtaaaacctgaattccaattttaggctattccaggcttatgcttaaatgacagtgccttggtaagagaaaaaataattgtgctgcctttttctcccatagtgcctgaaaacatattgggcatacatatattatatatattcttacaaatgtccaggtcatgtataccagctgaaattcttttaatgtgggggtgtttgcattgtgagatttaatcaagacattaacatgagtagaaggttgttgttttnagacagaagtttgagaatcanctca

SEQ ID NO: 1163 - DNA, 472 bp, Gallus gallus
acacctctaccccgacaagcattacatttctgaacagctccagccttccctccttgaccattacatgcactacaaaggacattcttgctaagttgtagtttagttgtctttccattatataaatcttctaaagagactttgagaggatgcatcatatcttctcctcttcttctaccattacgacttctactctgaccacccatgaagttgaacaatccaccaccaaagatgtgggagaaaatatcgtccattccactgcttccaccactgccttctcgaaggccctgttctccatatctatcatataactcacgtttctctggatttgacaatacttcataggcaaagcttatttctttaaatttgtcacctgcatttggattcttatcaggatggtattccttggccagttttctataagccttcttgagctcgttgtcggaggctccgggcggcacgcccaggatatcgt

SEQ ID NO: 1164 - DNA, 554 bp, Meleagris gallopavo
acagatcatccagcttgcggaaggcgctttcagtccaaatgcagaaacgcccaacgtggccaccaggagcaagtctcagcaggttcagcttgttcacatcaagaagagtaatccccgggatattccggaaagctctaatgatgccgttgtcctcgttgtagatgatgcaaggtcccctgcgctggatgcgacggcgattcctcatcttacccttcccggccctcatgcgctgagaggcataaaccttttttatgtcattccaagctttaagcttcttaagaaggagaacagcttcctttgttttcttgtaactctcaactttgtcctcaacaaccagaggaagttctgggatctcctcaatgcggtgacctttagacatgaccagtgctggaagagctgatgctgccaaggcagaacagatggcgtaacgtttctgagttacgttcactctgcggtgccagcgtcgccaagtcttggttggggcaaacatgcggcctccacggcacatatttccaaaggcaccctggccagagcggtgagttccaccacctcgt

SEQ ID NO: 1165 - DNA, 554 bp, Meleagris gallopavo; identical to SEQ ID NO: 1164

SEQ ID NO: 1166 - DNA, 273 bp, Gallus gallus
caggctcacgctctgctgatccagaagctcttggcttaggctcctgatttagcactggcaagttttgtttgcatttctgtcacaattaaaaagtgttcctgaaccgcaatcgccaaagcaggggtgaattacaggatatagcacgacaaatgcatttttctgagagcaacacaacctatgcatgtgctgactagatacagcttcctagaaaaagaatagctttttcaaaataagagatacgattcttcacttctgatagagtaacttcttctt

SEQ ID NO: 1167 - DNA, 109 bp, Gallus gallus
  misc_feature (66)..(66): n is a, c, g, or t
ctgcttgttatgttggtgtctgcgatacggatatagaaagcctcttcatcccttggaawgcytccntttkcaatgccctgagctcttgagtggatcgttgccyagttct

SEQ ID NO: 1168 - DNA, 465 bp, Gallus gallus
accacctgagaggacrttrttrgcatamagatccttwckratrtcwatrtcrcacttcatgatgctgttgtargtrgtttcatgaatrccagcagattccatrccratraargatggctggaakarrgtytctgggcagcggaagcgttcatttccgatggtgatgacctggccatcaggaagctcatagcttttttccagagaggaagaagaggcagcagtggccatctcattttcaaagtccagggccacataacacagtttctccttgatatcacggacaattccacgctccgcagtggtaacaaaggagtagccacgttctgtcaggatcttcatgaggtagtctgtcaggtcacggccagccaggtccagacgcatgatggcgtgtggcaaagcatagccttcataaatgggcacgttgtgggtgacaccatccccagagtcaagcacaatccctgtggt

SEQ ID NO: 1169 - DNA, 465 bp, Gallus gallus; identical to SEQ ID NO: 1168

SEQ ID NO: 1170 - DNA, 465 bp, Gallus gallus; identical to SEQ ID NO: 1168

SEQ ID NO: 1171 - DNA, 275 bp, Gallus gallus
cgcacaaactgtgtagtgtcagacctgattatgggcaatgagtatttctttcgagtcttcagtgaaaatttgtgtggattgagtgaaactgctgcaactaccaaaaatcctgcctatatccaaaaaacaggcaccacttacaagccacctagttacaaagaacacgacttctctgaacctcctaaattcactcaccctttagtaaaccggtctgtgattgcaggatacaacgctacactcagctgtgcagtgagaggaatccctaagccaaagat

SEQ ID NO: 1172 - DNA, 342 bp, Meleagris gallopavo
acttctgggaatcaaaagtgaaatggaactaatcctagtttaattgcaattgctattgttagtagcagacatgatgtggggttgtttagttgtgtaatgtctcactgtcctgtagatcaggcattggtaaggcttgagaataggattaaggctgatgcagttgattgggtgaggaagtatttaatggcagcttcaattgctcgagggtggtgggattttgagatgagtgggataatagctaaggtgttgacttctaagcctgtccaggccaagattcaatggttgctggacatagtaatgctggttcctataattaagcttatggtggagattagttttgcg

SEQ ID NO: 1173 - DNA, 682 bp, Gallus gallus
aggtactccttatgattttgggacatgctcattgcacaagctctccagtttctattaaagctgaggatagttgccatctcttctttagtcagctcaaagtcaaacaccttgaagttctccacaatgcgctgtggtgtgacagacttggggatcacaatcacatttctctggatgtggaaccgaatgagaacctgtgctgctgttttgttgtgcttggctgcaatctctttaattttagggtcatccagaagtgagggatcctctggcttagcccatggtctgtcaggagagccaagggggctatatgctgtcacagagatccctttggattgacagtagttgatcagcttctcctgggtcaggtatggatggcattcaacctggttgtttgcaggtttgtatttcagtcctggcttgttcaagattctttctatctgctcatggttgaagttggaaatcccaacagcttttgccagaccagcatccaccagttcttccatggcctcccatgtttgtagaagatccgtgttgccaggtattgacatgcctttgtcatctgcaggaaataggtcctctcctgccttaaatccaacaggccagtggatgaggtagagatccagataatccagtttcgggctggcaagagtcttctggcaggctcctttcaccaatgatttttcat

SEQ ID NO: 1174 - DNA, 217 bp, Gallus gallus
  misc_feature (198)..(198): n is a, c, g, or t
aggtcggaataattcttcagcttcagggttgggctctttcagccattttgccctgttatagtccacaaatctaatcaacaaagggtggaggggatagaagtcccgtgccaaagtggtgaactcatctaggatgagcagttcagcctccgacatgtcacctctgccttctgctcttagatgctctgcatccgataccanccactgctgcttttttctt

SEQ ID NO: 1175 - DNA, 656 bp, Gallus gallus
acagattttggtttcacagaaatttaactgcagaaacaggaaaagcactccaagatatcagttgtaggtcatgtagcggggagtagcactgaattttgcataggatttaatgactttgttcactgcttctaagaagtctttctcagttgcaatttttcggcgtgctcggattgcaaacatgcctgcttctgtgcagacactgcgaatctcagctcctgtgctattaggacacagtcgagccaacagctcaaatcttatgtccctttccacactcatggagcgagcgtgtatcttgaatatgtgagtccgcccctcaagatcaggcaagctaaactctatcttcctatccaacctcccaggcctcatcagagctggatccagagtatcaggcctgtttgtagccatcaacactttgatattgcctcgtgggtcaaaaccatccaactggttgatcagctccagcatcgtgcgctgcacttcattgtcacccccagcaccatcatcaaagcgagcacctccaatggcatcaatttcatcaaagaatataagacaagcttttttagttctggccatttcaaagagttcgcgaaccattcgagctccctctcccacatacttctgcaccagctcagatccaatgactctgatgaagcagg

SEQ ID NO: 1176 - DNA, 345 bp, Meleagris gallopavo
cgccggcggtgcggctgcagacatggcgatccgctaccctatggccgtcggcctcaacaagggctacaaggtgacgaagaacgtatccaagcccaggcactgccgccgccgagggcgcctgaccaaacacaccaagtttgtgcgagacatgatcagggaggtctgtggcttcgcgccctacgaacgacgtgctatggaactgctgaaagtttccaaagataaacgtgctctgaagttcatcaagaaacgggttggcactcacattcgggccaagcgaaagcgggaagaactcagcaatgtcctggcagccatgaggaaagctgctgcaaagaaggattgagttgt

SEQ ID NO: 1177 - DNA, 305 bp, Gallus gallus
  misc_feature (261)..(261): n is a, c, g, or t
  misc_feature (282)..(282): n is a, c, g, or t
  misc_feature (285)..(285): n is a, c, g, or t
ccatgaaaacctgcacctattgacaccaaggggagaaagaaaaacacrgggcacttcagaatggattcagggaatttccactgaccttttaagaaatggcttgtggccaccttgatcctgagagattgtggttttaatttgaaagaattcatagattgaacacttgtaaaaattaataagcacctacgacaacgaagagtacacacgaggatttaaagggtagggatttttttttacgggtctgacttatcttcccggggnaaaatggttttataaaacttncanagaacttttttaagagccgt

SEQ ID NO: 1178 - DNA, 467 bp, Gallus gallus
  misc_feature (427)..(427): n is a, c, g, or t
aggtaccacagccaggagctgattcacatttcggatttgcaatctgaggtgcctcccattcgccatccatatcctcatcccaatcttcaggcttctcagcatcagggtctgctacgtattctggctcatcatccagccagccctctggtttcacagcattttcatctgctatctttgcaggagcatcttcatcccagtcatctggtttaacagcatctggatctgggattttgggtctctcatcccaatcctcaggcttctggtcatttgggtcctcaatctctcgaggtggattcacaggaggagacatatcatttagcaaattcccactgttgacaaccatttgatcaaccagaatttcaaaactattatcaggattcaaaaccagagtataaaggtgtgtcttcttatcagagaagtaggtctncaagtctgcatctggacgcttcrcgtgcttctcctcatat

SEQ ID NO: 1179 - DNA, 312 bp, Gallus gallus
gtcctgctgaaggctgggattcctcttgggactttgttgacttgtggatagcaggcgtgctctgctgattcattttcatcactgtcaaaatggtcatcaaagtctttgtagccacatcttcggggtctacagcgatcaaaaagaaacagcaagaggtagtgggctttttagaggccaacaagattgactttcagcaaatggacatagcaggtgatgaggacaacaggaaatggatgagagagaatgttcctggagaaaaaaagcctcaaaacggaattcctcttcctccacagatcttcaatgaggagcggt

SEQ ID NO: 1180 - DNA, 362 bp, Gallus gallus
acttagcagcaatcccaaagcgcgtgttgttactgccagcagtccatgcaagattcactgatgtctcaaccttattattaaccttctgataaatagacccaccaaactctgtgccatcattcacgtttgtgtgcagctgaaagtctcctgccttatatcccaaggcaaagttgttttgggaaagcttagatttggctgtatcaaaagccatctgatagccagcaagccagccttcataacccagcactgcccagccatagatggttggtccagagagatcaatgtctatattgcagcctaggtttacgtattctcttttgtaggaagtcttcaattttccactcttcttccctgtatttggt

SEQ ID NO: 1181 - DNA, 485 bp, Gallus gallus
accaagtggaataaaatacctttatcttcgaaacaatatgattgaggccattgaagagaacgcatttgacaatgtaacagatctgcagtggctgatcctagatcacaatcatctggaaaattcaaaaattaagggaagagtcttctctaaactaaagaatctgaagaaacttcacattaactacaacaatttgactgaagctgttggaccgctccccaaaactctggatgacctgcaattaagtcacaacaagatcacaaaagtcaatcctggtgcacttgaggggctggtaaatctgactgtcattcatctccagaacaaccagctgaaagcagattctatttctggggcatttaaaggtctgaattcacttttgtatctagacttaagcttcaatcaacttacaaagctaccaacagggctgcctcactccttactcatgctgtattttgacaataaccagatctccaatattcctgatgagt

SEQ ID NO: 1182 - DNA, 204 bp, Gallus gallus
  misc_feature (20)..(20): n is a, c, g, or t
ctgattccagcsgccccccnggcaggtactttctgatctgatggttattacatcaccsttgatgctgatagttaaattaggtttggscacaccagaccatcttcctgggaggaaaccccactcccagctctttcatatagtcctcaaagttttcacttgaaaggagcttccaggtgcccacaaactgggtcgcacattttgtca

SEQ ID NO: 1183 - DNA, 346 bp, Gallus gallus
accttggctttagttttattagcatgaaacacctttggcatgcttagcttccaggtaactggtaactccctacctgtatcagaattcattttacgtagttccacagaacatgaagatccttatttgctaagcctttgaaagctgacattctttttcataagagggtgtatttaaatggatgttcaccaatagacaataggccagcttactagtggtgagctaaaactgctaaatgaatgtccaacatgattgtaaggcttatgtcactcaagattttatcctttggattttcatgatcaaatttcatatactattgtatagacttctgctttgtagtgtacctgca

SEQ ID NO: 1184 - DNA, 331 bp, Gallus gallus
acaggttgaagatttttgcagcattagtgctcgtcactgccacaaactggttttcatccatctttcctgtagccactgctttgtcccagatgacagacatccgttcctcgatgccattggtcccctctgggatcgctgtgaagttgtcttttccaattgctttctgcgcagtgctgaacgtgcagtgggcacttcctgacacctgcaggccaccactggccaggagggagttaatgtagtcaggggttgtgggatctgggcttagggggggcgagaccacaaatgctgcagccttggcccagttcttgctccagtagtgtgttccatcagt

SEQ ID NO: 1185 - DNA, 525 bp, Gallus gallus
  misc_feature (518)..(518): n is a, c, g, or t
aggtacttattgcacagctaattggctattaacatcactgccatgctarcatccatccagcaggaggaagcagctgttgaaggcactgaaggaactaaacttgctctattaaaaagaggaaaaacctgttacttagacatccacatctgccattgctctctgcagaggatcaacagaccagtagtcgtcatcccagcccaggaaccagccagagaaggttggaggctcaagcccttgcttaacgagtgtgactggagttctcttatcacggctggctggatcagtttcaatgtatcgyttagcagatttcaaagcctcggtcttttcttcctcctgggcatcttttccaatccatacaaacacctgatcccatgtgtcaaggatcataacatcatctgtagcaaggtcatcctgggtcaggtctccagggacttcttcaatagtgaagcgtccactcttgttggagcatgcaaaaagacgaggggggtgagcatccatcttcttgtccttcagccgangagaagt

SEQ ID NO: 1186 - DNA, 224 bp, Gallus gallus
acttgttacaatacagcatggagaagttaccaagcgattggacacaacaaccttttctactttcttctcaaggatatctttcattattttgcaaaggttttcaaacttggcttttttctcttcctgtttctttttctcctcttcatcttctggaagctctaagccctcttttgttatagaaaccagagtcttgccttcaaattccttcagctgttgcacacagt

SEQ ID NO: 1187 - DNA, 224 bp, Gallus gallus; identical to SEQ ID NO: 1186

SEQ ID NO: 1188 - DNA, 427 bp, Gallus gallus
acagacataggtgtaactgcagttcactaacagcagcttaactccttggtgttgacagtggacattgtgctgggggcactcgaatcccagtgctgaaattaacactagtggaatctgtccttcatctttgcactgtggtatatctatgccatgttattaatcccgttctgtgcaatcagcagtgtgctaacctgctttttttcttctgtaagcatttcgcattattgggcttcattacctgccttgctttgtataccaaggctggttctcttgcacatcttacgcttttatacctttaactttttgaatggtcagatactgaactggacagtcaaacaacttgtgttctttagggagtcgtagctactgttgtattttaacactacagctgagggcttctttgagggcgggtttcttcttggagagt

SEQ ID NO: 1189 - DNA, 501 bp, Gallus gallus
actggttctgagagagcttttaaggtcccagagaagcaagctgctccaacctaagtcattacaacaaactactatgtcatatacttgtttgtaaaacccagtagagttttttgttgttgttgtgttatttttaaatatttgtttcttggtttaagcaaaatgacaagcggttatggtgattagatatagagtggggcaaattaagtgagttgatttagttgtgtgtataaataagtagtgtgtgaaagtgctcaactgcctaatggaatttaggacttttctaaatgtttatgcagacttagctattgcataactattggcctgataaccagagcggctgaggatgtggaacaaactacatatcagagttcactgggatgaatatatggtatctttggatggaagaagttcggtaaggattagttatttcagctccacataaattactttgaaggagttaggctgtcagaaagtgccaaatactcacttttgggctccagt

SEQ ID NO: 1190 - DNA, 312 bp, Gallus gallus
acatcaggagaatgagatgcttatttgtcacttccacataaagccaccaggatggtttcataatcccctttgagttcatccataattgcttggcgaagagatataccatacagactcttataataggctttgatctcattcaagtctacttcatggcgtgaaaccatgattctgataagctgtttgtgtcgtgtcccacttcccttcatggccaagtggagtttttcagcaaagaaagctggcttgcttgtggcacacttcacaagggcagtcaagcagttttcaatatcacctttcagctccaaatcaagt

SEQ ID NO: 1191 - DNA, 592 bp, Gallus gallus
acaagttcttcaagggaaagaaccgccatgcttcctgcagtgcttccaaggagggatgattgtgcatgctggaagaagggaagaggaagaagaaaacgcacaaagtgactggagactatattgtgtgcgaggagaagttcccaatgaaggaaacttacttgaagtagcatgtcactgcagcagcctgcgttcccggacatccatgattgtcctcaatataaataaagctcttatctatctgtggcatggctgcaaagcacaatctcacaccaaggatgtaggaagaacagcagccaataaaataaaagaacaatgtccgctggaagcagggctgcacagcagcagcaaggtgacaatacatgaatgtgatgaagggtcagagcctttgggattctgggatgcattaggaaggcgagatcgaaaggcctatgattgcatgttgcaagatccaggaaagtttaatttcaccccccgcctgttcagcctcagtagttcttcaggagaattctcagccactgagttcgtttacccttcaagagaccctgctgtcatcaattctatgcccttcttgcaagaggatctttacactgcc

SEQ ID NO: 1192 - DNA, 260 bp, Meleagris gallopavo
accatcgaaagttgatagggcagacattcgaatgggtcgtcgccgccacgggggcgtgcgatcggctcgaggttatctagagtcaccaaagccgccgggcgagcccgggttggttttggtctgataaatgcacgcgtccccggaggtcggcgctcgtcggcatgtattagctctagaattaccacagttatccaaggaacgggaggggagcgaccaaaggaaccataactgatttaatgagccattcgcagtttcactgt

SEQ ID NO: 1193 - DNA, 305 bp, Gallus gallus
gactctgtccgctgtgggttcggtgccgccatggccaagtccaagaaccacaccacgcacaatcagtcccgtaagtggcacagaaatggcatcaagaagcccagatcccatagatatgagtccctcaagggggttgatcccaagtttctgagaaacatgagatttgccaagaaacacaacaagaaggggctgaagaagatgcaggccaacaatgccaagcaggcagctctacagaaaaaggactgacctggtttaagacaaagaaccagtttgccttttggcatgtgtgtttaaagcatttttgt

SEQ ID NO: 1194 - DNA, 361 bp, Gallus gallus
caggtactgaaaaacttctaggcttccagcttcaccgactctagaatggaacgtgcttctttatactcttcagcttcttccaactctttttcattttctagtagttgagtgagatctgcatgtgcaatttctaatctccgctgacagtctggaatcatcattcgagactcttgtaagatctcaacctgcttttttattccatagtcatcacatgcttcagctttcattttttcaatcctctcttcttgttgttttgcttcwttttcrtacataactttttcttttgccaatcgcttcacgacgccggttttgatcttgatctgcctcagacggggatcggmcatggcggggchgcagcgcg

SEQ ID NO: 1195 - DNA, 271 bp, Gallus gallus
ccgggcaggtacgttcttgaagggttaatggtatgtgatttatactgtgccttaattgttatgctatttaaaaacaaatatttattttgaaagttttactatgctgtgctctaaagaaagcaactttagatgtgacactgtataattatgtattcatctcatggcataaattatttagtagacttagatgtmgcatattaaatatkaacctaattaactaaggatgttgacttggatttatttaaattcwgtatgtgcactgtatgagggt

SEQ ID NO: 1196 - DNA, 270 bp, Gallus gallus
ctgcagctccagcagcgcccggtccatcttgttcatcatcaggacaggtttgatcctctcagcaatggcctgacgcagcacggtttctgtctgcacacacacaccagagacgcagtcgacaacaaccagggcaccgtcagtgacccgcagagcagcagtgacctctgaagagaaatccacgtgcccaggagagtcgatcaggttgatcaagaaaccagaaccatctttgctctgcttgatgaacgccagatcgttttcagagagctcgta

SEQ ID NO: 1197 - DNA, 515 bp, Gallus gallus
  misc_feature (428)..(428): n is a, c, g, or t
  misc_feature (430)..(430): n is a, c, g, or t
  misc_feature (460)..(460): n is a, c, g, or t
aggtacaggctagcatcttgcagaggaagagcttacttcctctggtctagtttccttacacttaaaatgaaaggcaatacagaatcttattctacttctgccttgagaaaaacaaaataatttactttccttatatagcttagtgctctgaaaacttagttcttaagttaaaccagaattattttacacgaaccttttcatcagatgcaatcttaccacttgtcagactcttccccagtatacattacaaagctgcttagtaagaaaagttgtgtgaaagcagcttctaattaatggatcacatgagatcctgcatcatccccagtagcagcagtctgctagcaaccrcagaaatacattagcaaaggttacaccgaagcagtcatgtctgacagctaatacagcactataacatacagacctttcrnangcaggtcagtatgtagaaataattctttancatgtaaacaggaaaactgatctgtcagttacrtagatcaacagctgaagctatt

SEQ ID NO: 1198 - DNA, 160 bp, Gallus gallus
  misc_feature (113)..(113): n is a, c, g, or t
aggtaccgcctgcagagggagaaggaattcaaagccaaggaagcagcggcgcttggatctcatggcagctgtgtacaagcttttttktttkttttttttttttttttkttggnttttttttttttttttttttccacaaaaaaaaaactttcttatgkkt

SEQ ID NO: 1199 - DNA, 1252 bp, Meleagris gallopavo
ctttctgttgacgagctttcatcttggaggaacgggttctgtgaagcattcttcagagtgaagtggtcctaattcttcctggaaccattgcaacccattccactcagggagccaatcctatcaattcttctgccgaagcagccagaatctctcatcatccggggcatctgcacccccctcagtctcttgaggaaggggttcctgtaggacagaggagtgttggatgctagcttgggttcagccttctgctcatcgctgtcatctatgagttctggtggaatctcctctcgggtttggggctcttgtaggtcaggattggactccagggcttcaatcagtgcgaatttgtcctccagtctctccagaagagcctccatgctggccagttctttggcagggctgaggttgtagatggggttggccctgctgggctgcagctggacaagaagcagcaagaggaaaccacaggaaaatgagcctctagtgtccatggcgctgggttcgttgggaatatgggaagttcaagctgtttcttctgagatggctcttcaggtctctctcttacttggacgaaggccggttcttcgaaagtgtggtatgggggtggacatccgtggattcattcaatgttggtagaggttagtwcaggrygtmgtccactctaacaaacctattgaccataactctatcctacataatcccaatcctaatcgccgtggccttcttaacacttgtagaacgaaaaatcctcagctacatacaggcccgaaagggcccaaacattgtgggcccttttggtctacttcaacccattgcagacggagtaaaactctttatcaaagagcccatccgcccatctacctcctcccctttcctcttcatcataacacccatcctagccctacttttagccctcacaatttgaacacccctcccactccctttcccccttgcagacttaaatctaggactactatttttattagcaatatcaagcctaactgtctactccttactttgatctgggtgagcctctaactccaaatatgctctaattggggccctccgagccgttgcccaaacaatctcatatgaagtcaccttagccatcatcttactagccacaattatactgagcgggaattacacrctaagtacaacacaaaaagcaagcaagctggagggcctgttgatgtaggtcccgagtttcagaaagacatgaatgaatcacttgccaggcttcagcggatgt

SEQ ID NO: 1200 - DNA, 544 bp, Gallus gallus
actacattcacaaagtcttccgatacgtccttcattacatctgcatgctccacattcaaatgtcccatttccgtcgtggcaggcggggctgtttggttctccttcgctttgacacagacaatcacggatgaactggagattgatttccacttcttcagtgaaccccagtggtttaattttaatggtttcattttgtcctttctttggacattcatttgctgttacattaatctcaaacctaacctcatctcctattgaaatgttggaacattttcttccgtcttcctgcgtgtcgttgactccattcttgcagtatgatttgtaactgattgtcactccttttggtagcttactgttttccaggatcacctctgaagaaagggaattgtatgcatcaatgatcaactgaatgacattgctggaattggaagacaacgttcctactgctgattttggtatgaggtttttcagttccttataaactgcctgaaactcttcagtaacagcaaaaattgtctgaatattgttctcactaagcttctgt

SEQ ID NO: 1201 - DNA, 624 bp, Gallus gallus
  misc_feature (621)..(621): n is a, c, g, or t
actgtataaaaacttgtgttgagttggaggtataaaagcccagttgtctgtatcaataatcaatgatgtttttgggaattttagaatagctgctgagaaattcacccacttactgataagaggcaacagctgctgctcatcgctttgatcacagattttgtaaggctttttttttccagcaactgtttgggcctacagcttctctatcaatattgcagaagcacctcctcctccattgcaaattcctgcaagaccatactgcccttgtttcagtgcatggaccatgtgaacgacaattctcgctccagacattcctatcggatgcccgagagagacagcgccgccattgatatttactttttgtggatcgatacccagcattttaatattggccagcaccacaacactgaaggcttcattgatttcccacattgcgatgtcttcttttttcagacctgtctcacttagaatcttgggaacagcgtgtgcaggtgcaatgggaaagtcaataggatcaacagcggcatctgcaaaagcaactacccgtgccagtggtttaactttcagtctcttggctgcctctgtagtcatcagaaccaaagcagctgctccatcattcanagt

SEQ ID NO: 1202 - DNA, 372 bp, Gallus gallus
aggtgaacgcattcaaggtgtttgatccagagggcaaagggctgaaatctgcctacatcaaagaaatgctgatgacacagggcgagaggttttcccaggaagagatcgatcagatgtttgctgccttccctccagatgtctctggcaacctcgactacaaaaacctcgtccacgtcatcacacatggagaggagaaggactaatccatggattcagcactggggttagcactgtgggatcacctccatgtgggtcacactgcaggttccctttgtccctctccctggagctgcagagctgttcttcatggggataacaacccagaacagcagccacatacaataaagtgcattttggtgagagtaaaaaaaa

SEQ ID NO: 1203 - DNA, 618 bp, Gallus domesticus
aggtactagaaacacatgctatgtatgtcatttagaaatgtagtgctgcttctagatgagacaactcttgaaggtgaagtatagtttcacgtagctctacgtcccttcccagagagtaaaacaattcccttcacccttaacttcccatttactttatccaaaatcaggaggaaccaacaacgcaccatagattctctacagtccacccttgattctgaagcccggagcagaaatgaggctatccgtctgaagaagaagatggaaggagacctcaacgagatggaaatccagctcagccatgctaacagacatgctgcagaagcaaccaagtcagcacgtggcctgcagacacaaattaaggagctccaggtgcagctggatgacttgggacacctgaatgaagacttgaaggagcagctggcagtctctgacaggaggaacaaccttctccagtcagagctggatgagctgagggctttgctggaccagactgaacgggcgaggaagctggcagagcatgagctgctggaagccactgaacgtgtgaacctgctgcacactcaggttggcttttcctgggttaaactgagcttcacctgttaagcactgacactggga

SEQ ID NO: 1204 - DNA, 581 bp, Gallus gallus
tgcaatggaaggagttttcacaagacgtgcttcctctgcatggcttgcaggaaggctctggacagcaccacagtggcagctcacgaatctgaaatctactgcaaaacttgctacgggagaaaatacggccccaaaggtgttggctttggacaaggggccggatgtctcagcaccgacactggggaccatctgggcctaaacctgcracagggatcaccaaagtctgctcgcccttctacaccaactaatccttcaaagtttgccaaaaagatcgttgatgtggataaatgtccccggtgtggcaaatcggtgtatgctgcagagaagataatgggaggaggaaaaccttggcataaaacatgcttccgctgtgctatctgtggaaagagtttagagtctacaaatgttacagacaaagatggagagctctactgtaaagtttgctacgcaaagaattttggtcccaaaggaattggttttggtggcctcactcaagtggaaaagaaagaatgaagccttctgaagccttctgaagaaaaagcaagttttcttagaatatagtgtttcagttttgttattgt

SEQ ID NO: 1205 - DNA, 1242 bp, Gallus gallus
cgctggggccgttgacgtgcagcaggaacactataaaggcgagatggtgaaagtcggagtcaacggatttggccgtattggccgcctggtcaccagggctgccgtcctctctggcaaagtccaagtggtggccatcaatgatcccttcatcgacctgaactacatggtttacatgttcaaatatgattccacacatggacacytcaagggcactgtcaaggctgagaatgggaaacttgtgattaatgggcatgccatcactatcttccaggagcgtgaccccagcaacatcaagtgggcagatgcaggtgctgagtatgttgtggagtccactggtgtctttactaccatggagaaggctggggctcatctgaagggtggtgctaagcgtgttatcatctcagctccctcagctgatgctcccatgtttgtgatgggtgtcaaccatgagaaatatgacaaatccctgaaaattgtcagcaatgcctcgtgcaccaccaactgcctggcacccttggccaaggtcatccatgacaactttggcattgtggagggtcttatgaccactgtccatgccatcacagccacgcagaagacagtggatggcccctctgggaagctgtggagggatggcagaggtgctgcccagaacatcatcccagcatccactggggctgctaaggctgtagggaaagtcatccctgagctcaatgggaagcttactggaatggctttccgtgtgccaacccccaatgtctctgttgttgacctgacctgccgtctggagaaaccagccaaatatgatgacatcaagagggtagtgaaggctgctgctgatgggcccctgaagggcatcctaggatacacagaggaccaggttgtctcctgtgacttcaatggtgacagccattcctccacctttgatgcgggtgctggcattgcactgaatgaccattttgtcaagcttgtttcctggtatgacaatgagtttggatacagcaaccgtgttgtggacttgatggtccacatggcatccaaggagtgagccaggcacacagcccccctgctgcctagggaagcaggaccctttgttggagccccttgctcttcaccaccgctcagttctgcatcctgcagtgagaggccagttctgttcccttctgtctcccccactcctccaatttcttcctcagcctgggggaggtgggagaggctgatagaaactgatctgtttgtgtacct

SEQ ID NO: 1206 - DNA, 573 bp, Gallus gallus
acaacattactaccagctttttgatgcagacaggactcagttaggagcaatatatattgatgcatcatgccttacgtgggaaggacagcagttccagggcaaagcagctatcgttgaaaaactctctagccttcctttccaaaaaatacaacacagcatcacagcacaagaccaccaacctacacctgacagctgtatactcagtatggtagtgggacagcttaaggctgatgaagatcctatcaygggattccaccagatatttctattaaagaacatcaacratgcctgggtttgcaccaatgacatgttcaggctagcattgcacaactttggctgagctggcgaccccgaggcacctgttctttttttcttcttctctcctcttactgatattattcacactcacagaacattccaaatatcatacacaaacctgcagcactgcagagcgtgagcaagcaagagctgtgacctgcccttctgctgagtttacattgtcactagatgagttccttgtgcatgatgtttggaagttagttagctgcatttgacaagagaaatttgtgttgt

SEQ ID NO: 1207 - DNA, 411 bp, Gallus gallus
aggtatgatcctccaatggaagctgctggcttcactgcacaggttattatcctgaatcaccctggccaaatcagtgctggttatgcccccgtgctggattgccacactgctcacattgcctgcaagtttgctgagctcaaagagaagattgatcgtcgttctggcaagaagctggaggatggccctaagttcctgaaatctggagatgctgccattgttgatatgattcctggcaaacccatgtgtgttgagagcttctctgattatcctcctctgggtcgttttgctgtgcgtgacatgaggcagacggttgctgttggtgtcatcaaggccgtcgacaagaaggctggtggagctggcaaggtcacaaagtctgctcagaaggcccagaaggctaaatgaaaattctgt

SEQ ID NO: 1208 - DNA, 411 bp, Gallus gallus; identical to SEQ ID NO: 1207

SEQ ID NO: 1209 - DNA, 259 bp, Meleagris gallopavo
actgggagaagctctccacacacatcggcttgccaggaatcatctccacgatggccgcatcgcctgatttcagggatttggggttgtcctccagcttcttgccggagcgccggtcgatcttctccttcagctcagcgaacttgcagacgatgtgtgcggtgtggcagtcgatgacaggtgagtatccagcactgatctgcccggggtggttcaggatgatcacctgagatgtgaactgtgctgcctcctgcggcggatc

SEQ ID NO: 1210 - DNA, 625 bp, Gallus gallus
gagatgaagatcacatatgcacaatgtggagatgtcttgagggctttggggcagaatccaacccaggctgaggtcatgaaggtccttggcagacccaaacaagaagacatgaactccaagatgattgactttgagaccttcctgcccatgctccagcatatcgccaagacaaaagacacrggcacctatgaagactttgtggagggtctrcgtgtgtttgacaaggaaggaaatggaacagtgatgggggctgaactccgccacgttttggctacactgggtgaaaggttgactgaagaggaagttgataarctaatggctggccaggaagatgccaatggttgtatcaactatgaagcttttgtgaaacrtatcatggctaactgaacaccaggacaagacaggcgtggagaagcccggattctggccttggattttgatttattggaatgtcctctcatttttcagtccagattcctacttcaaagctataaaatgtattgtccctgaagttatttggataaatgcttgtttgttttgtcttgtttcctcatgggaagaaaaaaggaaattgaacaaacagaaccagaaccatgaataccttattgcattgtatgcaataagg

SEQ ID NO: 1211 - DNA, 453 bp, Gallus gallus
gaggatggcagcggcactgtggactttgatgagttccttgttatgatggtccggtgtatgaaagatgatagcaaagggaaaactgaagaggaactatcagatcttttcaggatgtttgataagaatgctgatggctacattgatcttgaggaactgaagatcatgctgcaggcaactggagagacgatcactgaggatgacatagaagaactgatgaaagatggggacaaaaacaatgatggcaggattgactatgacgagttcctggagttcatgaagggagttgaataaatctgaggccagatggacagcccgaatctctgaaactccttctgctctctgactcagctccttggttccatcccctggctgccagcatgaagactgagcactgagaagggtggccgtagggaaaataaagcacattgctgtcaaaaaaaaaaaaaaaaaaaa

SEQ ID NO: 1212 - DNA, 644 bp, Gallus gallus
acctgattcttcttaacaaatggaggaaatgatgccccatcagtgccgttaaccaaatcgcagtaaccttcccagtaagacagattccttttgtttttataactttcaattattgctgttttgcttatgtcttctttcccagtatacactctgtaaagtccatcagatgtcccattatacgggtagaagactcccagaactgggtccaaggggaagggaaccttgcttaagaaggggtctttgtatccccatagtatttctttcactgttctgttctgcagcatgtttgatttagaagatttaatccaagtatttaaaagtaggaggatgaaattgtttgtatacagggcaggtgcagcaacaacagcgaggttgaggcacgtgatggtgtcattttctgtcccaacagacatatcaggttcaaaacgagcagcgttaggcaacatgtaagatattgtgccattagagttttctgtaatattttctttaggtaaatatcgcaccctatatgtgtaaggtcctctttgttcaagttttggacgtgctccatagttcaaaacctctgatggattttccacattaaagatccaaaattgcctgtaaacagagcttcctggcacaagccaattatcatatgcaatggt

SEQ ID NO: 1213 - DNA, 268 bp, Gallus gallus
actgccaccaaacccagaaccaaggccagcatcttccatgactcaaagcttgttttcaagaaaaggcagtgagctttggagcggagagaaggtaaaaagcagcagctatcttgtagaacttcatcatgagattggcaatggacatcctcttgttactgcacaaccttctatcaccagcacattttattctgaaccaacctctcaccctacaattgctgaatgagagtagaaacacaggtttgcagattattctgtcaactgcagaagt

SEQ ID NO: 1214 - DNA, 208 bp, Gallus gallus
acttatcttcaaaaacagccatagctgccagtgagccagaacccatggtaacataaggcaacttgtcagttgatccatggggatatatgctgtaaaggtgaggtccagtgacatctacacctcctaaaaccaaggcagcaccaatgtagccttgatacctgaaaagcatttgctttagcattcgattagctgtgaccacacgtggaag

SEQ ID NO: 1215 - DNA, 395 bp, Gallus gallus
acccttcctattaaagatcctcacgtagacagtgcatctccagtgtatcaggctgttctcaaaactcaaaacaagcctgaagatgaaactgaagattggagccgccgttctgccaacctgcagtctaagtcttttcgcatccttgcccagatgactggaacggagttcatgcaagatccagatgaagaagccctgaggagatcaagggaaaggtttgaaacggaacgtaacagcccacgctttgccaaattgcgcaactggcatcacggcctgtcggcgcaaatccttaatgttaagagttaaaagcccacgttcagtgggcaaagatgtgagagagaattacaggaaagaaataactgctatcctgagttagagcctaacaacgtaacacacgt

SEQ ID NO: 1216 - DNA, 287 bp, Gallus gallus
acattgaaggtctcaaacattatctgggtcatcttttcacgattggctttagggttcaagggtgcttctgtgagcaaggtggggtgctcctcaggggccacacggagttcattgtagaaagtgtggtgccagatcttttccatatcatcccagttggtgatgatgccatgttcaatgggatatttcaaagtaaggatacctcttttgctctgtgcttcatcacccacataggaatctttttgacccataccaaccataacaccctggtgcctggggcggccaacgat

SEQ ID NO: 1217 - DNA, 114 bp, Gallus gallus
atattcattccaagaacttcatccaccgggatgtgaagccagacaacttccttatggggcttggtaaaaaaggcaacctagtatacatcattgattttggtttggccaagaagt

SEQ ID NO: 1218 - DNA, 582 bp, Gallus gallus
acacaatgcagggtgcacgagcctgtgcttctctgaagaggctccggactcgtgcggctccaagacctcctatcacctccacaaattcagagcctgccatggccaagaaaggcacctgtgcttctgtggccactgcctttgccaacaacgtcttcccgcagcctggtggtccaagcaacaaggcacccttgggcactttagcaccgagctgaaggtagcgatcaggattctttaggtagtccacaaattctttgacttccatttttgcctcgtgcattcctgctacgtccttgaagccaattcctttcccggattttccgtccacaatggtgaaacgagccattttcagctgattaaaagcattgaatcctcctgcccggcccgcaaccctgataaggcggaagatgctccacaacatggacagagccaccagtgtcactatcagggaaatgacatcatttccgtaaaagccggggtgtttgtaggaaacagggattctctctctctcatcaatattcagctcgtcctccgcagctctcagcttctcttcgaacttgtcgatgtttgccactcgcatggtgt

SEQ ID NO: 1219 - DNA, 329 bp, Gallus gallus
cagctttggaaaacactatctttaacattaaggtgtaaaggatgaacaacacaaaattaaagtgtgtgctgtattgctagaatgcatcccttctctctgttctccacaaggatatgttcccattaacagtctagtctatgaaacaaatgtttttcccaatgaaaacttgaaattgttccattgtggaccaattcttaagagagcagtagcaggagatgcctctgaatctgcacttctgaaatgcattgaattgtgctgtggttctgtcaaagaaatgagagaaagatatcccaaagtggtggaaataccgtttaactctaccaataagt

SEQ ID NO: 1220 - DNA, 661 bp, Gallus gallus
acgggtcaagcaaagaagtcacagttaggggccataactgtccaaaaccaataataaacttctatgaagctaactttcctgcaaatgttatggaagtgattcagaggcagaacttcaccgagcctactgcaattcaggcacaaggatggcctgttgccttgagtggattggacatggttggagttgcacagactggatcagggaaaacactgtcttacttgttgcctgctattgtgcatataaatcatcagccattcctggaaagaggagatggacctatttgtcttgtgctggcaccaactcgtgaactggctcagcaagtgcagcaggtagctgctgaatatagcagagcatgtcgcttgaagtctacatgtatttatggaggtgctccaaagggaccacaaattcgtgatttagaaagaggtgtggaaatttgcattgcaacacctggaagacttatagacttcttagaagctggaaagaccaatctcaggaggtgcacttaccttgtccttgatgaagctgacaggatgcttgacatgggatttgaacctcaaatcagaaaaattgtggatcagataagacctgacaggcagactctgatgtggagtaccacatggccgaaggaagttaggcagctggctgaagactttttaaaaga

SEQ ID NO: 1221 - DNA, 343 bp, Gallus gallus
acttgagcacgacaagtttaaccttcttcctcttatgcttgttcttcttgggggtggtgtaagacttcttctttcttttcttagcaccaccacgcagtctcagcacaaggtgaagagttgattctttctggatgttgtagtcagacagcgtgcggccatcttccagctgcttcccagcaaaaatcagtcgctgctgatcaggaggaattccttccttatcctggatcttagctttcacattttctatagtatcagagggctcgacctcgagggtgatggtcttccccgtgagggtcttcacgaagatctgcatgtcgaggcccgcacccgcggggaagaggcg

SEQ ID NO: 1222 - DNA, 343 bp, Gallus gallus; identical to SEQ ID NO: 1221

SEQ ID NO: 1223 - DNA, 383 bp, Gallus gallus
ccgggcaggtaccttttaaccccatggaaaaaatatctaacgttcattactaccaataacaggaagaagattttgcttcgagaatgacaaacccatcatggtgaagtttaggcacgctccccacgaatgcggcgtgctagctggatatcttttggcatgattgtgacacgtttggcatggatagcacacaggttggtatcttcaaacaggccaaccaagtaggcttcacttgcctcctgcaaagcaccgatagcagcgctctggaagcgcagatctgttttgaagtcctgagcaatttcacgcaccagacgttggaagggaagtttgcggatcaaaagttcggtagacttctgatagcgcctgatttcacggagggccacagt

SEQ ID NO: 1224 - DNA, 473 bp, Gallus gallus
acatgaggactccaactgctcctgcctctttggcatttgcaaccttctcagcaagtgttatttttccagctctgacaatgactatggttccattcaatggagtcactgacttctgtattgtctcaatatcttttttcagtccatagttcacatagacaggtttgccagaaaaagagccactctcactgtaggccacgtatgcatcaggcatctccaagatctcctcgctatcattgatcaaaacggacactttgttcttggtgctgcctctgatttgcaacttaatatagtgttcatcgttccacactttatccaagaagaaactgttgaattgctcatgaatgtaggtggccatgtttgtatcttcagcctcaccagcctcaaaggagtccaaacctgccctttgcctcaagcggtctccaaggttcttggctaacagcttatctgacaacatggcttttaattgaggccag

SEQ ID NO: 1225 - DNA, 185 bp, Gallus gallus
gtttgttgctggaacacatcaattgtatcttcatcctccatttccaactgkgcgggggtgtctgtttcattaattggctgcccatcgaaccggaatctgatttgcctcatcgacaacccctgtcgttcacaataggctttcattagtttactaagkggggtatgcctcttaatcttaaactgcac

SEQ ID NO: 1226 - DNA, 337 bp, Gallus gallus
accctcgggcagcttaggcagtctcaccgtttctgcatcgagcaaaagcacaccatcactgctgtgcagtttgatgtccatctgggaaagagcttcaatattccctgcttgcgctttgatgttaatgcctcttggagcatccatgctcagagaccgagttggtgactccagccttagctgtttaaaagcttctgccttcacgagaggtgtttctacagagtgttcaaaaagtgctccttcaggccctgtgactcgaagtttatctgttccaatgacaacctcattttcatccactgtaaaaagtggcttgccatcctttgagttgatctgaaattgc

SEQ ID NO: 1227 - DNA, 606 bp, Gallus gallus
actccttcatgacctgaataaagtatggcatggcaaagtccatgatgttgtgcctccacgctgtttccaagacaacgtcaggccttaaaagatcatagcaggtgaagagacaagcaccgaagcactctttcttgttctcctgcaagaaccactgcaacaattcttctgccaactcagtatctttggattctgaagcatattgcattgcatccttatacagtctgtccttcttacagagttccacactttgcttccagcggttgtttcctttgaacagatatgcagcaatccttcggaactctatcagctcgtgtttctccaaacgttgagcaagagaaatgttgtcaaagttgtcataagcatctatagaagtcctaagggcctggtagtcttcttcaataatgaagaggttgttcagggactcattcactgatttgttgttgtgattttgaacagagcgcaagtaaggtttaaccaggggtaactgtttaaccttggtgaagaaggtaacagcacgagtatggtcaagtcgaggggacaataccatcagcagatcattgagcaacagaggtttgaattccaaatagaattgaactgctttgtagt

SEQ ID NO: 1228 - DNA, 363 bp, Gallus gallus
actgagcctgctcaggaggcagctctcgacgtaactcatctgccaggatatacggcttatctgaagccaggattctgaaggaagctatcacttgctcagccgtgtccgtatctgctgtttctctggtcatgaagtcaatgaaggactggaaagtgacggttccttgtccattggggtcaaccagagacatgattctagcaaactcagcttcgcccaaatcataacccattgaaatcaagcaagctctgaaatcatcatggtccatcagtccattcttcctcctgtcaaagtgattaaatgatgctctgaagtcattcatttgctcctgggtaatacccttggcatctcttgtgaggatctgag

SEQ ID NO: 1229 - DNA, 441 bp, Gallus gallus
actgtgatgagactgccttactcaacaccttcttttgaaagaggaggtgttaaataaagattaaggtttctgagtcattacagttcttgtaaatgcactcacttattcttatgaatggctgagagacttatactttatccactggttggggccacgcacttcaaagggcggctcattgaagtaaatgtggttaccgagttcttccagtgggggctctgggtcagtggtagcaaactgagcagcttcctctatctcttttctcactgccacatcgatttcctttaattcttcaacgctagcaaggttattgttgatcattctgtccttcagcaaagtaatgggatcacttttgcttctcacttcttgaatttcctctctagtactttttttctcatcctgtgcttatgcccaggaaggaatttctgttatctattattttgt

SEQ ID NO: 1230 - DNA, 219 bp, Gallus gallus
acattcatgacaggtttcttttttctttctaaagaaaaaggcctttctgttttgttagcactttggtgtttcaatgtgataatatttaaaaactggatttaaataagaggttagtcaggaaaaacacacaacaatgcttgcaaagtgctccacaccgctgtagccacaggagtgtgaacacactctagaacacgggggcatccagccacggtgctctgt

SEQ ID NO: 1231 - DNA, 575 bp, Gallus gallus
gtatcattgtatttttcttctgcaattttcttgatcatatcaagagttttacgaacaagcttctttctgatcacctttaataacttatgctgctgaagtgtttcacgagatacattcaaaggaagatcatcagaatccacaacacccttaacaaagttaagatatttgggcatcatgtcatggaagtcatcagtgatgaacactcttctaacatacagcttaatgaaatcactttttttggatccatactcatcaaacaagccacgtggagcagaattaggaacaaacaagattgatttgaaagttacttccccttcagcagtaaaatggatgtaagccattggatcatcatgttccttggaaaatgttttgtaaaaagctttatattcatcctcttcaacttctttagatggtctctgccagattggttttatgtcattcatgagctcccaatcccagacagtcttctcaaccttcttagtttttggtttcttctcttcctcctcttcttcaactgcagcttcatcatcatctgtttcttctttttcctcctttgctccctcctcttcaatggg

SEQ ID NO: 1232 - DNA, 619 bp, Gallus gallus
  misc_feature (598)..(598): n is a, c, g, or t
accagtaagcatatgaacttcaaaatgcacaattgccacagacagttgacttgaatacagtaatggtggttggttgcacacttagagacgacttttagattcttccactctcaaatggctttgcatttctggatcatctagtcatgcactggagaggaattccacagctgtctccttctcttcagttaactccttagcagtcagatccatcttctcacgagaaaagtcattaataggaagaccctcaacaaacttccaggtcttgtccttgatcacaacagggaatgaatatagcaagtcttcaggaacaccataagaattgccatcagaaatgactcccatggaaacaaattctcccgctggagtgccaaaccagatgtctctcacatgatcacagattgctttggcagctgacattgcactggacagcttcctagccttaataacagctgctccacgttgctgaacagtcaggataaagtctcccttcagccagctgtcatcttttatagcttcataaactccaacttcctttcctttcacattcacctttgcatggttaacatctggatattgagtggaggagtggttgccccanatgatgacattcttcacatcg

SEQ ID NO: 1233 - DNA, 426 bp, Gallus gallus
acgttgacaaccatattggtatctcaattgccggacttacagctgatgcaagactcttgtgcaattttatgcgtcaggagtgtctggattctagatttgtgtttgatagacctcttccagtgtctcgtttggtgtcactaatcggaagcaaaacgcagataccaacacagcgctatggcagaagaccatatggtgtaggactgctcattgcaggttatgatgatatgggccctcacatcttccaaacttgtccctcagcaaactattttgactgtaaagcaatgtccattggtgctcgttcgcagtcagcacgaacttacttggaaaggcacatgactgaatttactgactgtaatctaaatgagctagttaaacatggactgcgtgccctgagagagactcttcctgctgaacaggatctgacca